(19) United States
(12) Patent Application Publication
(10) Pub. No.: US 2006/0168696 A1
(43) Pub. Date: Jul. 27, 2006

(54) Nucleotide sequences and corresponding polypeptides conferring modulated plant size and biomass and other characteristics

(75) Inventors: Kenneth Feldmann, Newbury Park, CA (US); Gregory Nadzan, Woodland Hills, CA (US); Peter Mascia, Thousand Oaks, CA (US)

Correspondence Address: BIRCH STEWART KOLASCH & BIRCH, PO BOX 747, FALLS CHURCH, VA, 22040-0747, US

(73) Assignee: CERES, INC., Thousand Oaks, CA (US)

(21) Appl. No.: 11/317,789

(22) Filed: Dec. 22, 2005

Related U.S. Application Data
(63) Continuation-in-part of application No. 11/241,673, filed on Sep. 30, 2005.
(60) Provisional application No. 60/639,228, filed on Dec. 22, 2004.

(51) Int. Cl. C07H 21/04; C12N 15/82; C12N 5/04; A01H 1/00

(52) U.S. Cl. 800/287; 800/289; 435/419; 435/468; 536/23.6

(57) ABSTRACT

The present invention relates to isolated nucleic acid molecules and their corresponding encoded polypeptides able confer the trait of modulated plant size, vegetative growth, organ number, plant architecture, sterility or seedling lethality in plants. The present invention further relates to the use of these nucleic acid molecules and polypeptides in making transgenic plants, plant cells, plant materials or seeds of a plant having such modulated growth or phenotype characteristics that are altered with respect to wild type plants grown under similar conditions.

This application is a Continuation-In-Part of co-pending application Ser. No. 11/241,673 filed on Sep. 30, 2005, the entire contents of which are hereby incorporated by reference and for which priority is claimed under 35 U.S.C. § 120. This non-provisional application claims priority under 35 U.S.C. § 119(e) on U.S. Provisional Application No. 60/639,228 filed on Dec. 22, 2004, the entire contents of which are hereby incorporated by reference.

### FIELD OF THE INVENTION

The present invention relates to isolated nucleic acid molecules and their corresponding encoded polypeptides able to modulate plant size, vegetative growth, organ number, architecture, biomass, lethality, sterility and other characteristics in plants. The present invention further relates to using the nucleic acid molecules and polypeptides to make transgenic plants, plant cells, plant materials or seeds of a plant having modulated phenotypic and growth characteristics as compared to wild-type plants grown under similar conditions.

### BACKGROUND OF THE INVENTION

Plants specifically improved for agriculture, horticulture, biomass conversion, and other industries (e.g. paper industry, plants as production factories for proteins or other compounds) can be obtained using molecular technologies. As an example, great agronomic value can result from modulating the size of a plant as a whole or of any of its organs or the number of any of its organs.

Similarly, modulation of the size and stature of an entire plant, or a particular portion of a plant, allows production of plants better suited for a particular industry. For example, reductions in the height of specific crops and tree species can be beneficial by allowing easier harvesting. Alternatively, increasing height, thickness or organ number may be beneficial by providing more biomass useful for processing into food, feed, fuels and/or chemicals (http://www.eere.energy.gov/biomass/publications.html). Other examples of commercially desirable traits include increasing the length of the floral stems of cut flowers, increasing or altering leaf size and shape or enhancing the size of seeds and/or fruits. Changes in organ size, organ number and biomass also result in changes in the mass of constituent molecules such as secondary products and convert the plants into factories for these compounds.

Availability and maintenance of a reproducible stream of food and feed to feed people has been a high priority throughout the history of human civilization and lies at the origin of agriculture. Specialists and researchers in the fields of agronomy science, agriculture, crop science, horticulture, and forest science are even today constantly striving to find and produce plants with an increased growth potential to feed an increasing world population and to guarantee a supply of reproducible raw materials. The robust level of research in these fields of science indicates the level of importance leaders in every geographic environment and climate around the world place on providing sustainable sources of food, feed and energy for the population.

Manipulation of crop performance has been accomplished conventionally for centuries through plant breeding. The breeding process is, however, both time-consuming and labor-intensive. Furthermore, appropriate breeding programs must be specially designed for each relevant plant species.

On the other hand, great progress has been made in using molecular genetics approaches to manipulate plants to provide better crops. Through introduction and expression of recombinant nucleic acid molecules in plants, researchers are now poised to provide the community with plant species tailored to grow more efficiently and produce more product despite unique geographic and/or climatic environments. These new approaches have the additional advantage of not being limited to one plant species, but instead being applicable to multiple different plant species (1).

Despite this progress, today there continues to be a great need for generally applicable processes that improve forest or agricultural plant growth to suit particular needs depending on specific environmental conditions. To this end, the present invention is directed to advantageously manipulating plant size, organ number, plant architecture and/or biomass to maximize the benefits of various crops depending on the benefit sought and the particular environment in which the crop must grow, characterized by expression of recombinant DNA molecules in plants. These molecules may be from the plant itself, and simply expressed at a higher or lower level, or the molecules may be from different plant species.

### SUMMARY OF THE INVENTION

The present invention, therefore, relates to isolated nucleic acid molecules and polypeptides and their use in making transgenic plants, plant cells, plant materials or seeds of plants having life cycles, particularly plant size, vegetative growth, organ number, plant architecture, biomass, lethality, sterility and other characteristics that are altered with respect to wild-type plants grown under similar or identical conditions (sometimes hereinafter collectively referred to as “modulated growth and phenotype characteristics”).

Unless otherwise defined, all technical and scientific terms used herein have the same meaning as commonly understood by one of ordinary skill in the art to which this invention belongs.

### BRIEF DESCRIPTION OF THE FIGURES

FIGS. 1-73: The Figures set forth amino acid sequence alignment showing homologues of Lead polypeptide sequences, SEQ ID NO. ***. Conserved regions are enclosed in a box. A consensus sequence is shown below the alignment.

### DETAILED DESCRIPTION OF THE INVENTION

1. The Invention

The invention of the present application may be described by, but not necessarily limited to, the following exemplary embodiments.

The present invention discloses novel isolated nucleic acid molecules, nucleic acid molecules that interfere with these nucleic acid molecules, nucleic acid molecules that hybridize to these nucleic acid molecules, and isolated nucleic acid molecules that encode the same protein due to the degeneracy of the DNA code. Additional embodiments of the present application further include the polypeptides encoded by the isolated nucleic acid molecules of the present invention.

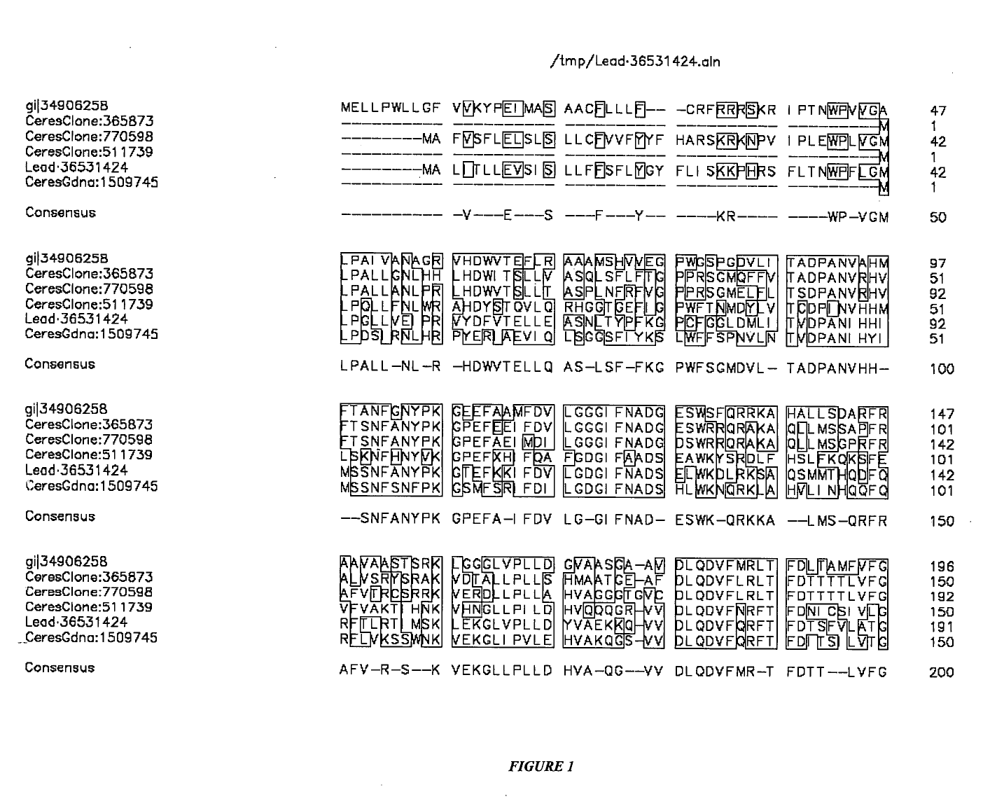

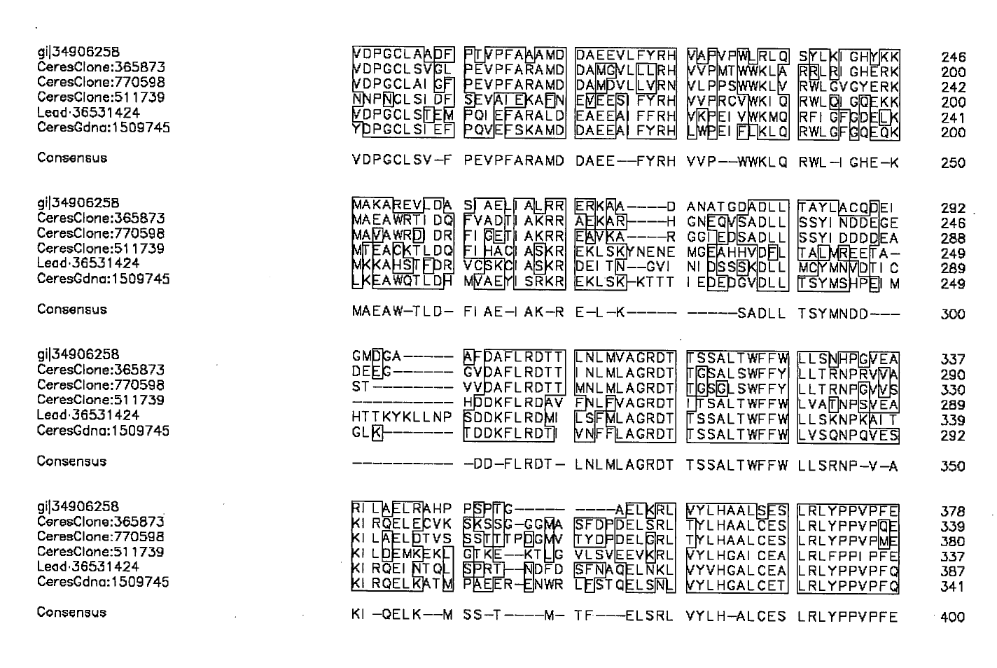

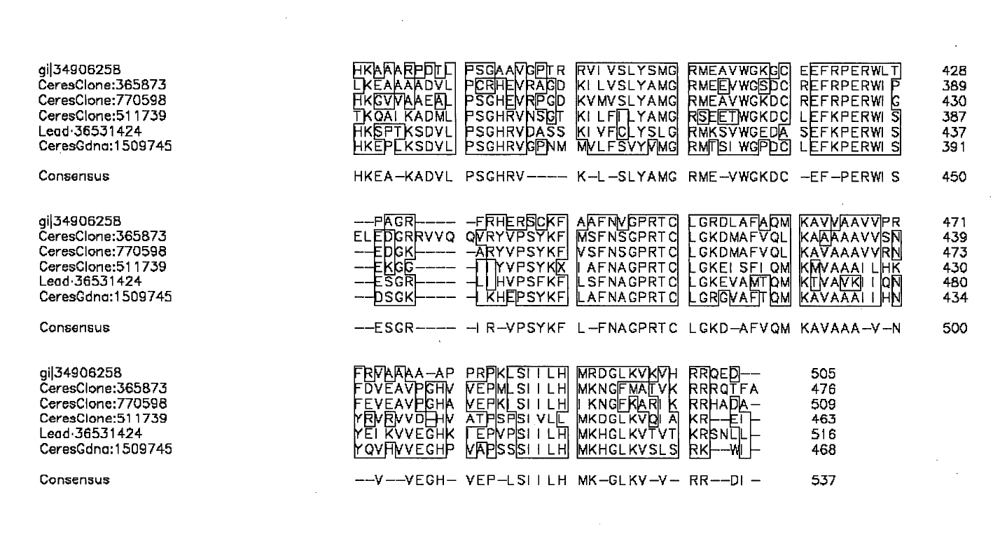

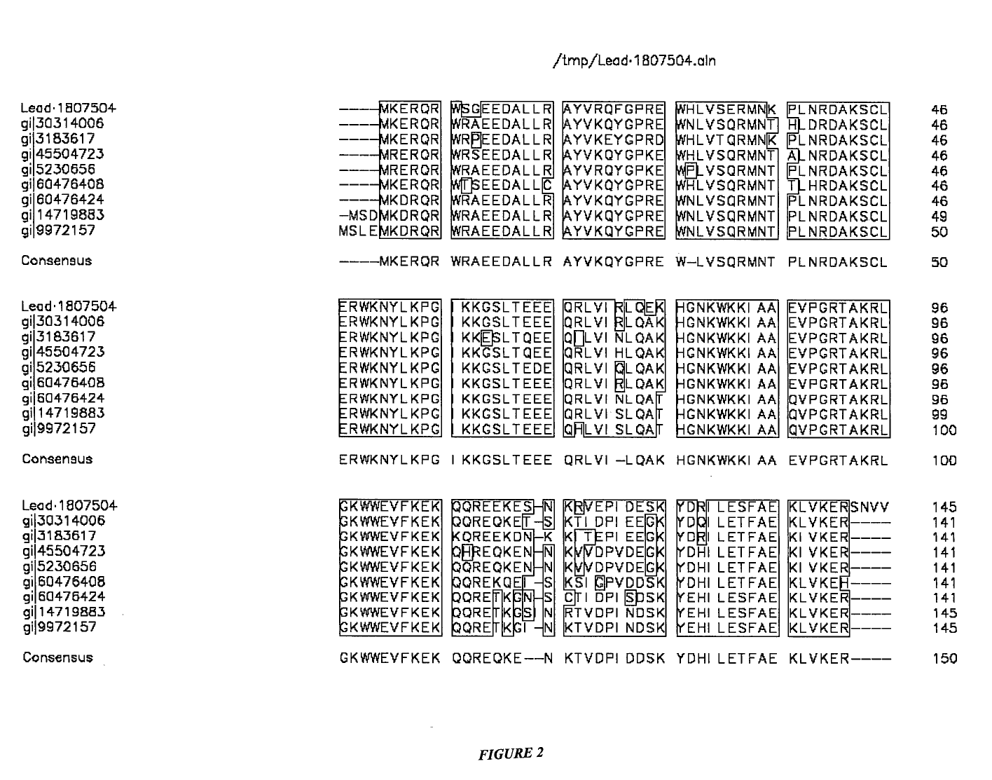

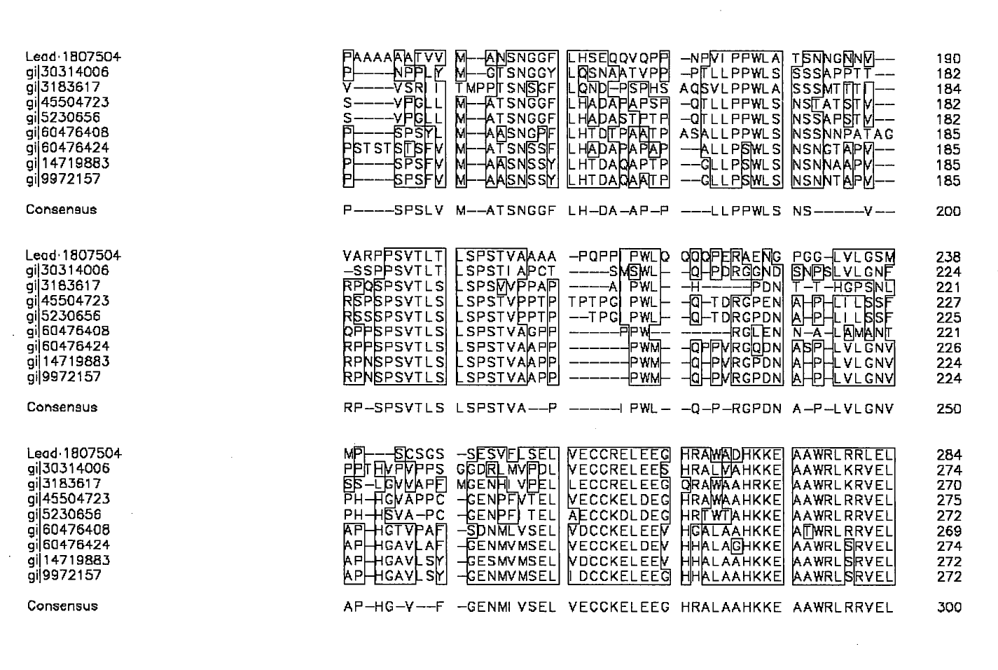

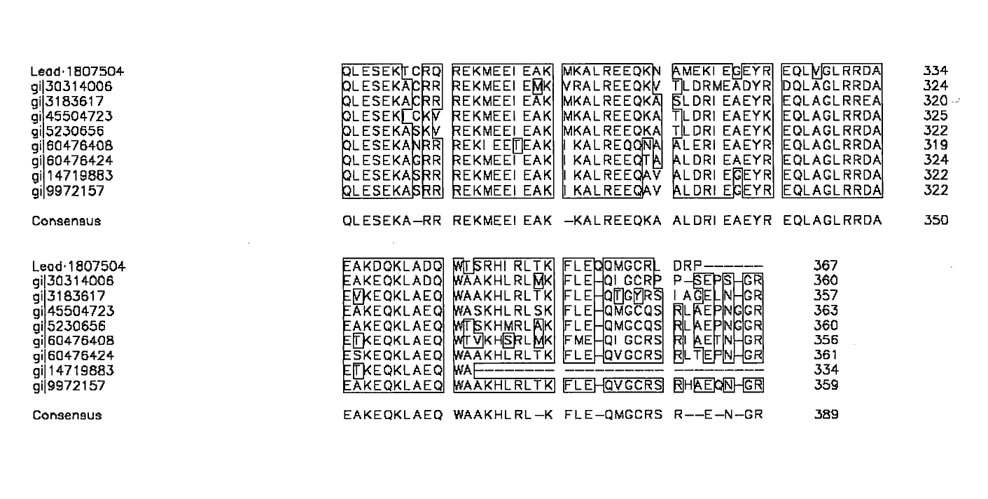

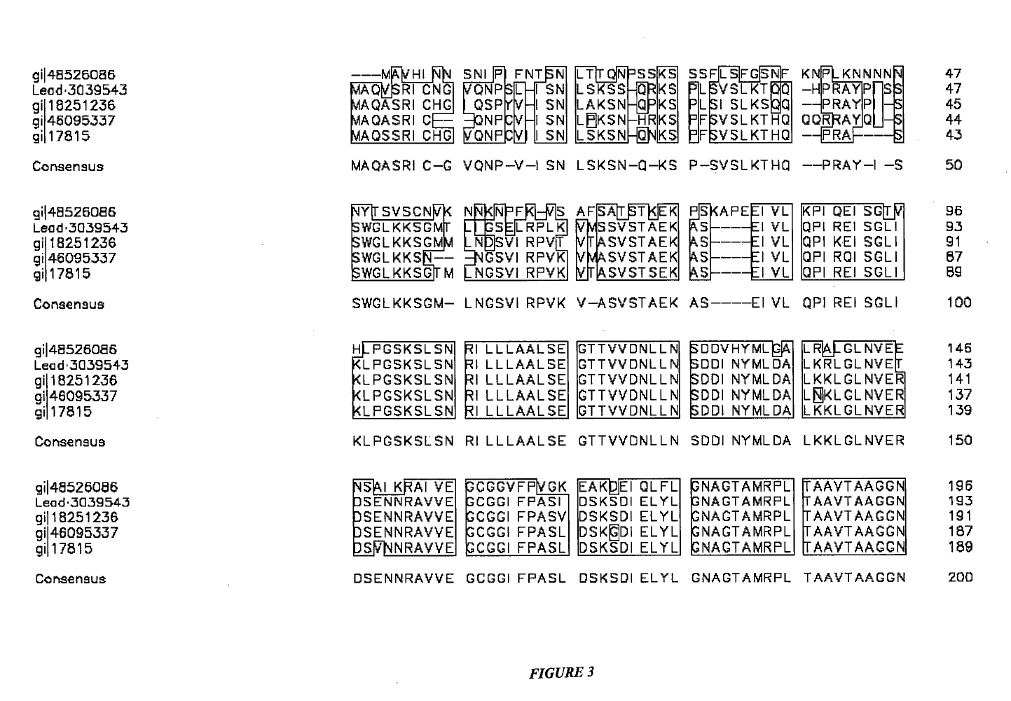

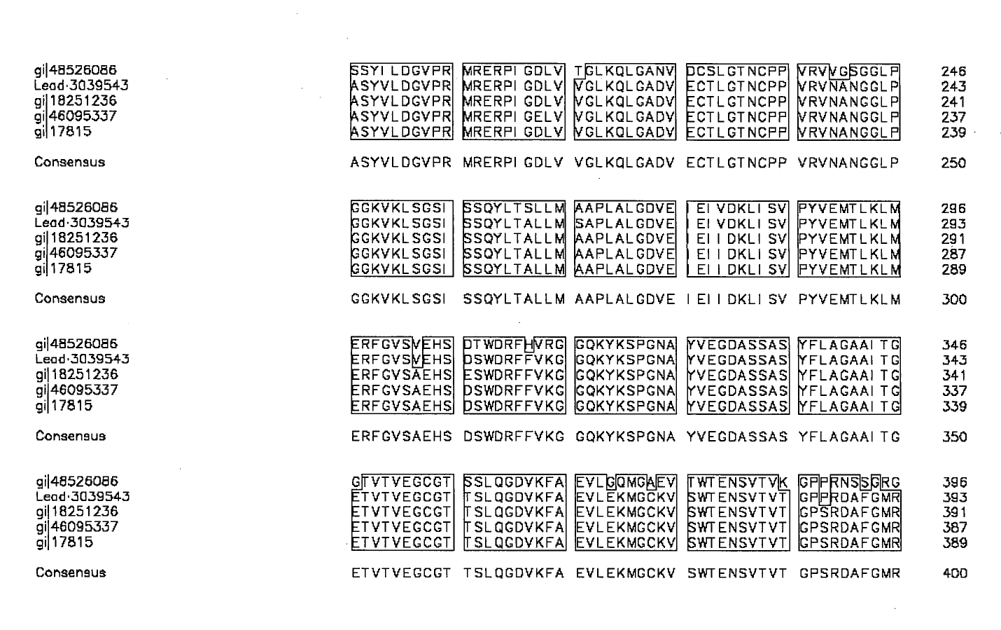

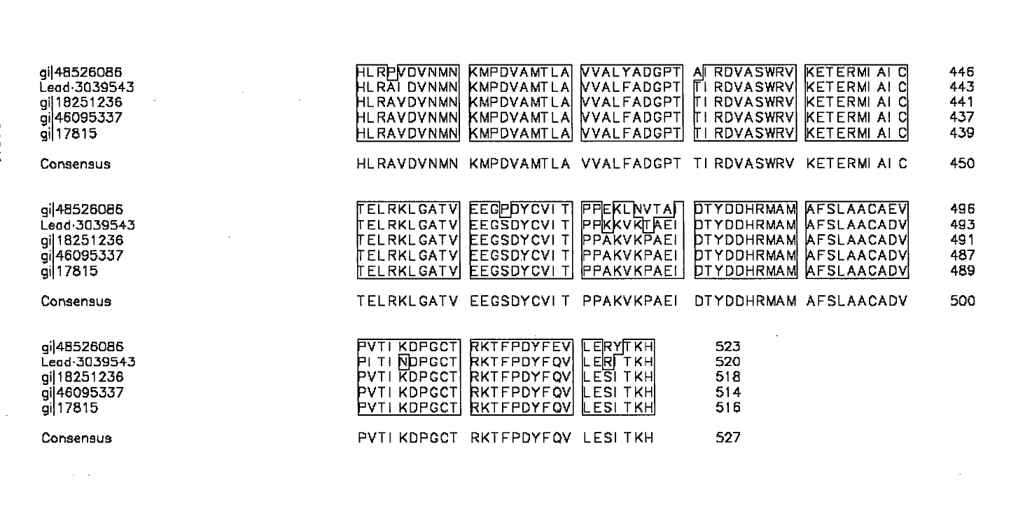

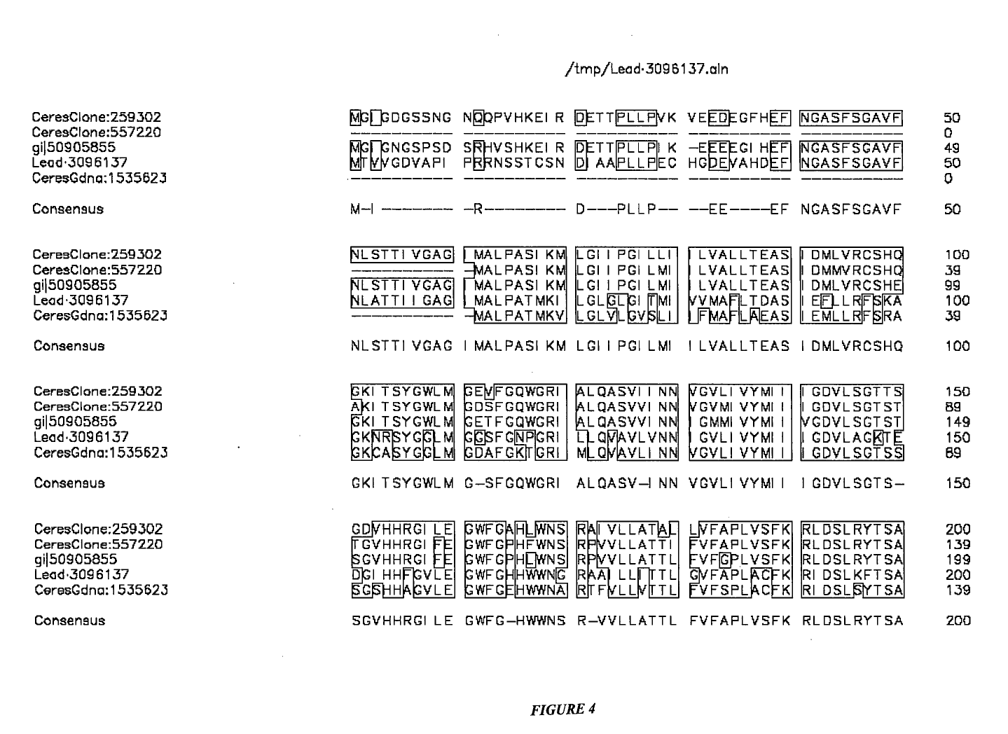

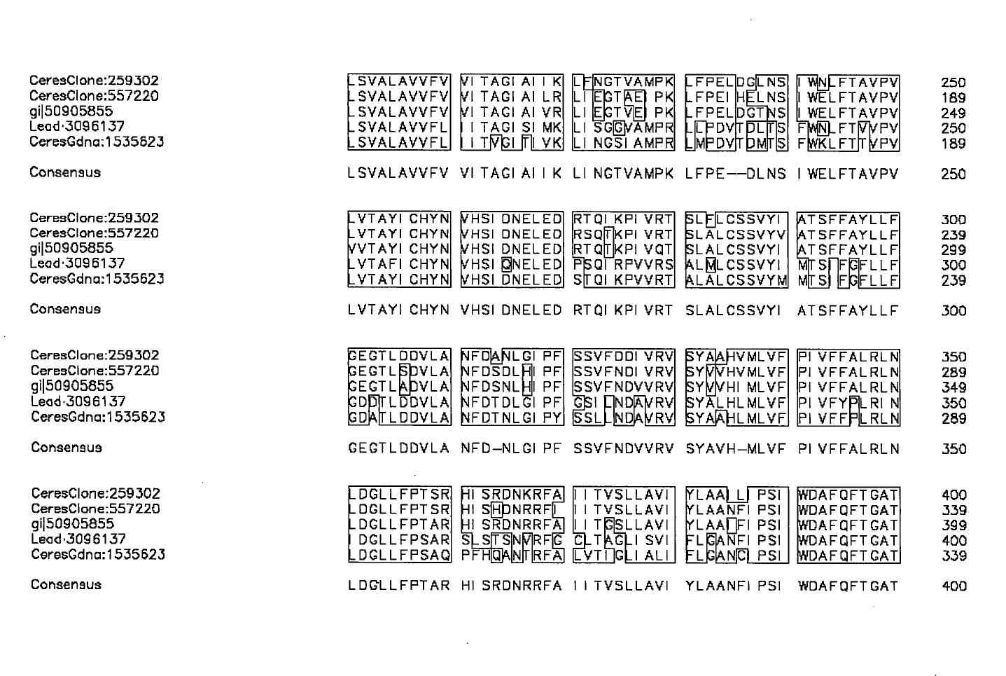

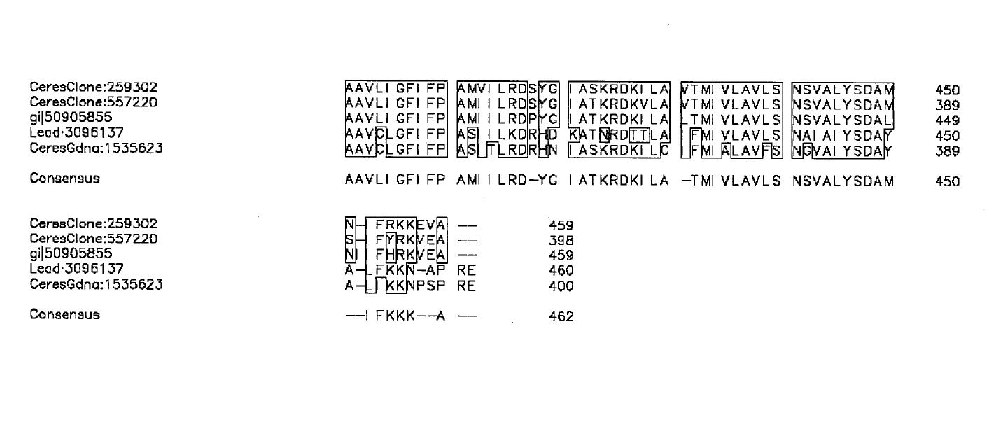

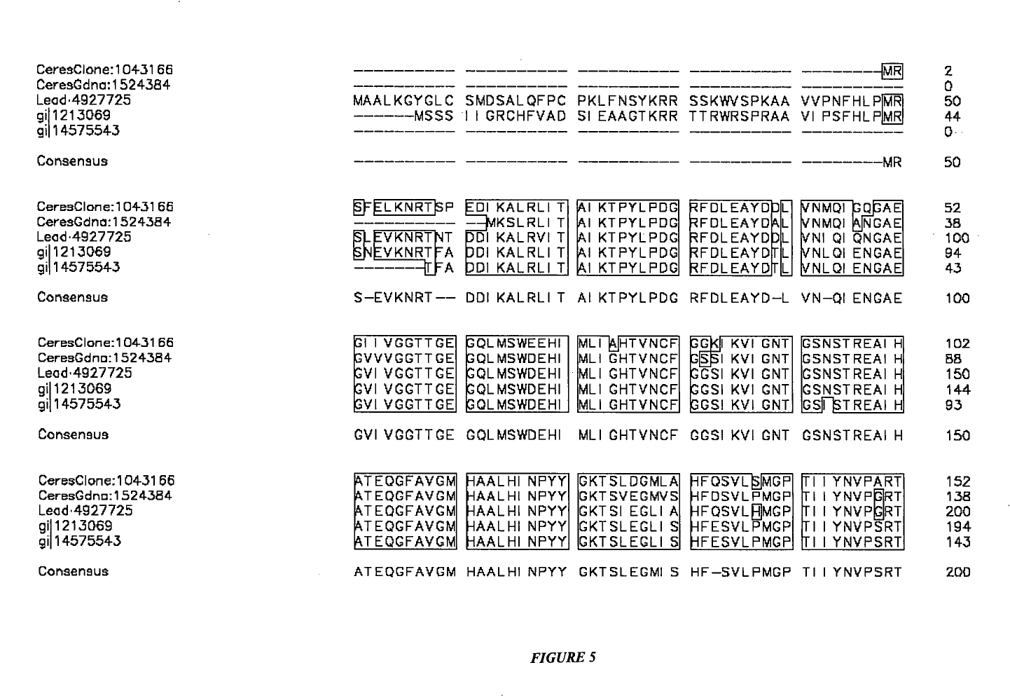

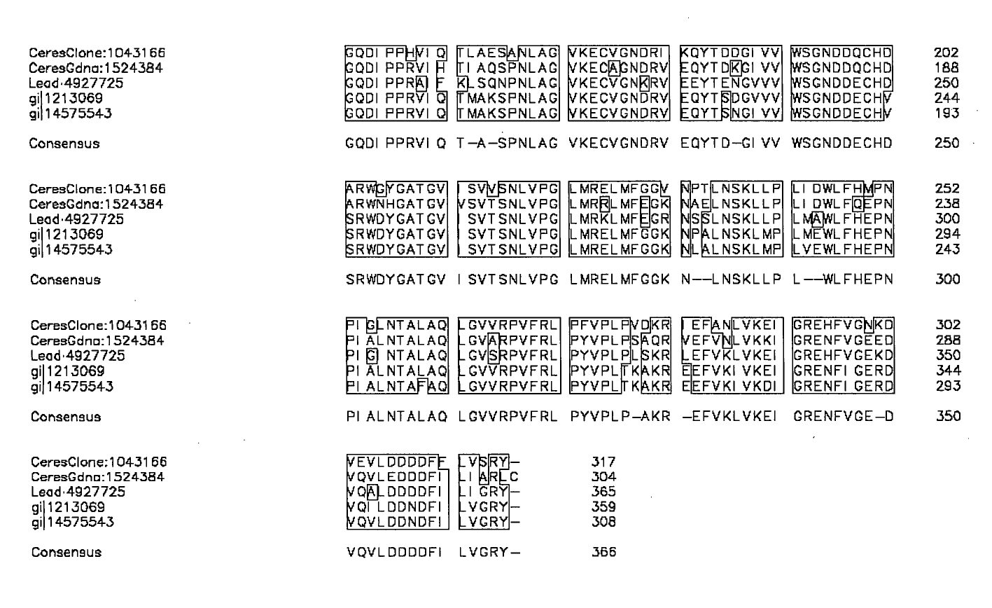

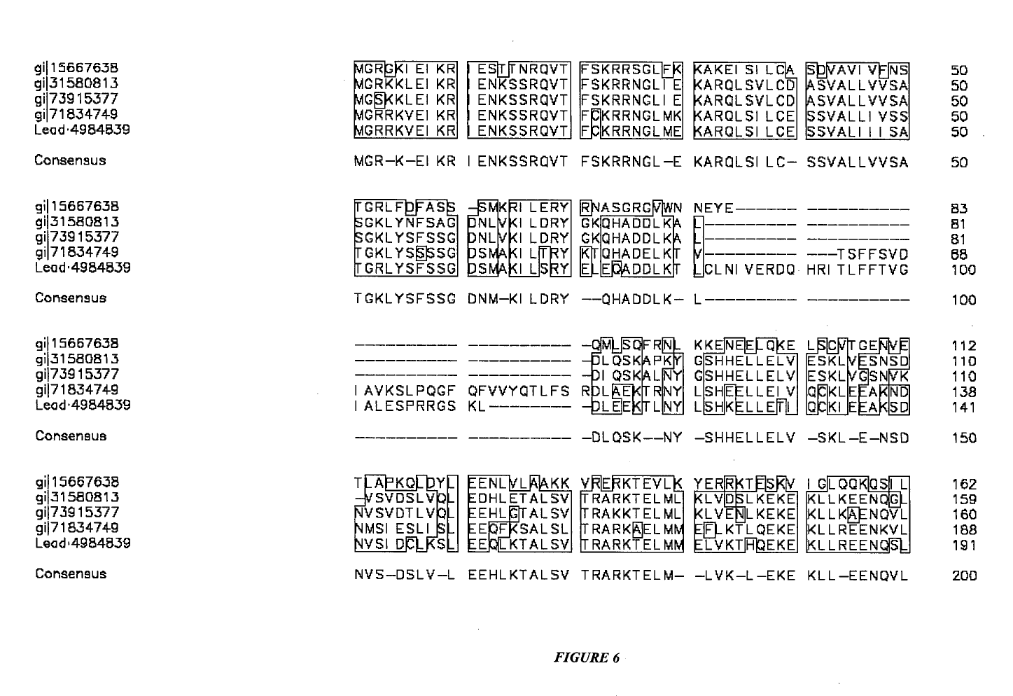

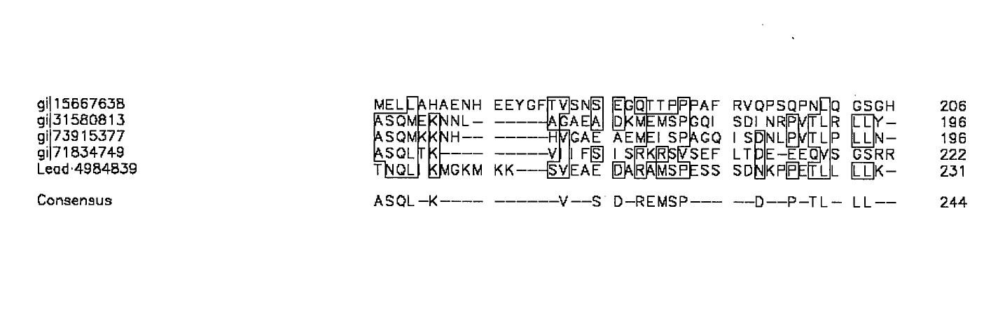

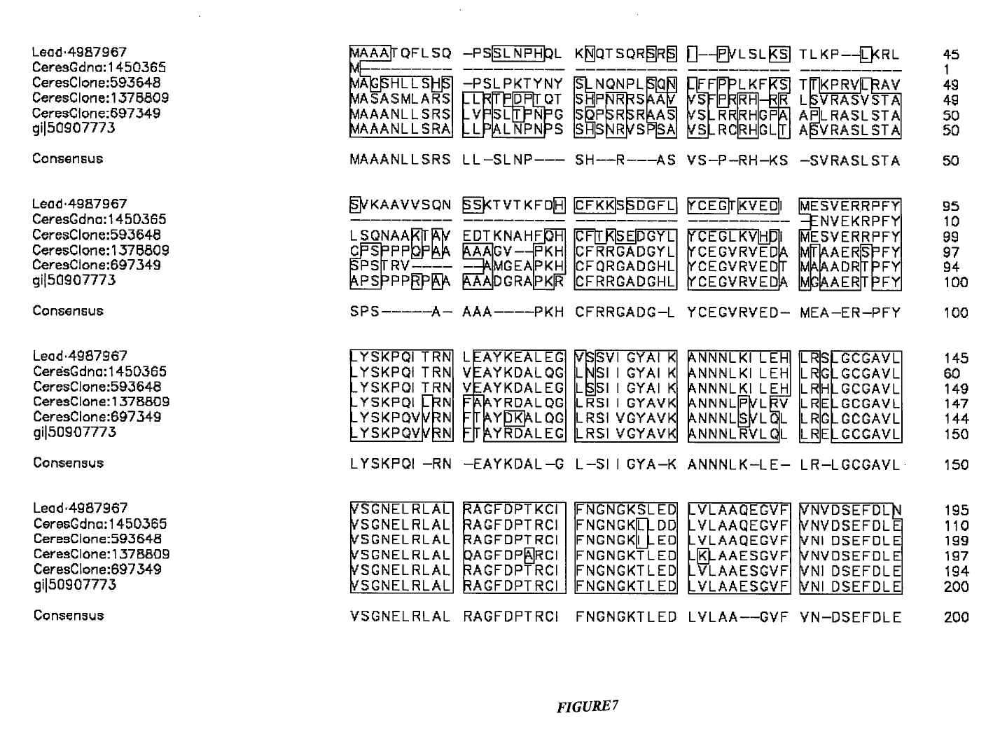

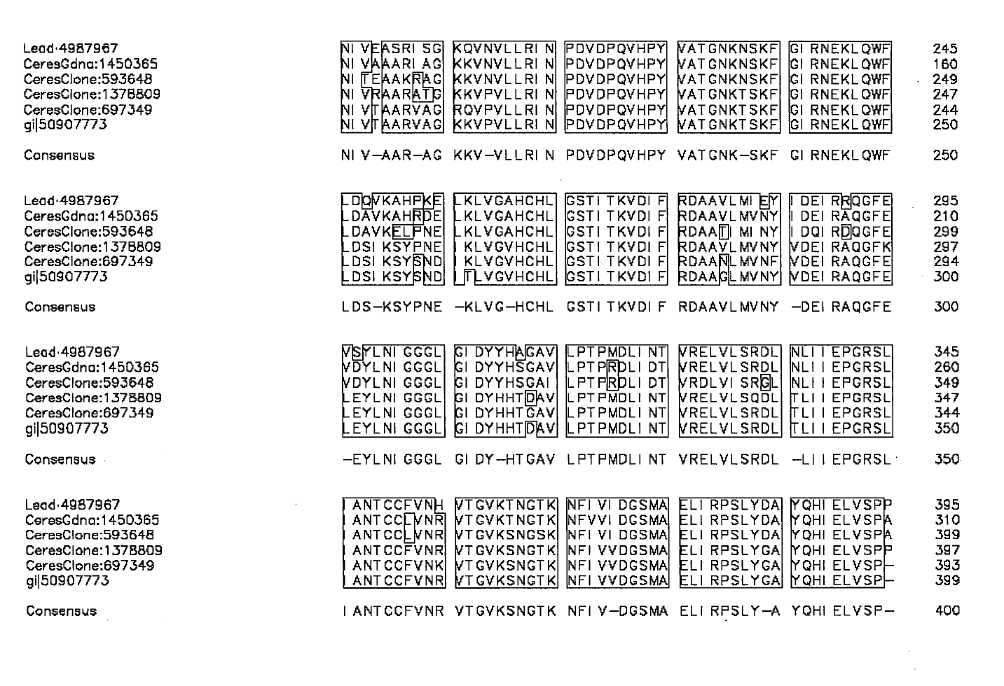

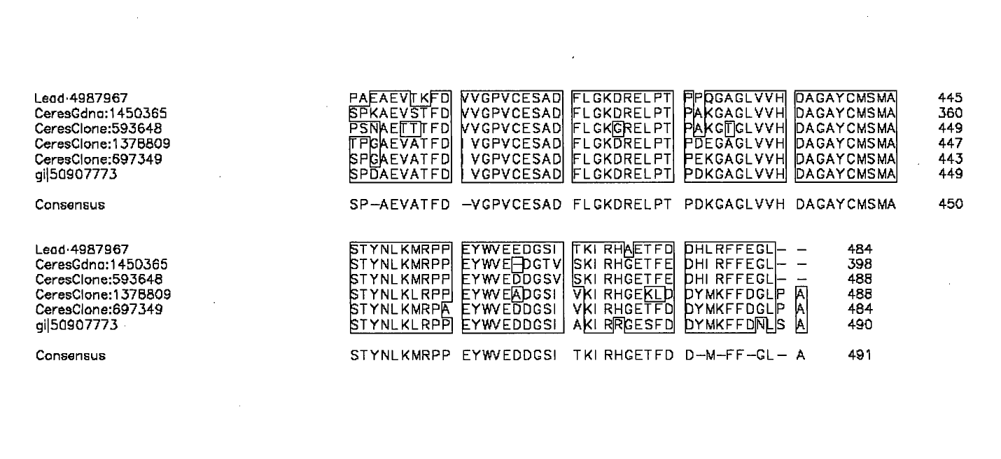

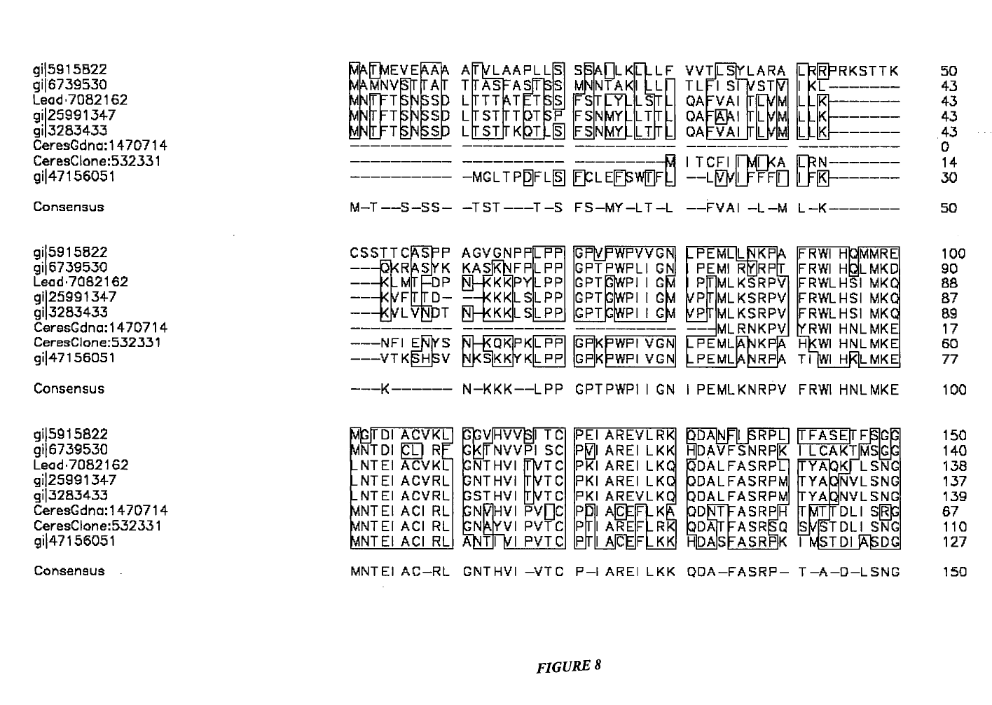

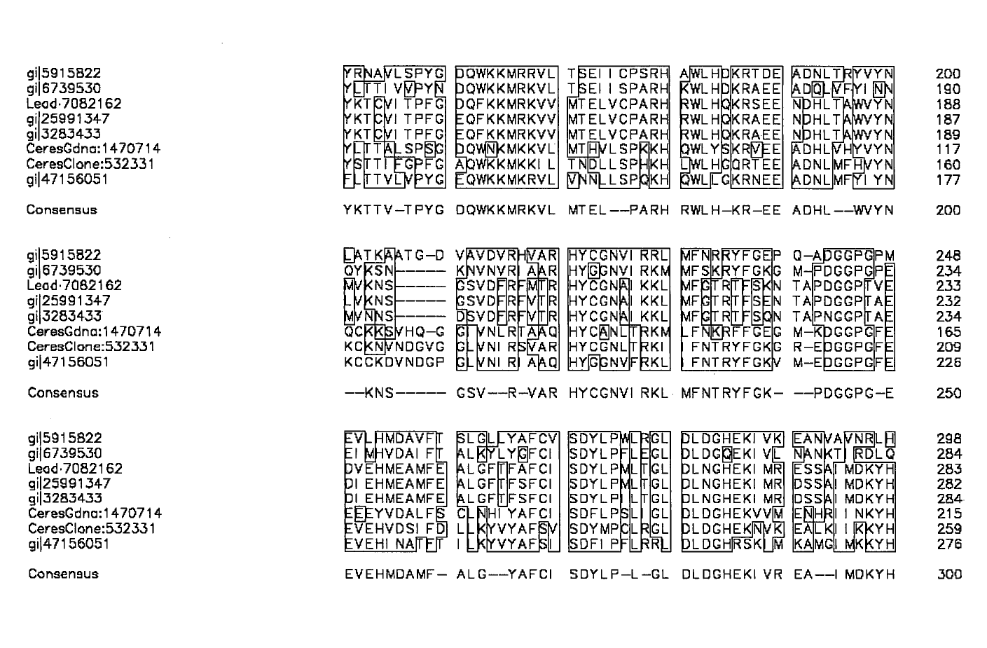

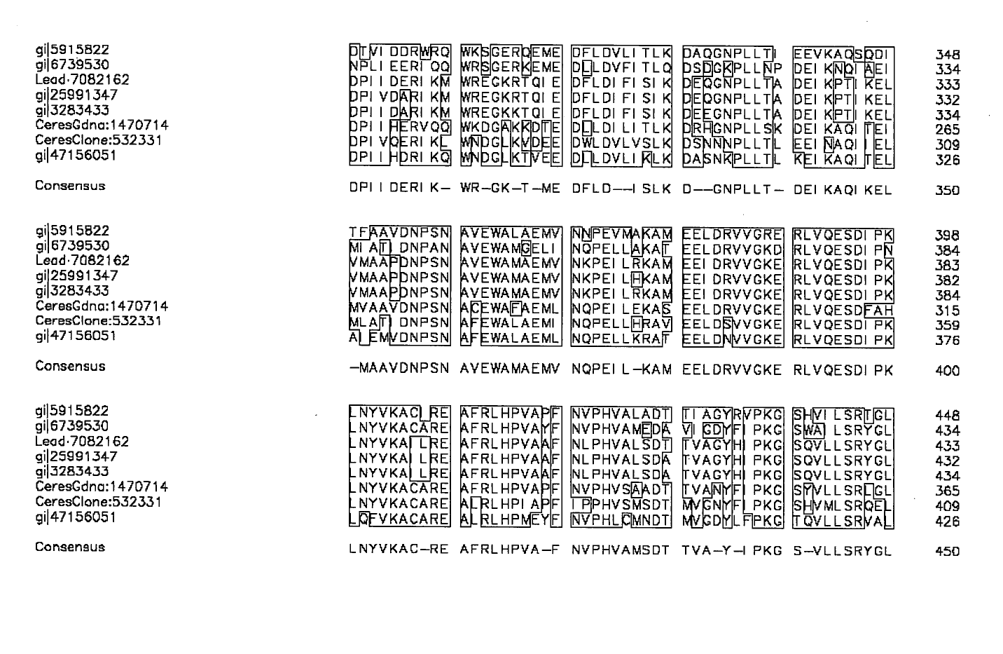

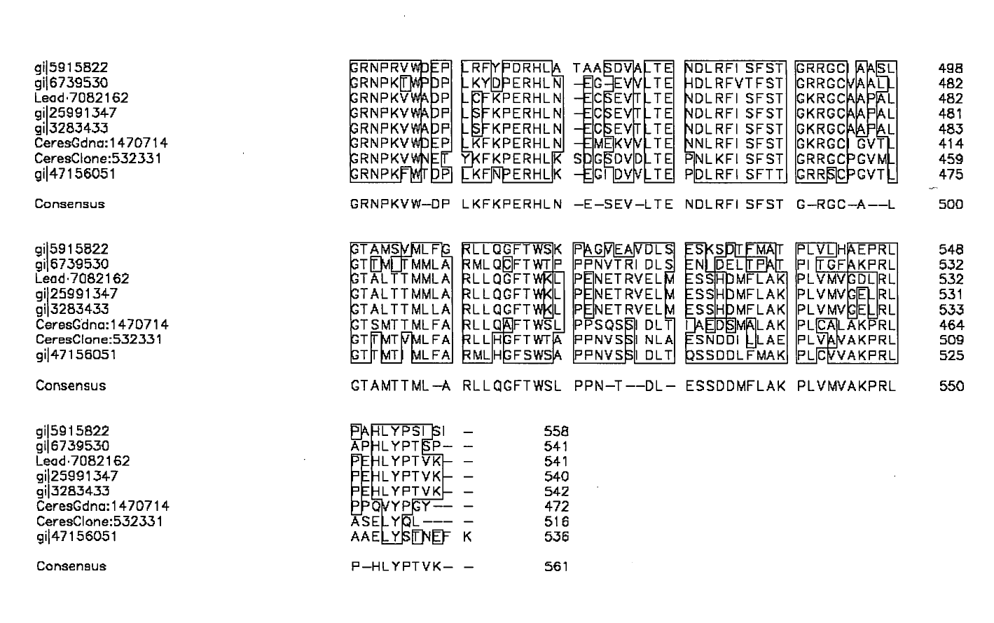

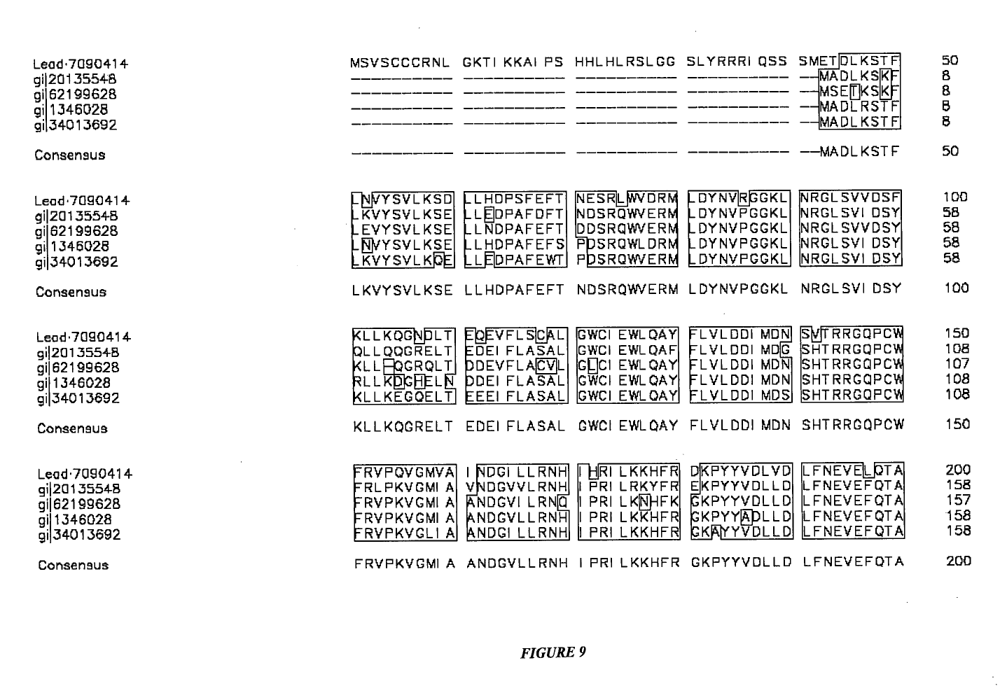

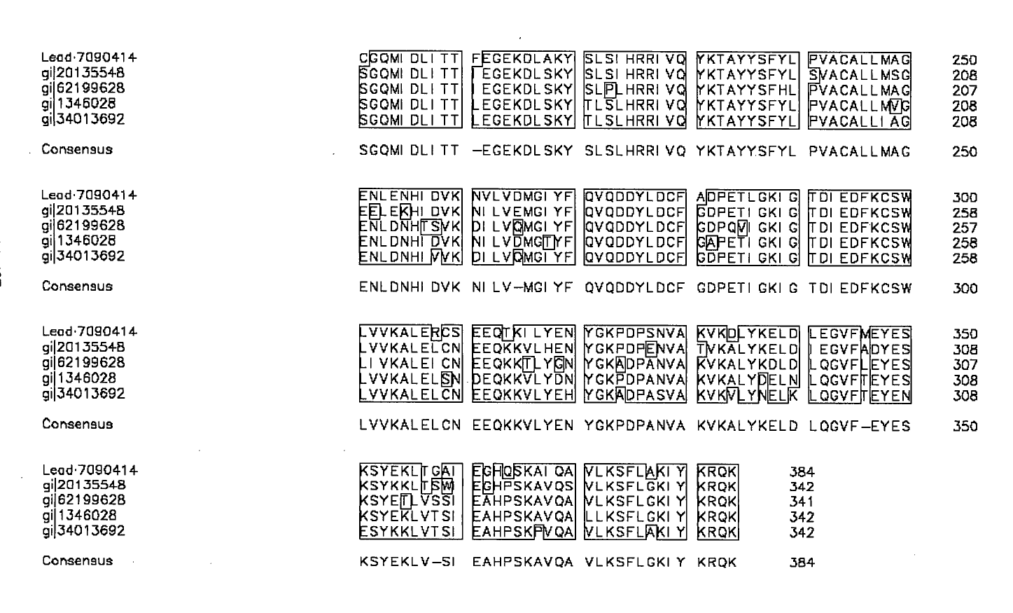

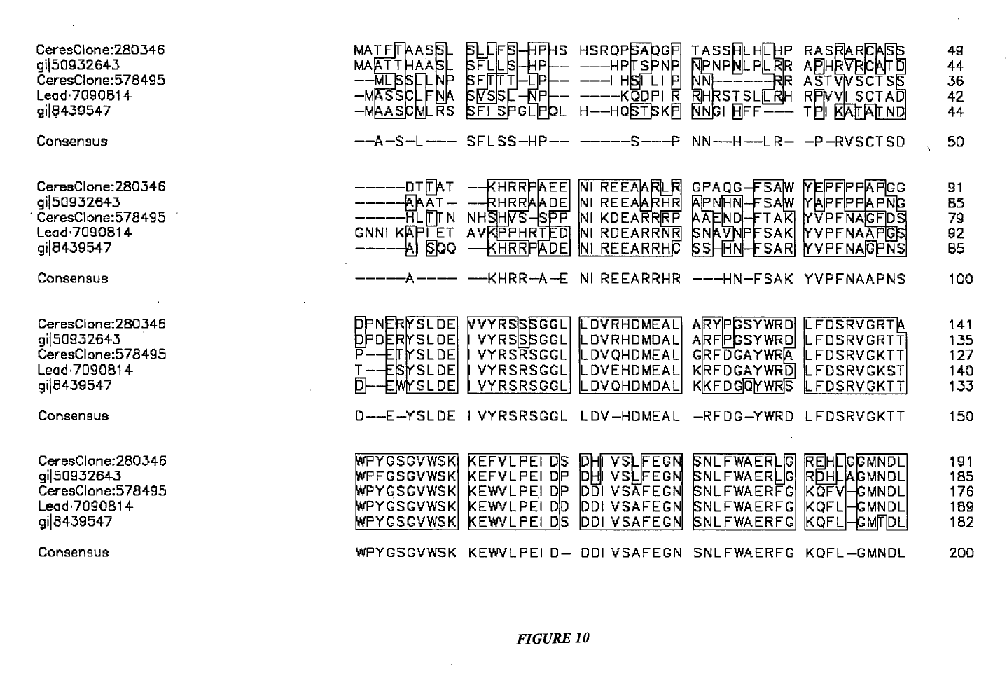

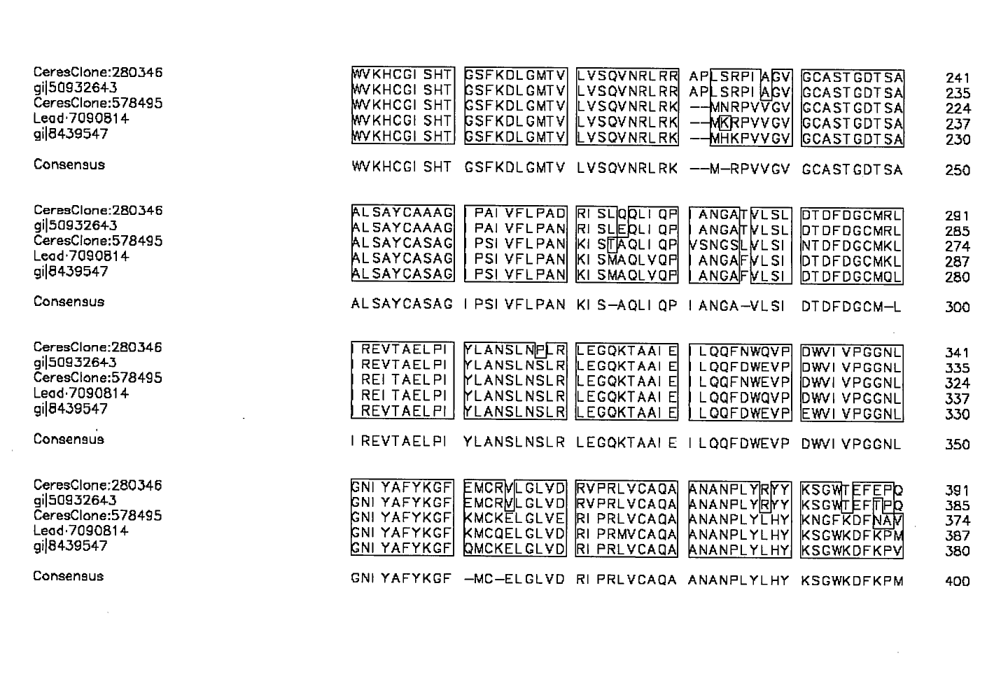

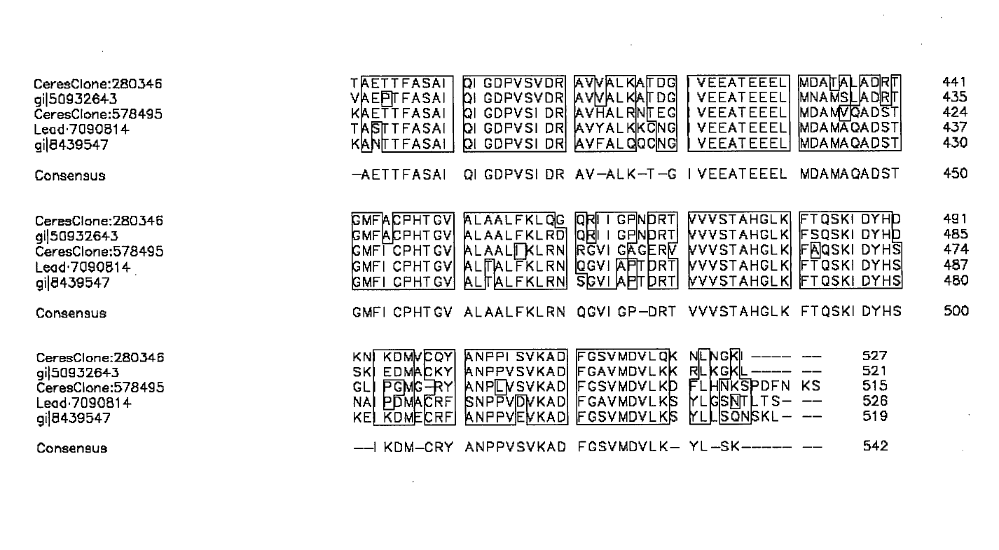

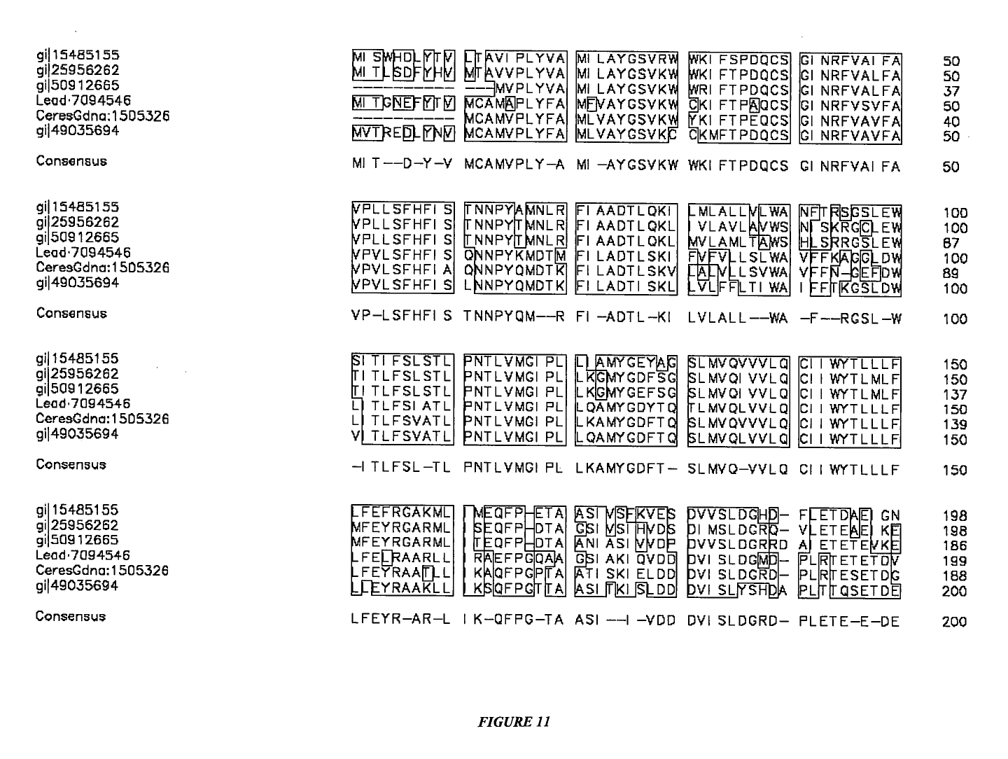

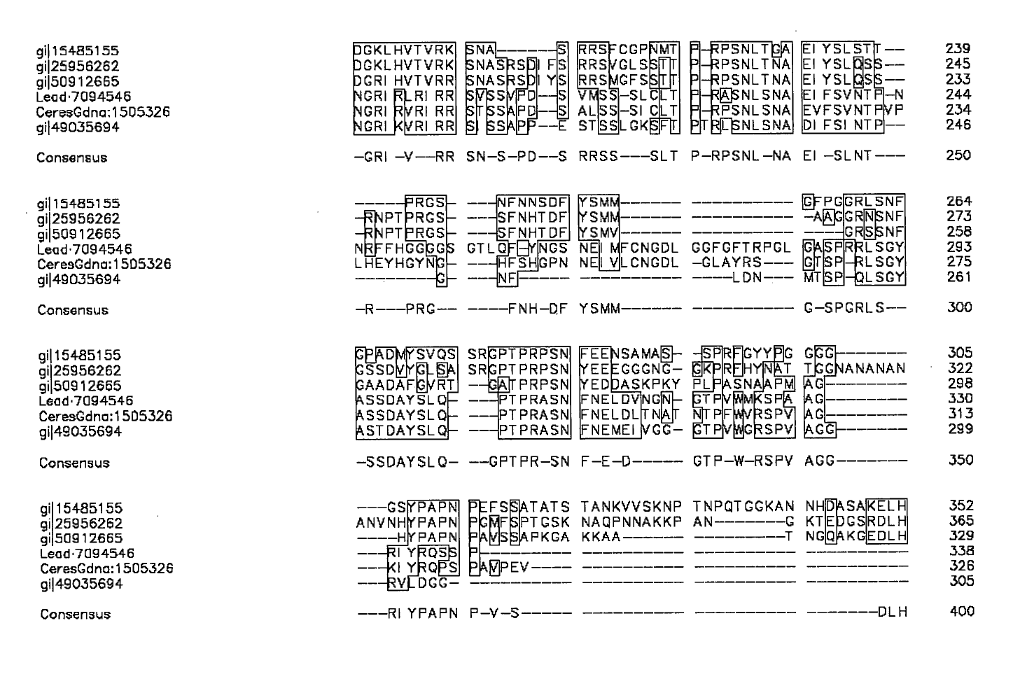

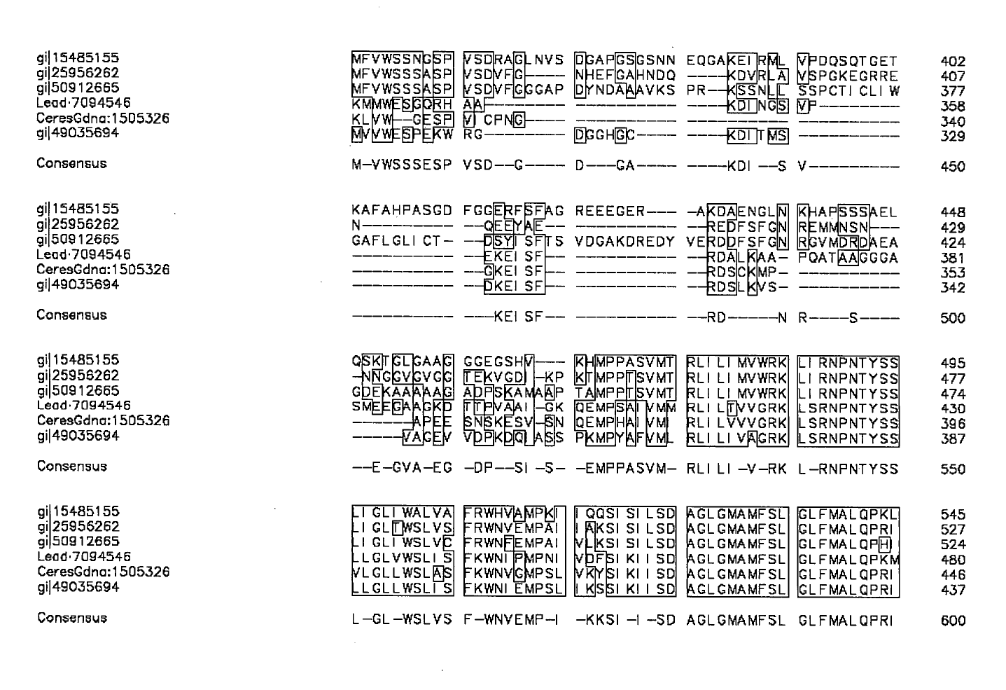

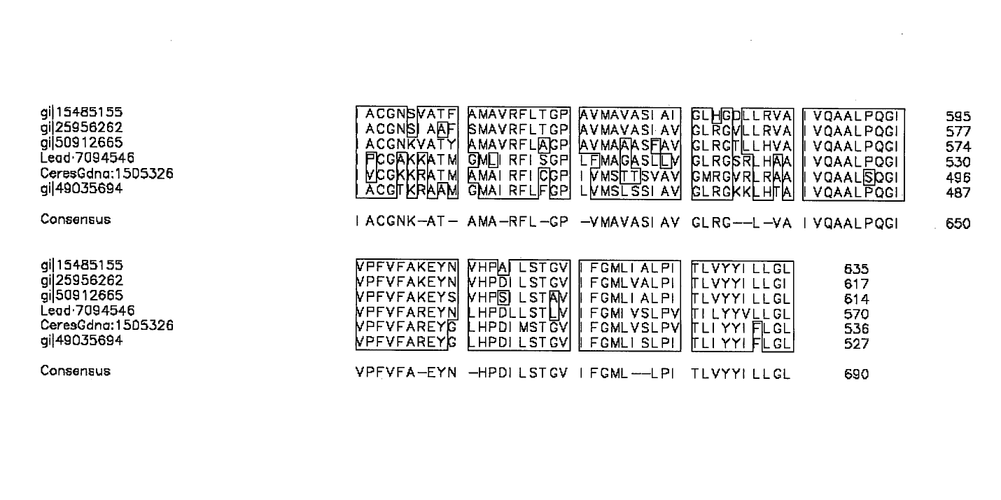

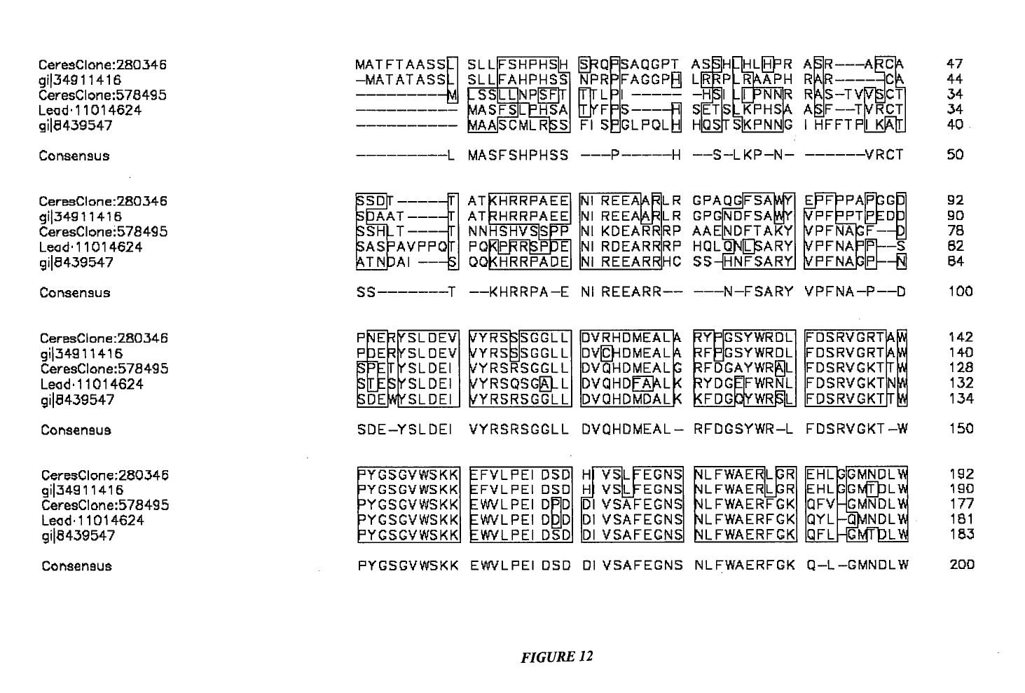

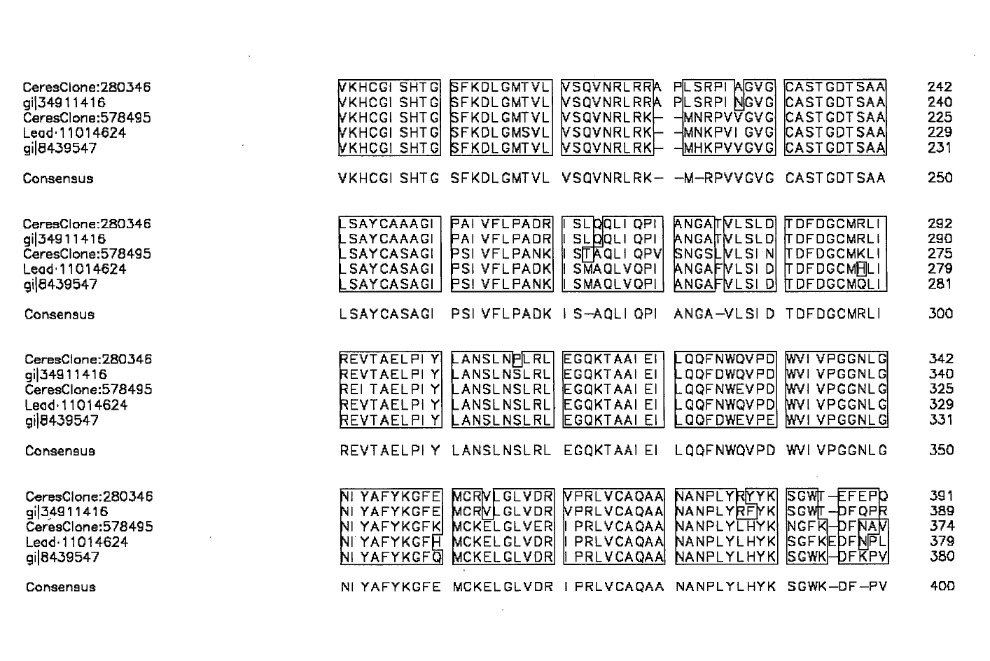

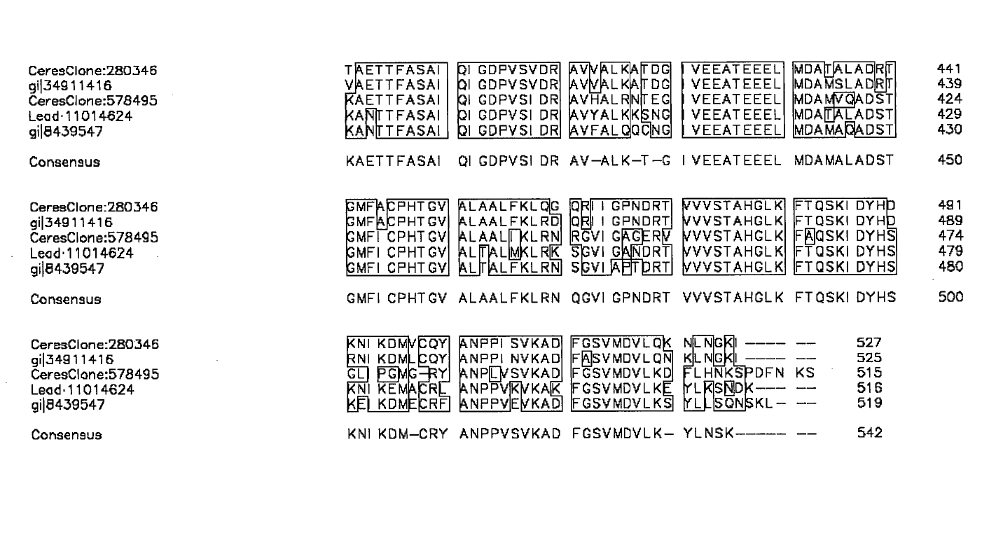

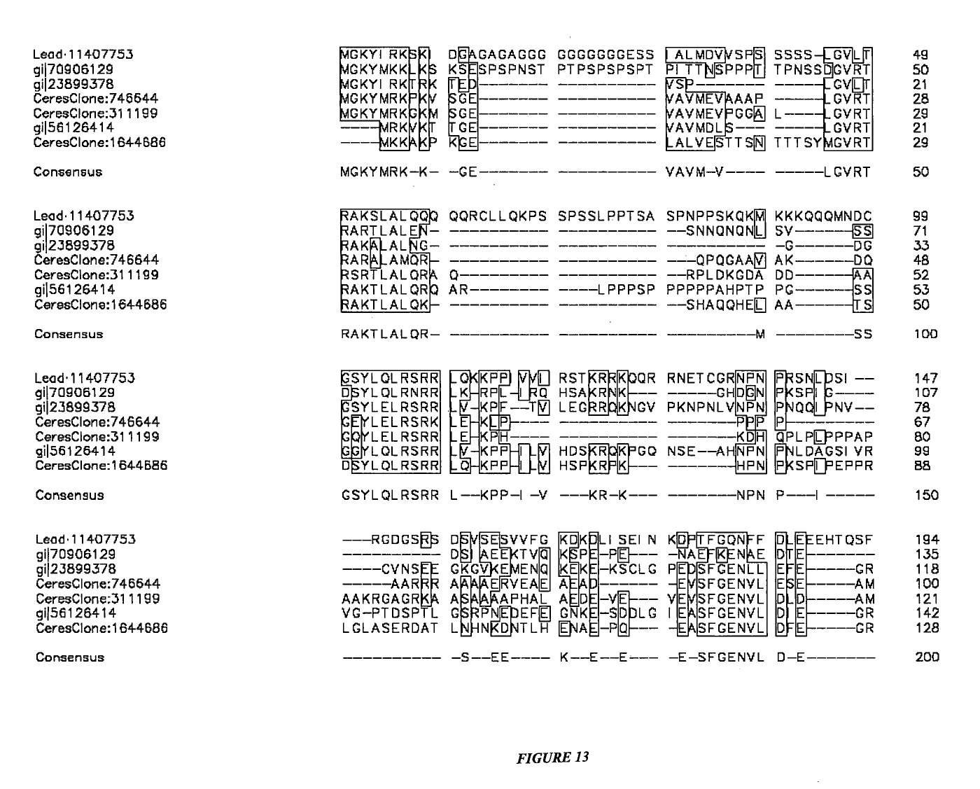

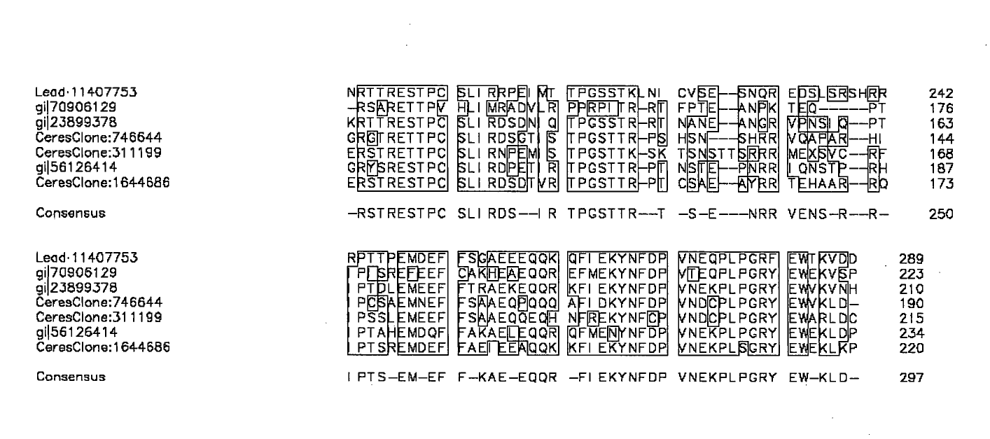

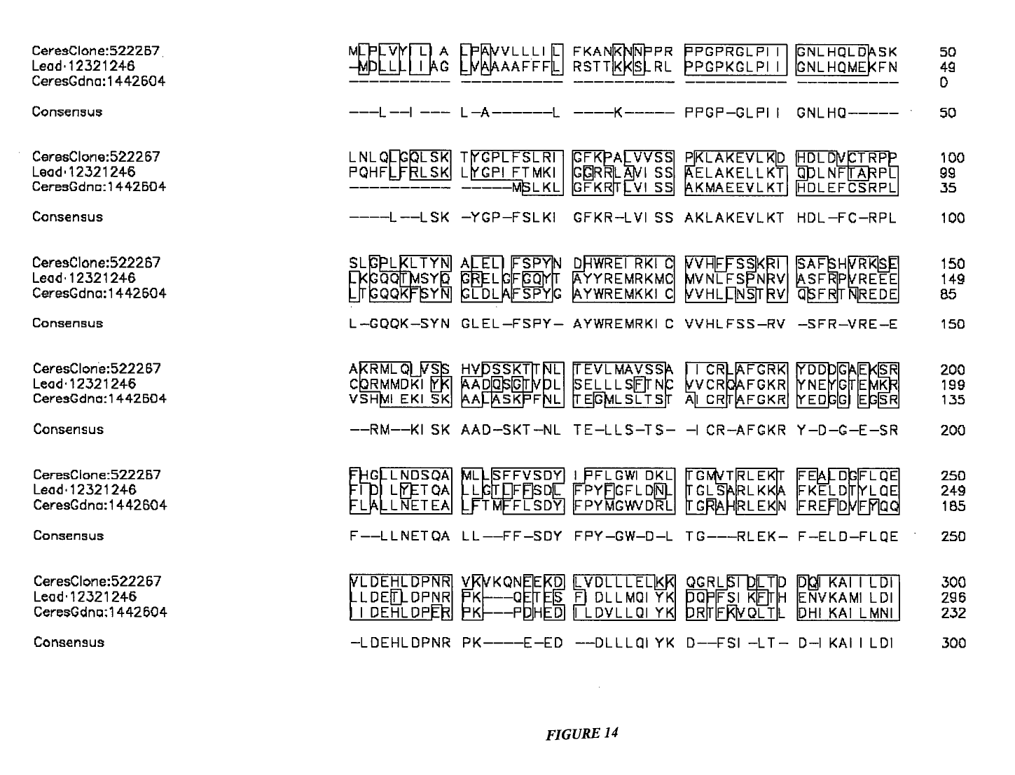

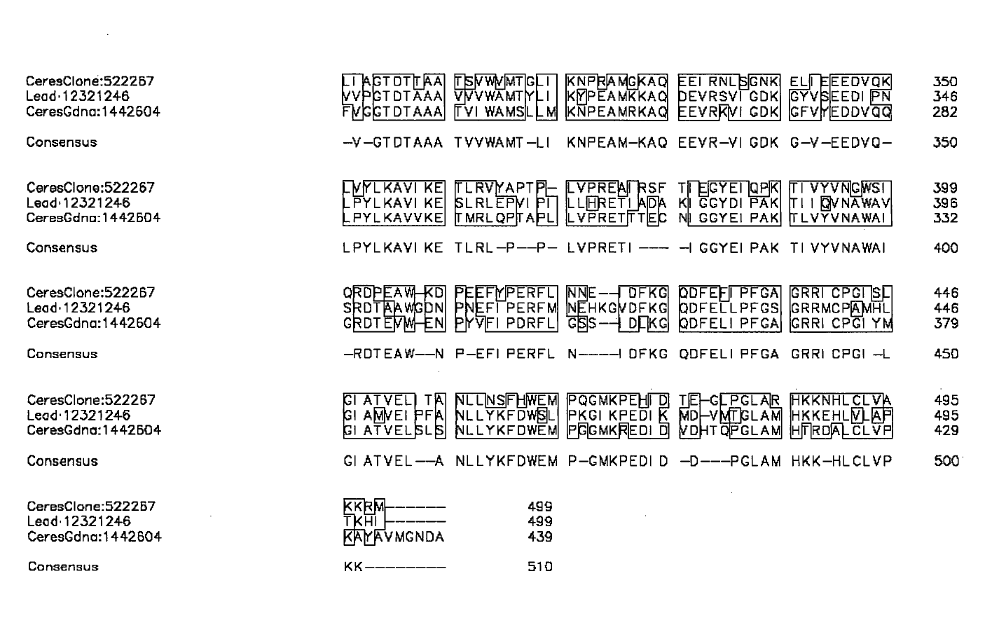

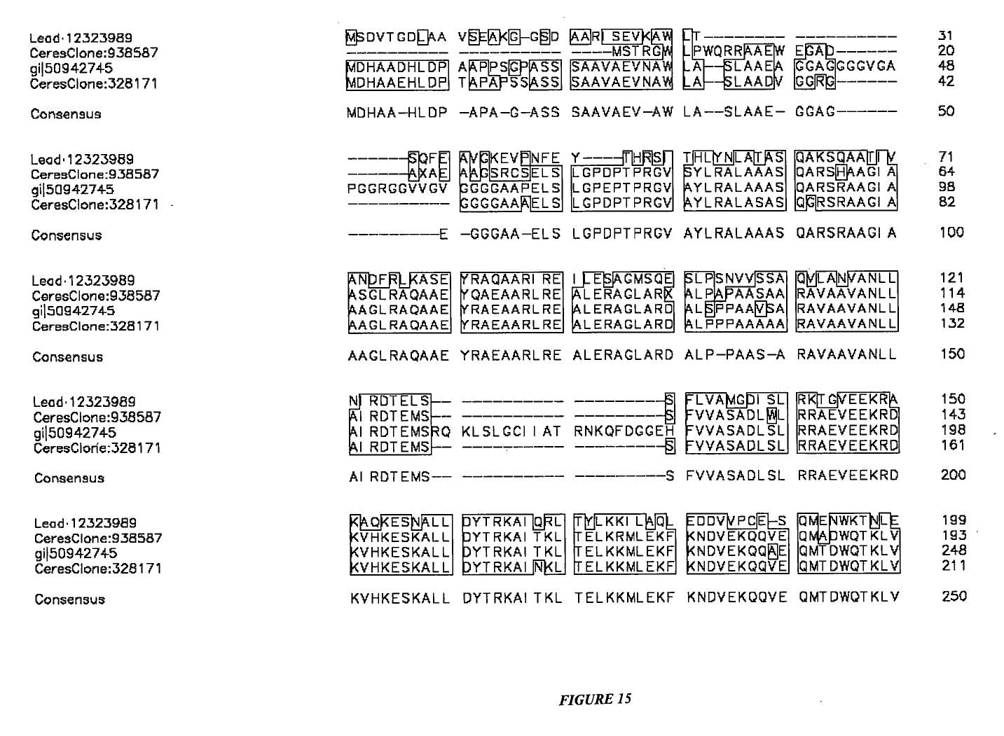

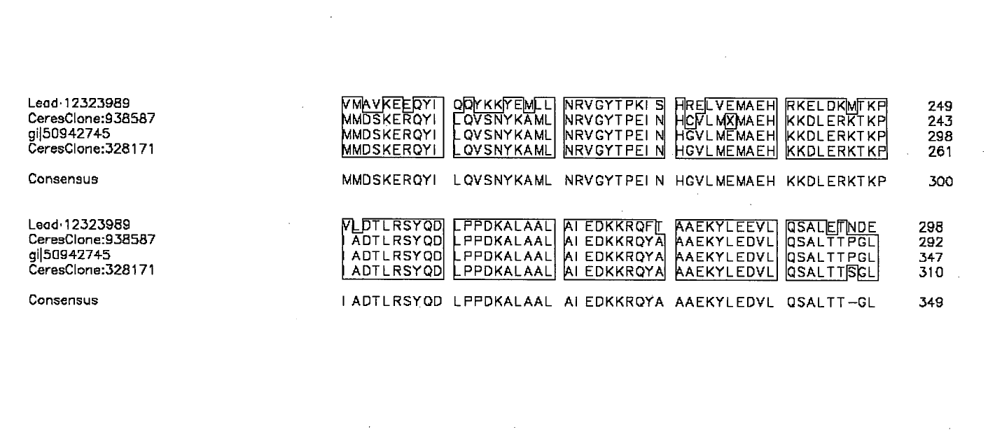

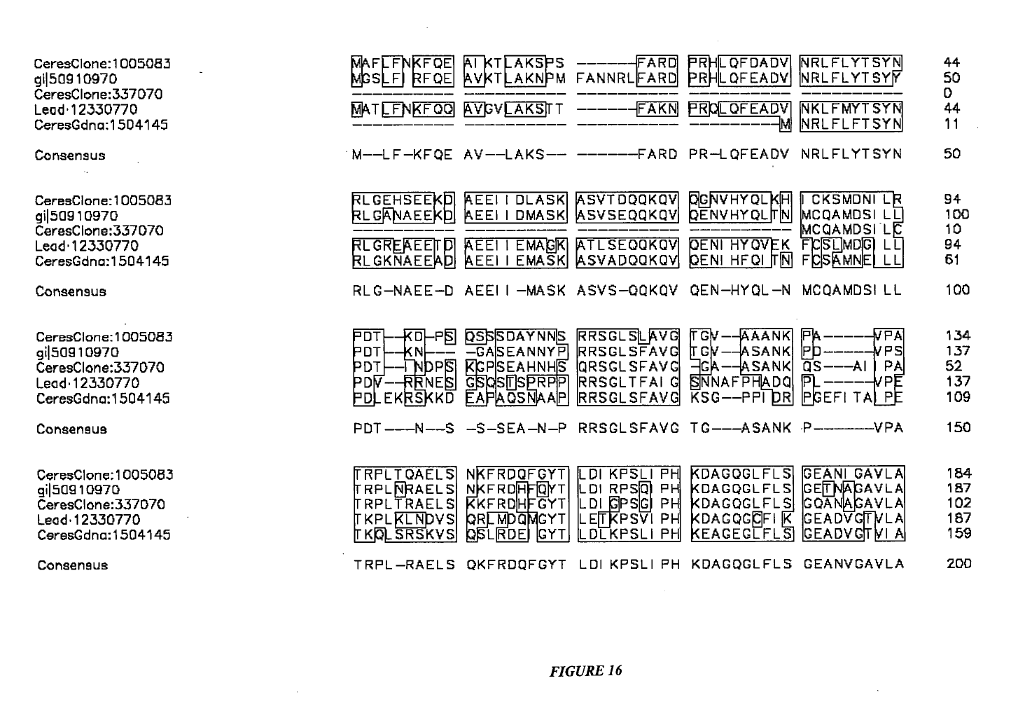

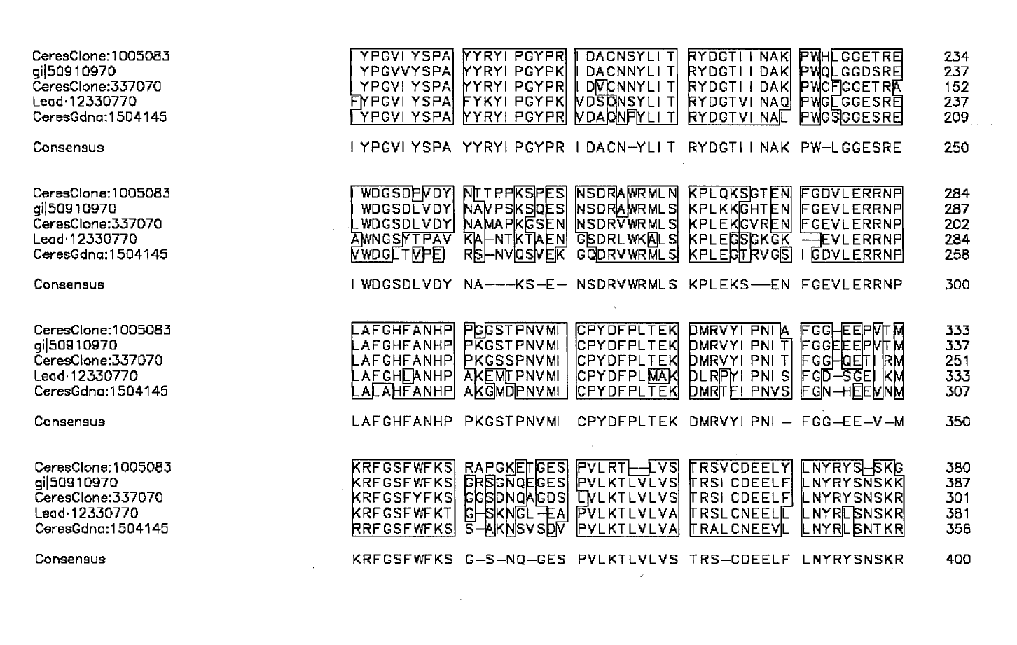

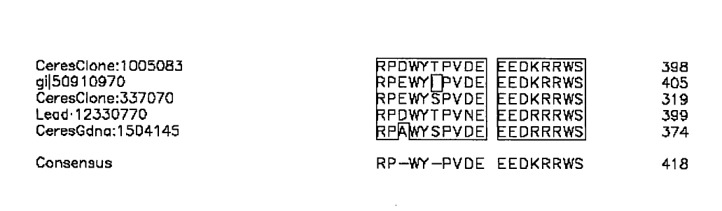

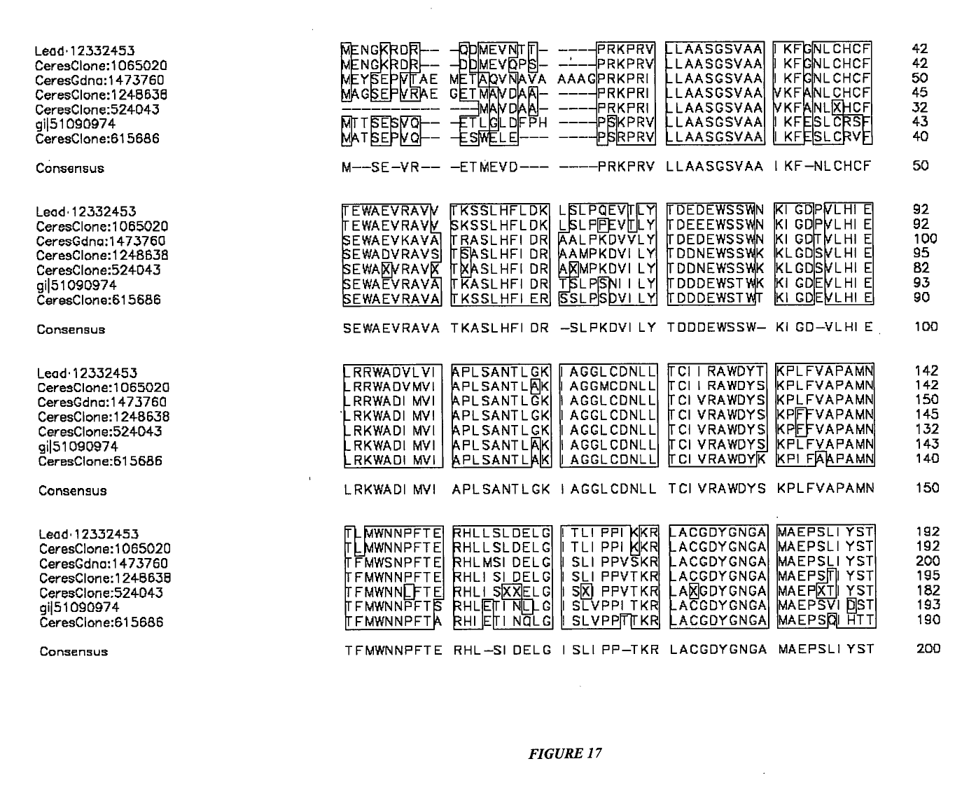

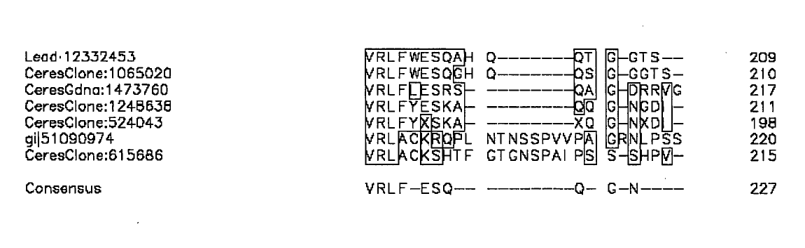

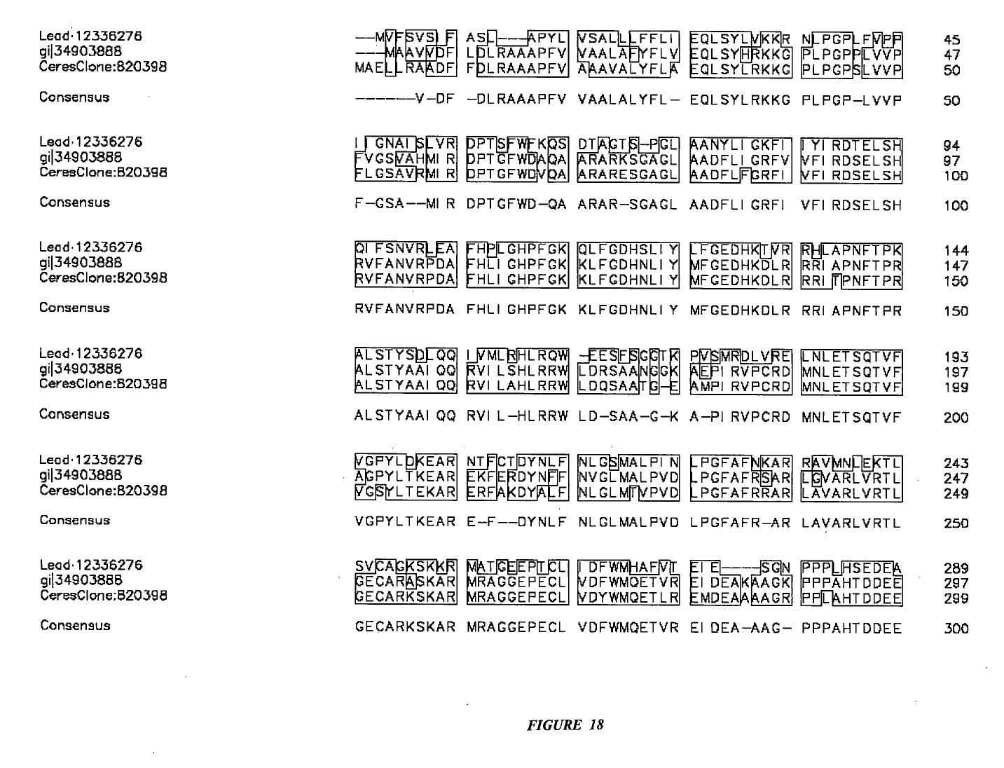

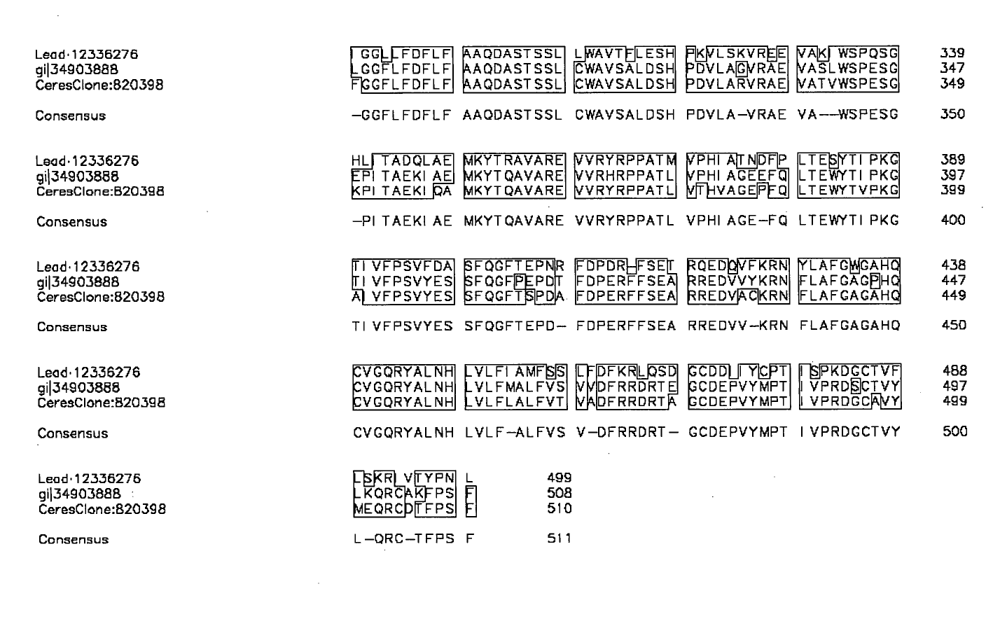

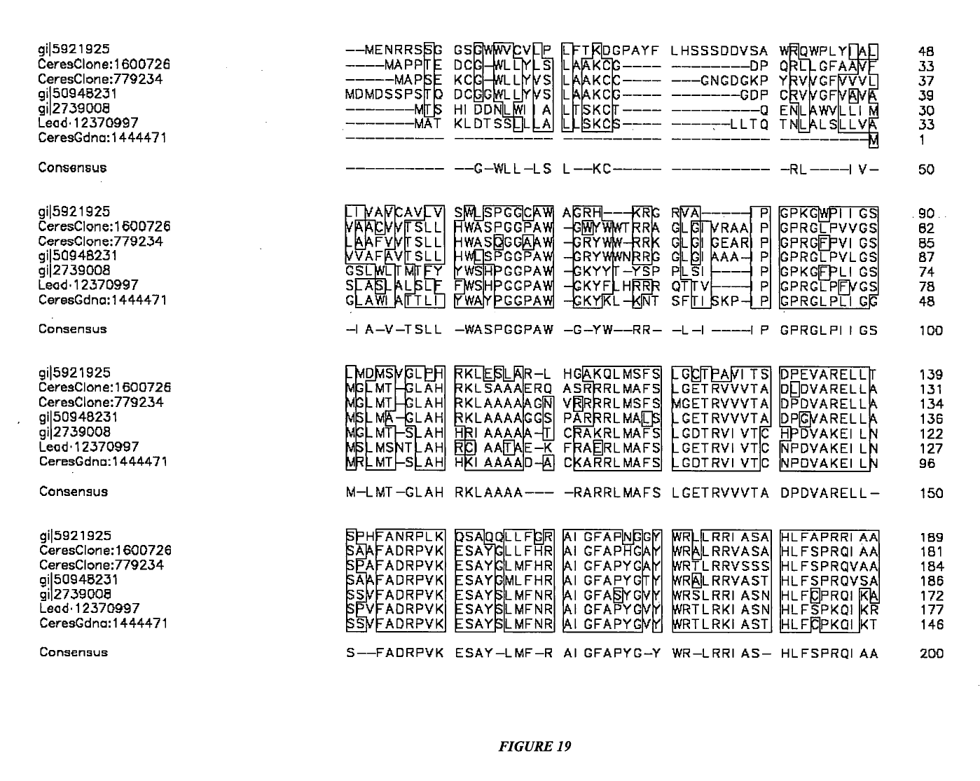

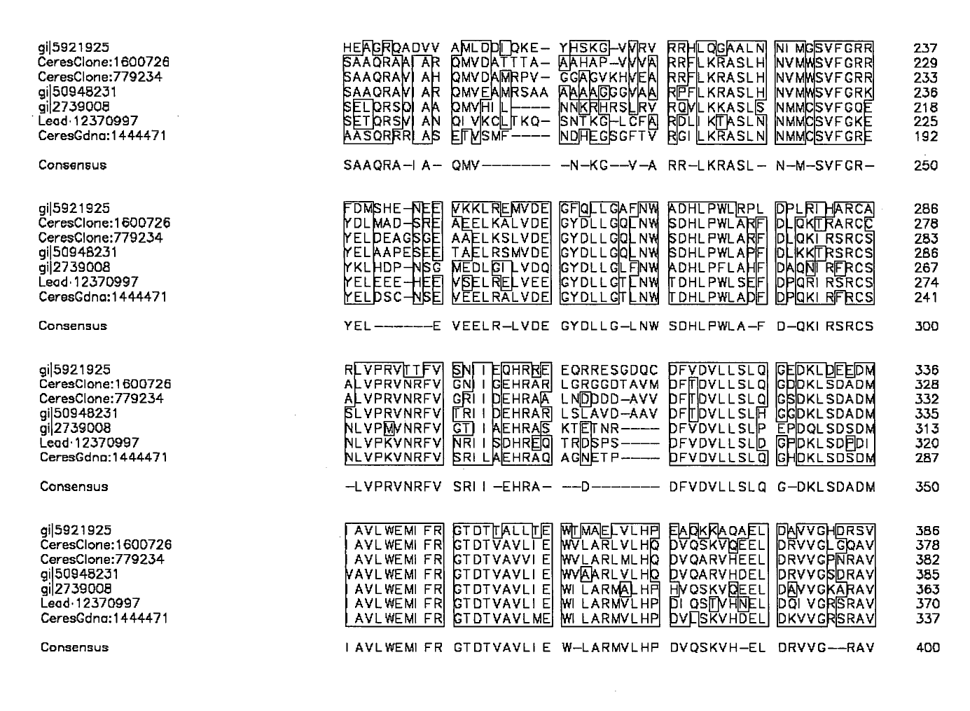

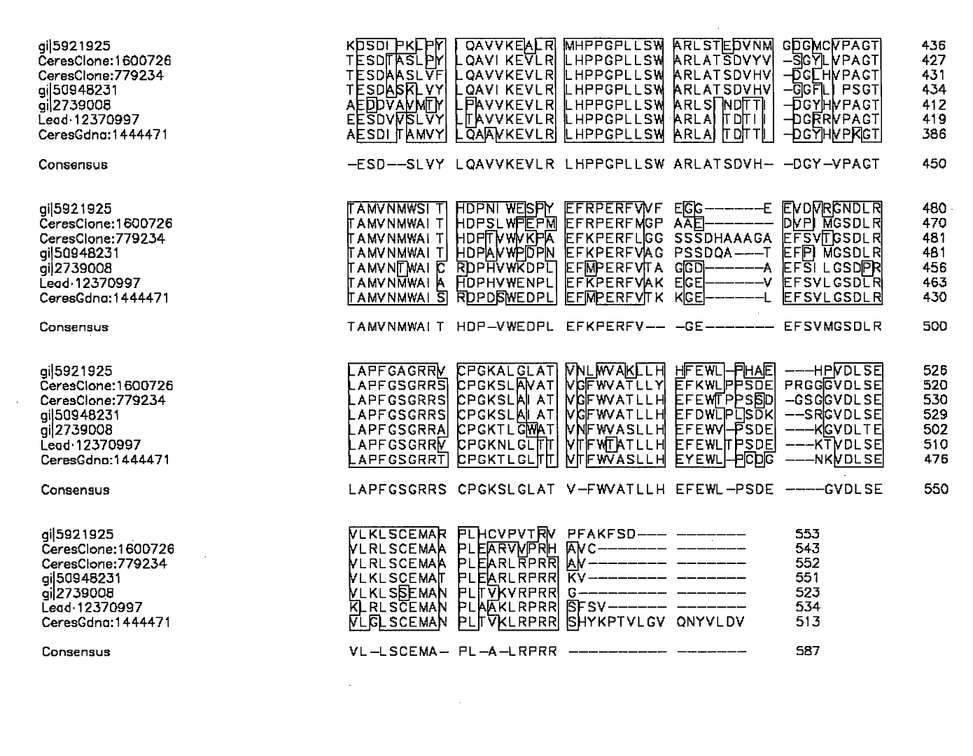

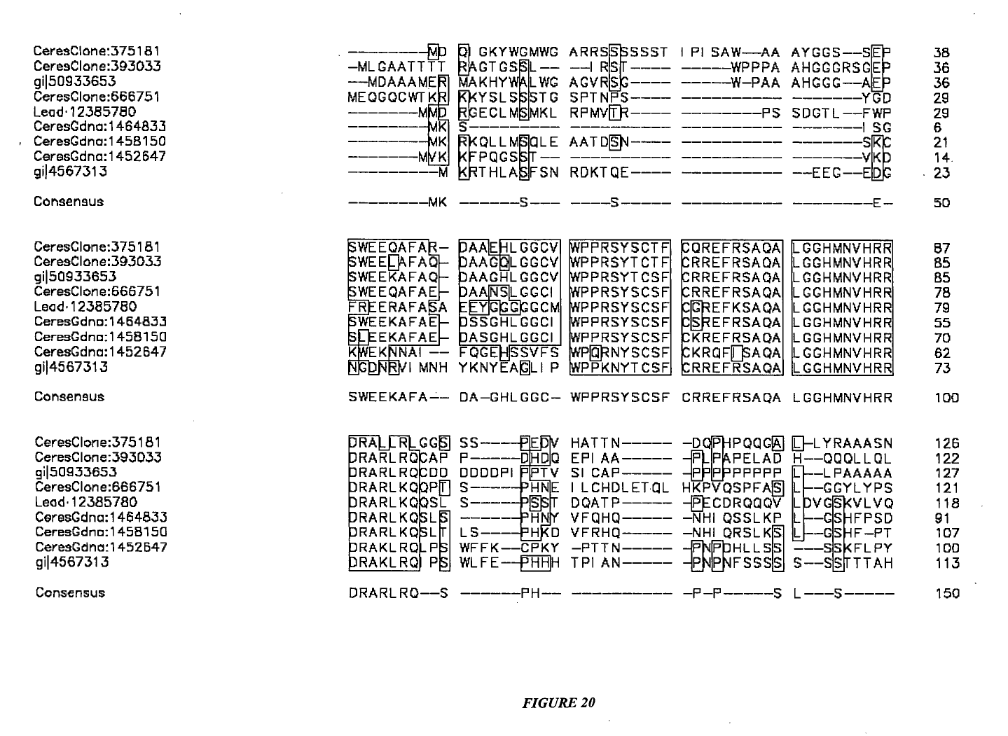

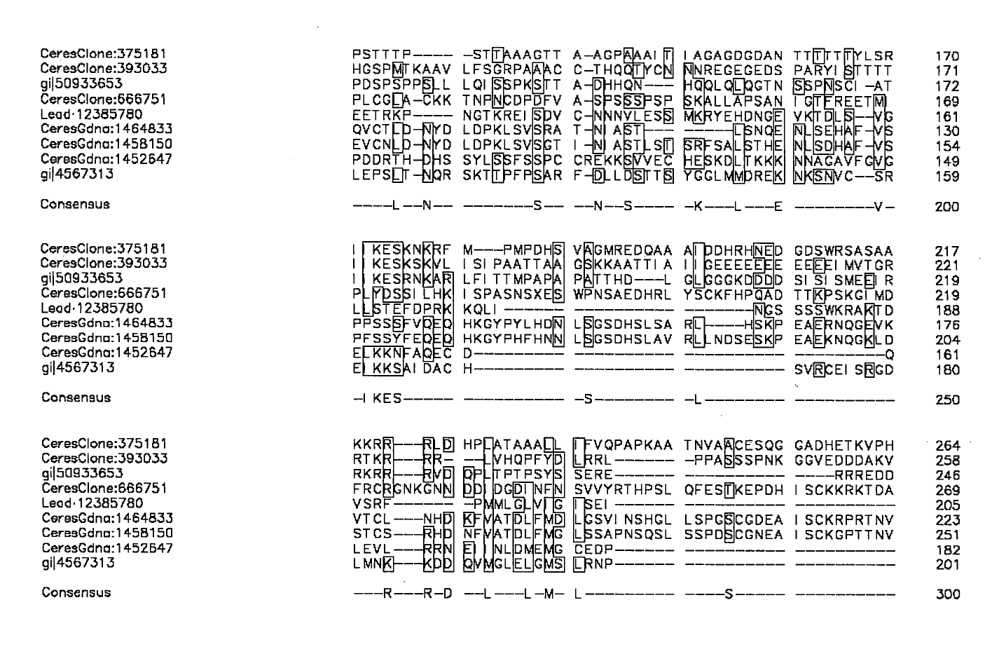

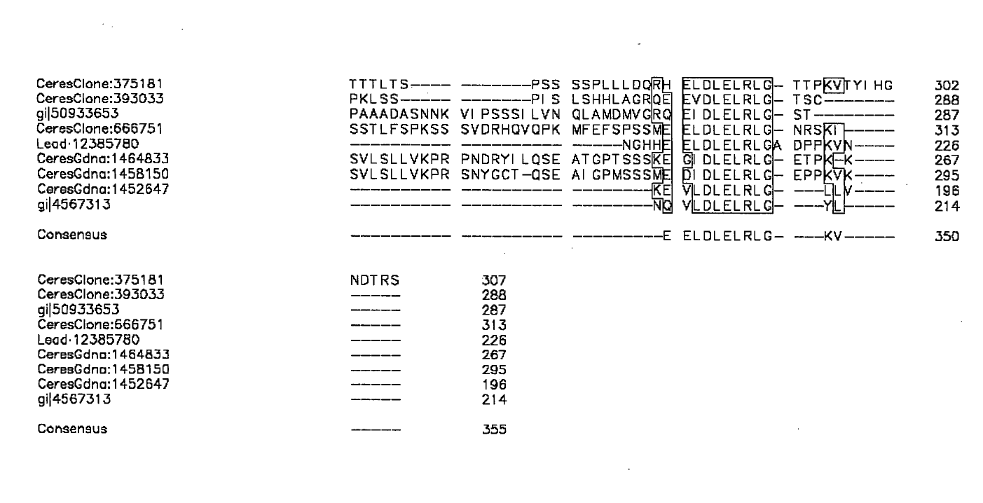

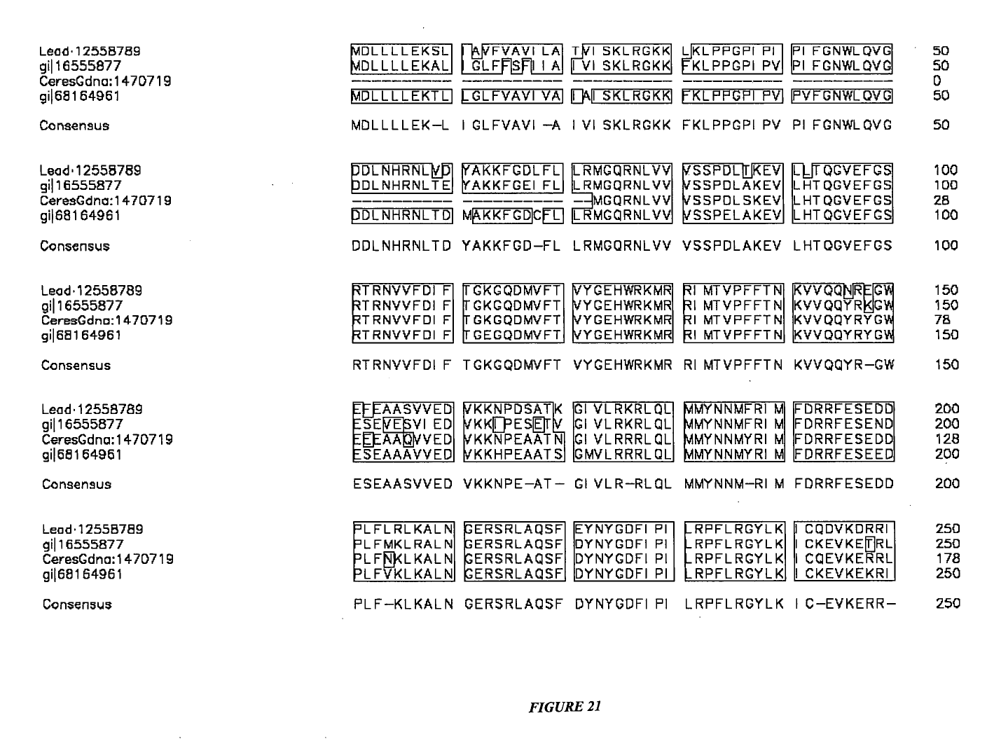

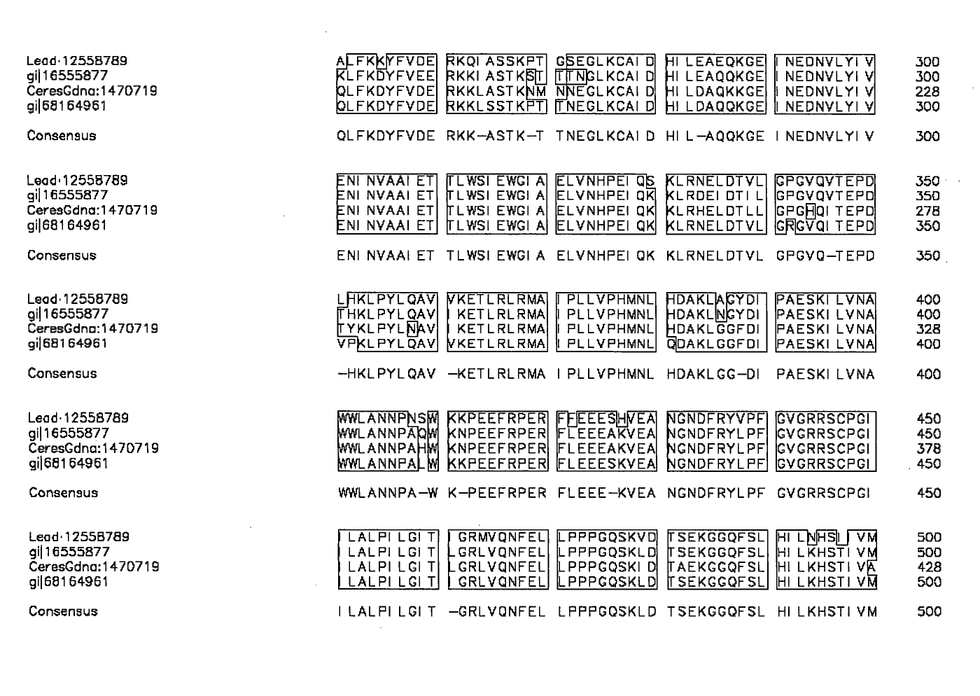

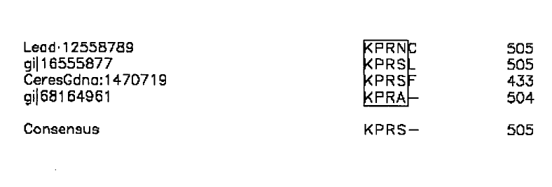

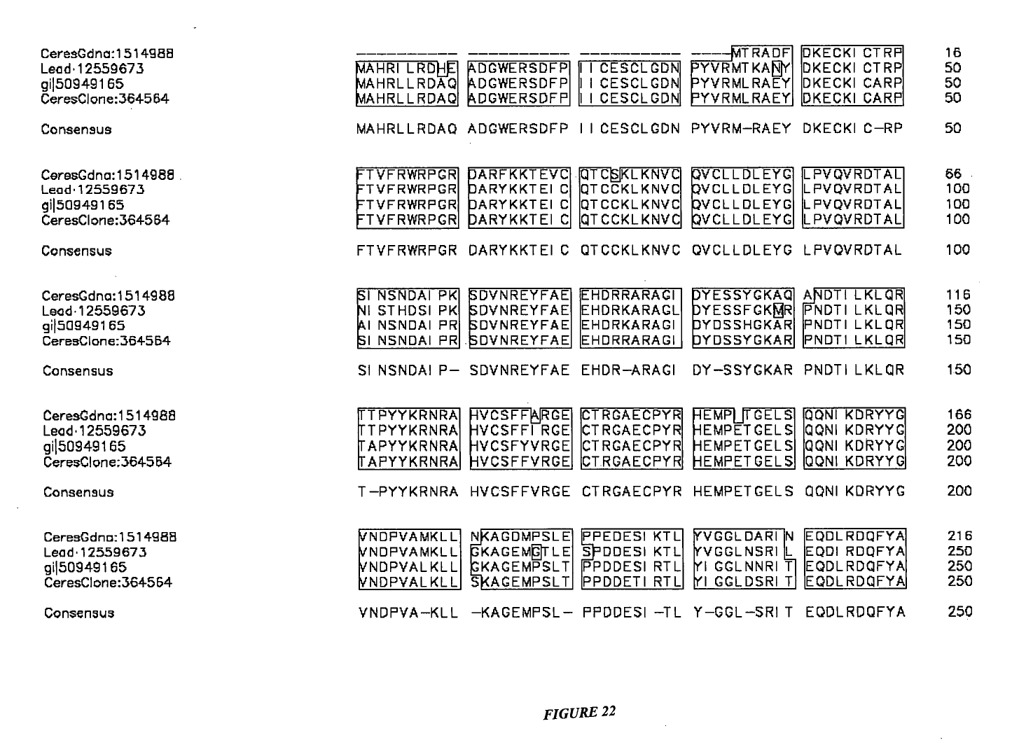

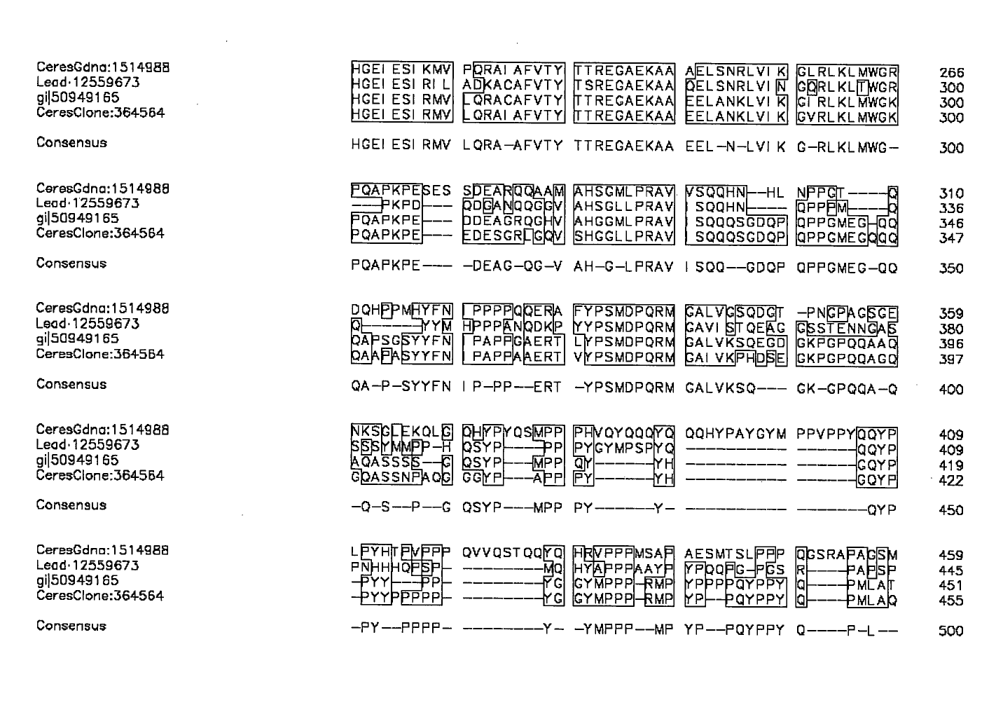

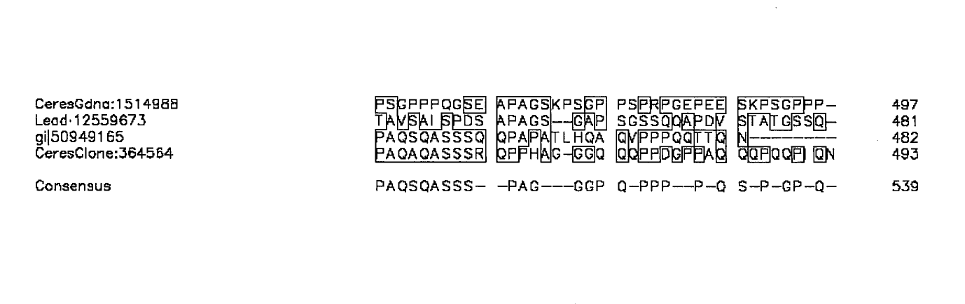

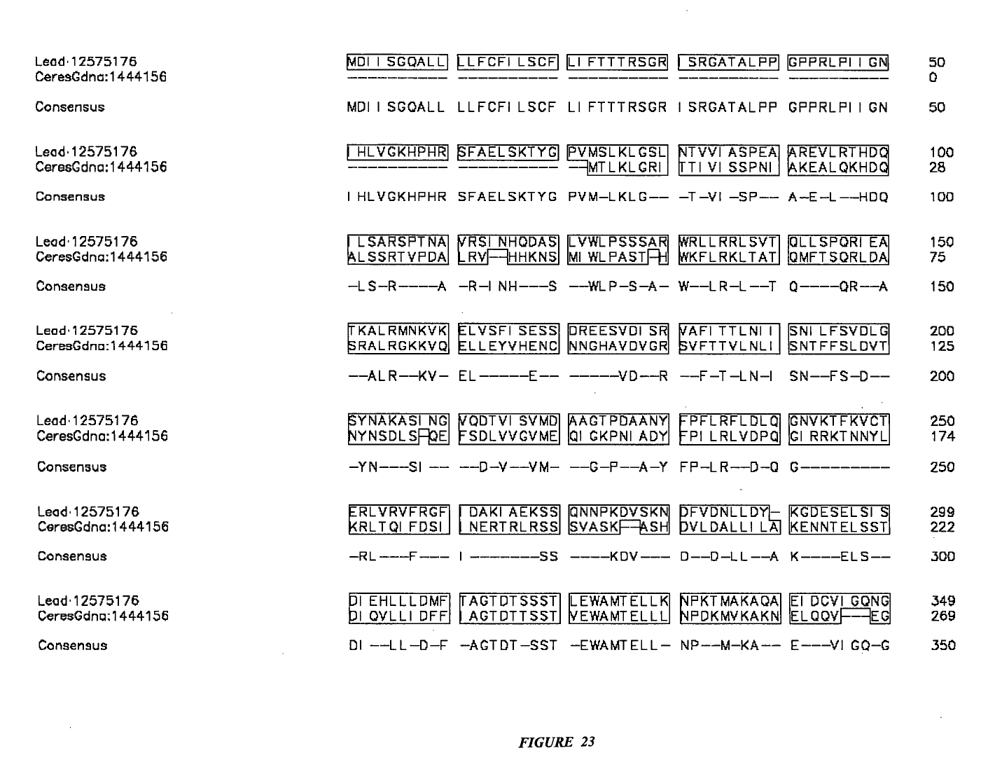

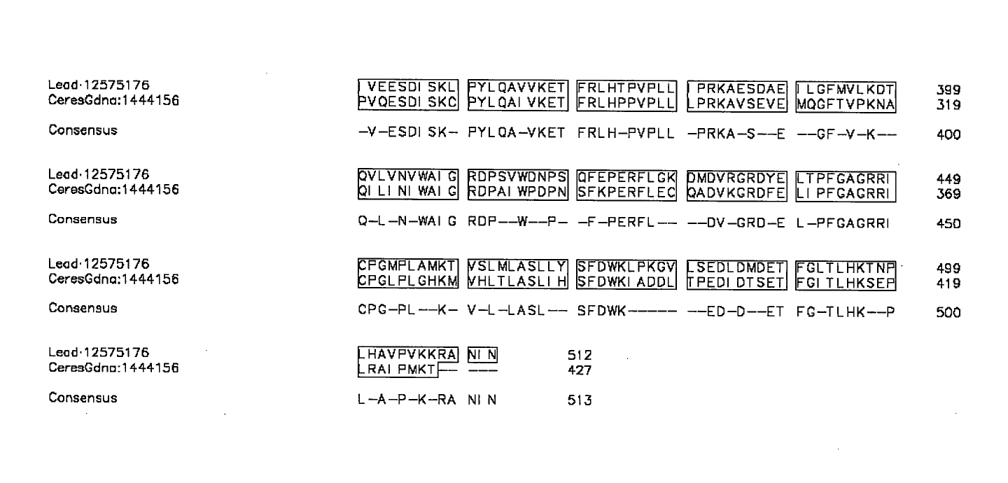

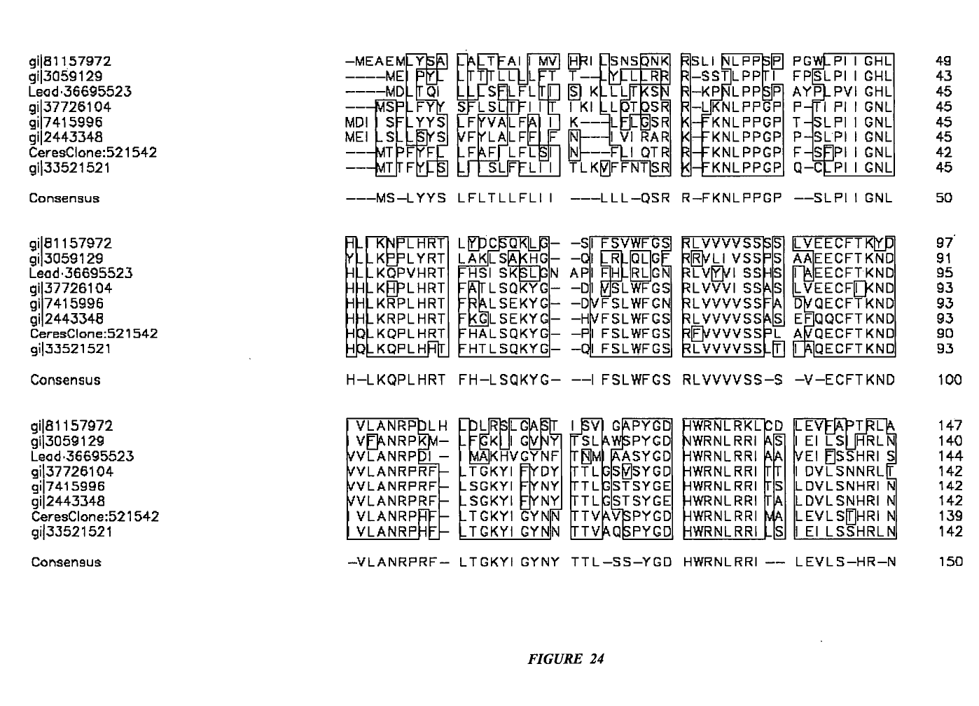

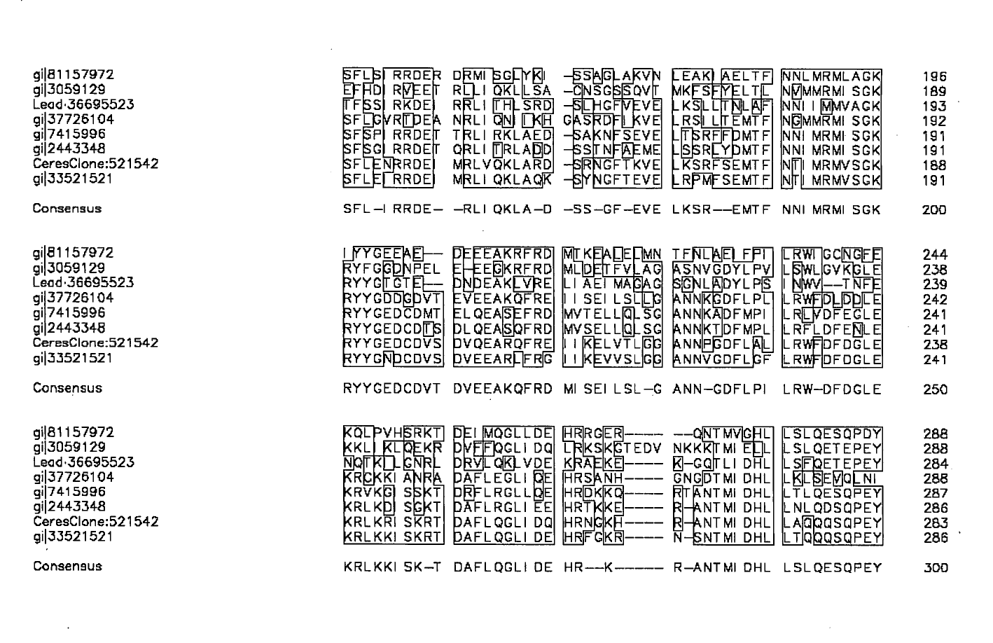

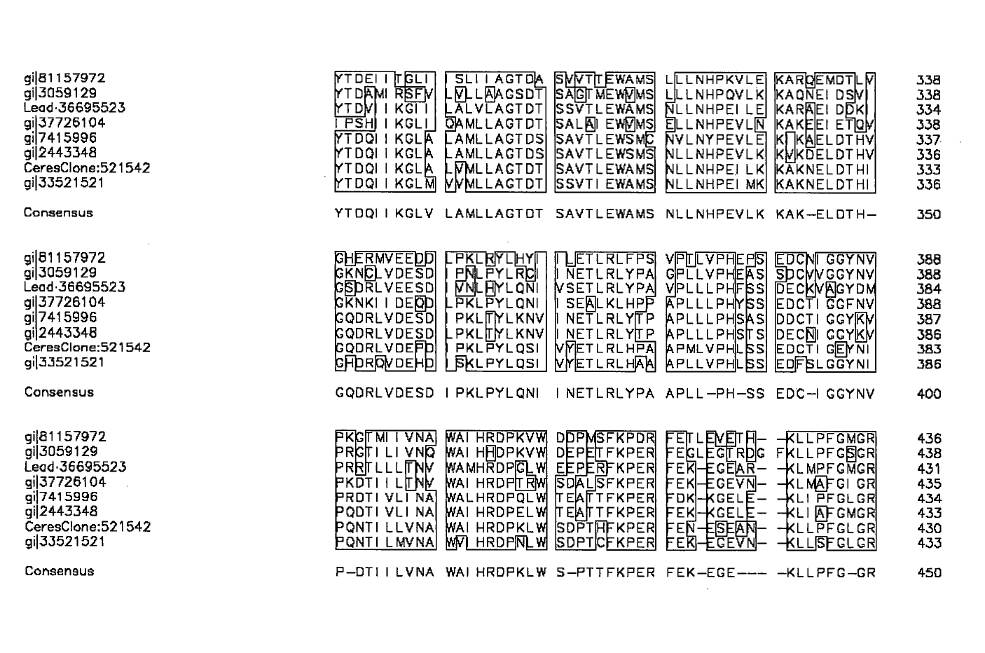

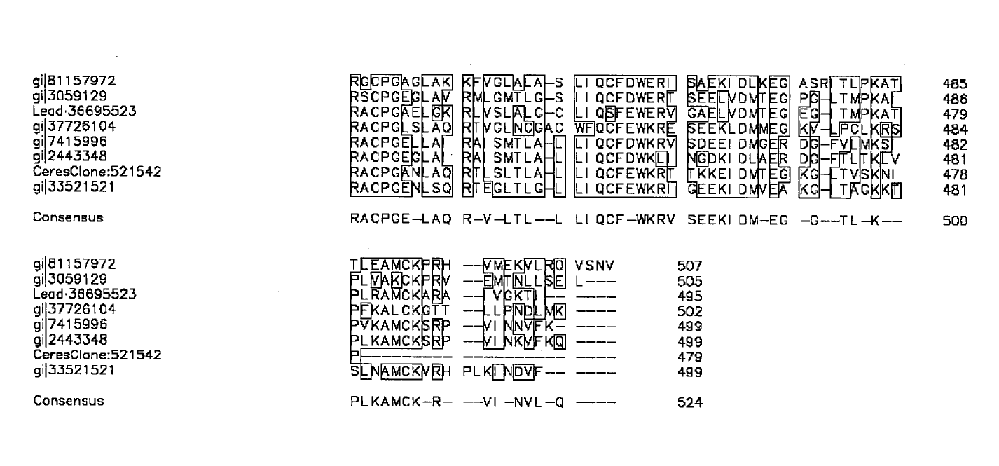

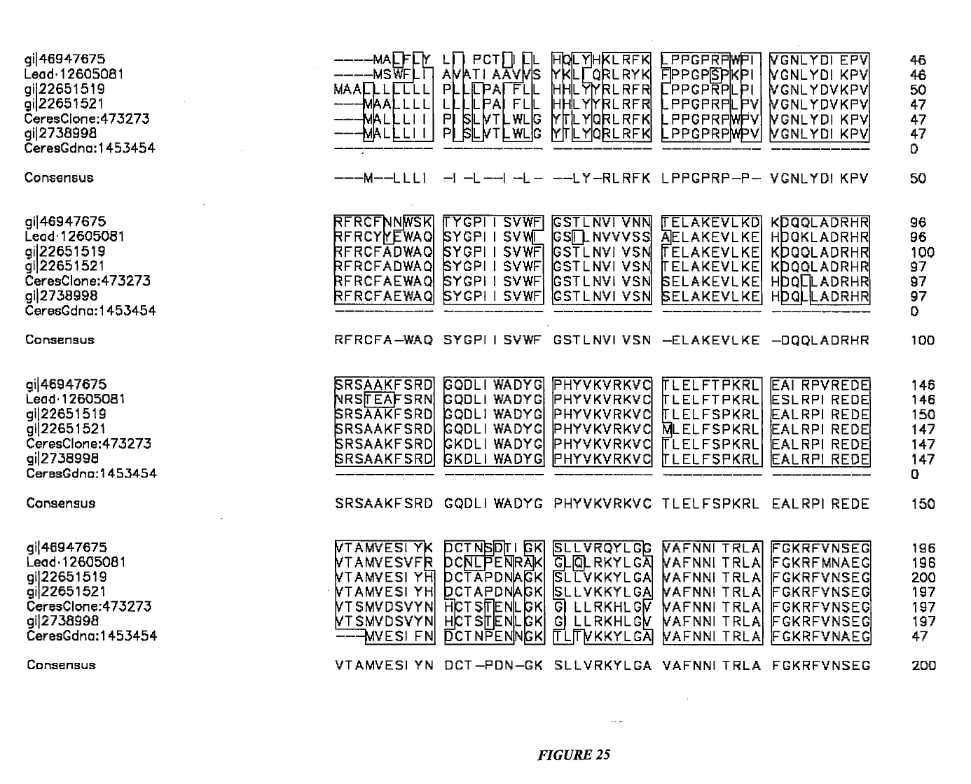

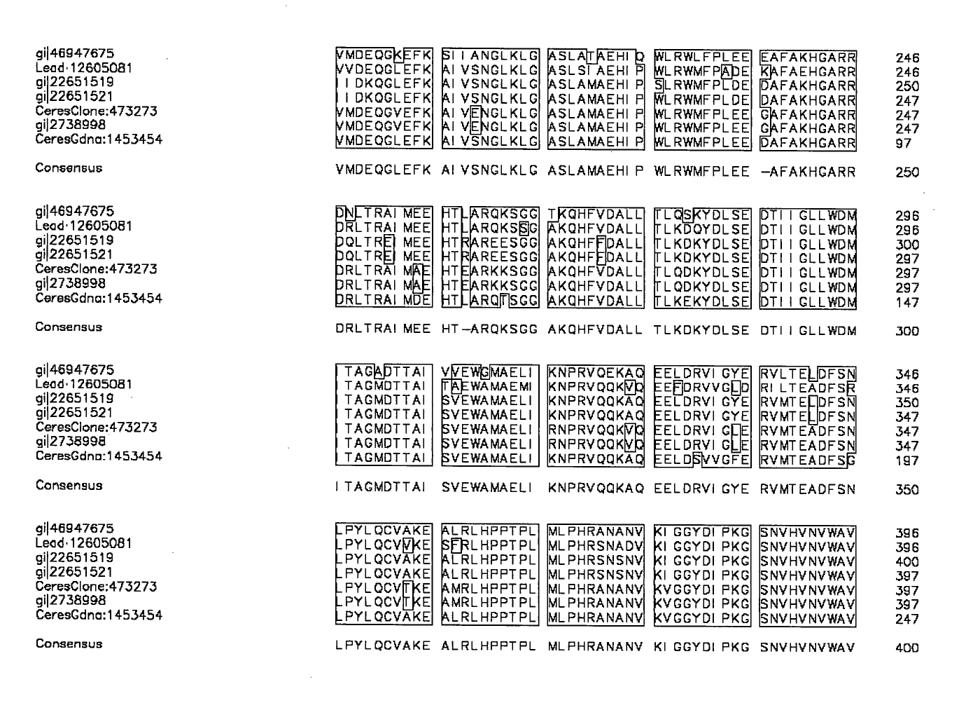

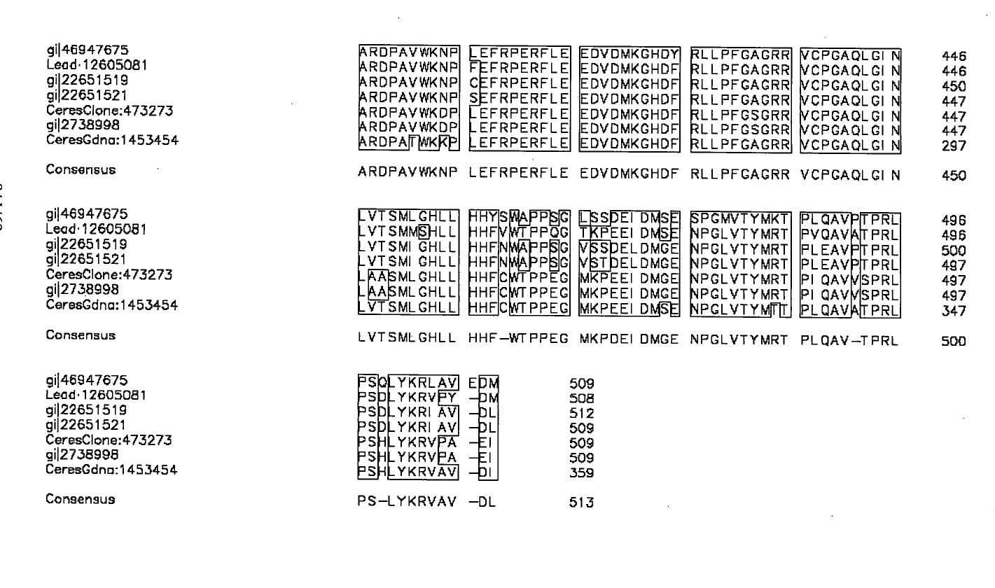

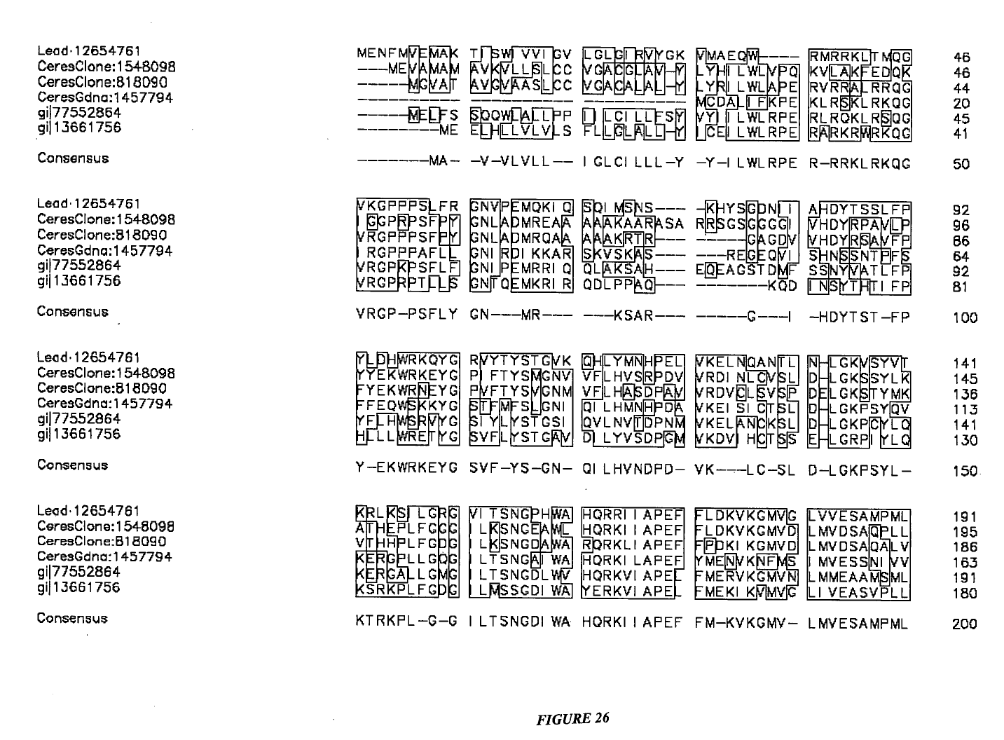

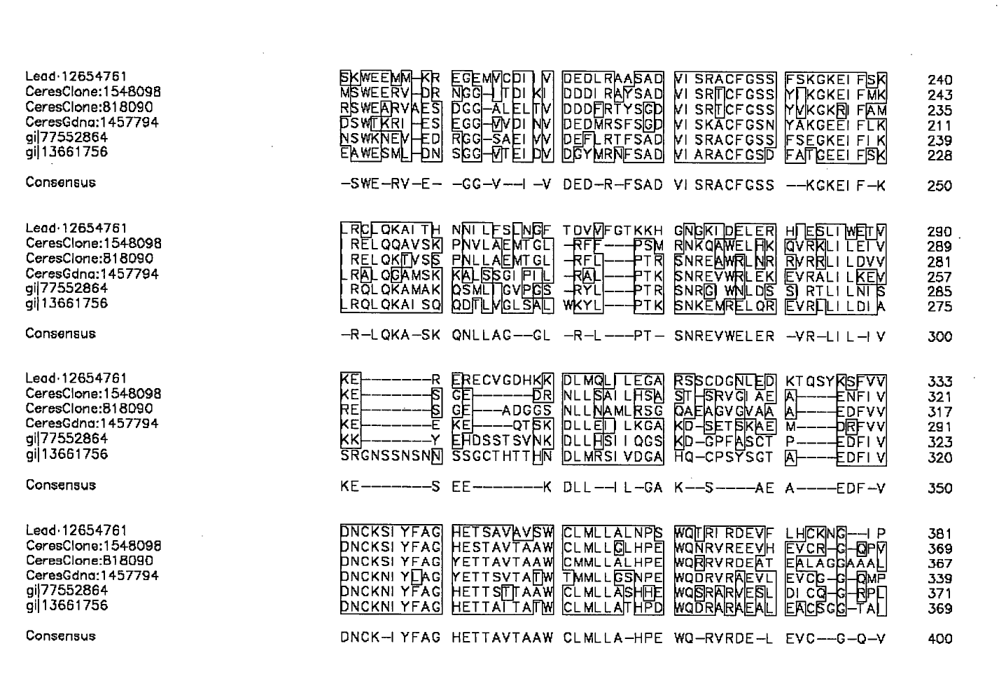

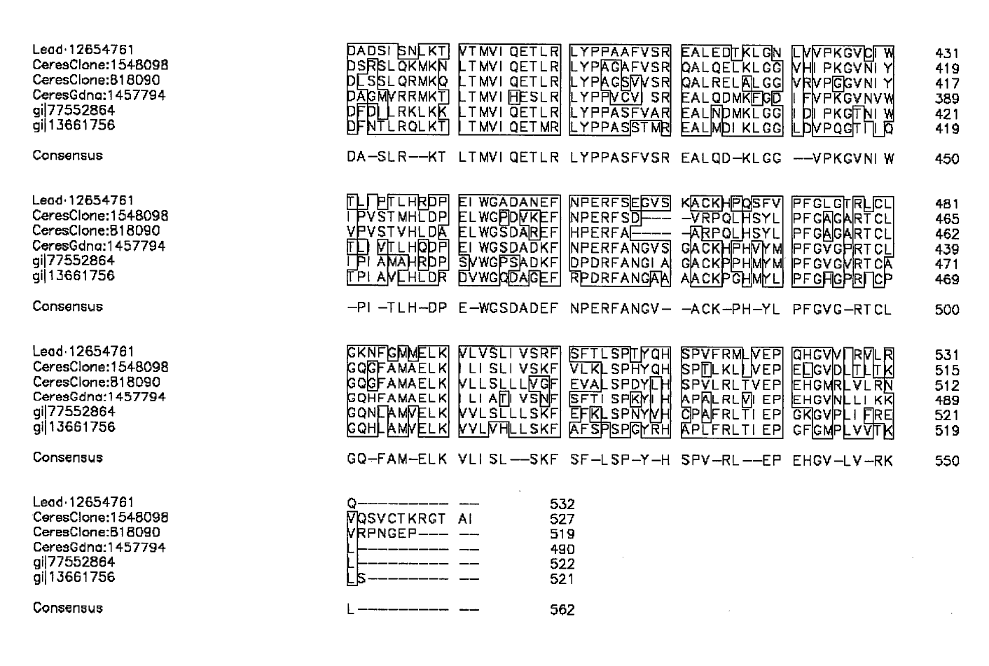

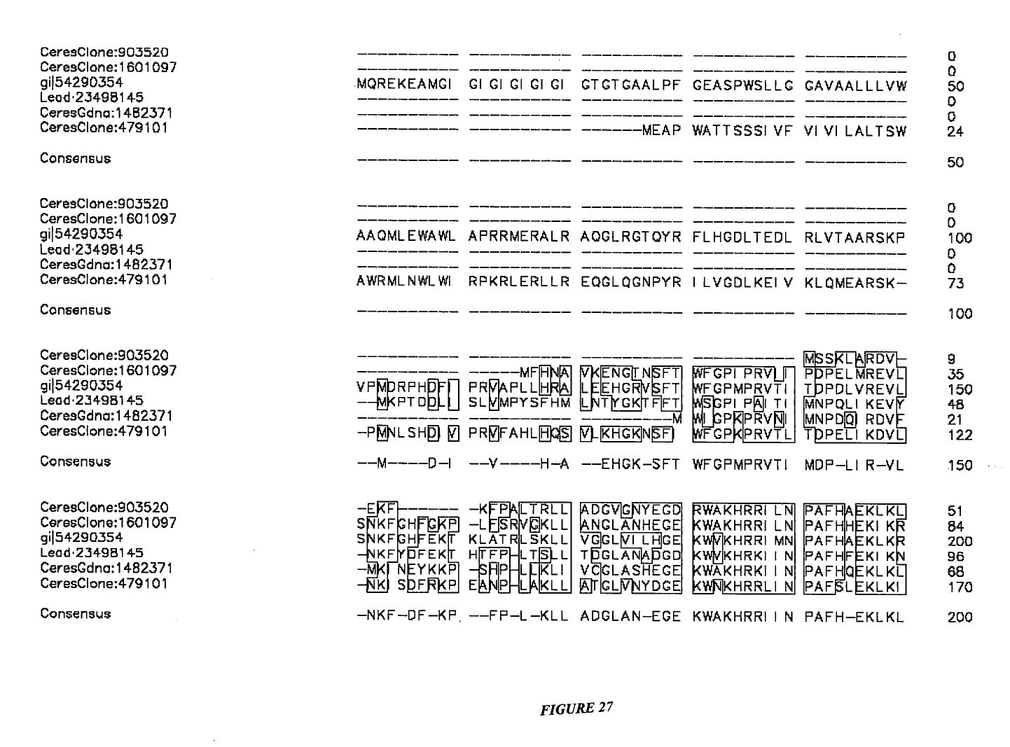

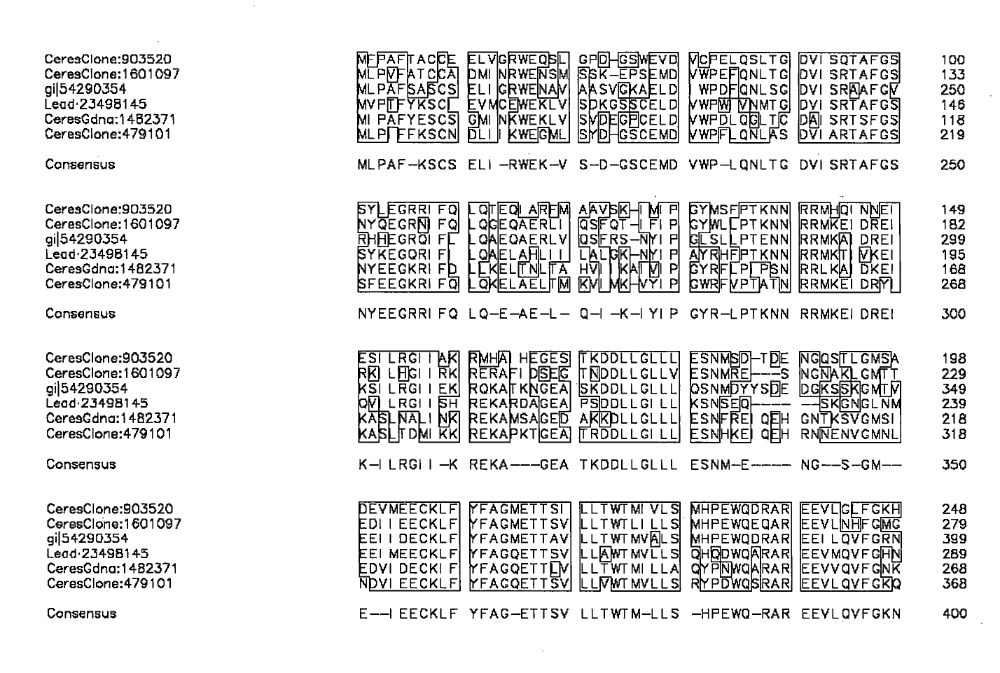

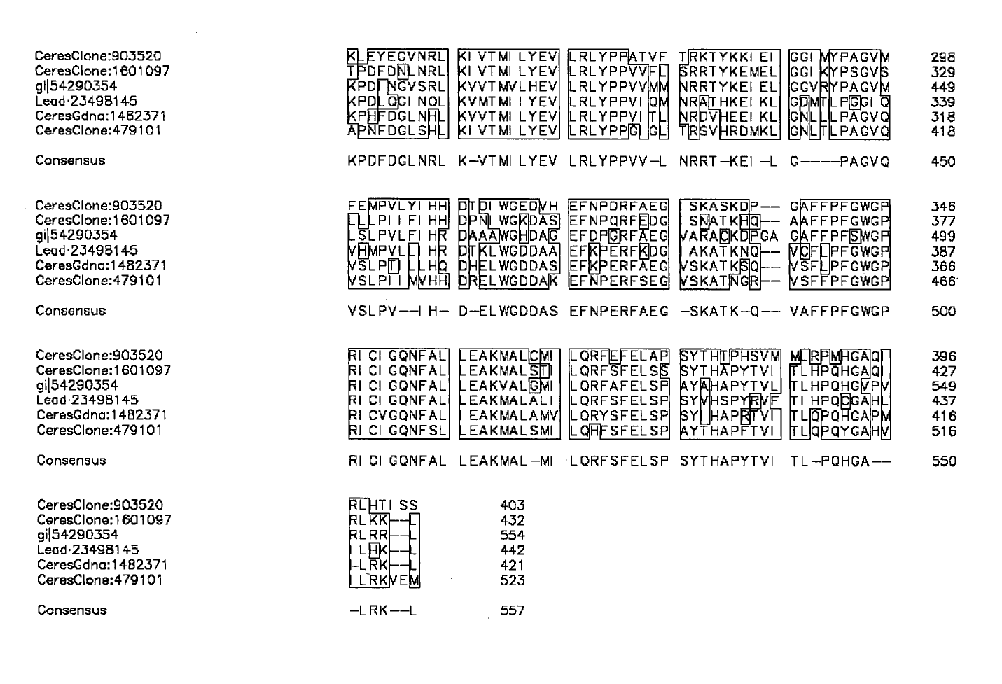

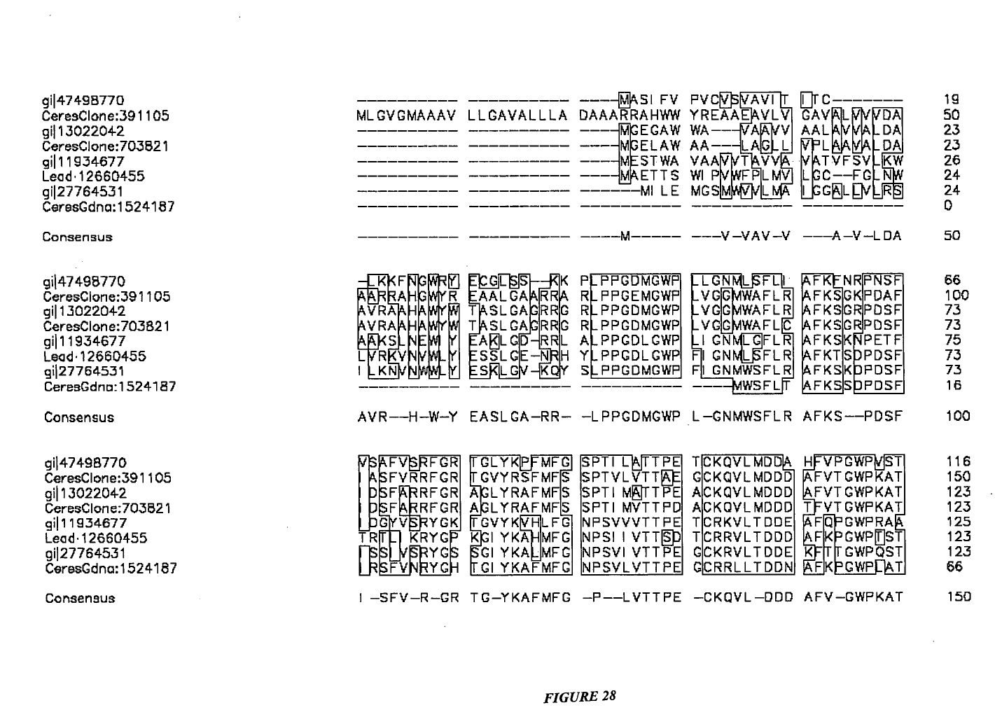

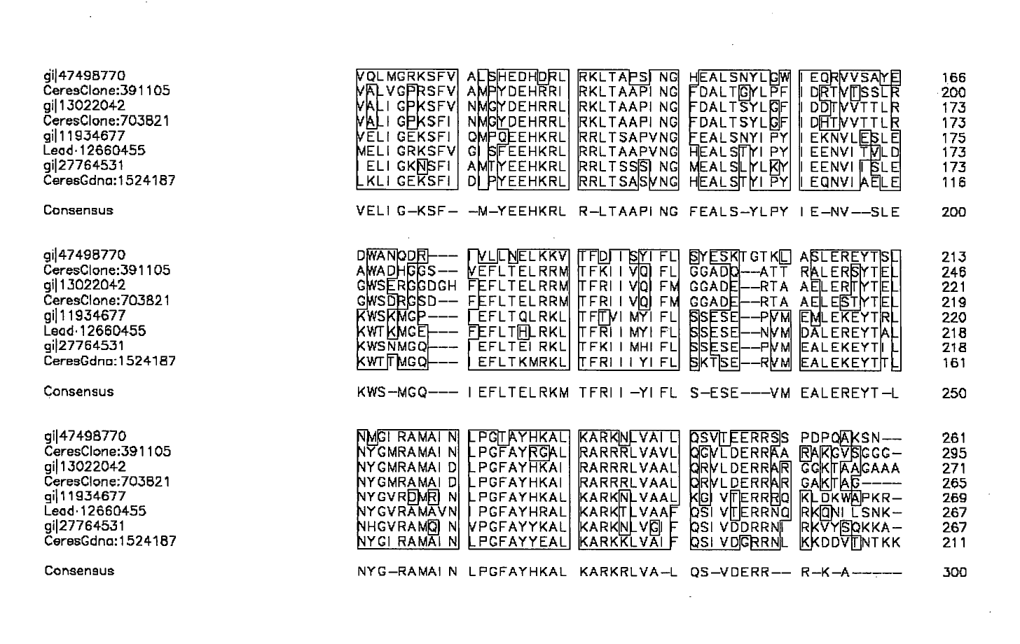

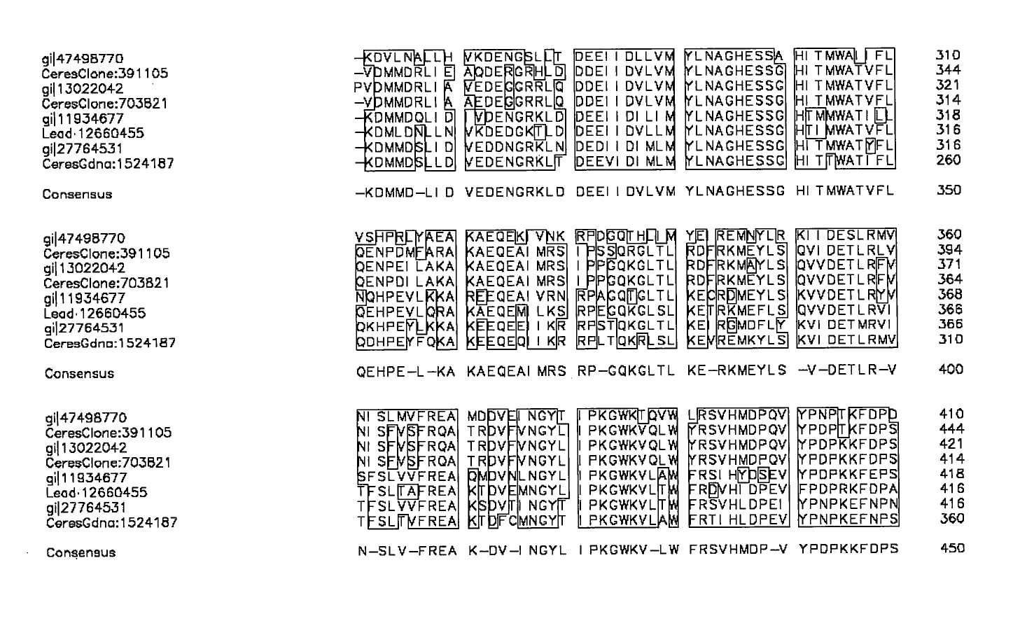

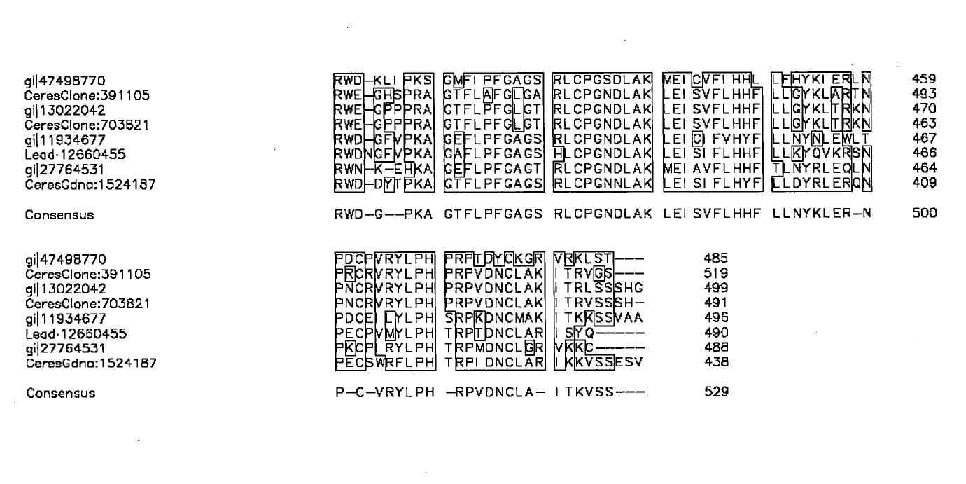

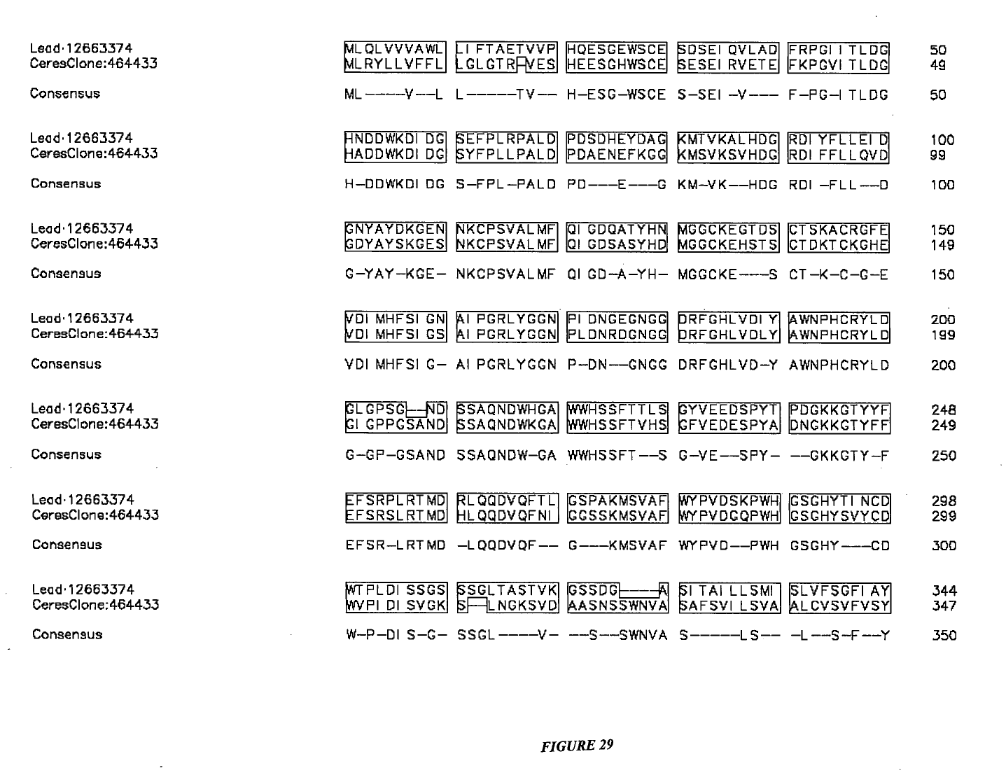

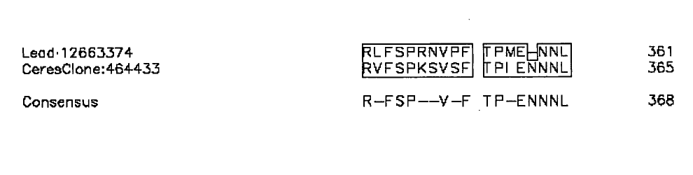

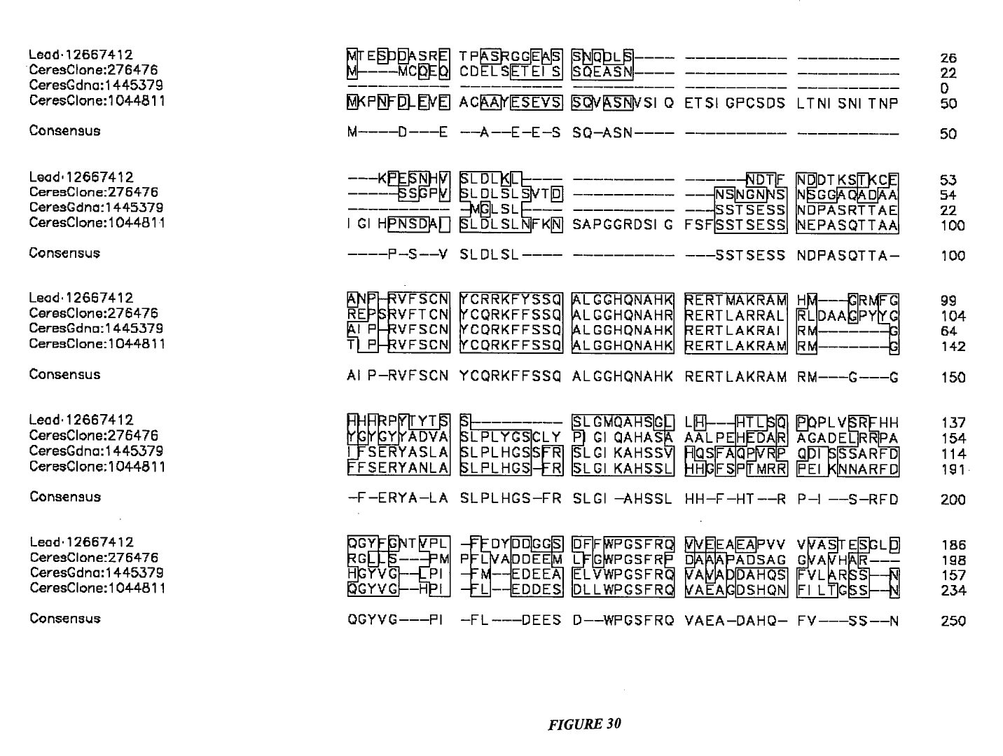

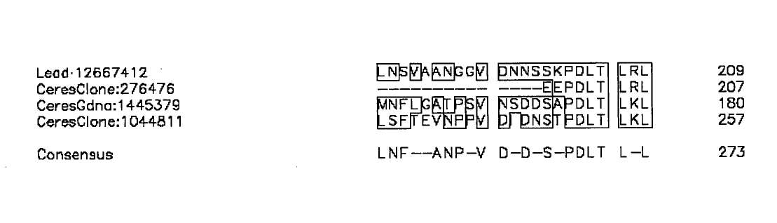

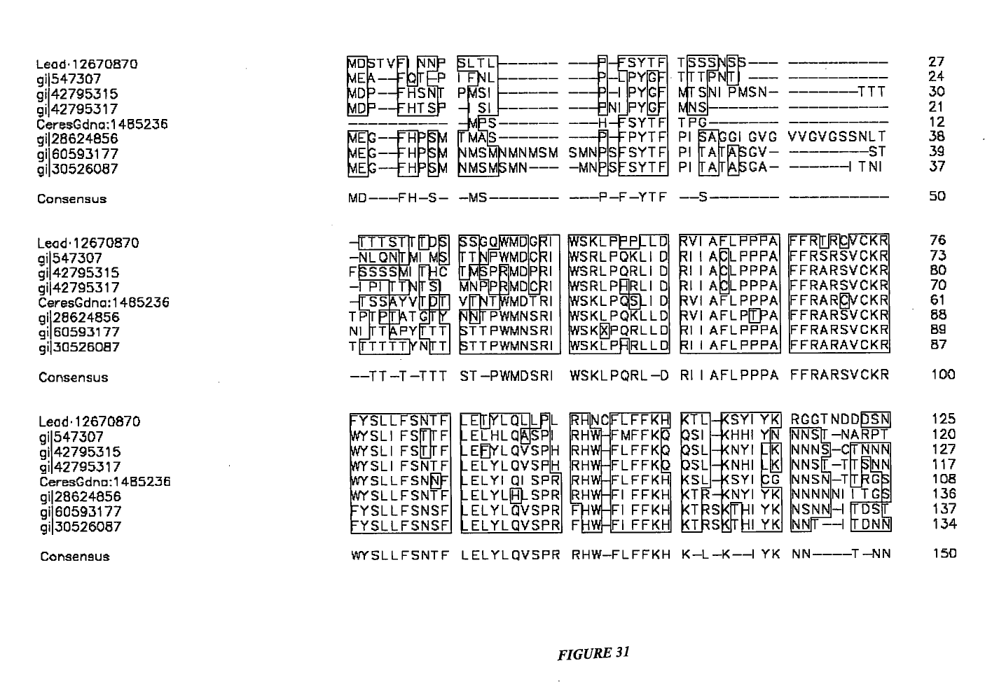

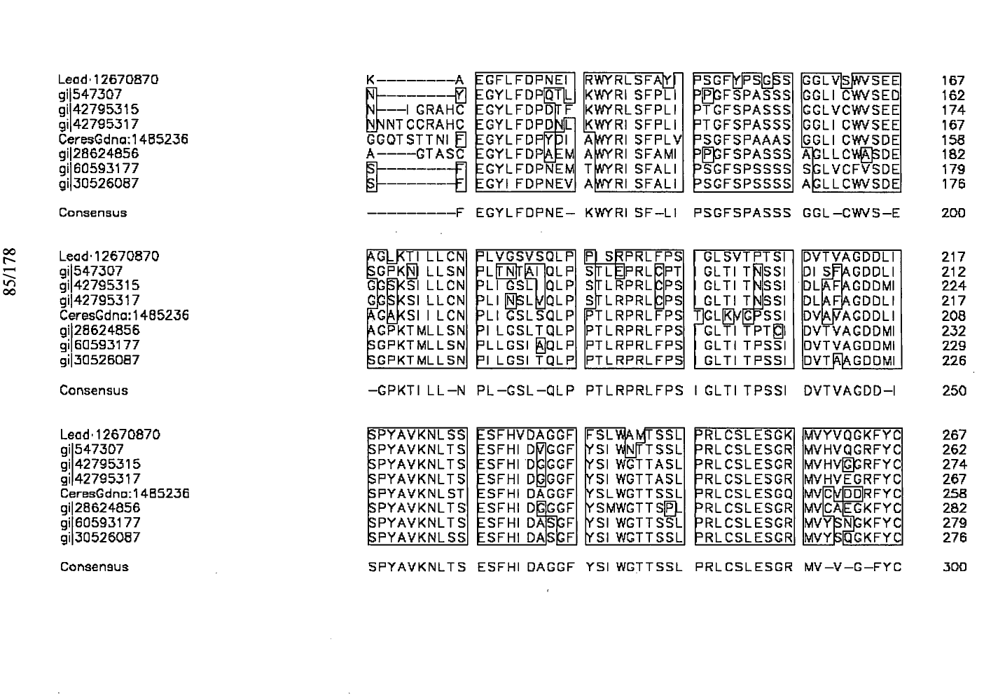

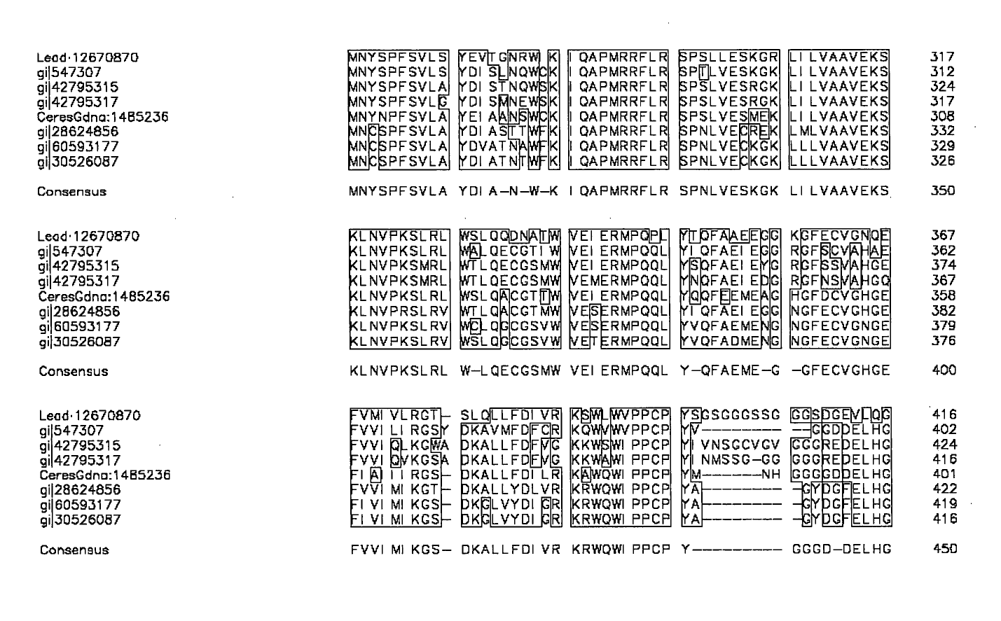

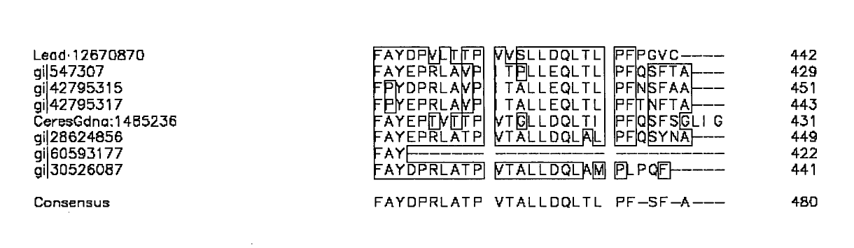

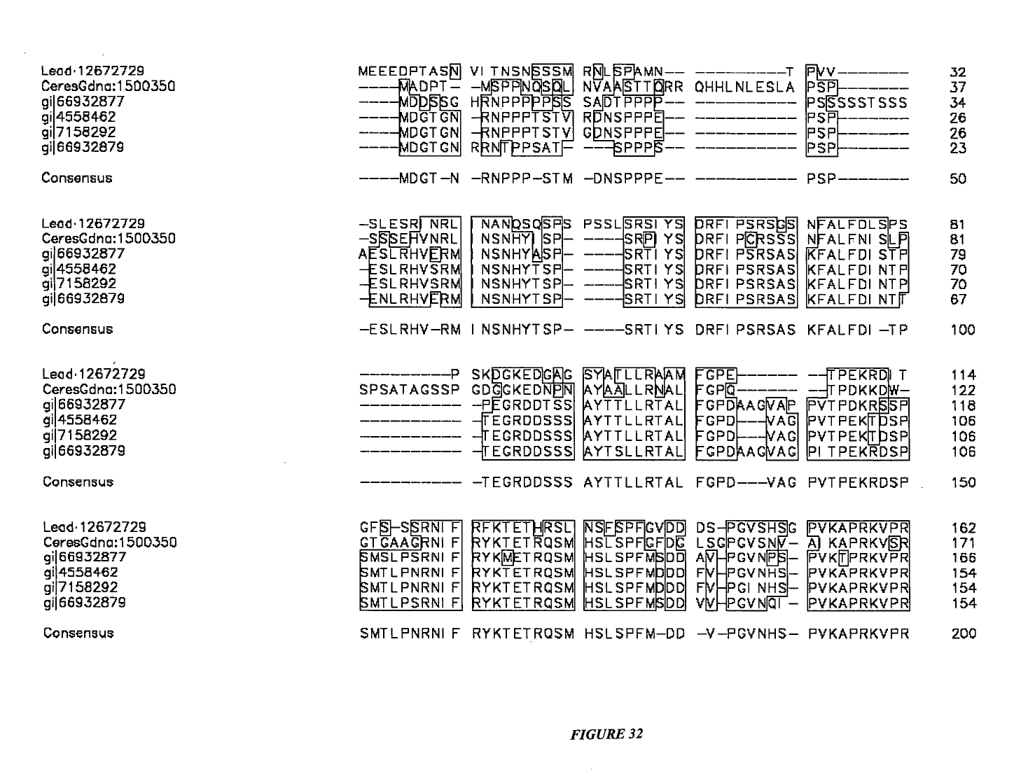

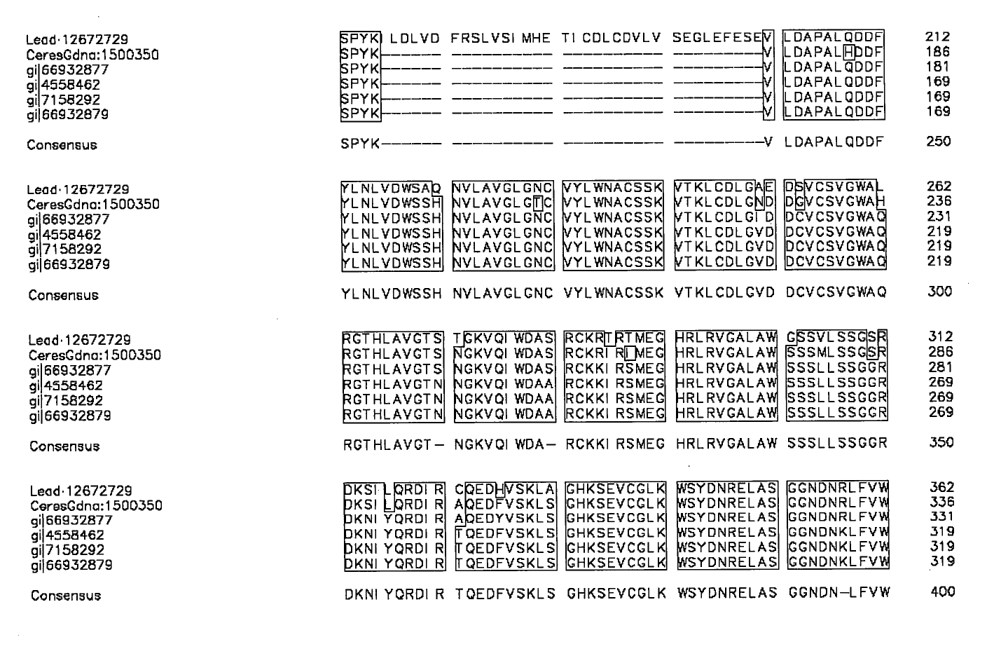

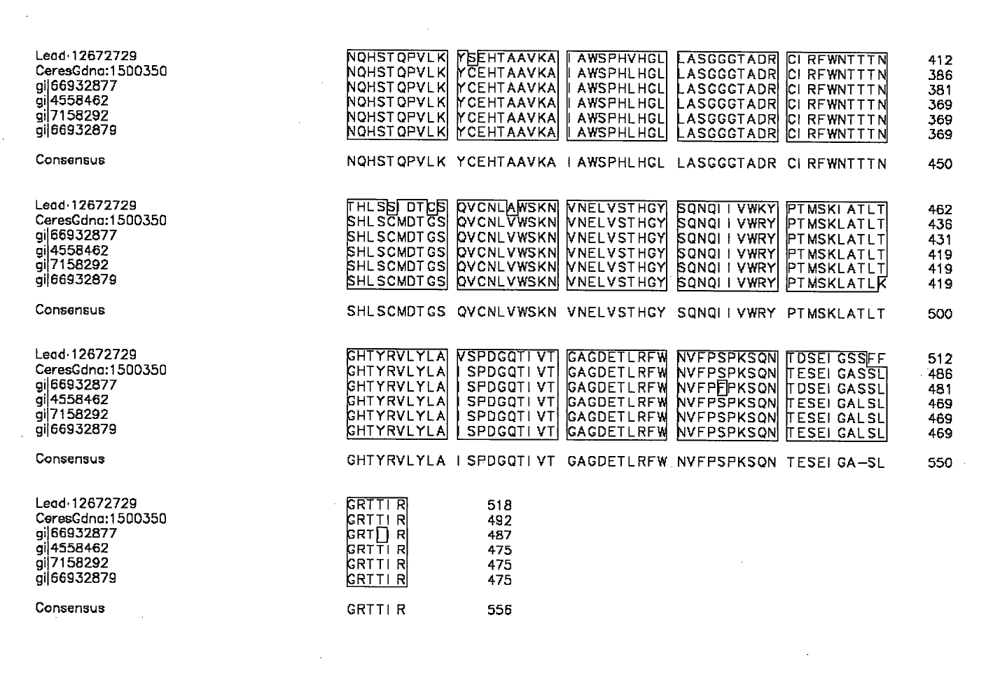

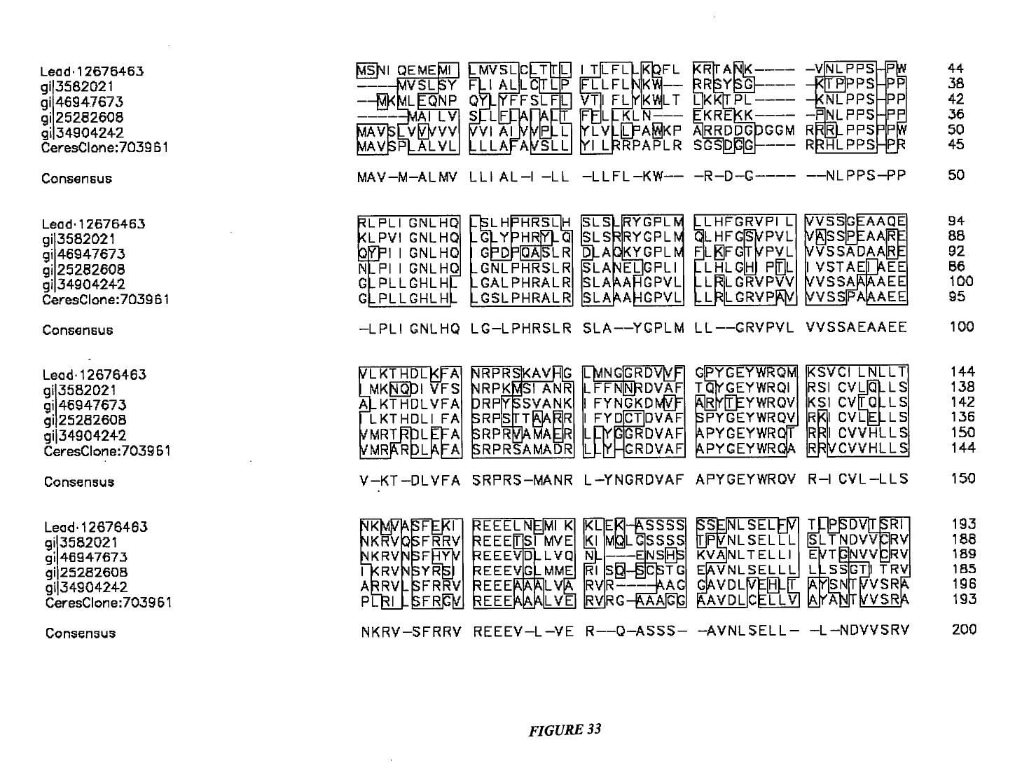

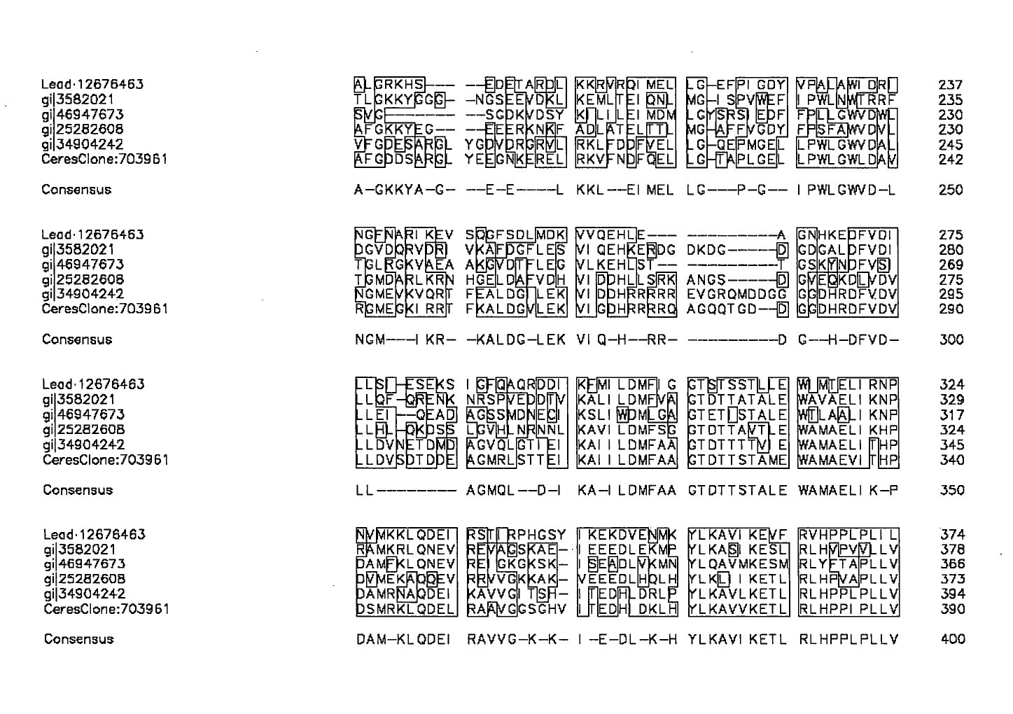

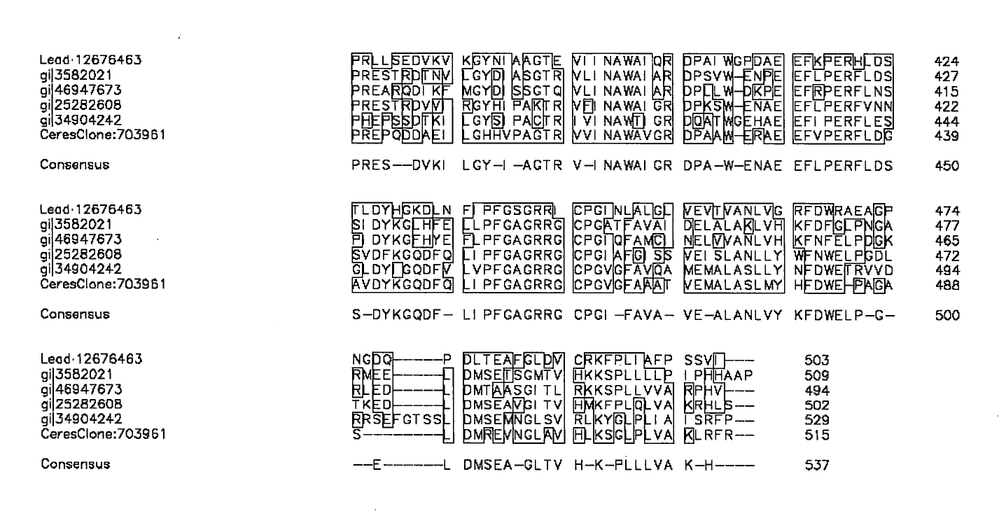

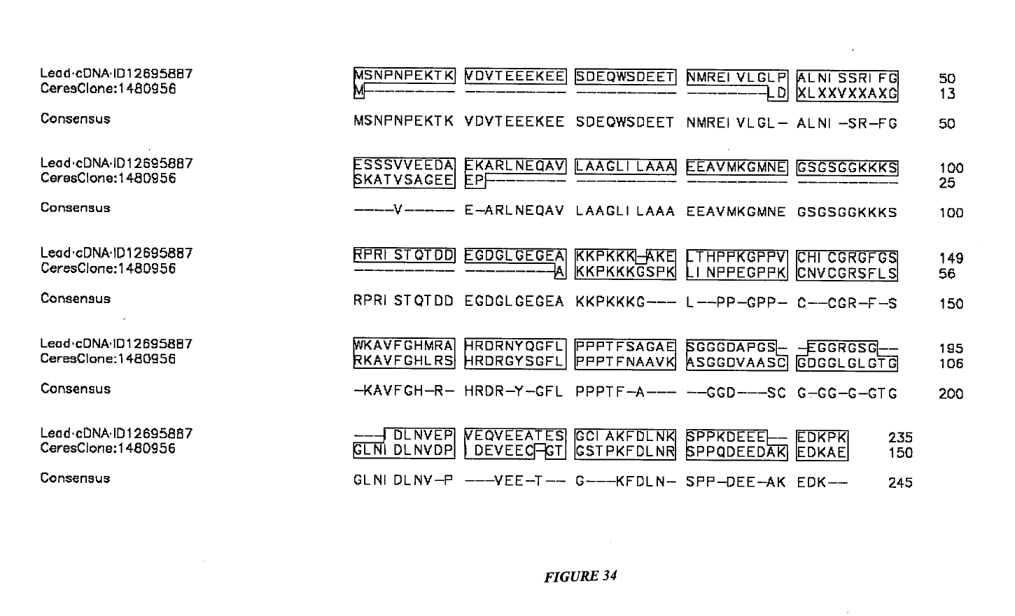

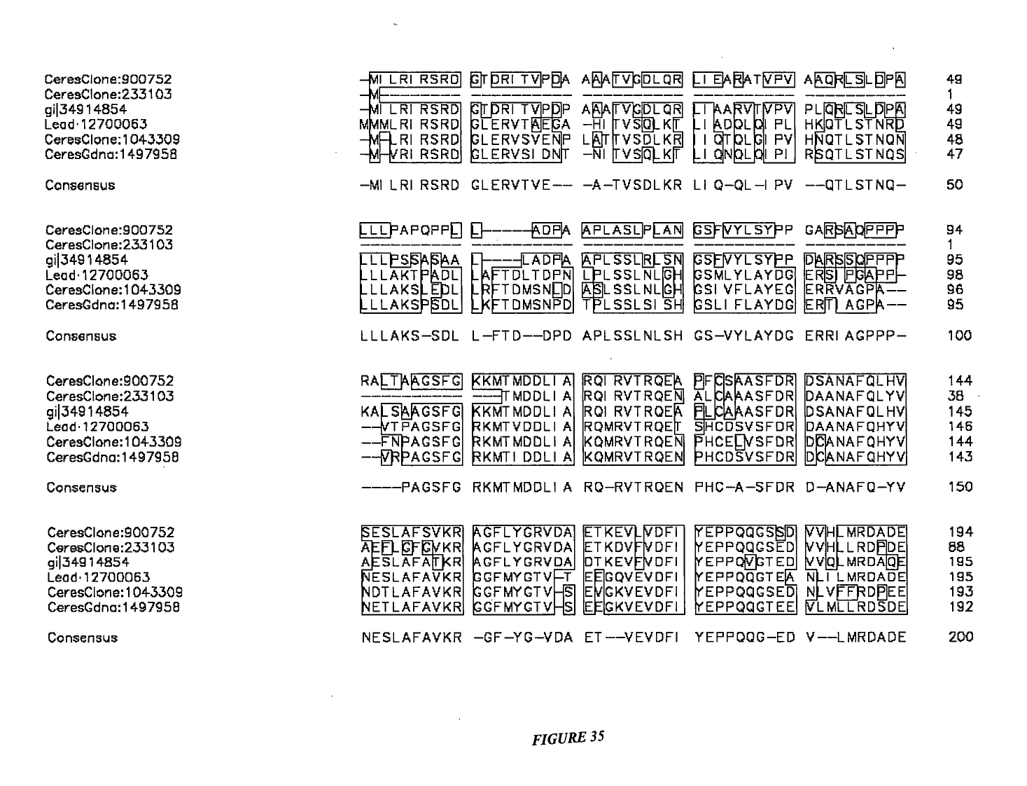

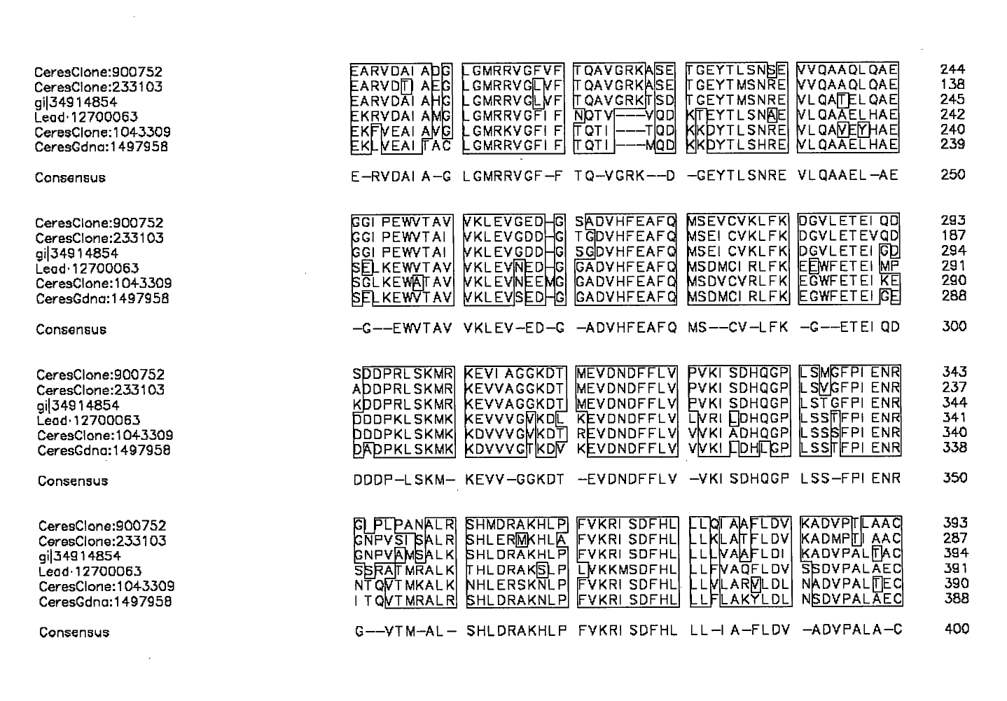

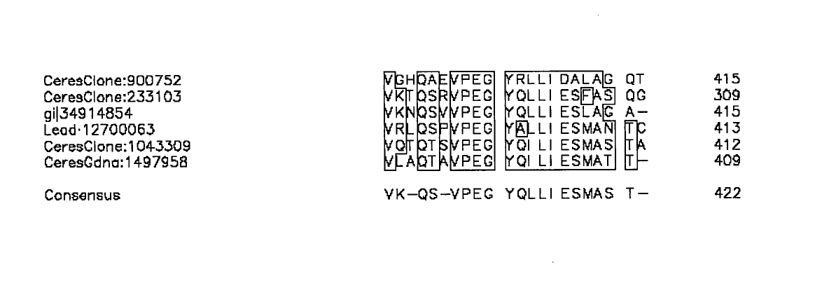

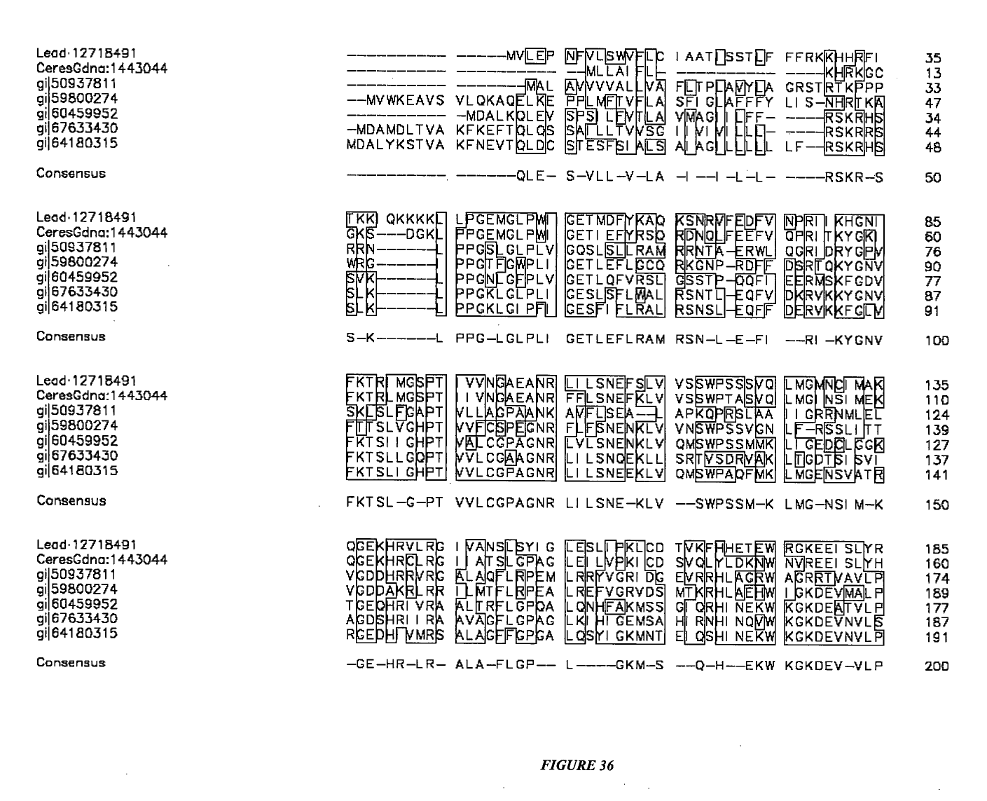

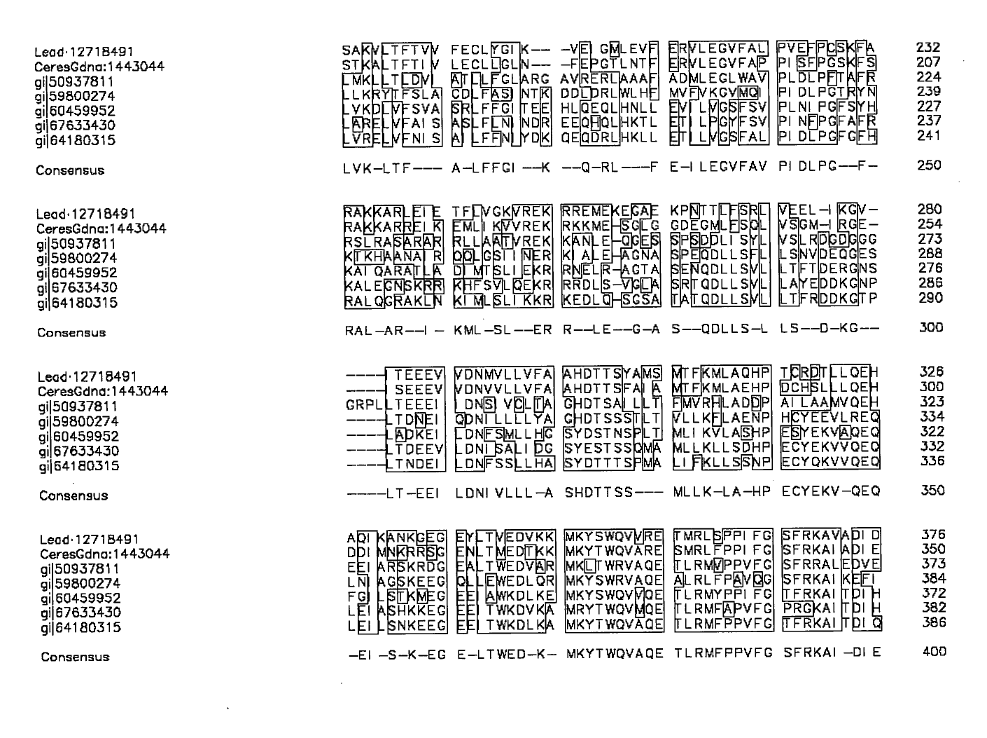

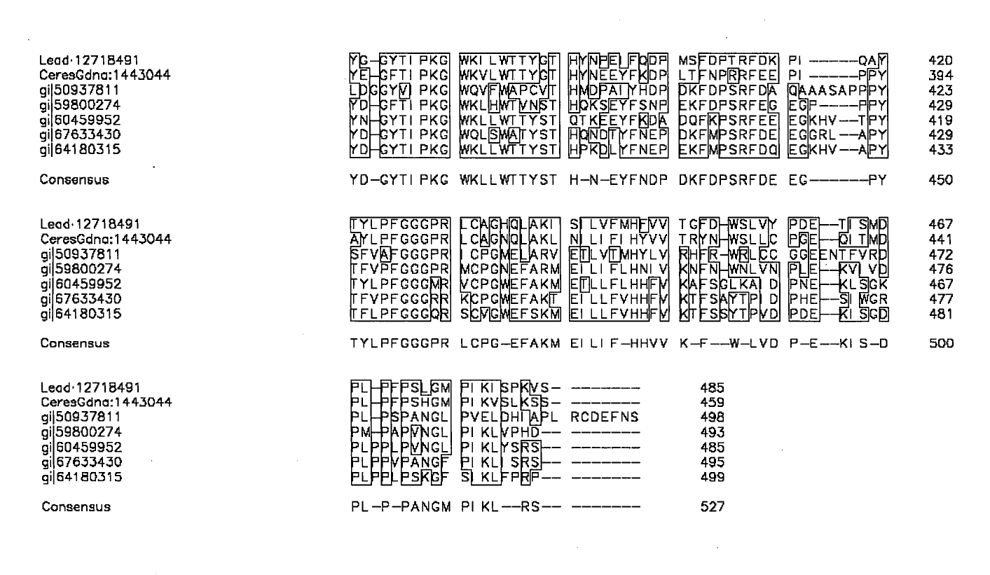

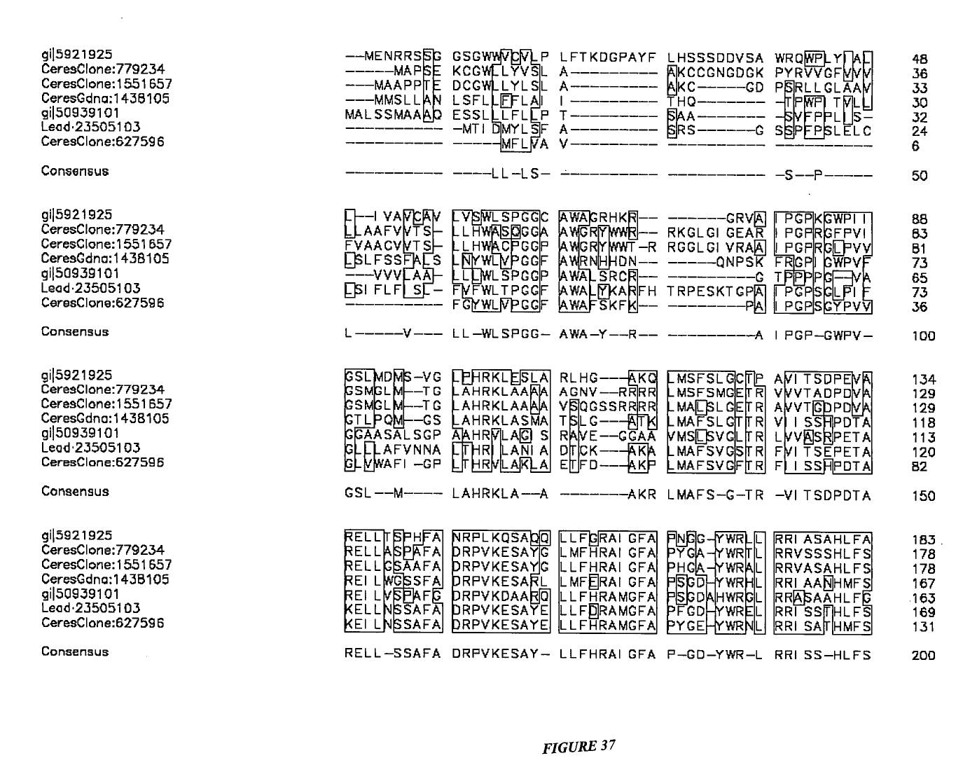

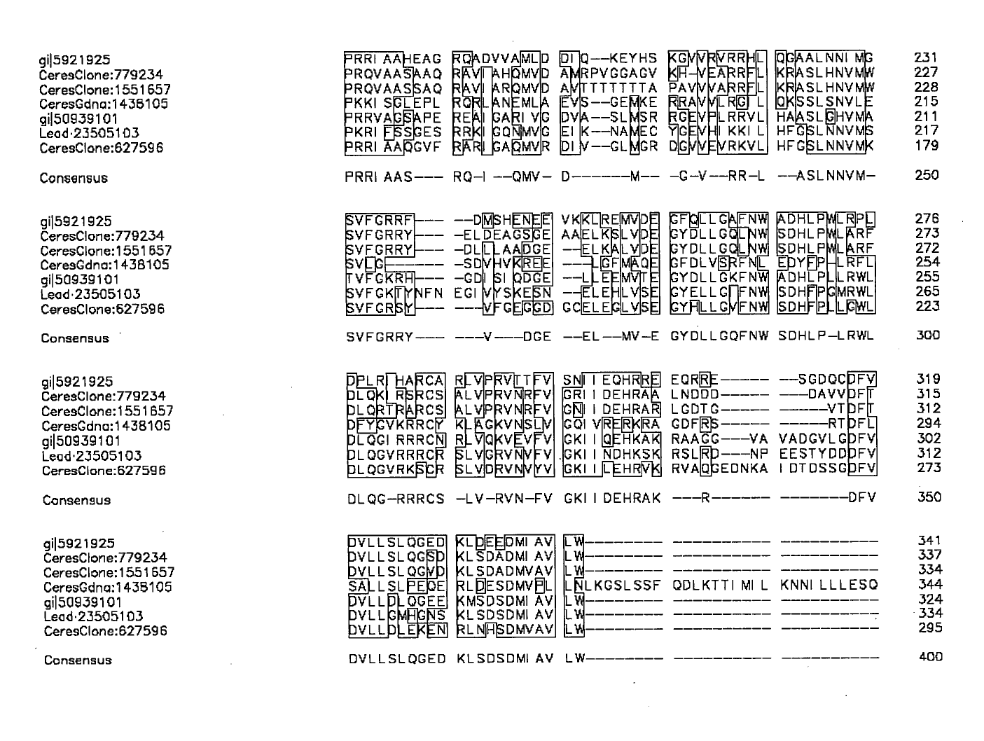

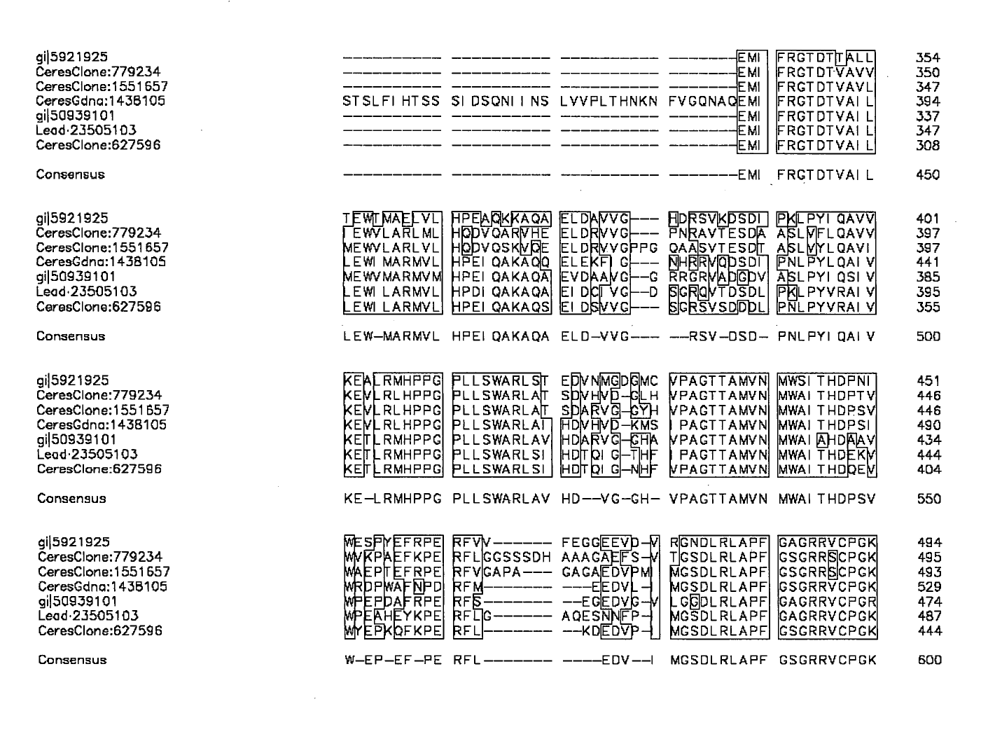

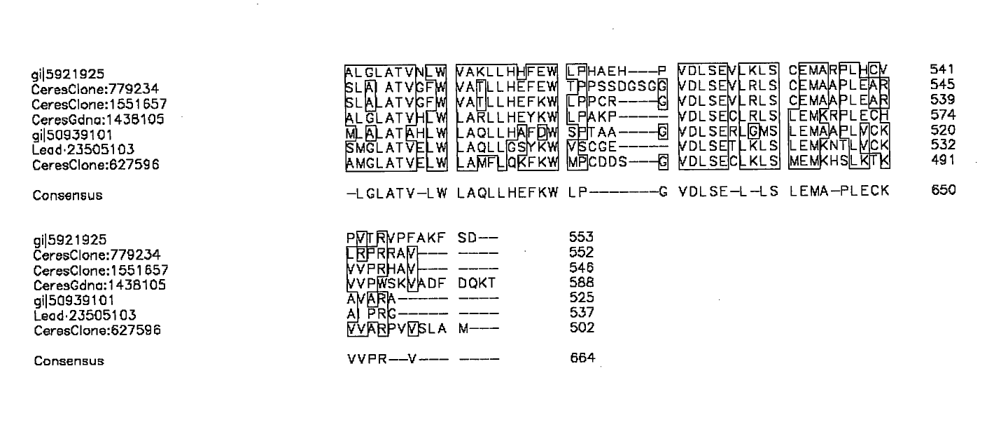

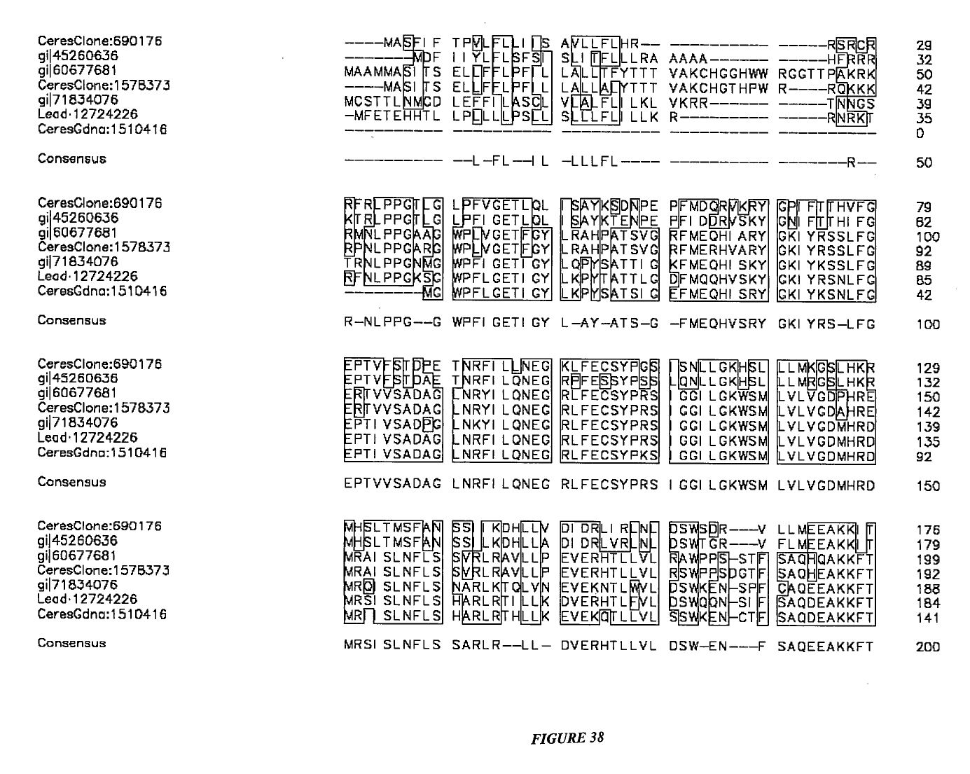

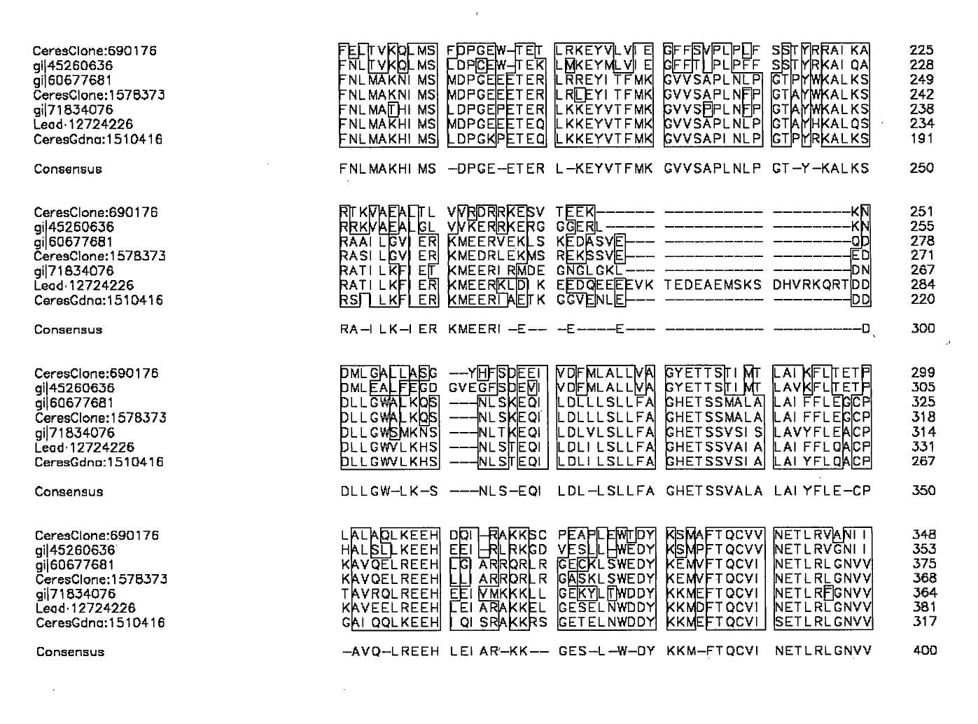

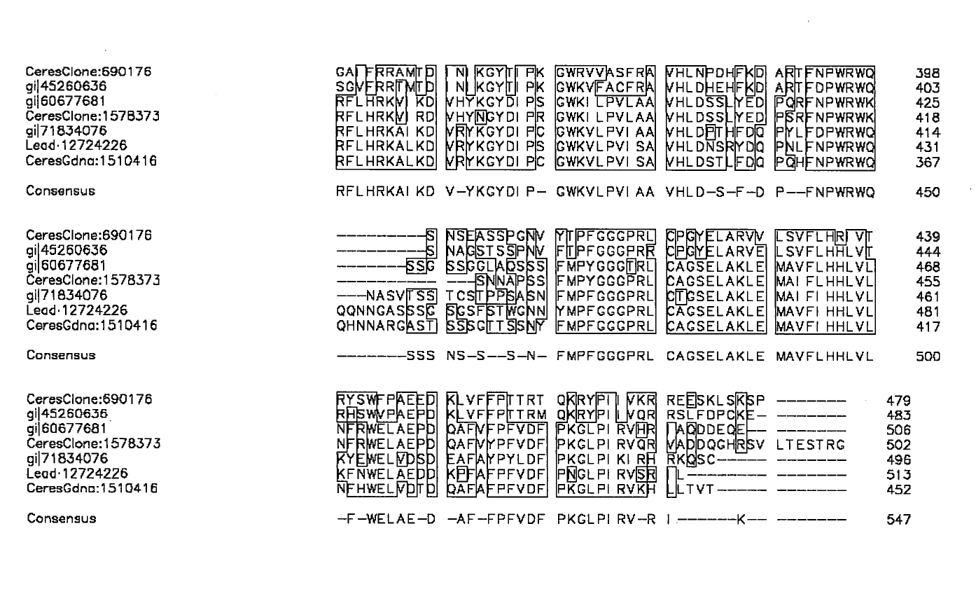

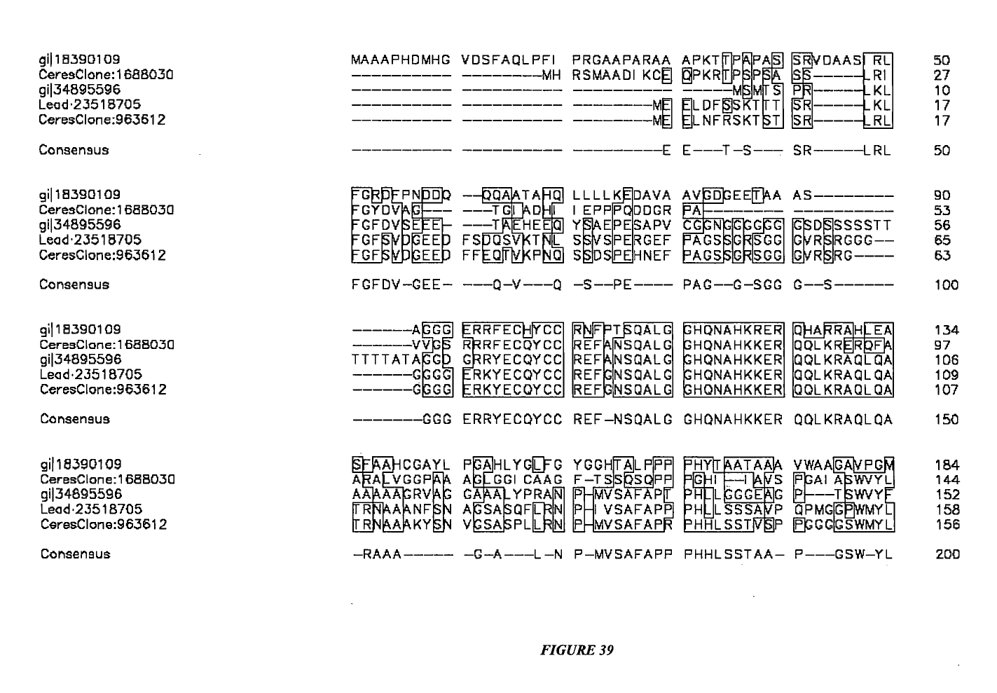

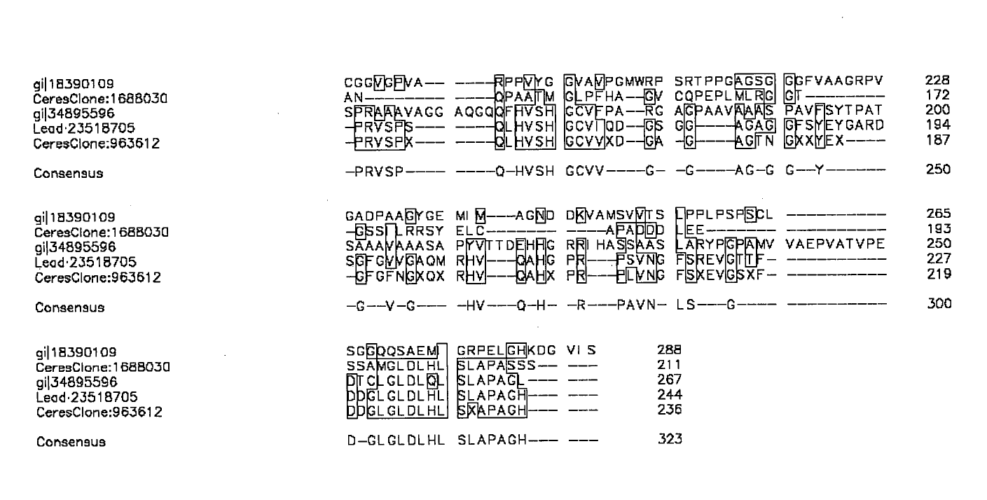

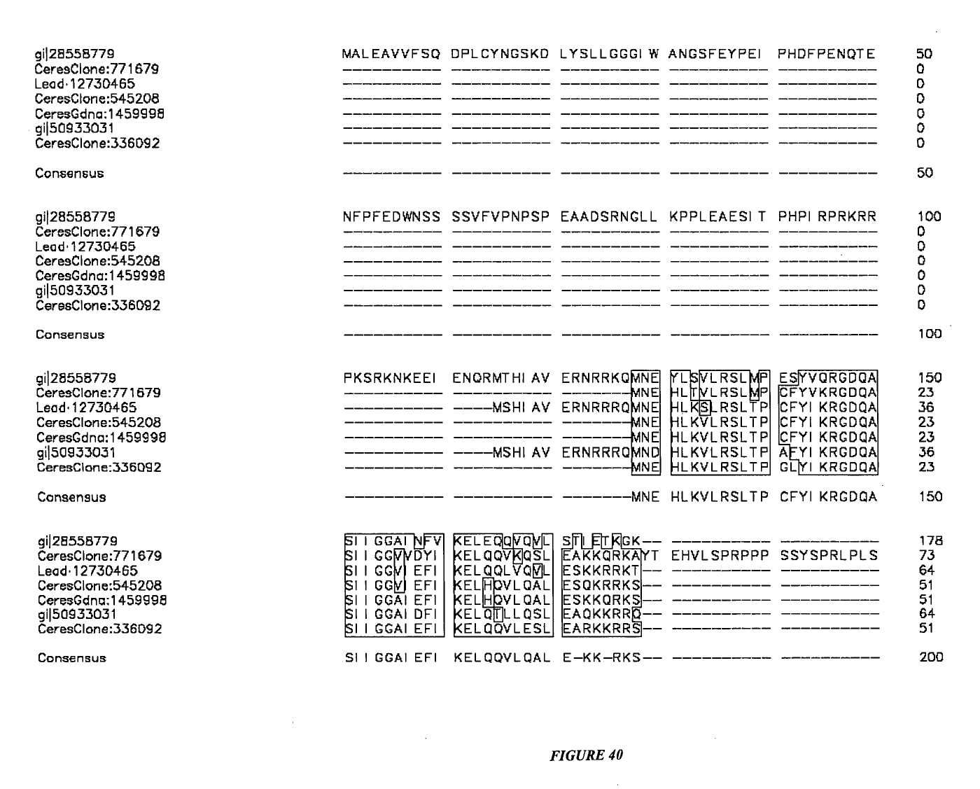

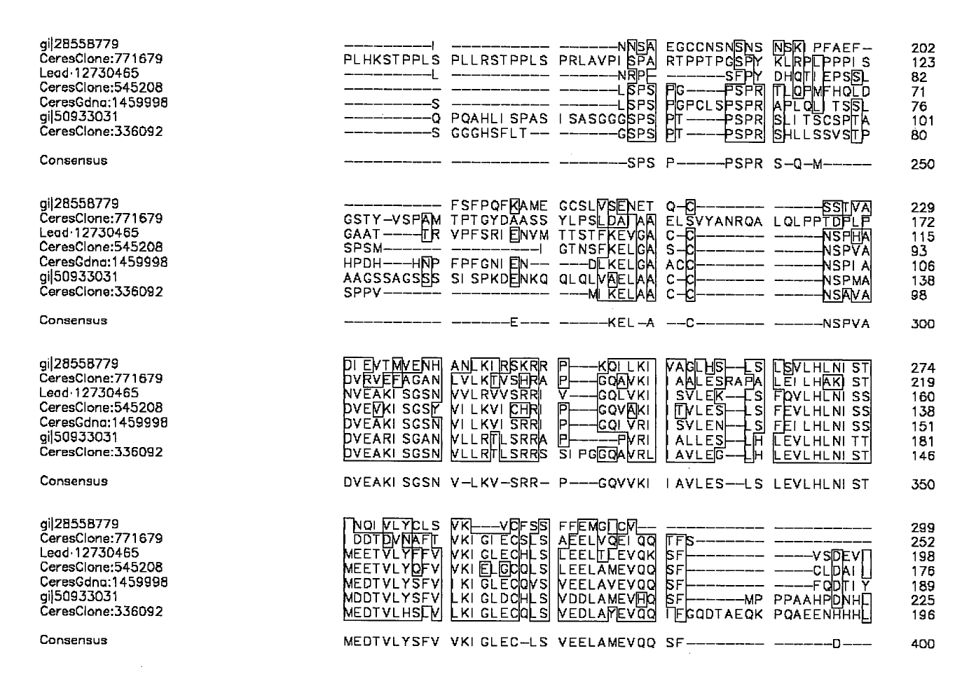

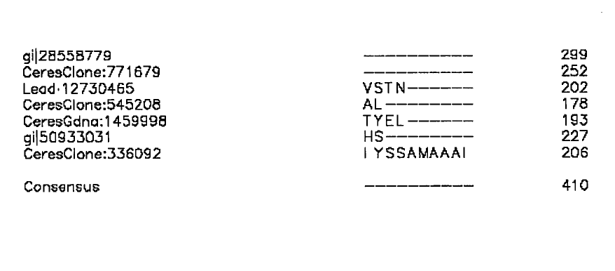

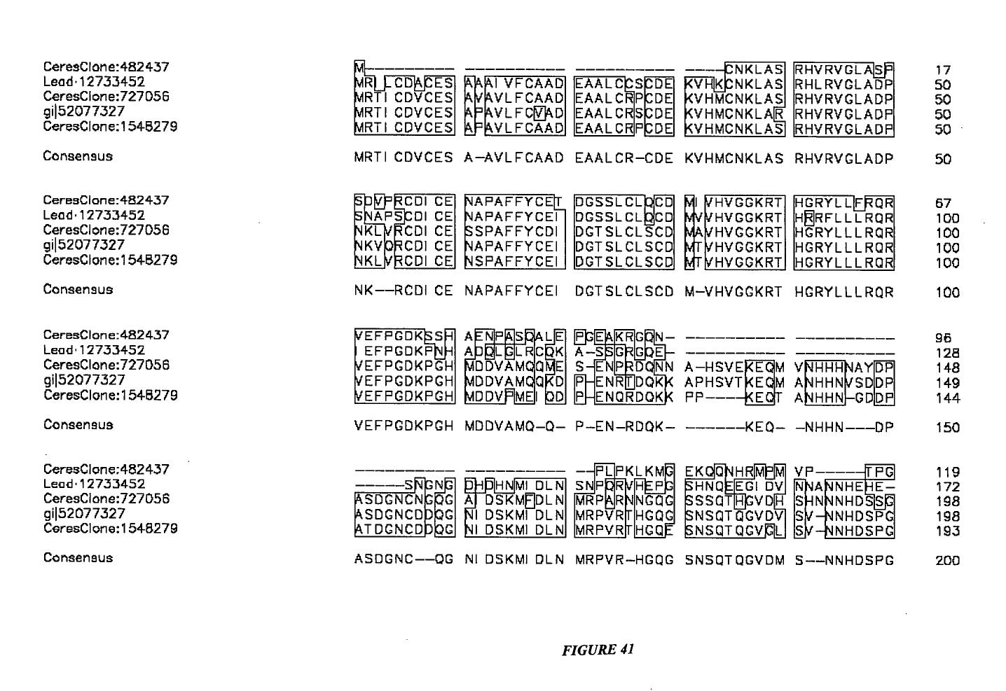

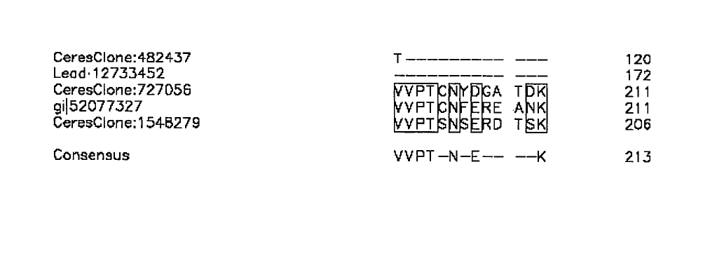

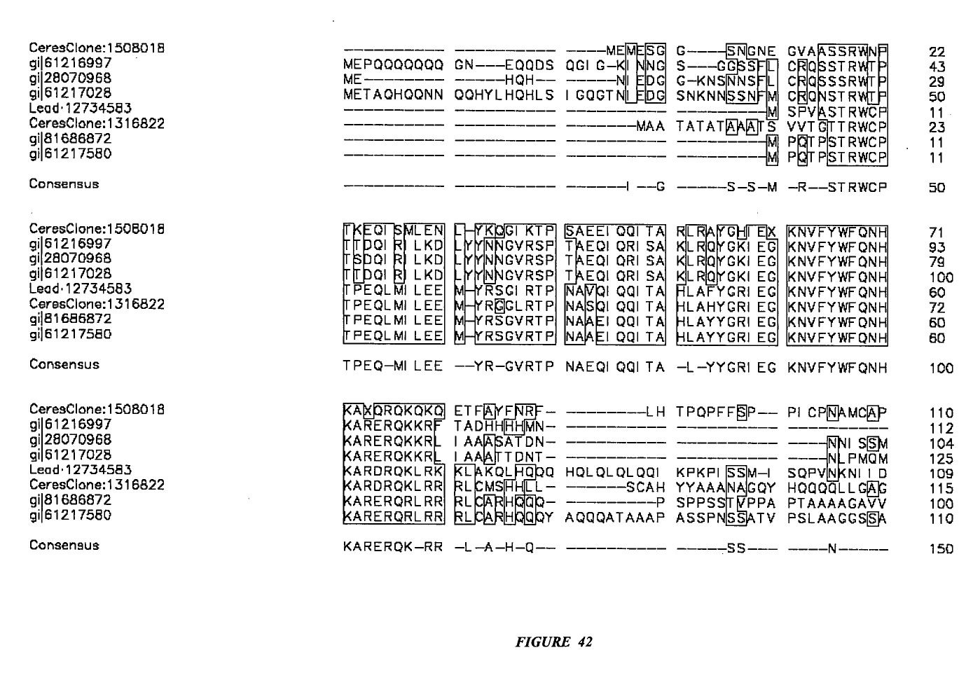

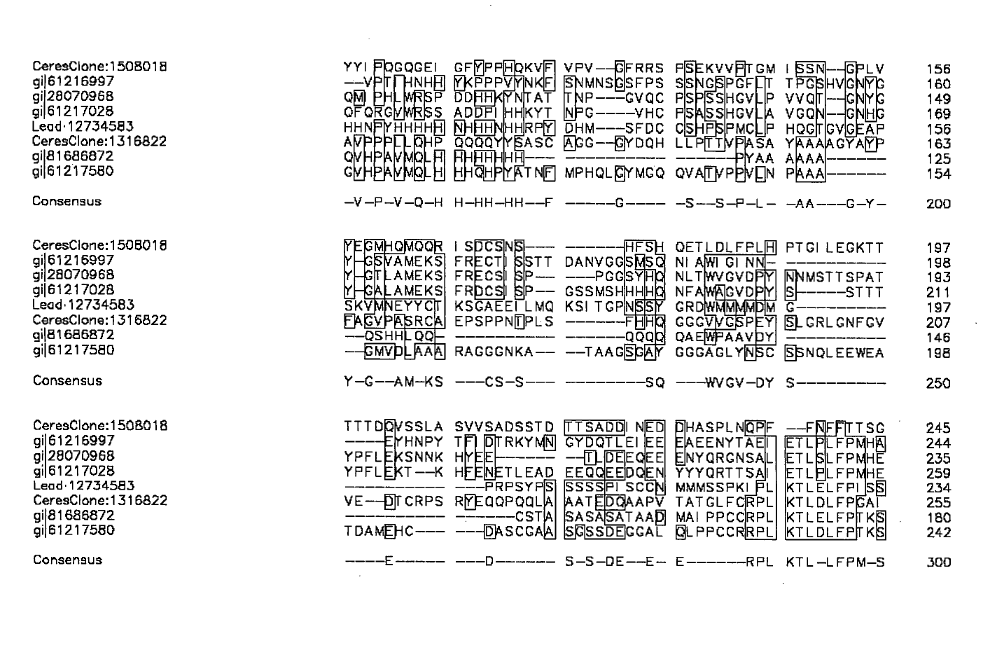

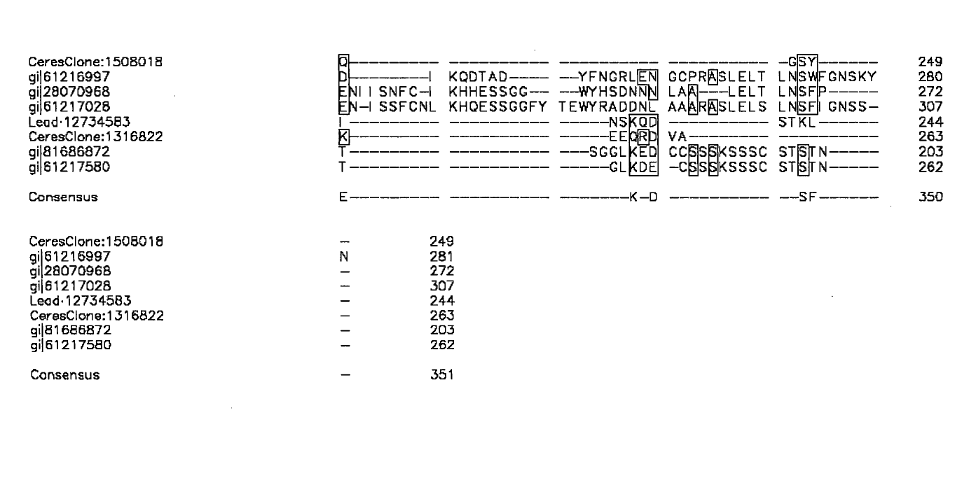

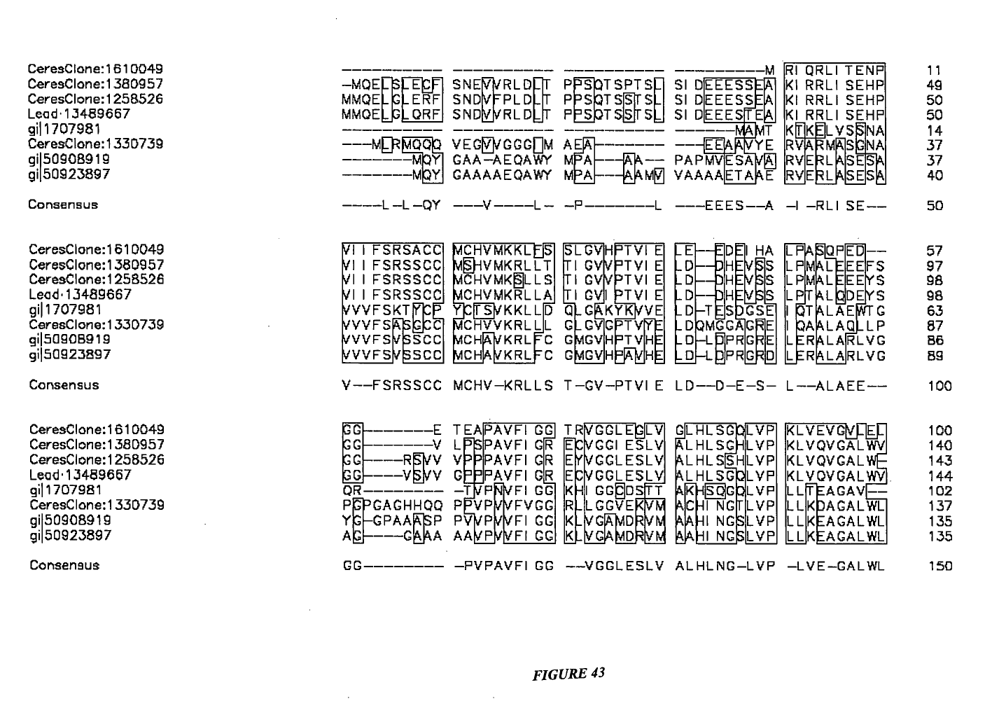

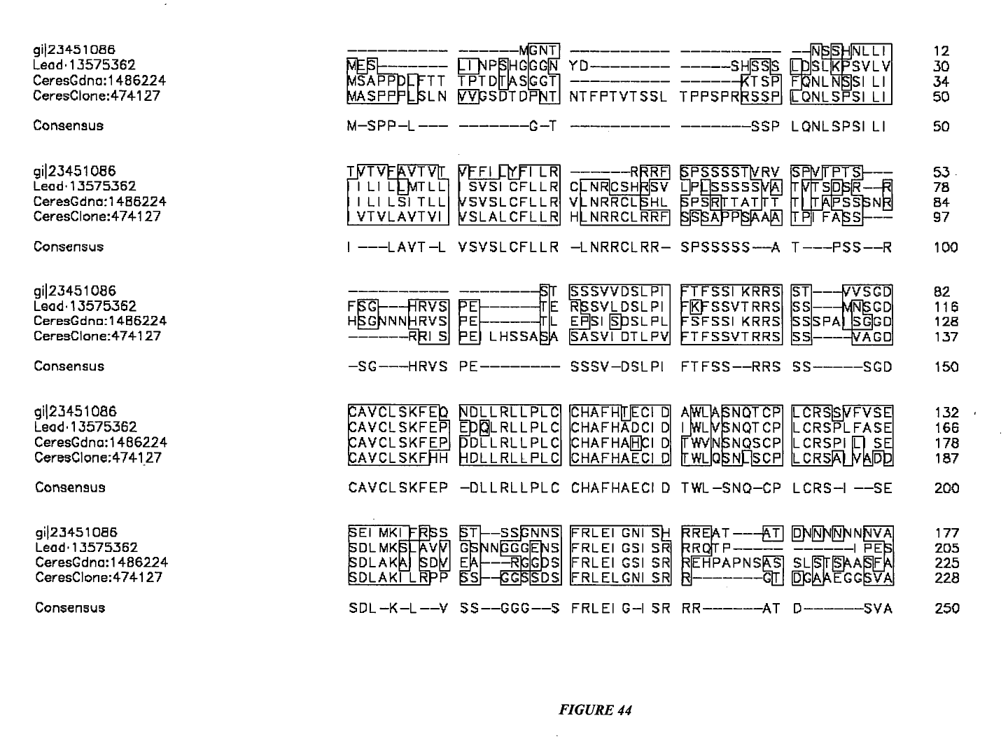

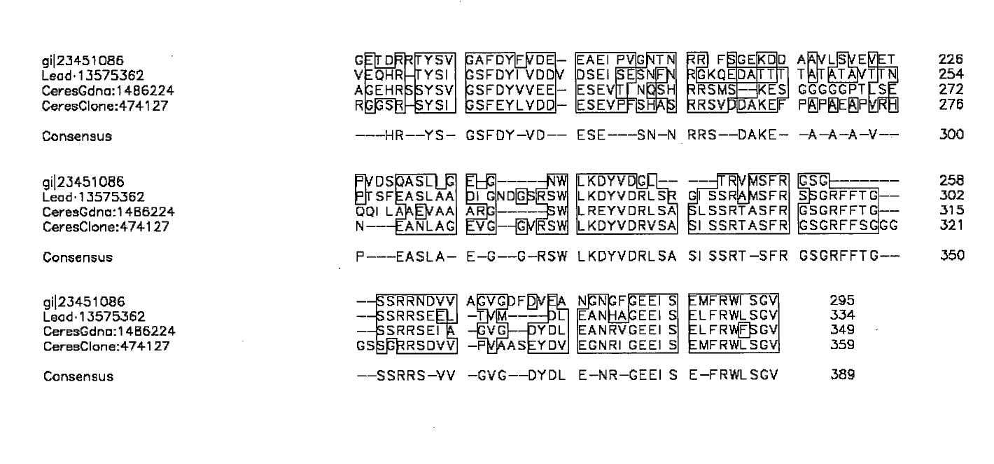

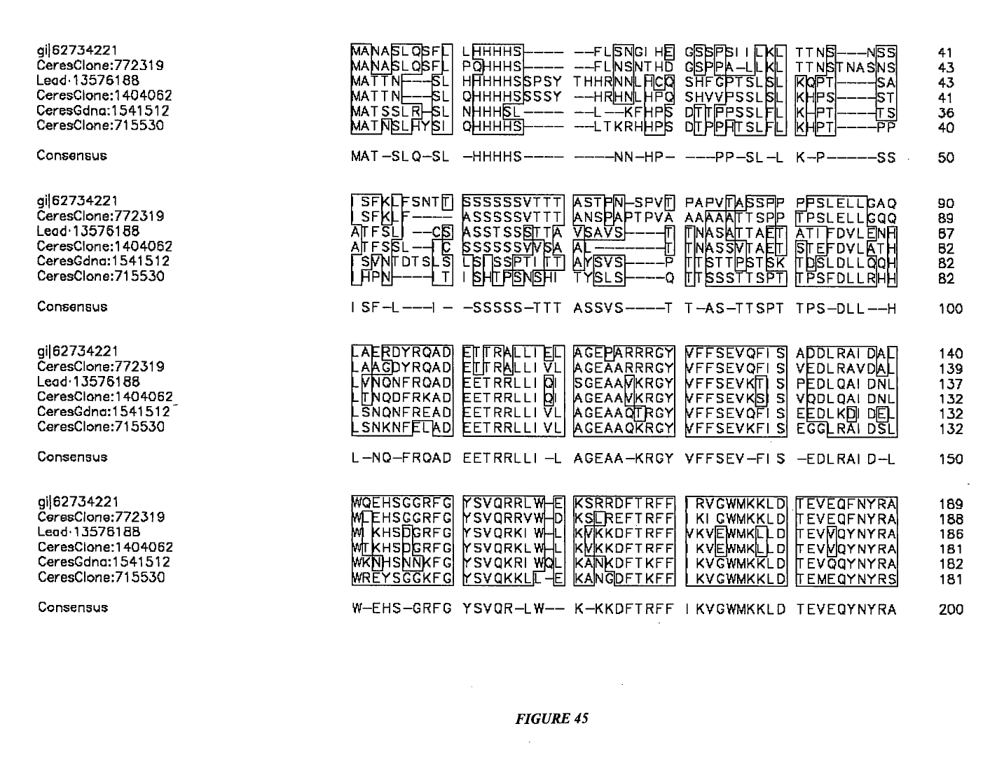

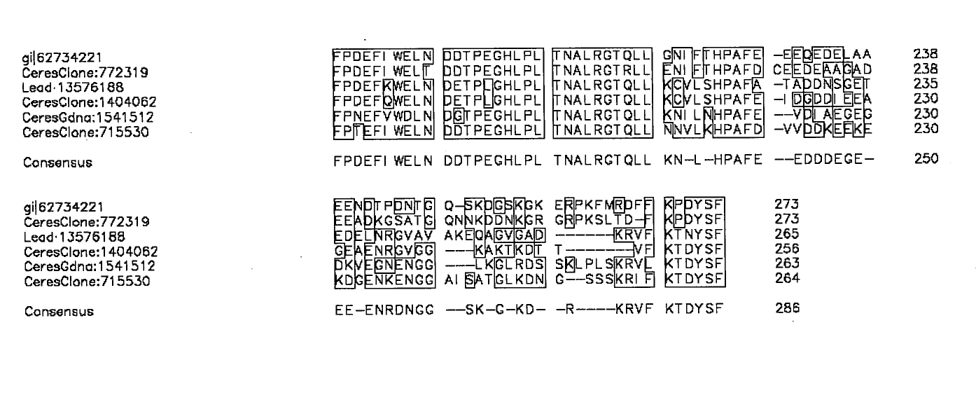

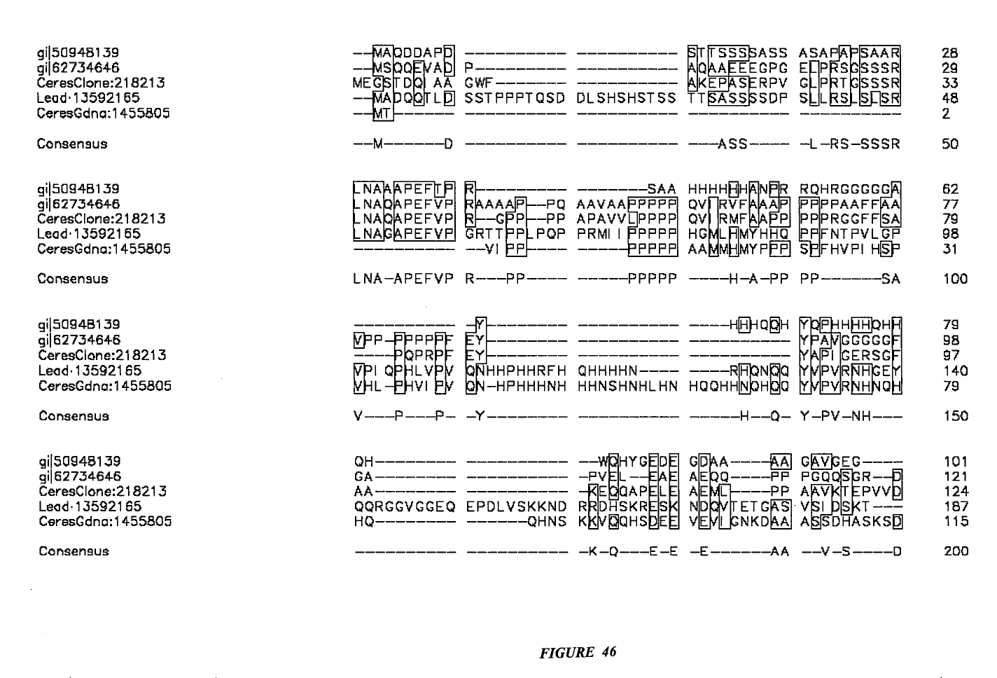

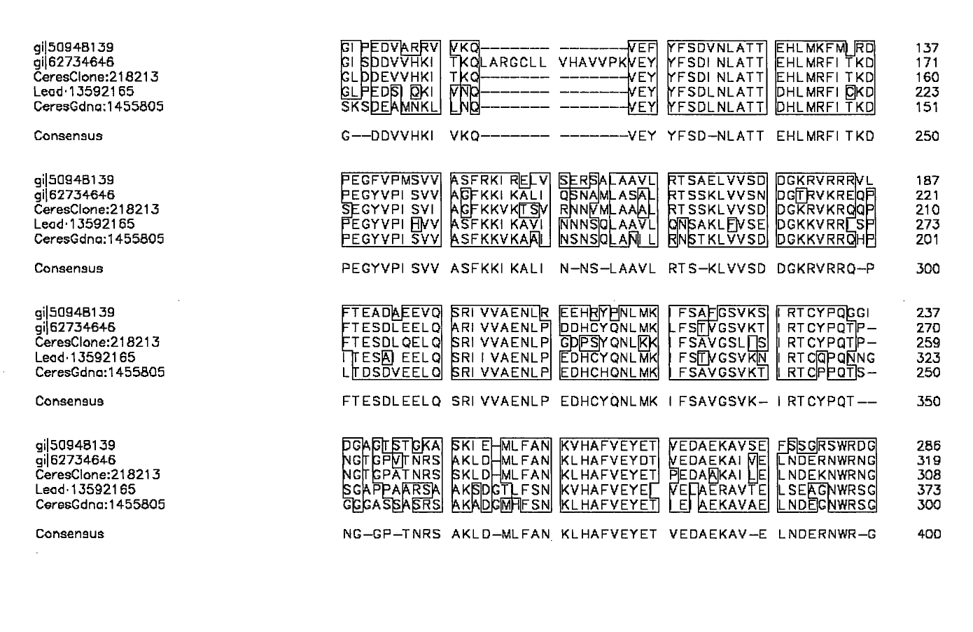

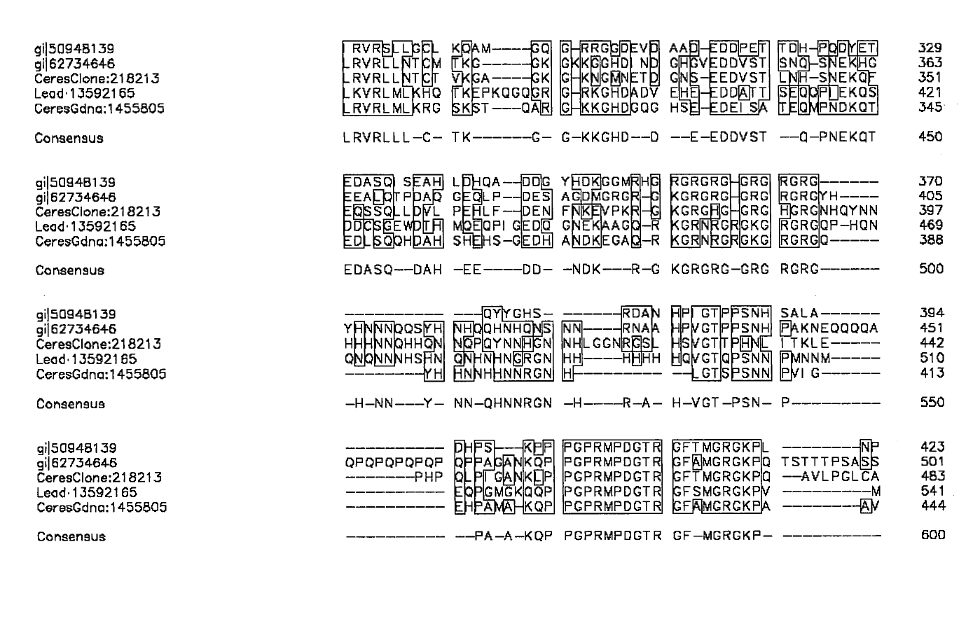

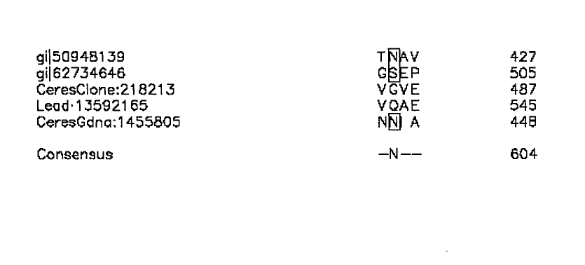

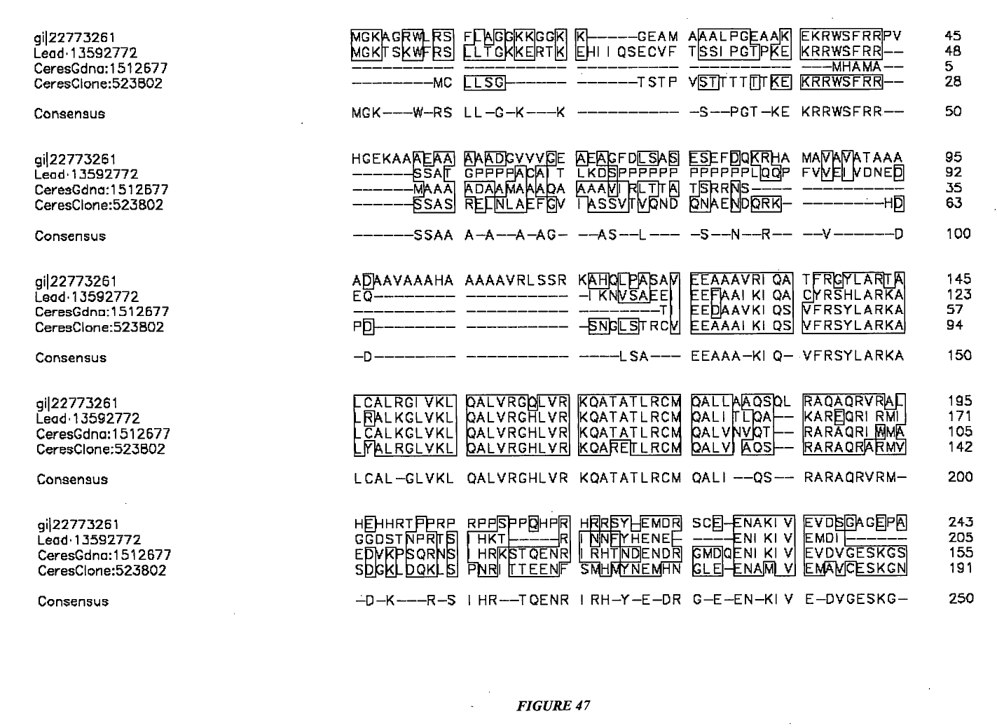

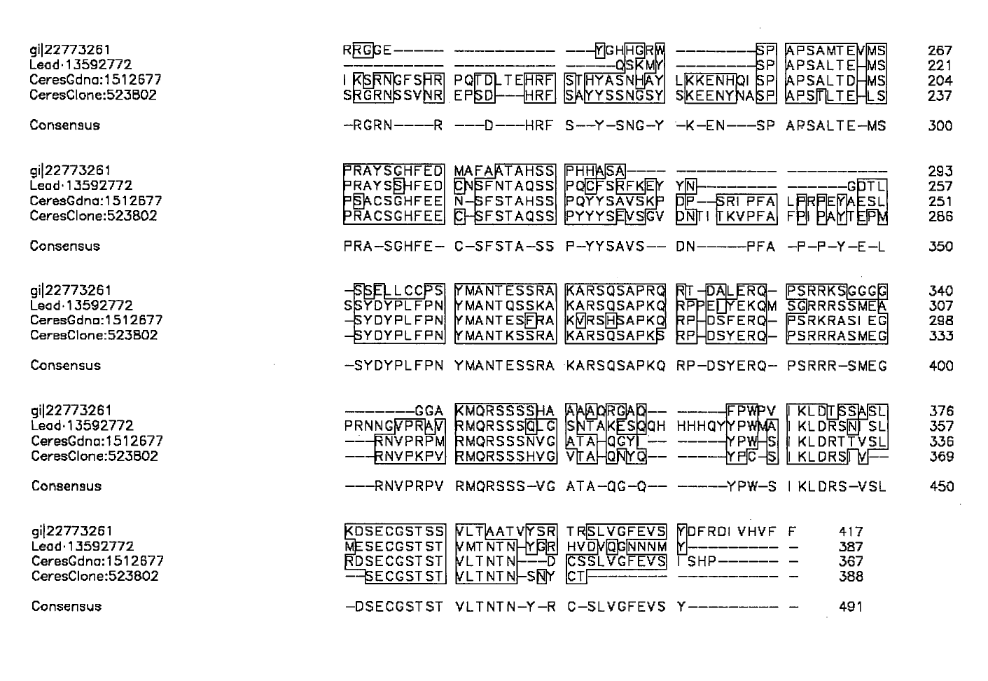

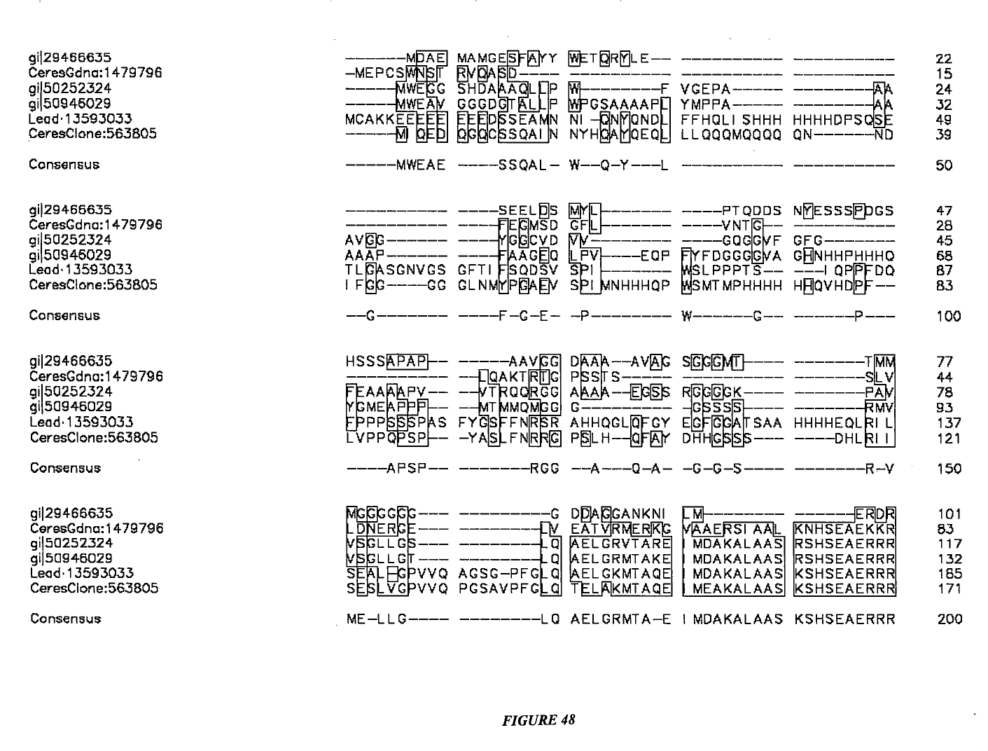

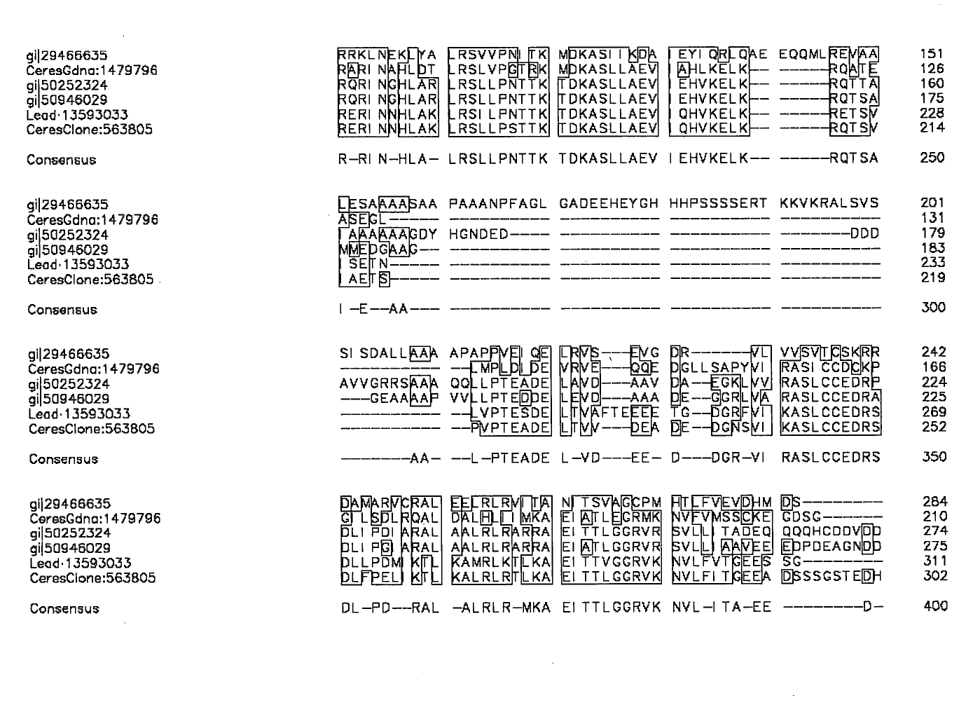

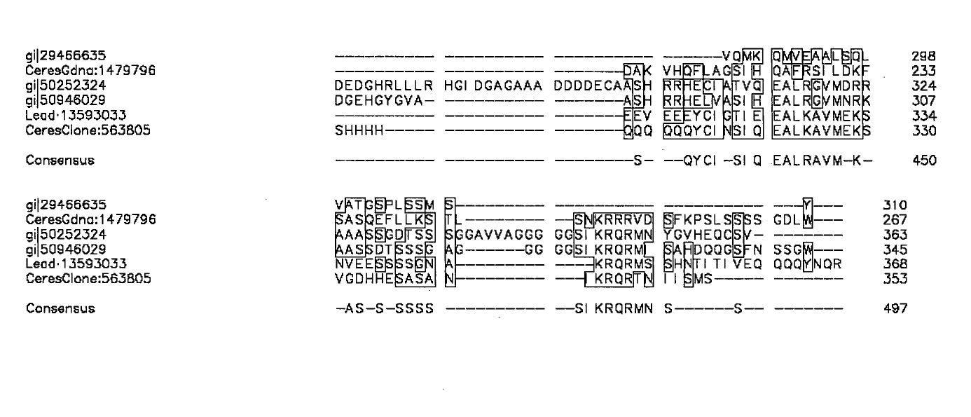

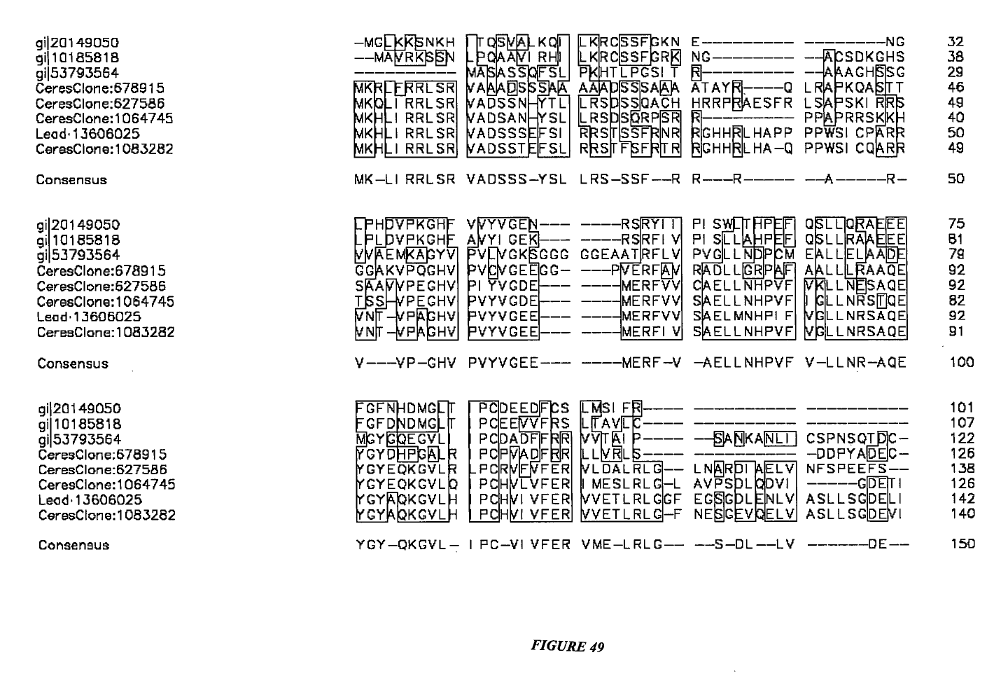

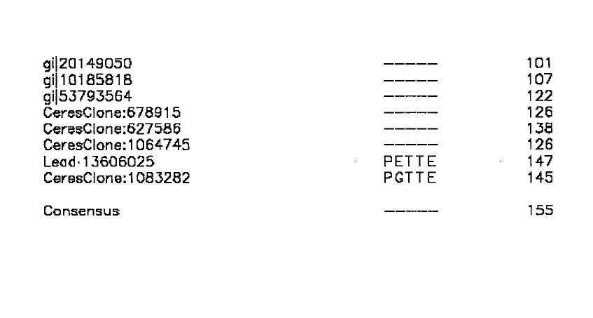

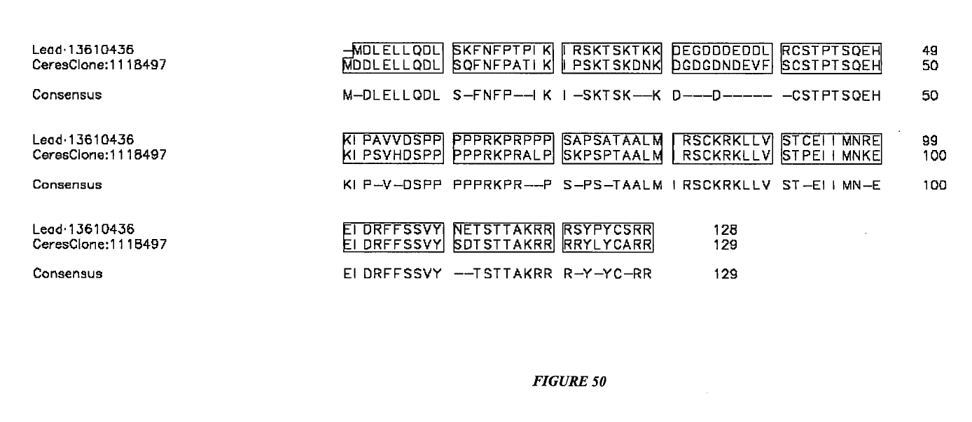

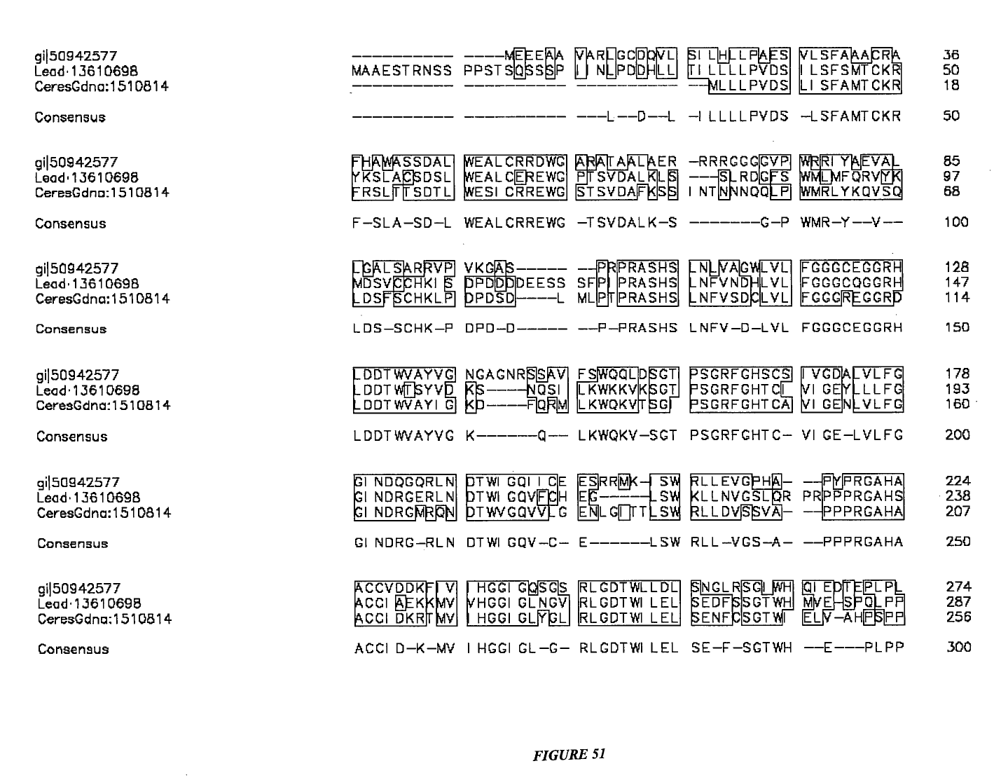

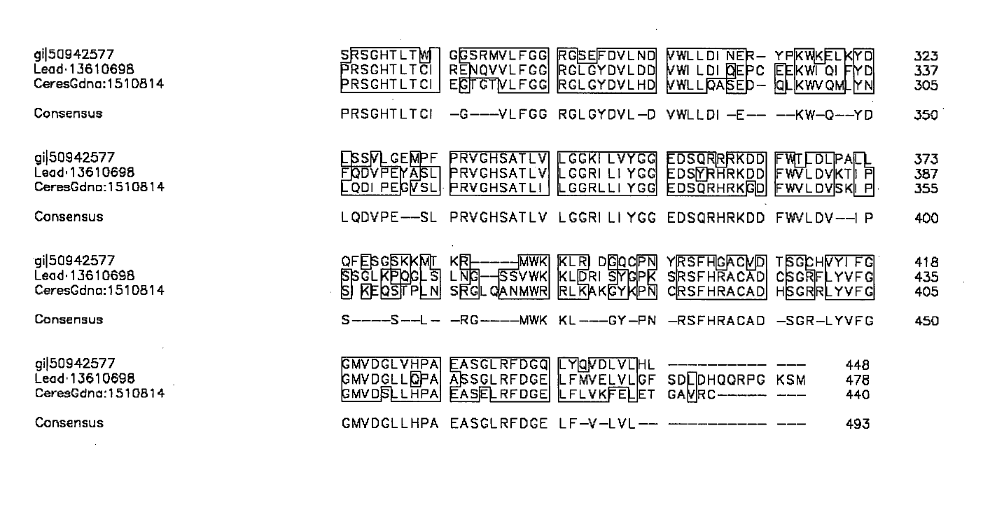

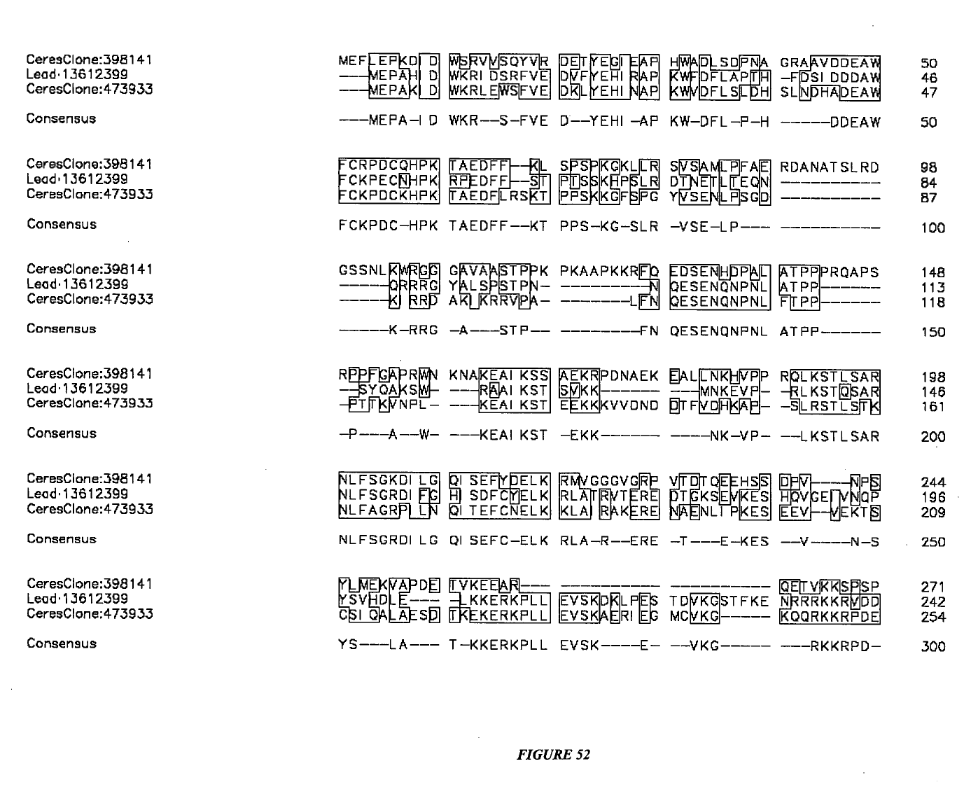

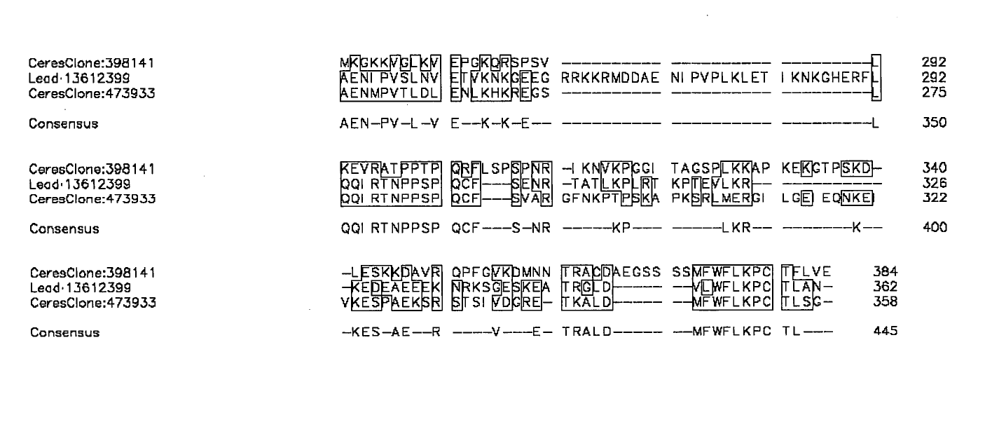

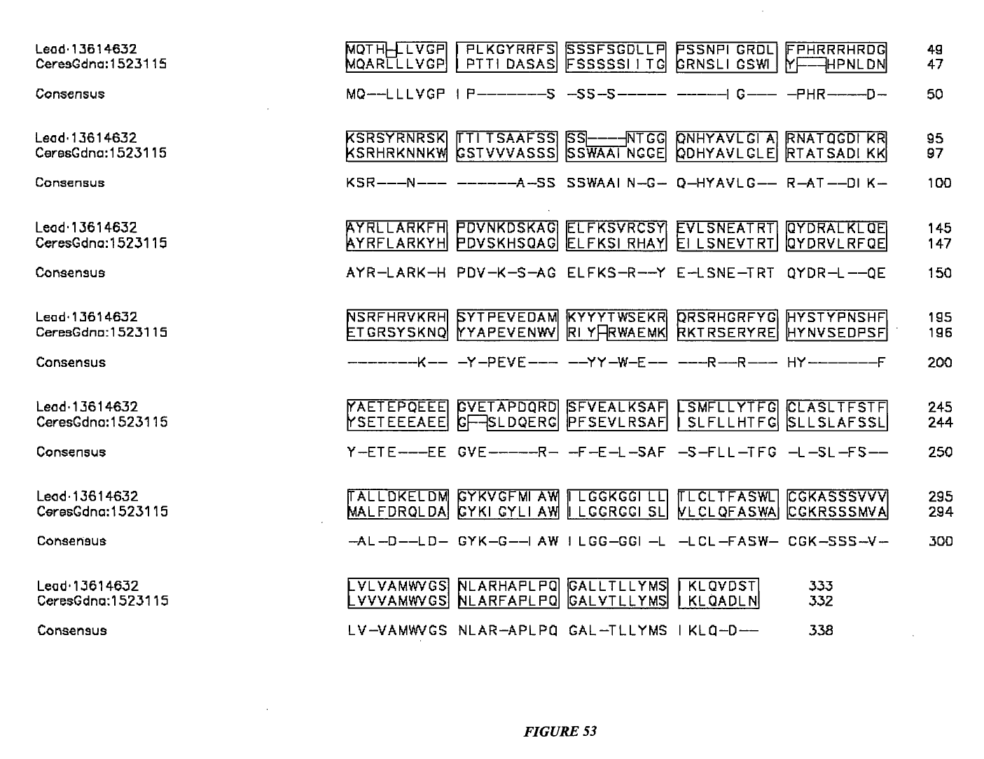

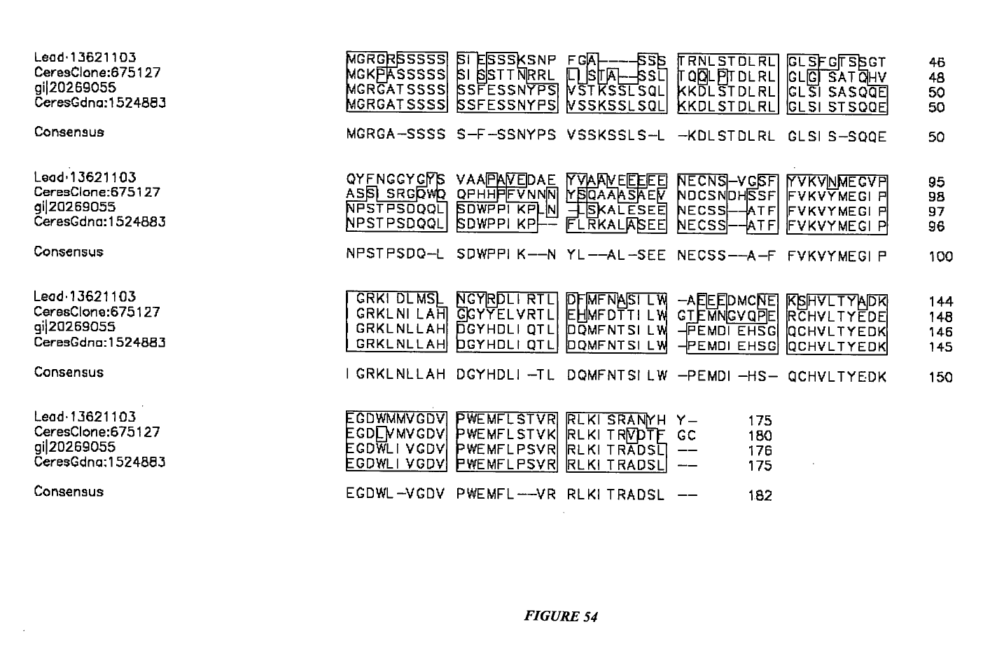

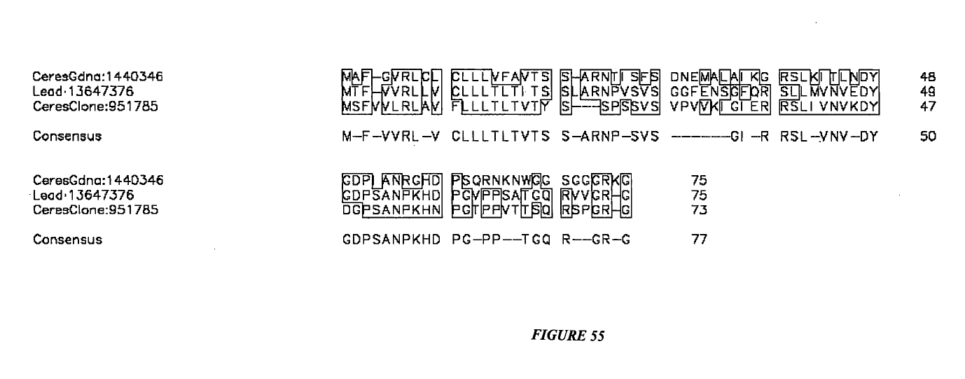

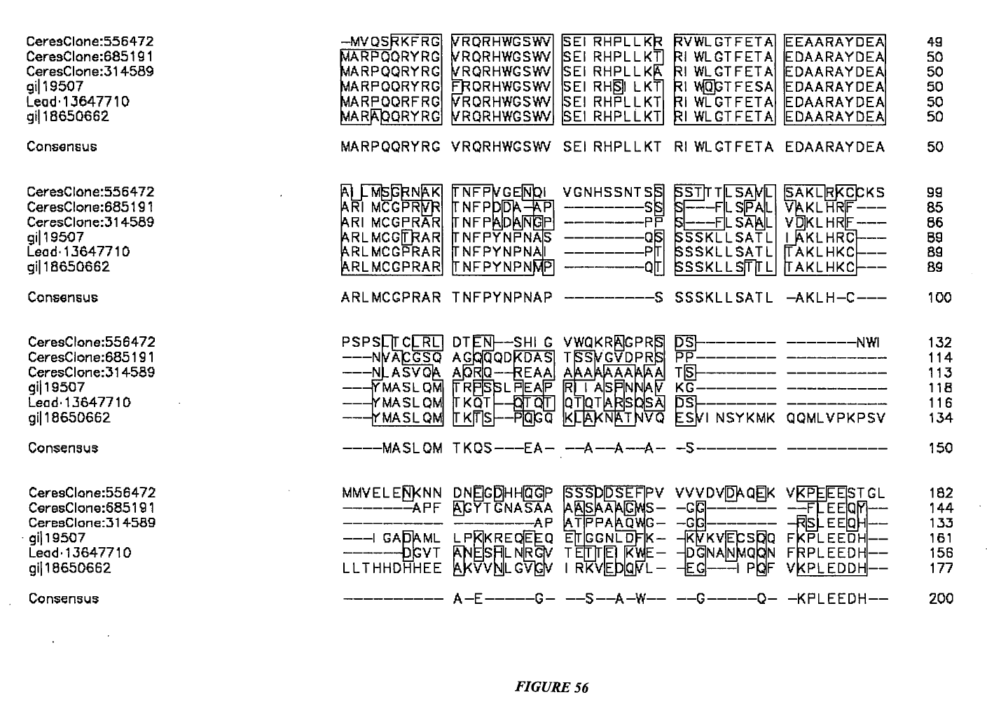

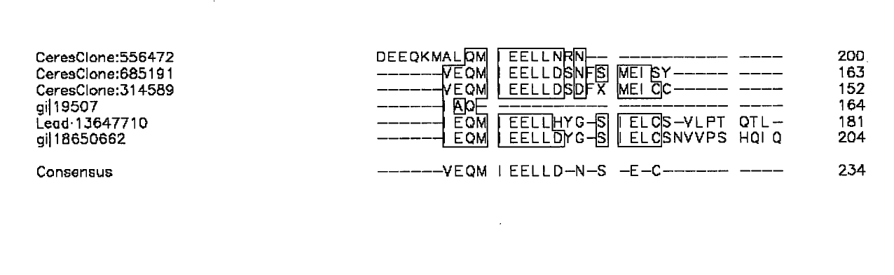

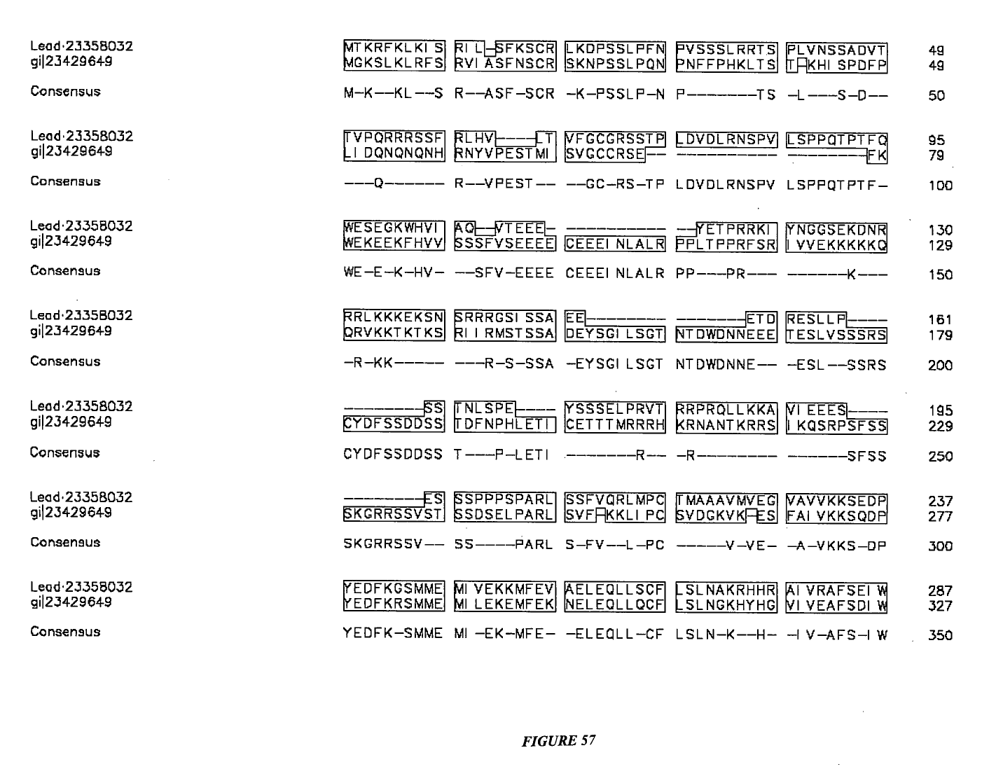

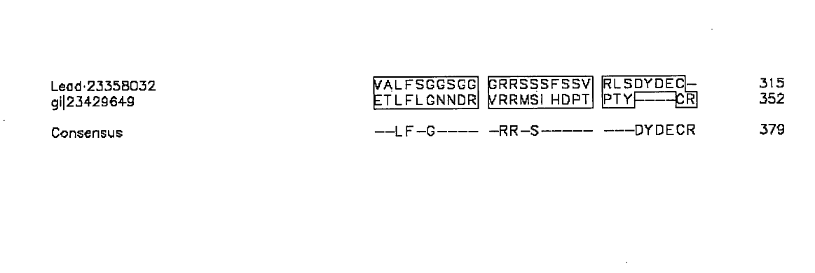

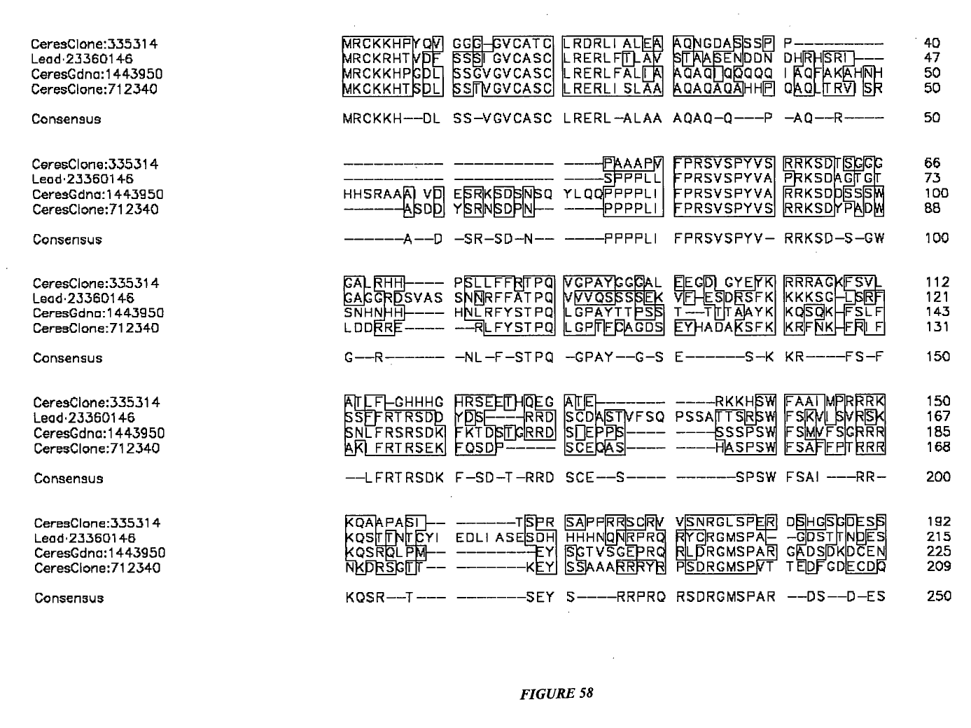

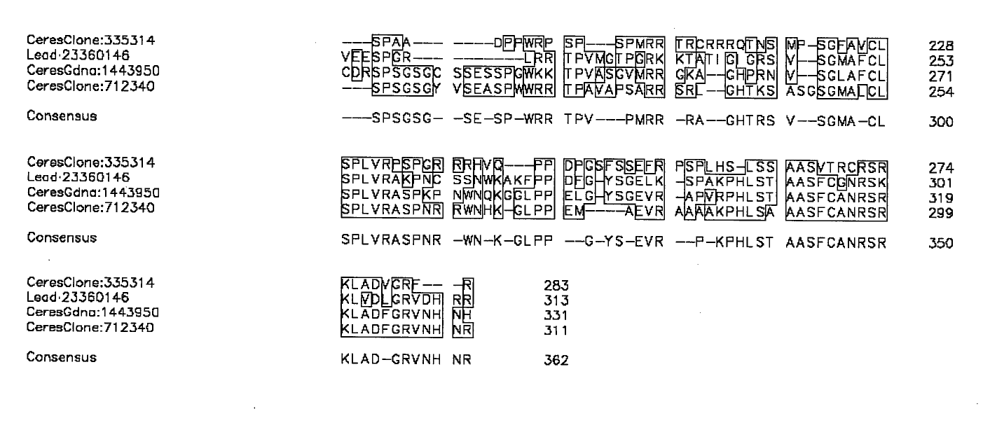

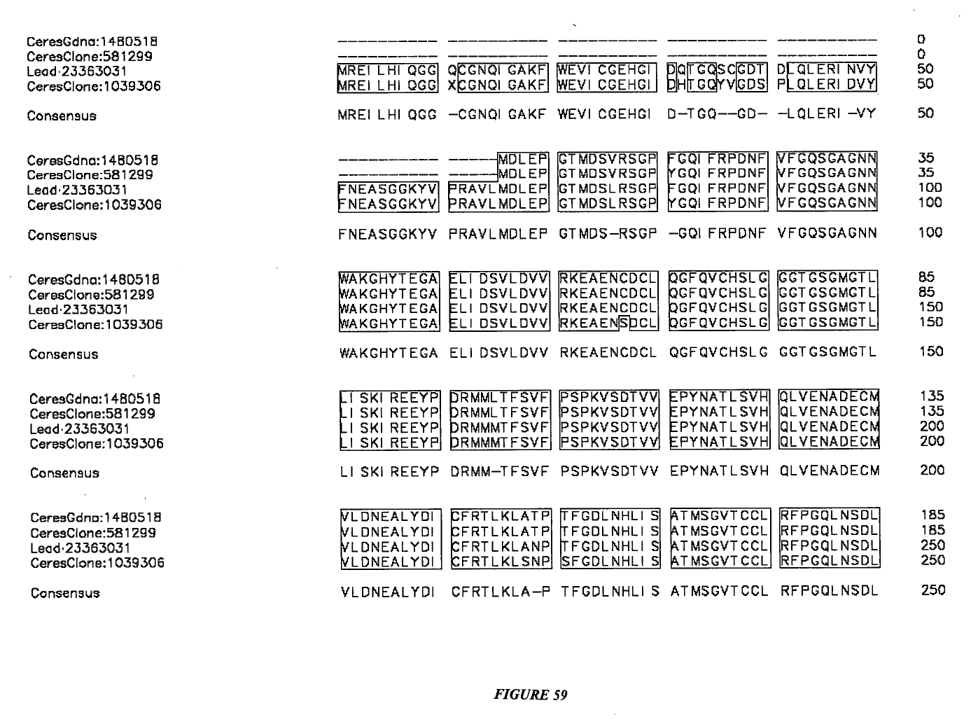

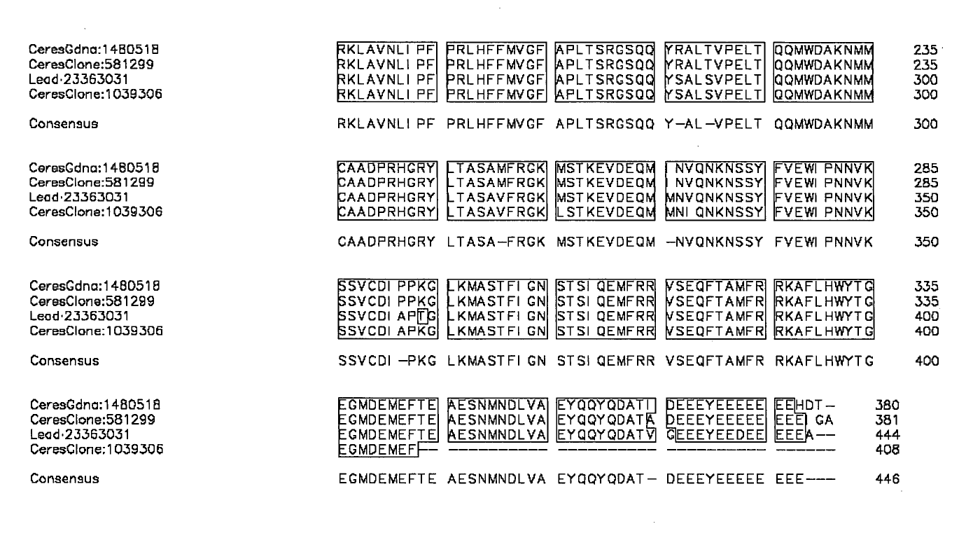

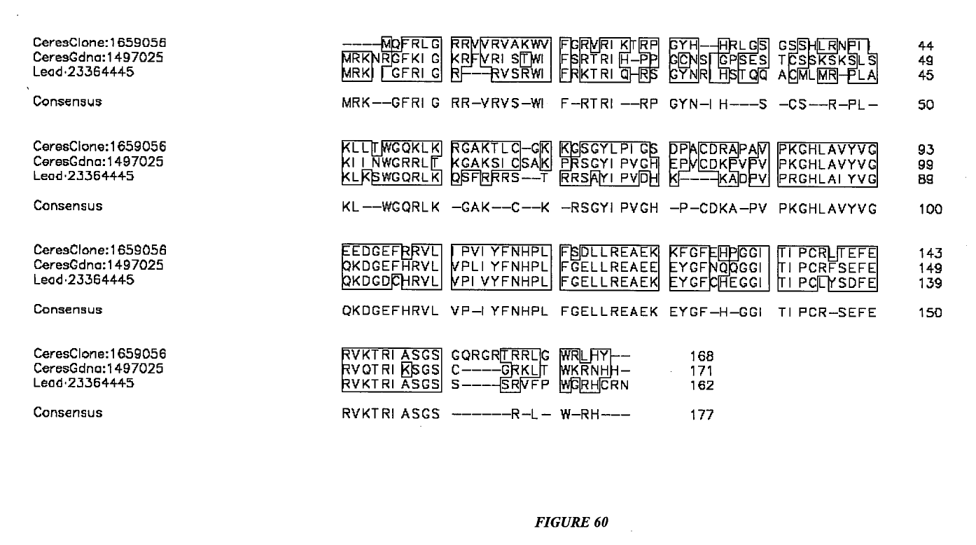

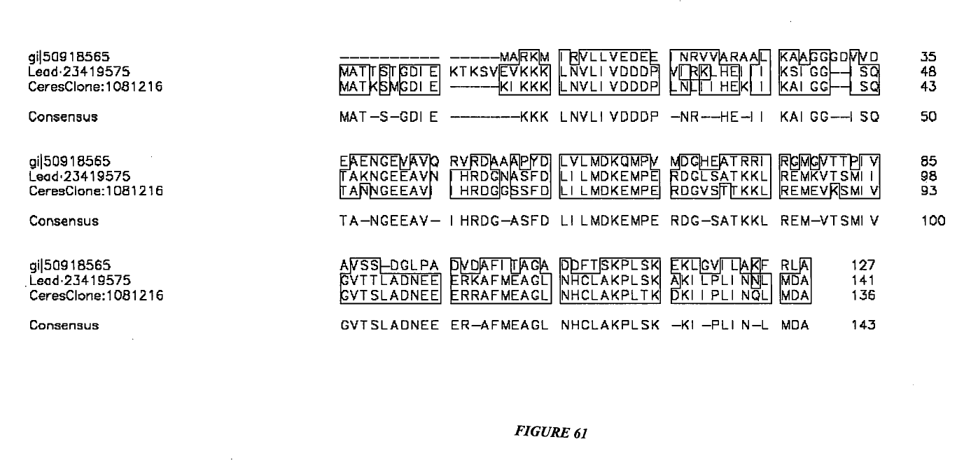

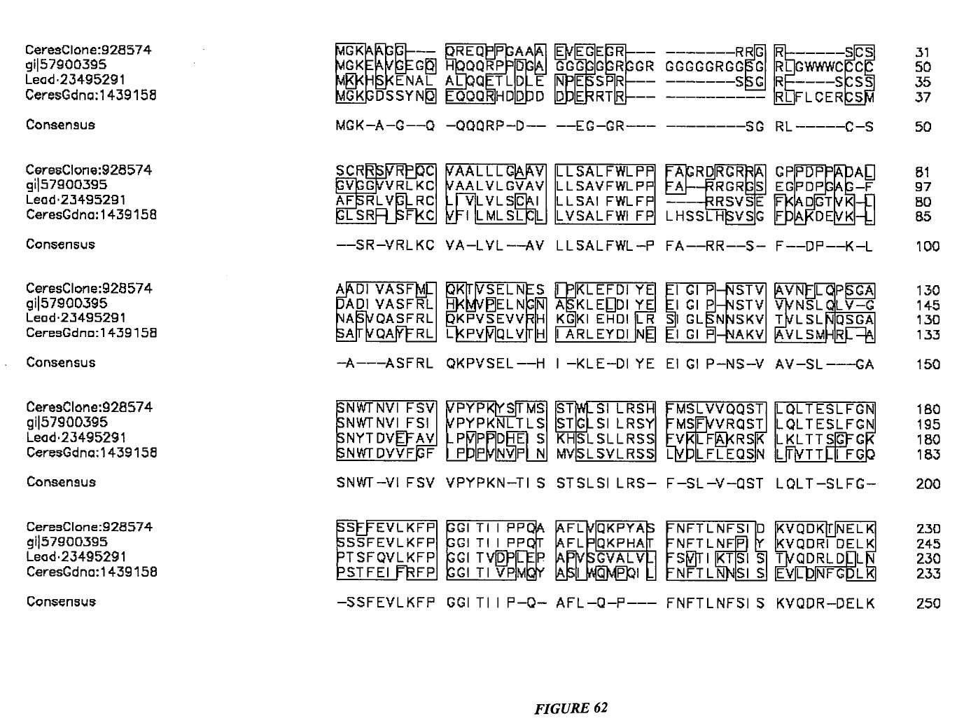

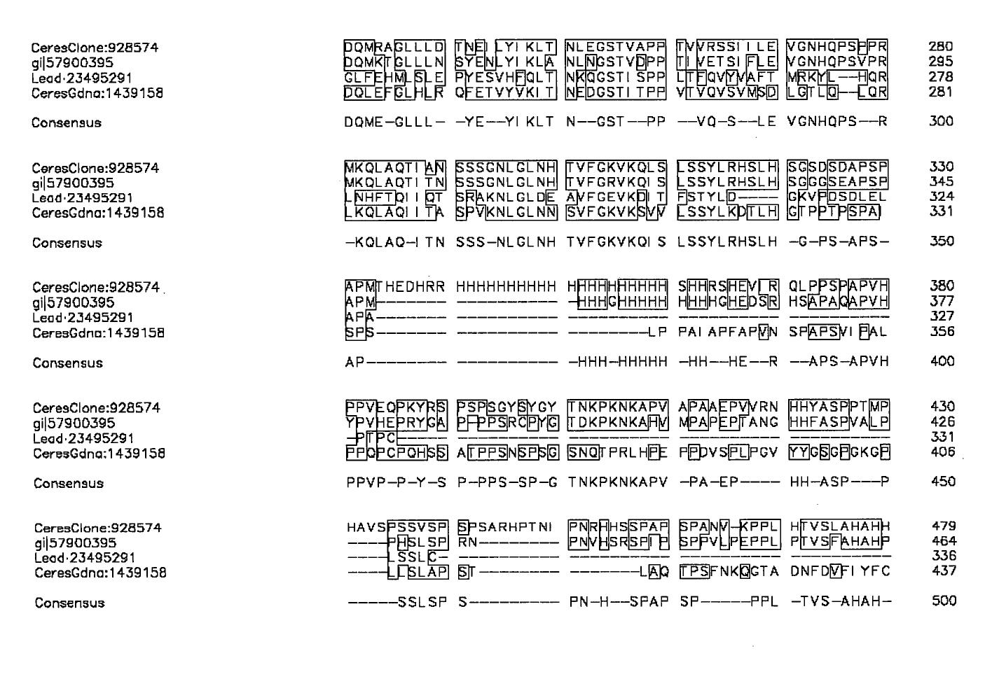

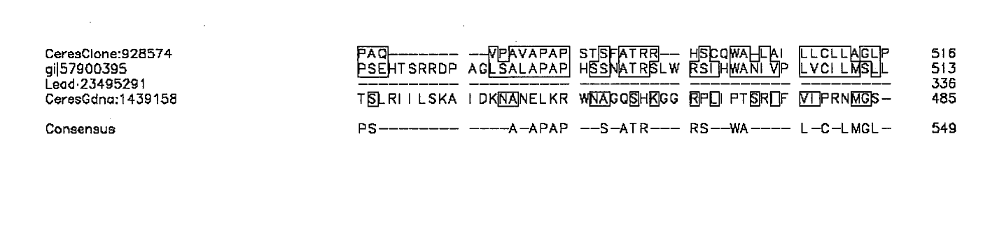

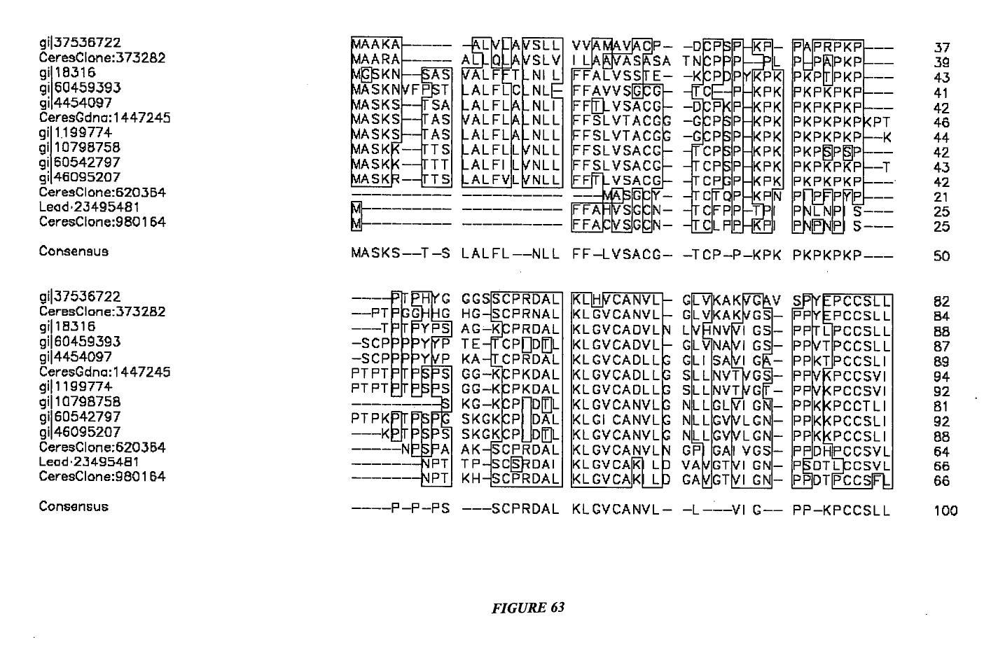

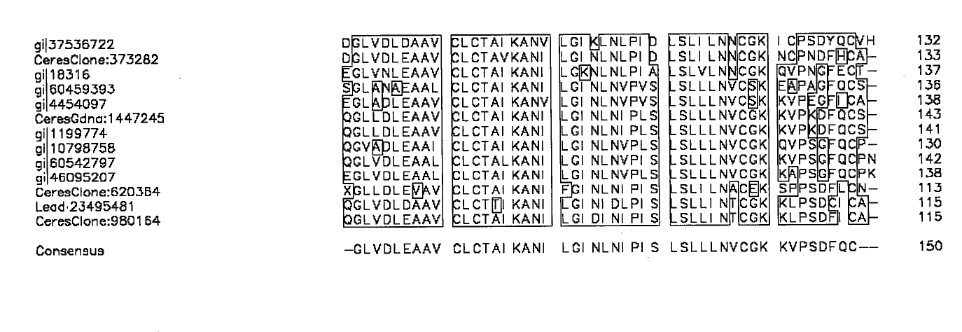

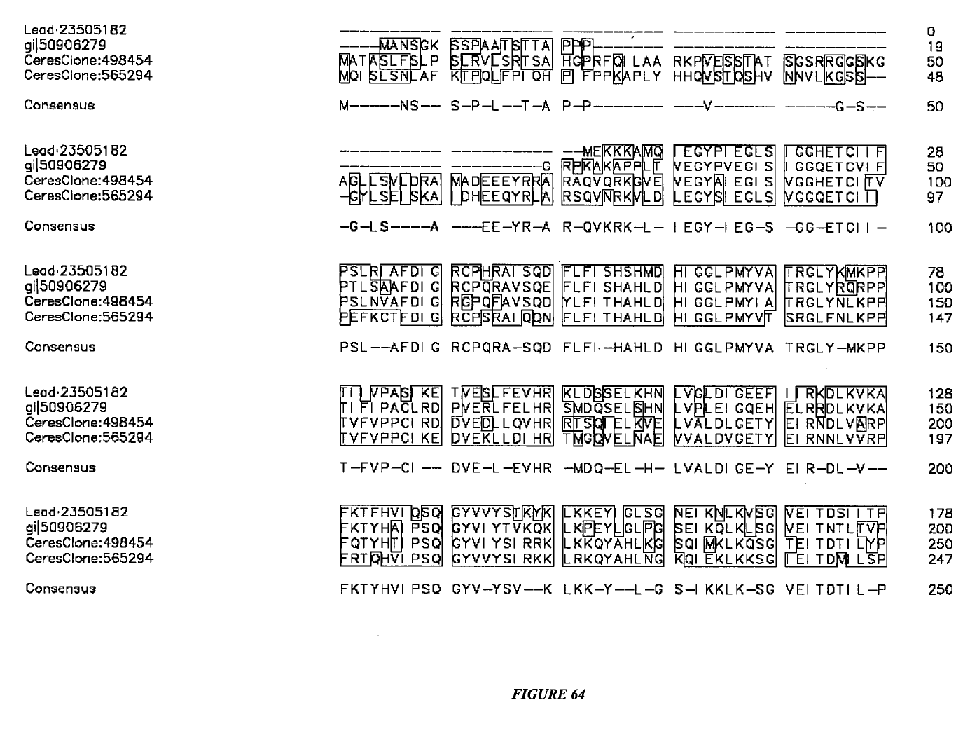

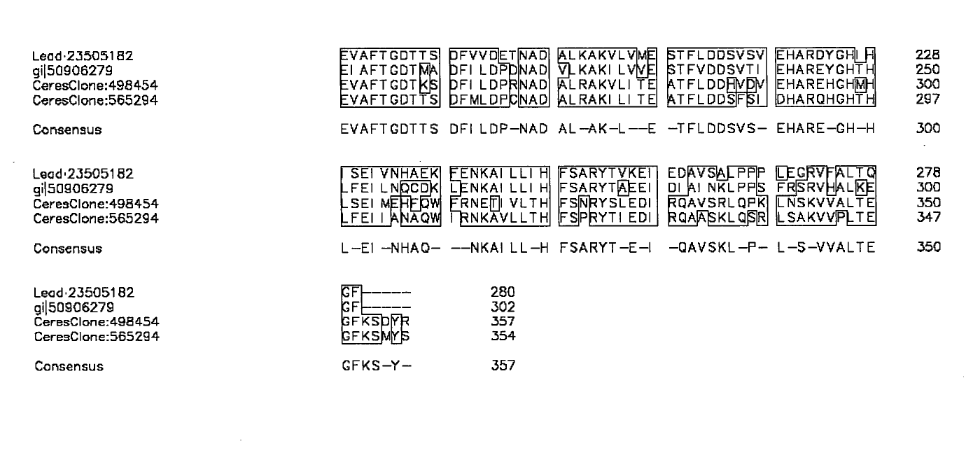

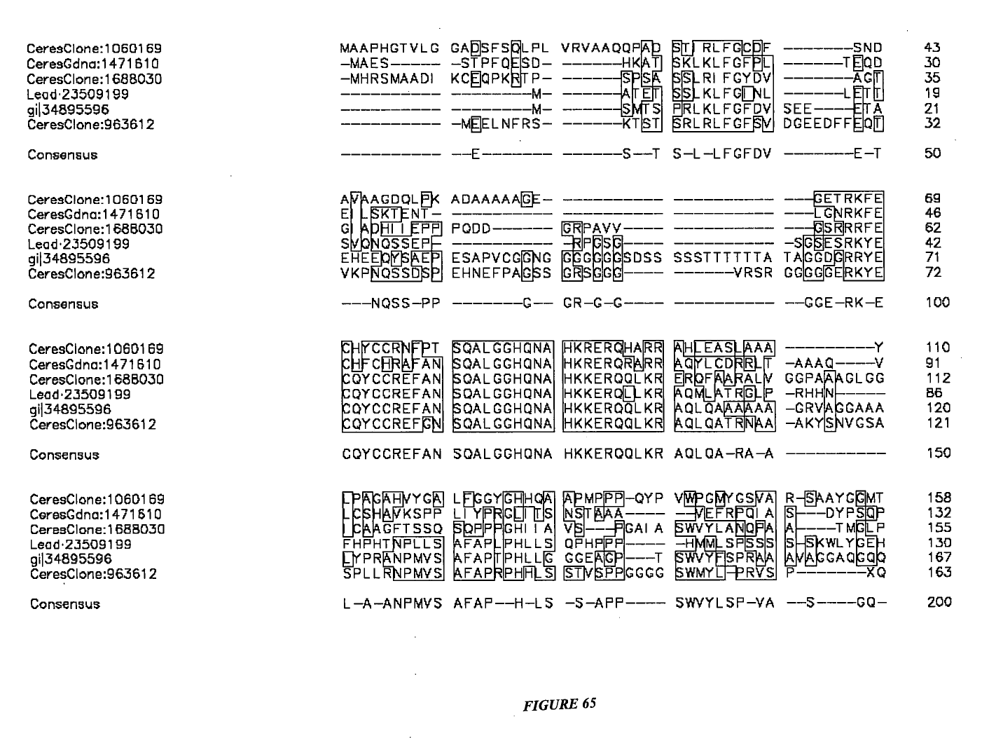

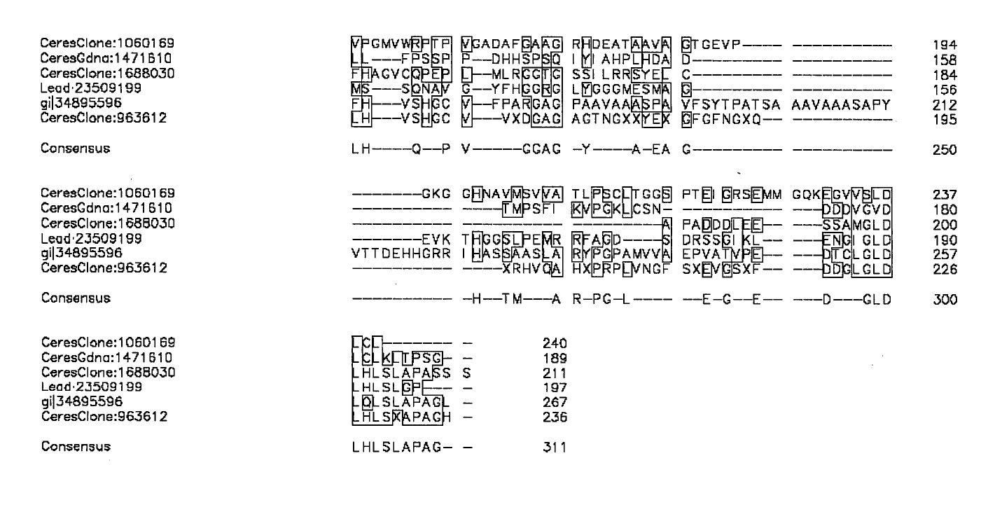

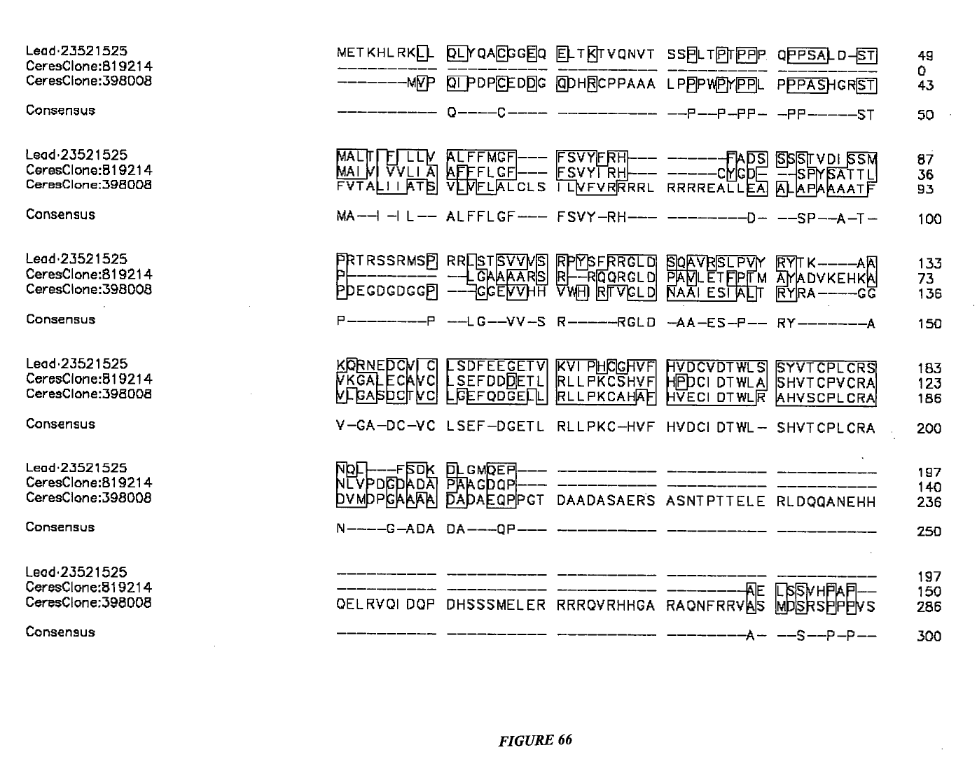

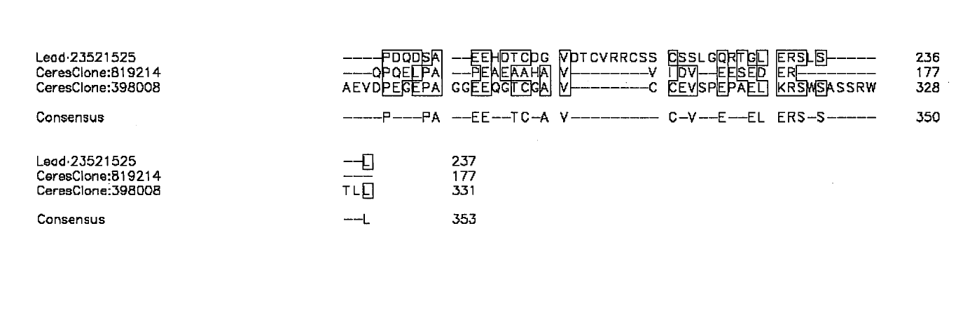

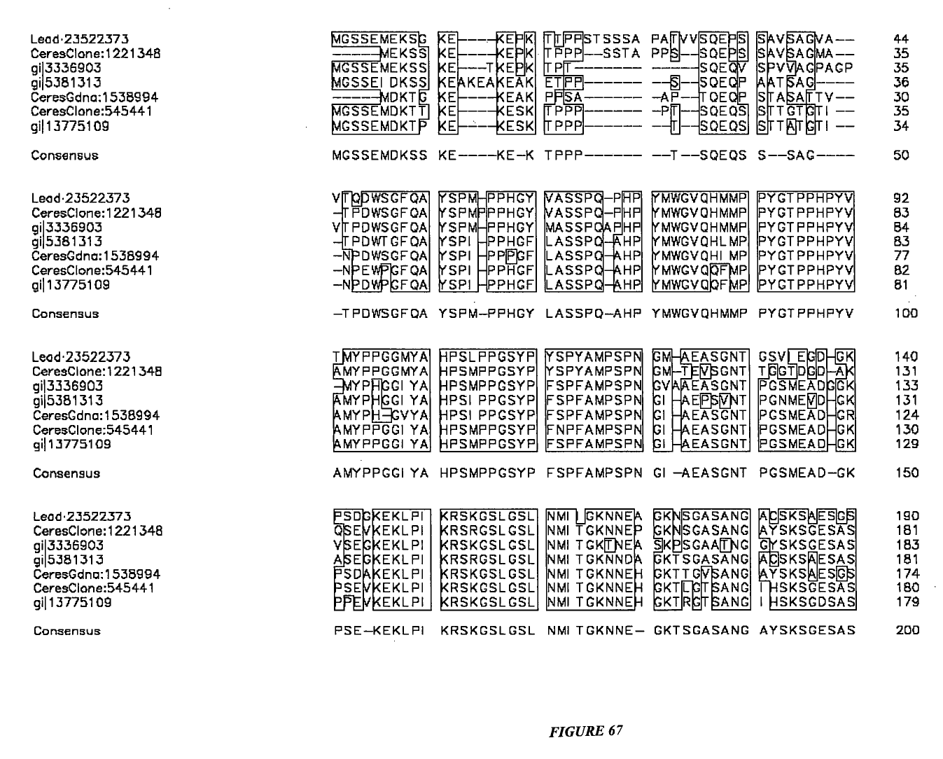

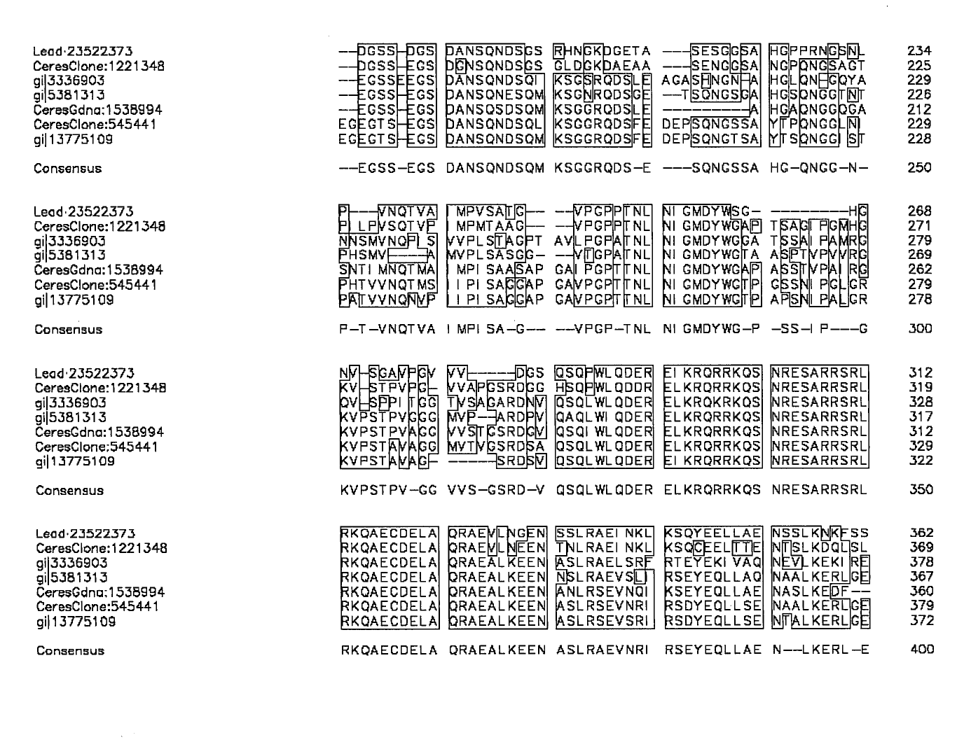

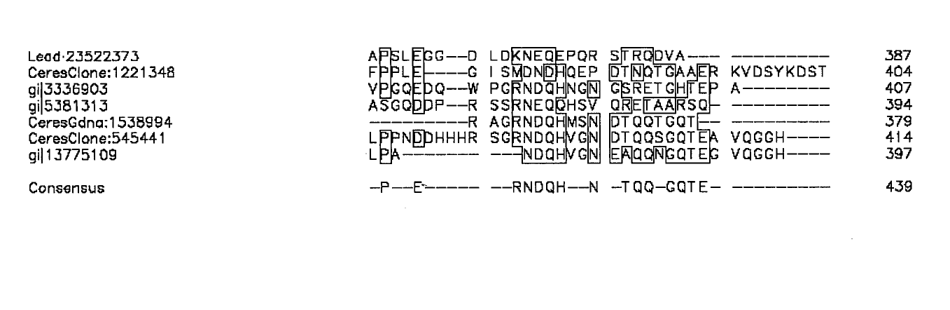

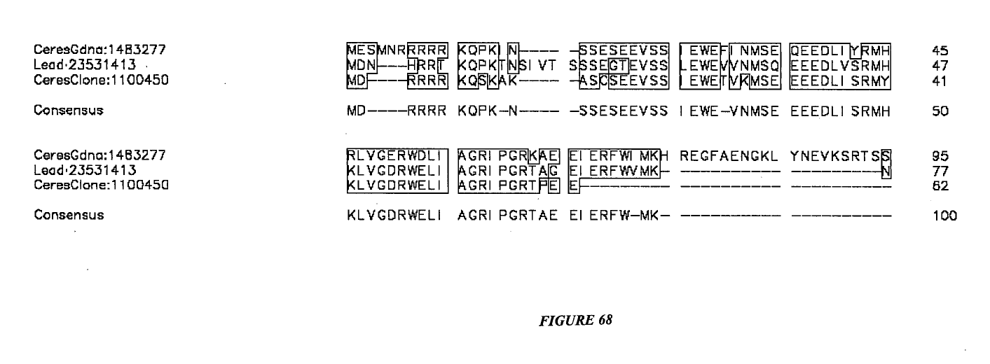

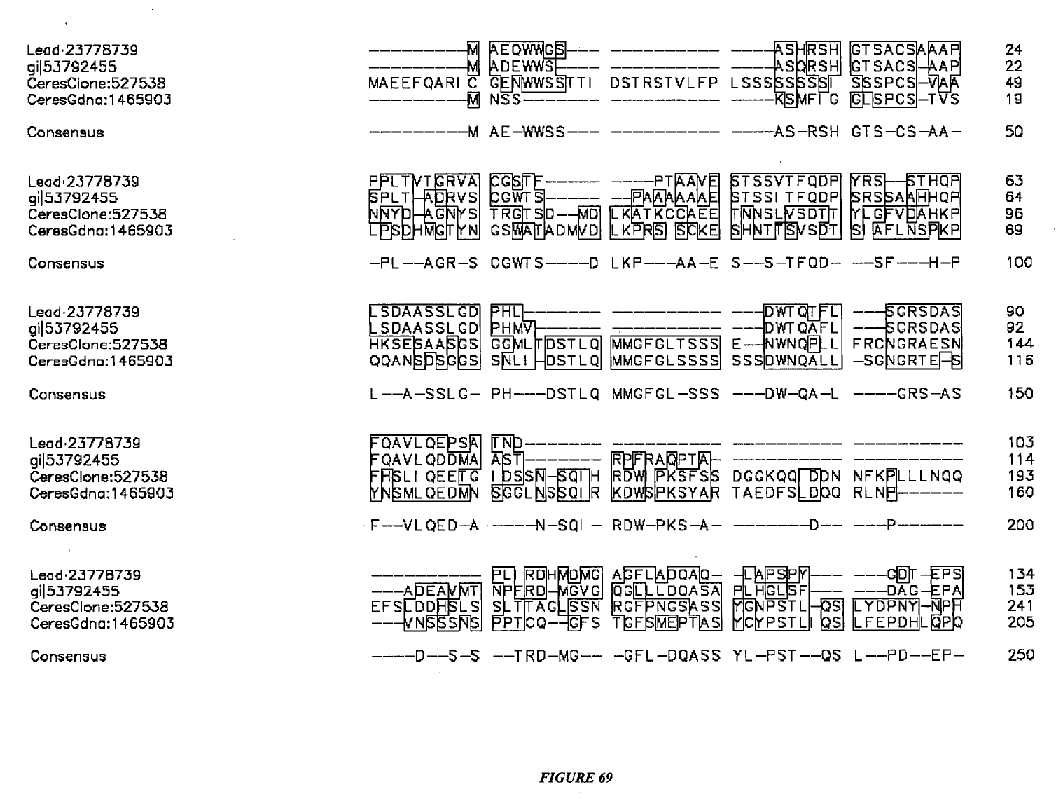

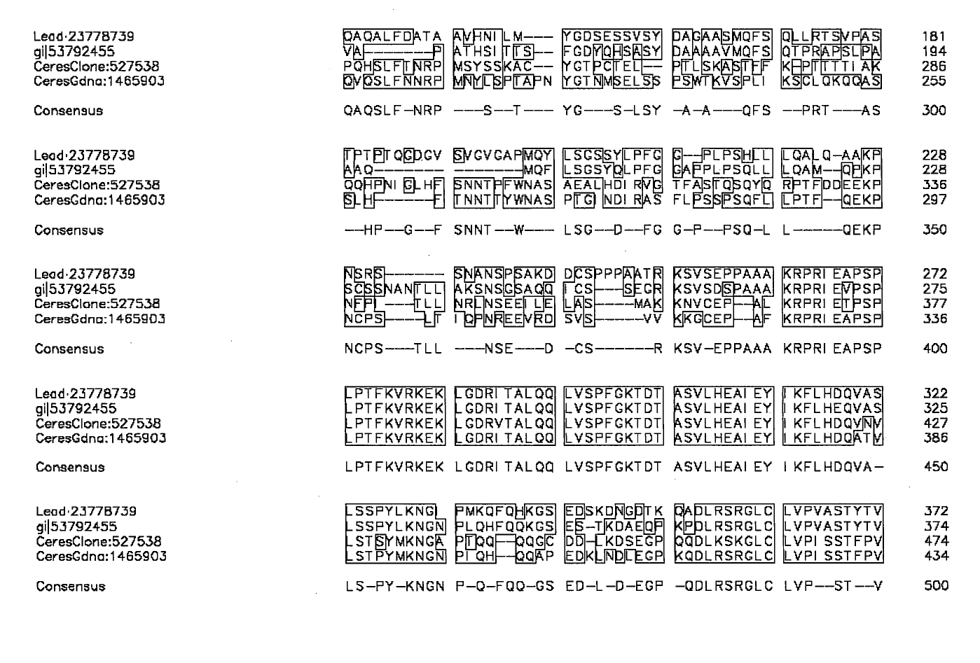

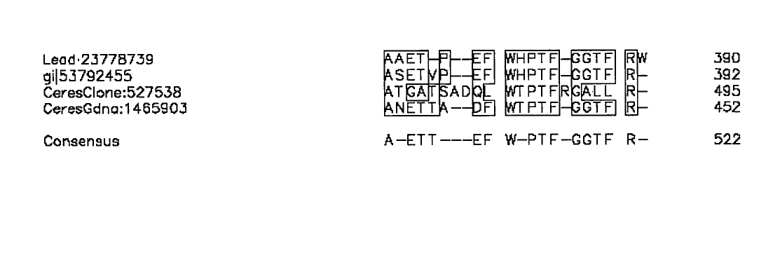

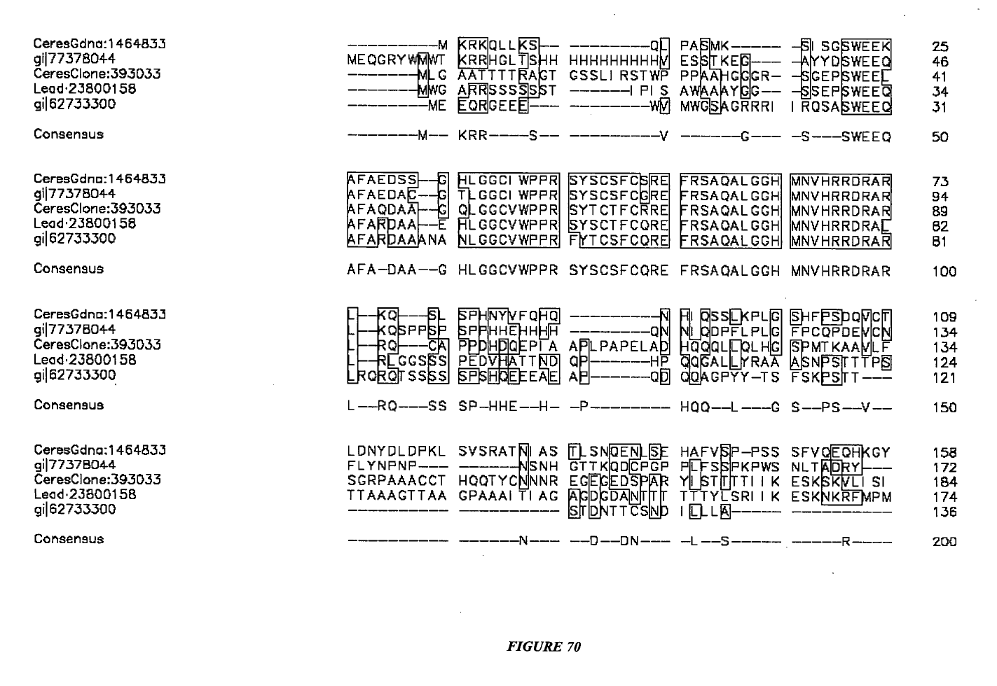

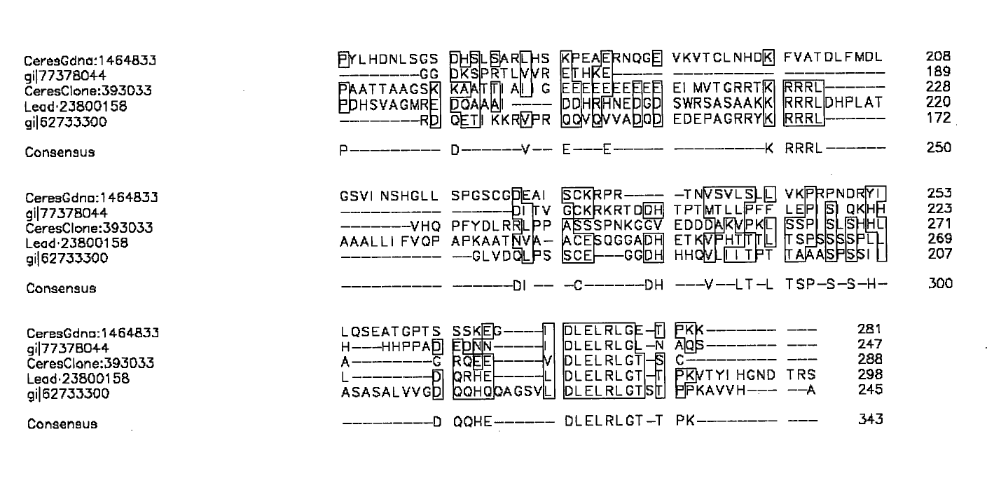

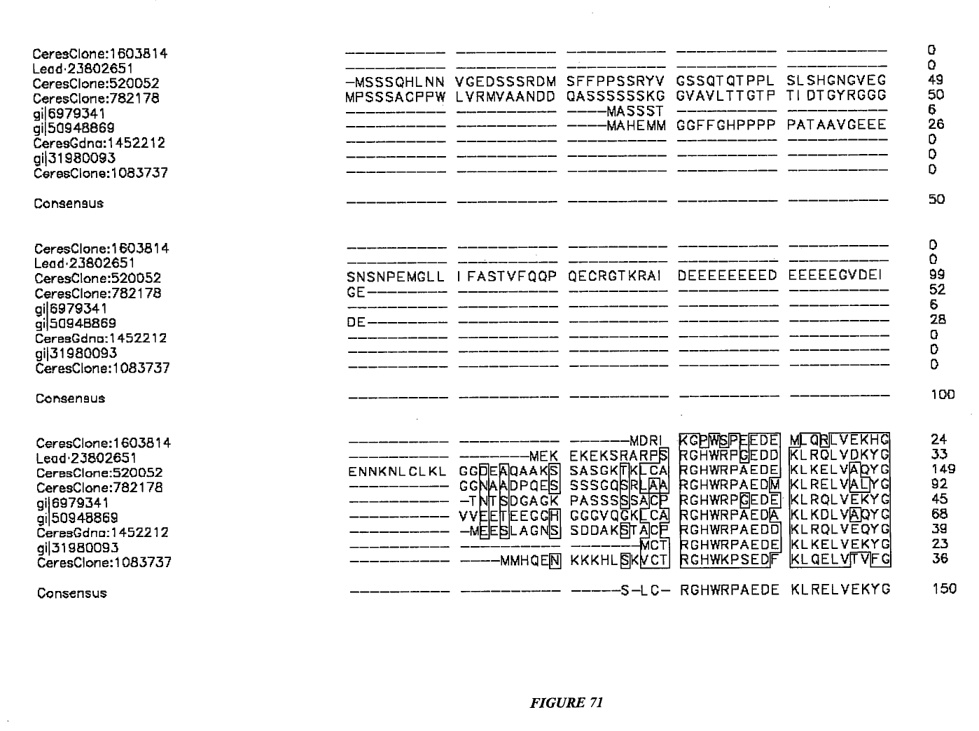

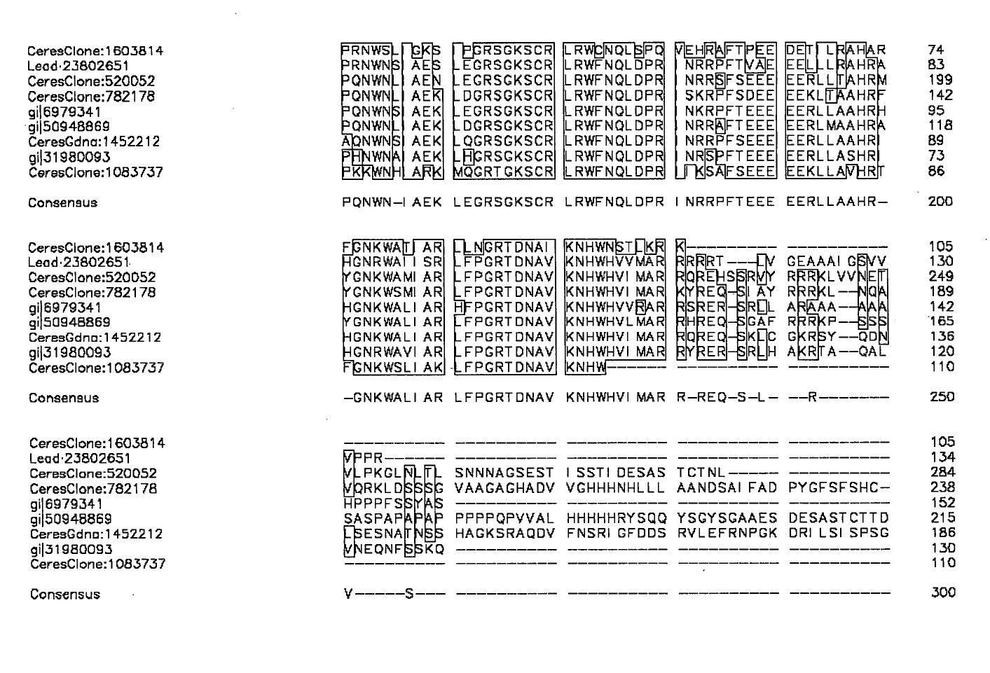

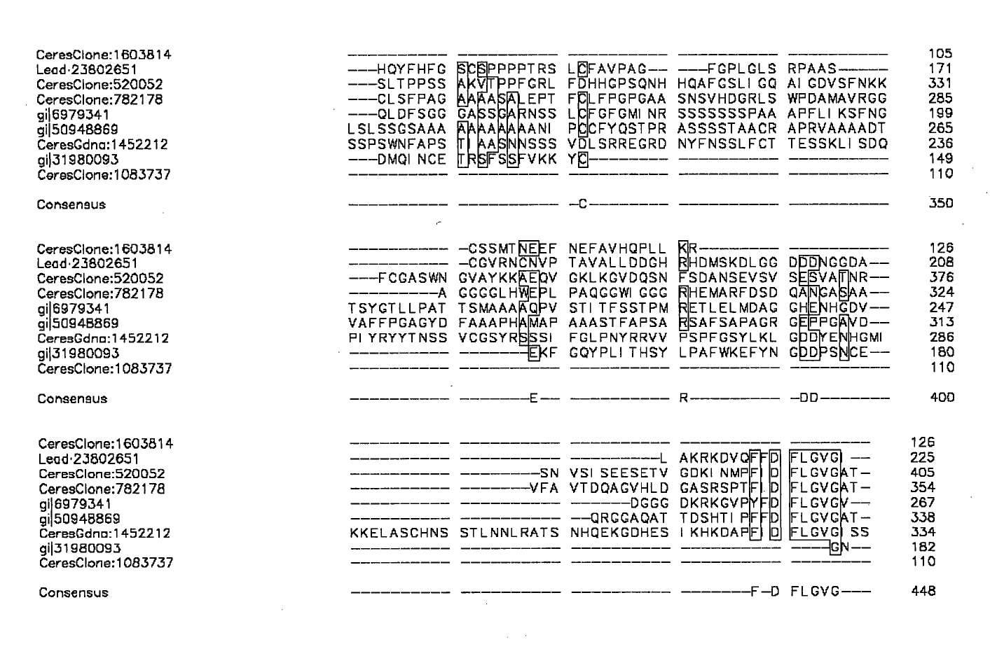

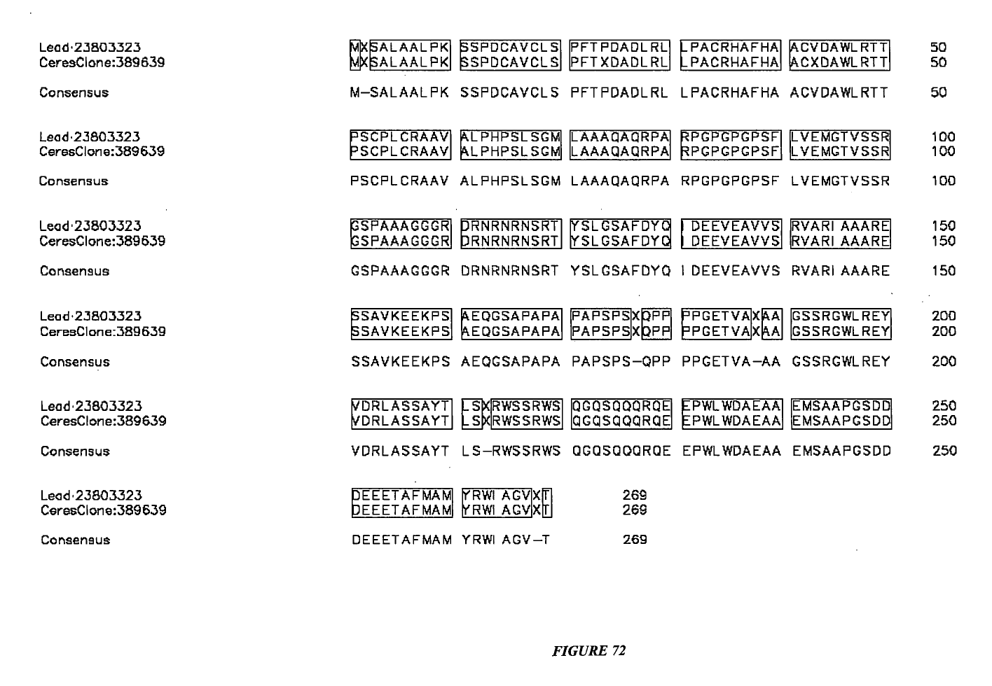

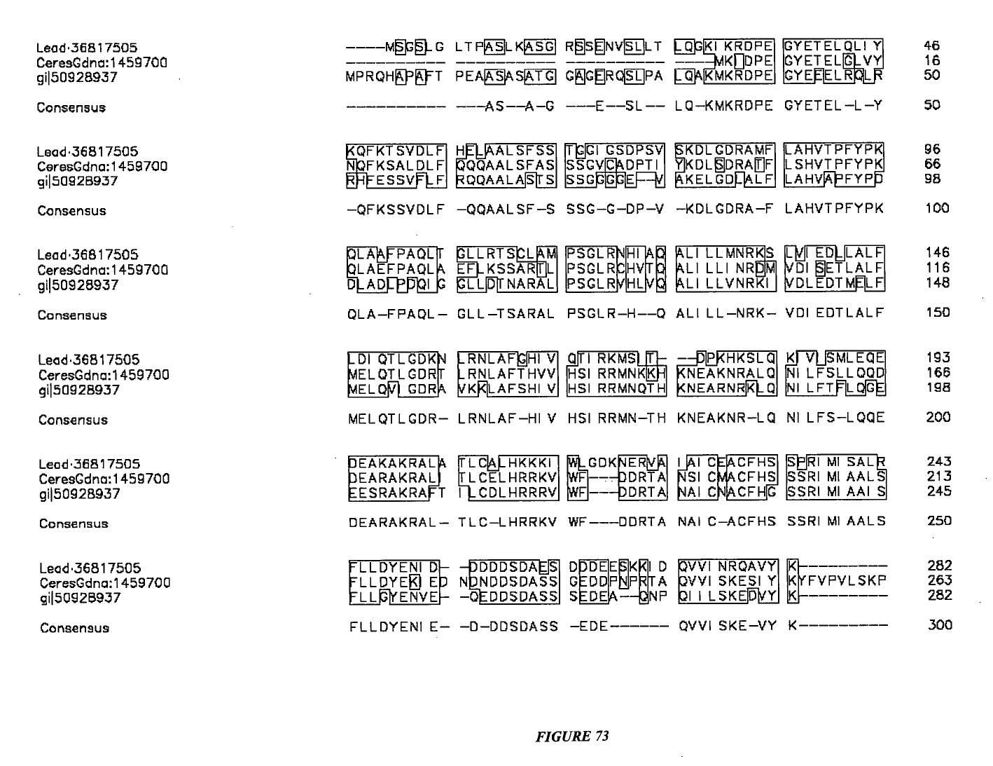

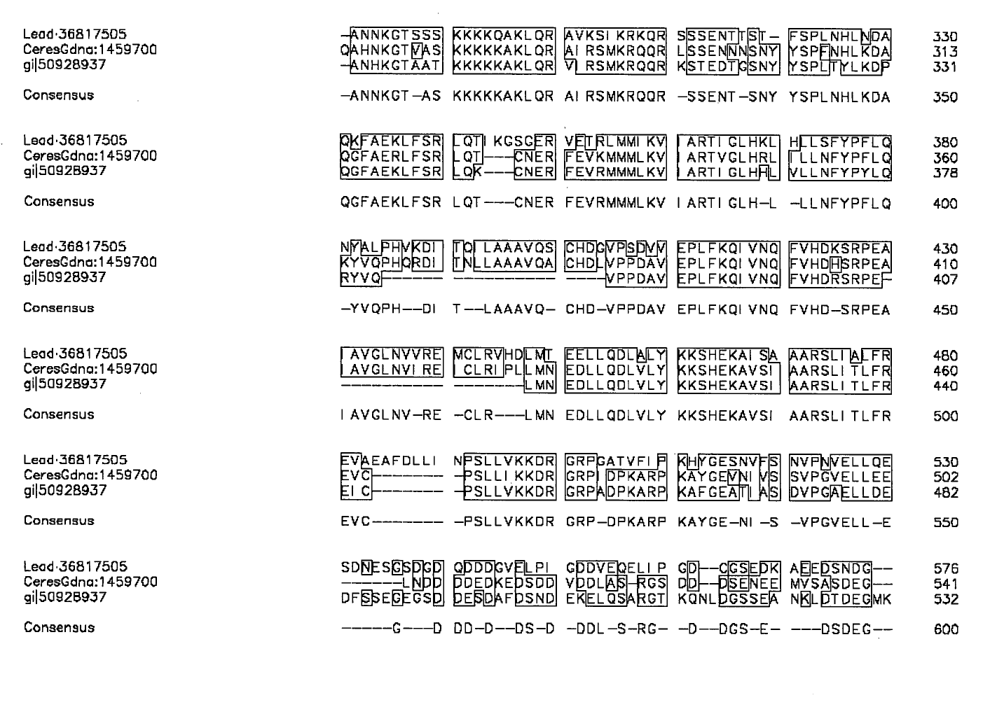

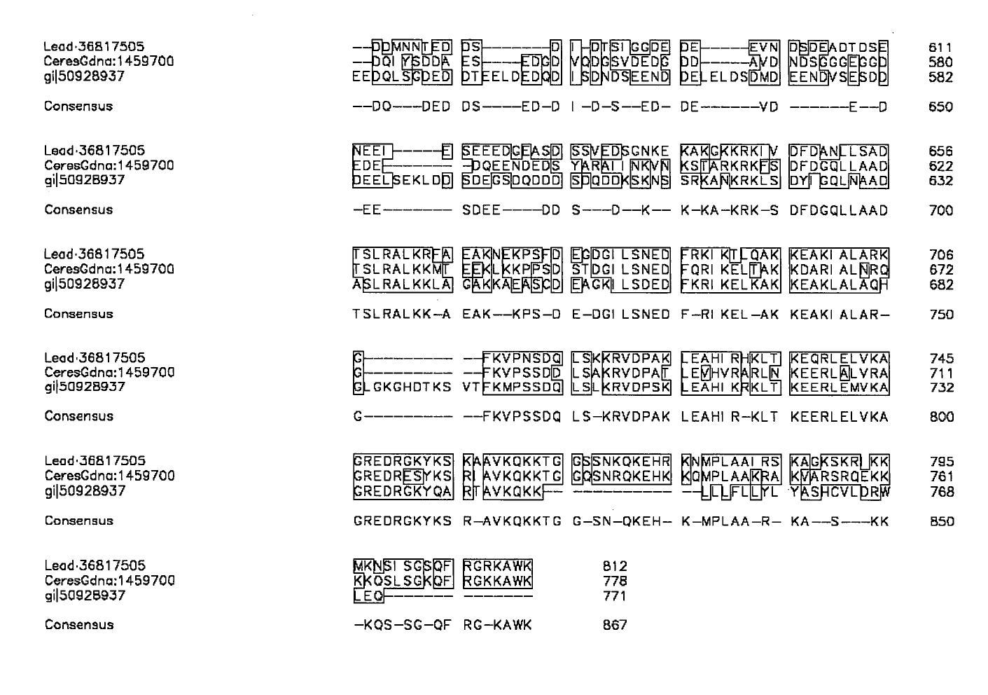

More particularly, the nucleic acid molecules of the present invention comprise: (a) a nucleotide sequence encoding an amino acid sequence that is at least 85% identical to any one of the polypeptides in the sequence listing or in the Alignment Tables of FIGS. 1-73 (SEQ ID Nos. ***), (b) a nucleotide sequence that is complementary to any one of the nucleotide sequences according to (a), (c) a nucleotide sequence according to any one of the nucleotides in the sequence listing SEQ ID Nos. ***, (d) a nucleotide sequence that is in reverse order of any one of the nucleotide sequences according to (c) when read in the 5′ to 3′ direction, (e) a nucleotide sequence able to interfere with any one of the nucleotide sequences according to (a), (f) a nucleotide sequence able to form a hybridized nucleic acid duplex with the nucleic acid according to any one of paragraphs (a)-(e) at a temperature from about 40° C. to about 48° C. below a melting temperature of the hybridized nucleic acid duplex, and (g) a nucleotide sequence encoding any one of amino acid sequences in the sequence listing or the alignment tables in FIGS. 1-73, corresponding to SEQ ID Nos. **-**

Additional embodiments of the present invention include those polypeptide and nucleic acid molecule sequences disclosed in SEQ ID NOS: **-**

The present invention further embodies a vector comprising a first nucleic acid having a nucleotide sequence encoding a plant transcription and/or translation signal, and a second nucleic acid having a nucleotide sequence according to the isolated nucleic acid molecules of the present invention. More particularly, the first and second nucleic acids may be operably linked. Even more particularly, the second nucleic acid may be endogenous to a first organism, and any other nucleic acid in the vector may be endogenous to a second organism. Most particularly, the first and second organisms may be different species.

In a further embodiment of the present invention, a host cell may comprise an isolated nucleic acid molecule according to the present invention. More particularly, the isolated nucleic acid molecule of the present invention found in the host cell of the present invention may be endogenous to a first organism and may be flanked by nucleotide sequences endogenous to a second organism. Further, the first and second organisms may be different species. Even more particularly, the host cell of the present invention may comprise a vector according to the present invention, which itself comprises nucleic acid molecules according to those of the present invention.

In another embodiment of the present invention, the isolated polypeptides of the present invention may additionally comprise amino acid sequences that are at least 85% identical to any one of the polypeptides in the sequence listing or in FIGS. 1-73 (SEQ ID Nos. **-**).

Other embodiments of the present invention include methods of introducing an isolated nucleic acid of the present invention into a host cell. More particularly, an isolated nucleic acid molecule of the present invention may be contacted to a host cell under conditions allowing transport of the isolated nucleic acid into the host cell. Even more particularly, a vector as described in a previous embodiment of the present invention, may be introduced into a host cell by the same method.

Methods of detection are also available as embodiments of the present invention. Particularly, methods for detecting a nucleic acid molecule according to the present invention in a sample. More particularly, the isolated nucleic acid molecule according to the present invention may be contacted with a sample under conditions that permit a comparison of the nucleotide sequence of the isolated nucleic acid molecule with a nucleotide sequence of nucleic acid in the sample. The results of such an analysis may then be considered to determine whether the isolated nucleic acid molecule of the present invention is detectable and therefore present within the sample.

A further embodiment of the present invention comprises a plant, plant cell, plant material or seeds of plants comprising an isolated nucleic acid molecule and/or vector of the present invention. More particularly, the isolated nucleic acid molecule of the present invention may be exogenous to the plant, plant cell, plant material or seed of a plant.

A further embodiment of the present invention includes a plant regenerated from a plant cell or seed according to the present invention. More particularly, the plant, or plants derived from the plant, plant cell, plant material or seeds of a plant of the present invention preferably has increased size (in whole or in part), increased vegetative growth, increased organ number and/or increased biomass (sometimes hereinafter collectively referred to as increased biomass), lethality, sterility or ornamental characteristics as compared to a wild-type plant cultivated under identical conditions. Furthermore, the transgenic plant may comprise a first isolated nucleic acid molecule of the present invention, which encodes a protein involved in modulating growth and phenotype characteristics, and a second isolated nucleic acid molecule which encodes a promoter capable of driving expression in plants, wherein the growth and phenotype modulating component and the promoter are operably linked. More preferably, the first isolated nucleic acid may be mis-expressed in the transgenic plant of the present invention, and the transgenic plant exhibits modulated characteristics as compared to a progenitor plant devoid of the gene, when the transgenic plant and the progenitor plant are cultivated under identical environmental conditions. In another embodiment of the present invention the modulated growth and phenotype characteristics may be due to the inactivation of a particular sequence, using for example an interfering RNA.

A further embodiment consists of a plant, plant cell, plant material or seed of a plant according to the present invention which comprises an isolated nucleic acid molecule of the present invention, wherein the plant, or plants derived from the plant, plant cell, plant material or seed of a plant, has the modulated growth and phenotype characteristics as compared to a wild-type plant cultivated under identical conditions.

Another embodiment of the present invention includes methods of modulating growth and phenotype characteristics in plants. More particularly, these methods comprise transforming a plant with an isolated nucleic acid molecule according to the present invention.

In yet another embodiment, lethality genes of the invention can be used to control transmission and expression of transgenic traits, thereby facilitating the cultivation of transgenic plants without the undesired transmission of transgenic traits to other plants. Such lethality genes can be also be utilized for selective lethality, by combining the lethal gene with appropriate promoter elements for selective expression, to thereby cause lethality of only certain cells or only under certain conditions.

Polypeptides of the present invention include consensus sequences. The consensus sequences are those as shown in FIGS. 1-73.

2. Definitions

The following terms are utilized throughout this application:

Biomass: As used herein, “biomass” refers to useful biological material including a product of interest, which material is to be collected and is intended for further processing to isolate or concentrate the product of interest. “Biomass” may comprise the fruit or parts of it or seeds, leaves, or stems or roots where these are the parts of the plant that are of particular interest for the industrial purpose. “Biomass”, as it refers to plant material, includes any structure or structures of a plant that contain or represent the product of interest.

Transformation: Examples of means by which this can be accomplished are described below and include Agrobacterium-mediated transformation (of dicots (9-10), of monocots (11-13), and biolistic methods (14)), electroporation, in planta techniques, and the like. Such a plant containing the exogenous nucleic acid is referred to here as a T₀ for the primary transgenic plant and T₁ for the first generation.

Functionally Comparable Proteins or Functional Homologs: This term describes those proteins that have at least one functional characteristic in common. Such characteristics include sequence similarity, biochemical activity, transcriptional pattern similarity and phenotypic activity. Typically, the functionally comparable proteins share some sequence similarity or at least one biochemical. Within this definition, analogs are considered to be functionally comparable. In addition, functionally comparable proteins generally share at least one biochemical and/or phenotypic activity.

Functionally comparable proteins will give rise to the same characteristic to a similar, but not necessarily the same, degree. Typically, comparable proteins give the same characteristics where the quantitative measurement due to one of the comparables is at least 20% of the other; more typically, between 30 to 40%; even more typically, between 50-60%; even more typically between 70 to 80%; even more typically between 90 to 100% of the other.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Misexpression: The term “misexpression” refers to an increase or a decrease in the transcription of a coding region into a complementary RNA sequence as compared to the wild-type. This term also encompasses expression and/or translation of a gene or coding region or inhibition of such transcription and/or translation for a different time period as compared to the wild-type and/or from a non-natural location within the plant genome, including a gene coding region from a different plant species or from a non-plant organism.

Percentage of sequence identity: As used herein, the term “percent sequence identity” refers to the degree of identity between any given query sequence and a subject sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.trnc.edu/multi-align/multi-align.html) and at the European Bioinformatics Institute site on the World Wide Web (ebi.ac.uk/clustalw).

In case of the functional homolog searches, to ensure a subject sequence having the same function as the query sequence, the alignment has to be along at least 80% of the length of the query sequence so that the majority of the query sequence is covered by the subject sequence. To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Regulatory Regions: The term “regulatory region” refers to nucleotide sequences that, when operably linked to a sequence, influence transcription initiation or translation initiation or transcription termination of said sequence and the rate of said processes, and/or stability and/or mobility of a transcription or translation product. As used herein, the term “operably linked” refers to positioning of a regulatory region and said sequence to enable said influence. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Regulatory regions can be classified in two categories, promoters and other regulatory regions.

Stringency: “Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship between hybridization conditions and Tₘ (in ° C.) is expressed in the mathematical equation:
Tₘ=81.5−16.6(log₁₀[Na⁺])+0.41(%G+C)−(600/N) (I)
where N is the number of nucleotides of the nucleic acid molecule probe. This equation works well for probes 14 to 70 nucleotides in length that are identical to the target sequence. The equation below, for Tₘ of DNA-DNA hybrids, is useful for probes having lengths in the range of 50 to greater than 500 nucleotides, and for conditions that include an organic solvent (form amide):
Tₘ=81.5+16.6 log {[Na⁺]/(1+0.7[Na⁺])}+0.41(%G+C)−500/L 0.63(%formamide) (II)
where L represents the number of nucleotides in the probe in the hybrid (21). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (22), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ medium or moderate stringency is 26-29° C. below Tₘ and low stringency is 45-48° C. below Tₘ.

T₀: The term “T₀” refers to the whole plant, explant or callus tissue, inoculated with the transformation medium.

T₁: The term T₁ refers to either the progeny of the T₀ plant, in the case of whole-plant transformation, or the regenerated seedling in the case of explant or callous tissue transformation.

T₂: The term T₂ refers to the progeny of the T₁ plant. T₂ progeny are the result of self-fertilization or cross-pollination of a T₁ plant.

T₃: The term T₃ refers to second generation progeny of the plant that is the direct result of a transformation experiment. T₃ progeny are the result of self-fertilization or cross-pollination of a T₂ plant.

3. Important Characteristics of the Polynucleotides and Polypeptides of the Invention

The nucleic acid molecules and polypeptides of the present invention are of interest because when the nucleic acid molecules are mis-expressed (i.e., when expressed at a non-natural location or in an increased or decreased amount relative to wild-type) they produce plants that exhibit modulated growth and phenotype characteristics as compared to wild-type plants, as evidenced by the results of various experiments disclosed below. This trait can be used to exploit or maximize plant products. For example, the nucleic acid molecules and polypeptides of the present invention are used to increase the expression of genes that cause the plant to have modulated growth and phenotype characteristics.

Because some of the disclosed sequences and methods increase vegetative growth, the disclosed methods can be used to enhance biomass production. For example, plants that grow vegetatively have an increase biomass production, compared to a plant of the same species that is not genetically modified for substantial vegetative growth. Examples of increases in biomass production include increases of at least 5%, at least 10%, at least 20%, or even at least 50%, when compared to an amount of biomass production by a plant of the same species not growing vegetatively.

The life cycle of flowering plants in general can be divided into three growth phases: vegetative, inflorescence, and floral (late inflorescence phase). In the vegetative phase, the shoot apical meristem (SAM) generates leaves that later will ensure the resources necessary to produce fertile offspring. Upon receiving the appropriate environmental and developmental signals the plant switches to floral, or reproductive, growth and the SAM enters the inflorescence phase (I) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase (12) where the floral organs are produced. If the appropriate environmental and developmental signals the plant switches to floral, or reproductive, growth are disrupted, the plant will not be able to enter reproductive growth, therefore maintaining vegetative growth.

As more and more transgenic plants are developed and introduced into the environment, it can be important to control the undesired spread of the transgenic triat(s) from transgenic plants to other traditional and transgenic cultivars, plant species and breeding lines, thereby preventing cross-contamination. The use of a conditionally lethal gene, i.e. one which results in plant cell death under certain conditions, has been suggested as a means to selectively kill plant cells containing a recombinent DNA (see e.g., WO 94/03619 and US patent publication 20050044596A1). The use of genes to control transmission and expression of transgenic traits is also described in U.S. application Ser. No. 10/667,295, filed on Sep. 17, 2003, which is hereby incorporated by reference. Some of the nucleotides of the invention are lethal genes, and can therefore be used as conditionally lethal genes, namely genes to be expressed in response to specific conditions, or in specific plant cells. For example, a gene that encodes a lethal trait can be placed under that control of a tissue specific promoter, or under the control of a promoter that is induced in response to specific conditions, for example, a specific chemical trigger, or specific environmental conditions.

Male or female sterile genes can also be used to control the spread of certain germplasm, such as by selective destruction of tissue, such as of the tapetum by fusing such a gene to a tapetum-specific promoter such as, TA29. Further examples of such promoters are described below.

4. The Genes of the Invention

The polynucleotides of the present invention and the proteins expressed via translation of these polynucleotides are set forth in the Sequence Listing, specifically SEQ ID Nos. 1-**. The Sequence Listing consists of functionally comparable proteins. Polypeptides comprised of a sequence within and defined by one of the consensus sequences in FIGS. 1-73 can be utilized for the purposes of the invention, namely to make transgenic plants with modulated growth and phenotype characteristics, including ornamental characteristics.

5. Use of the Genes to Make Transgenic Plants

To use the sequences of the present invention or a combination of them or parts and/or mutants and/or fusions and/or variants of them, recombinant DNA constructs are prepared that comprise the polynucleotide sequences of the invention inserted into a vector and that are suitable for transformation of plant cells. The construct can be made using standard recombinant DNA techniques (see, 16) and can be introduced into the plant species of interest by, for example, Agrobacterium-mediated transformation, or by other means of transformation, for example, as disclosed below.

The vector backbone may be any of those typically used in the field such as plasmids, viruses, artificial chromosomes, BACs, YACs, PACs and vectors such as, for instance, bacteria-yeast shuttle vectors, lamda phage vectors, T-DNA fusion vectors and plasmid vectors (see, 17-24).

Typically, the construct comprises a vector containing a nucleic acid molecule of the present invention with any desired transcriptional and/or translational regulatory sequences such as, for example, promoters, UTRs, and 3′ end termination sequences. Vectors may also include, for example, origins of replication, scaffold attachment regions (SARs), markers, homologous sequences, and introns. The vector may also comprise a marker gene that confers a selectable phenotype on plant cells. The marker may preferably encode a biocide resistance trait, particularly antibiotic resistance, such as resistance to, for example, kanamycin, bleomycin, or hygromycin, or herbicide resistance, such as resistance to, for example, glyphosate, chlorosulfuron or phosphinotricin.

It will be understood that more than one regulatory region may be present in a recombinant polynucleotide, e.g., introns, enhancers, upstream activation regions, transcription terminators, and inducible elements. Thus, more than one regulatory region can be operably linked to said sequence.

To “operably link” a promoter sequence to a sequence, the translation initiation site of the translational reading frame of said sequence is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell 1:977-984 (1989).

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to said sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Examples of various classes of promoters are described below. Some of the promoters indicated below are described in more detail in U.S. Patent Application Ser. Nos. 60/505,689; 60/518,075; 60/544,771; 60/558,869; 60/583,691; 60/619,181; 60/637,140; 10/950,321; 10/957,569; 11/058,689; 11/172,703; 11/208,308; and PCT/US05/23639. It will be appreciated that a promoter may meet criteria for one classification based on its activity in one plant species, and yet meet criteria for a different classification based on its activity in another plant species.

Other Regulatory Regions: A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

Various promoters can be used to drive expression of the genes of the present invention. Nucleotide sequences of such promoters are set forth in SEQ ID NOs: **-**. Some of them can be broadly expressing promoters, others may be more tissue preferential.

A promoter can be said to be “broadly expressing” when it promotes transcription in many, but not necessarily all, plant tissues or plant cells. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:), YP0144 (SEQ ID NO:), YP0190 (SEQ ID NO:), p13879 (SEQ ID NO:), YP0050 (SEQ ID NO:), p32449 (SEQ ID NO:), 21876 (SEQ ID NO:), YP0158 (SEQ ID NO:), YP0214 (SEQ ID NO:), YP0380 (SEQ ID NO:), PT0848 (SEQ ID NO:), and PTO633 (SEQ ID NO:). Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV ³⁵S promoter is excluded from the category of broadly expressing promoters.

Root-active promoters drive transcription in root tissue, e.g., root endodermis, root epidermis, or root vascular tissues. In some embodiments, root-active promoters are root-preferential promoters, i.e., drive transcription only or predominantly in root tissue. Root-preferential promoters include the YP0128 (SEQ ID NO: **), YP0275 (SEQ ID NO: **), PT0625 (SEQ ID NO: **), PT0660 (SEQ ID NO: **), PT0683 (SEQ ID NO: **), and PT0758 (SEQ ID NO: **). Other root-preferential promoters include the PT0613 (SEQ ID NO: **), PT0672 (SEQ ID NO: **), PT0688 (SEQ ID NO: **), and PT0837 (SEQ ID NO: **), which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA 86:7890-7894 (1989)), root cell specific promoters reported by Conkling et al., Plant Physiol. 93:1203-1211 (1990), and the tobacco RD2 gene promoter.

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin gene promoter (Bustos et al., Plant Cell 1(9):839-853 (1989)), the soybean trypsin inhibitor promoter (Riggs et al., Plant Cell 1(6):609-621 (1989)), the ACP promoter (Baerson et al., Plant Mol Biol, 22(2):255-267 (1993)), the stearoyl-ACP desaturase gene (Slocombe et al., Plant Physiol 104(4):167-176 (1994)), the soybean α′ subunit of β-conglycinin promoter (Chen et al., Proc Natl Acad Sci USA 83:8560-8564 (1986)), the oleosin promoter (Hong et al., Plant Mol Biol 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol. 13:5829-5842 (1993)), the beta-amylase gene promoter, and the barley hordein gene promoter. Other maturing endosperm promoters include the YP0092 (SEQ ID NO: **), PT0676 (SEQ ID NO: **), and PT0708 (SEQ ID NO: **).

Promoters that drive transcription in ovary tissues such as the ovule wall and mesocarp can also be useful, e.g., a polygalacturonidase promoter, the banana TRX promoter, and the melon actin promoter. Other such promoters that drive gene expression preferentially in ovules are YP0007 (SEQ ID NO: **), YP0111 (SEQ ID NO: **), YP0092 (SEQ ID NO: **), YP0103 (SEQ ID NO: **), YP0028 (SEQ ID NO: **), YP0121 (SEQ ID NO: **), YP0008 (SEQ ID NO: **), YP0039 (SEQ ID NO: **), YP0115 (SEQ ID NO: **), YP0119 (SEQ ID NO: **), YP0120 (SEQ ID NO: **) and YP0374 (SEQ ID NO: **).

In some other embodiments of the present invention, embryo sac/early endosperm promoters can be used in order drive transcription of the sequence of interest in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO: 64), YP0101 (SEQ ID NO: 71), YP0102 (SEQ ID NO: 72), YP0110 (SEQ ID NO: 75), YP0117 (SEQ ID NO: 78), YP0119 (SEQ ID NO: 79), YP0137 (SEQ ID NO: 83), DME, YP0285 (SEQ ID NO: 94), and YP0212 (SEQ ID NO: 90). Other promoters that may be useful include the following rice promoters: p530c10, pOsFIE2-2, pOsMEA, pOsYp102, and pOsYp285.

Promoters that preferentially drive transcription in zygotic cells following fertilization can provide embryo-preferential expression and may be useful for the present invention. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654, YP0097 (SEQ ID NO: **), YP0107 (SEQ ID NO: **), YP0088 (SEQ ID NO: **), YP0143 (SEQ ID NO: **), YP0156 (SEQ ID NO: **), PT0650 (SEQ ID NO: **), PT0695 (SEQ ID NO: **), PT0723 (SEQ ID NO: **), PT0838 (SEQ ID NO: **), PT0879 (SEQ ID NO: **) and PT0740 (SEQ ID NO: **).

Promoters active in photosynthetic tissue in order to drive transcription in green tissues such as leaves and stems are of particular interest for the present invention. Most suitable are promoters that drive expression only or predominantly such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol. 35:773-778 (1994)), the Cab-1 gene promoter from wheat (Fejes et al., Plant Mol. Biol. 15:921-932 (1990)), the CAB-1 promoter from spinach (Lubberstedt et al., Plant Physiol. 104:997-1006 (1994)), the cab1R promoter from rice (Luan et al., Plant Cell 4:971-981 (1992)), the pyruvate orthophosphate dikinase (PPDK) promoter from corn (Matsuoka et al., Proc Natl Acad. Sci USA 90:9586-9590 (1993)), the tobacco Lhcb1*2 promoter (Cerdan et al., Plant Mol. Biol. 33:245-255 (1997)), the Arabidopsis thaliana SUC2 sucrose-H+ symporter promoter (Truernit et al., Planta 196:564-570 (1995)), and thylakoid membrane protein promoters from spinach (psaD, psaF, psaE, PC, FNR, atpC, atpD, cab, rbcS. Other promoters that drive transcription in stems, leafs and green tissue are PT0535 (SEQ ID NO: **), PT0668 (SEQ ID NO: **), PT0886 (SEQ ID NO: **), PR0924 (SEQ ID NO: **), YP0144 (SEQ ID NO: **), YP0380 (SEQ ID NO: **) and PT0585 (SEQ ID NO: **).

In some other embodiments of the present invention, inducible promoters may be desired. Inducible promoters drive transcription in response to external stimuli such as chemical agents or environmental stimuli. For example, inducible promoters can confer transcription in response to hormones such as giberellic acid or ethylene, or in response to light or drought. Examples of drought inedible promoters are YP0380 (SEQ ID NO: **), PT0848 (SEQ ID NO: **), YP0381 (SEQ ID NO: **), YP0337 (SEQ ID NO: **), YP0337 (SEQ ID NO: **), PT0633 (SEQ ID NO: **), YP0374 (SEQ ID NO: **), PT0710 (SEQ ID NO: **), YP0356 (SEQ ID NO: **), YP0385 (SEQ ID NO: **), YP0396 (SEQ ID NO: **), YP0384 (SEQ ID NO: **), YP0384 (SEQ ID NO: **), PT0688 (SEQ ID NO: **), YP0286 (SEQ ID NO: **), YP0377 (SEQ ID NO: **), and PD1367 (SEQ ID NO: **). Examples of promoters induced by nitrogen are PT0863 (SEQ ID NO: **), PT0829 (SEQ ID NO: **), PT0665 (SEQ ID NO: **) and PT0886 (SEQ ID NO: **). An example of a shade inducible promoter is PR0924.

Other Promoters: Other classes of promoters include, but are not limited to, leaf-preferential, stem/shoot-preferential, callus-preferential, guard cell-preferential, such as PT0678 (SEQ ID NO: **), and senescence-preferential promoters. Promoters designated YP0086 (SEQ ID NO: **), YP0188 (SEQ ID NO: **), YP0263 (SEQ ID NO: **), PT0758 (SEQ ID NO: **), PT0743 (SEQ ID NO: **), PT0829 (SEQ ID NO: **), YP0119 (SEQ ID NO: **), and YP0096 (SEQ ID NO: **), as described in the above-referenced patent applications, may also be useful.

Alternatively, misexpression can be accomplished using a two component system, whereby the first component consists of a transgenic plant comprising a transcriptional activator operatively linked to a promoter and the second component consists of a transgenic plant that comprise a nucleic acid molecule of the invention operatively linked to the target-binding sequence/region of the transcriptional activator. The two transgenic plants are crossed and the nucleic acid molecule of the invention is expressed in the progeny of the plant. In another alternative embodiment of the present invention, the misexpression can be accomplished by having the sequences of the two component system transformed in one transgenic plant line.

Another alternative consists in inhibiting expression of a growth or phenotype-modulating polypeptide in a plant species of interest. The term “expression” refers to the process of converting genetic information encoded in a polynucleotide into RNA through transcription of the polynucleotide (i.e., via the enzymatic action of an RNA polymerase), and into protein, through translation of mRNA. “Up-regulation” or “activation” refers to regulation that increases the production of expression products relative to basal or native states, while “down-regulation” or “repression” refers to regulation that decreases production relative to basal or native states.

A number of nucleic-acid based methods, including anti-sense RNA, ribozyme directed RNA cleavage, and interfering RNA (RNAi) can be used to inhibit protein expression in plants. Antisense technology is one well-known method. In this method, a nucleic acid segment from the endogenous gene is cloned and operably linked to a promoter so that the antisense strand of RNA is transcribed. The recombinant vector is then transformed into plants, as described above, and the antisense strand of RNA is produced. The nucleic acid segment need not be the entire sequence of the endogenous gene to be repressed, but typically will be substantially identical to at least a portion of the endogenous gene to be repressed. Generally, higher homology can be used to compensate for the use of a shorter sequence. Typically, a sequence of at least 30 nucleotides is used (e.g., at least 40, 50, 80, 100, 200, 500 nucleotides or more).

Thus, for example, an isolated nucleic acid provided herein can be an antisense nucleic acid to one of the aforementioned nucleic acids encoding a biomass-modulating polypeptide. A nucleic acid that decreases the level of a transcription or translation product of a gene encoding a growth or phenotype-modulating polypeptide is transcribed into an antisense nucleic acid similar or identical to the sense coding sequence of the growth or phenotype-modulating polypeptide. Alternatively, the transcription product of an isolated nucleic acid can be similar or identical to the sense coding sequence of a growth or phenotype-modulating polypeptide, but is an RNA that is unpolyadenylated, lacks a 5′ cap structure, or contains an unsplicable intron.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman, et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C, Humana Press Inc., Totowa, N. J. RNA endoribonucleases such as the one that occurs naturally in Tetrahymena thermophila, and which have been described extensively by Cech and collaborators can be useful. See, for example, U.S. Pat. No. 4,987,071.

Methods based on RNA interference (RNAi) can be used. RNA interference is a cellular mechanism to regulate the expression of genes and the replication of viruses. This mechanism is thought to be mediated by double-stranded small interfering RNA molecules. A cell responds to such a double-stranded RNA by destroying endogenous mRNA having the same sequence as the double-stranded RNA. Methods for designing and preparing interfering RNAs are known to those of skill in the art; see, e.g., WO 99/32619 and WO 01/75164. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of the biomass-modulating polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., 1996, Bioorgan. Med. Chem., 4: 5-23. In addition, the deoxyphosphate backbone can be replaced with, for example, a phosphorothioate or phosphorodithioate backbone, a phosphoroamidite, or an alkyl phosphotriester backbone.

Transformation

Nucleic acid molecules of the present invention may be introduced into the genome or the cell of the appropriate host plant by a variety of techniques. These techniques, able to transform a wide variety of higher plant species, are well known and described in the technical and scientific literature (see, e.g., 28-29).

A variety of techniques known in the art are available for the introduction of DNA into a plant host cell. These techniques include transformation of plant cells by injection (30), microinjection (31), electroporation of DNA (32), PEG (33), use of biolistics (34), fusion of cells or protoplasts (35), and via T-DNA using Agrobacterium tumefaciens (36-37) or Agrobacterium rhizogenes (38) or other bacterial hosts (39), for example.

In addition, a number of non-stable transformation methods that are well known to those skilled in the art may be desirable for the present invention. Such methods include, but are not limited to, transient expression (40) and viral transfection (41).

Seeds are obtained from the transformed plants and used for testing stability and inheritance. Generally, two or more generations are cultivated to ensure that the phenotypic feature is stably maintained and transmitted.

A person of ordinary skill in the art recognizes that after the expression cassette is stably incorporated in transgenic plants and confirmed to be operable, it can be introduced into other plants by sexual crossing. Any of a number of standard breeding techniques can be used, depending upon the species to be crossed.

The nucleic acid molecules of the present invention may be used to confer the trait of an altered flowering time.

The nucleic acid molecules of the present invention encode appropriate proteins from any organism, but are preferably found in plants, fungi, bacteria or animals.

The methods according to the present invention can be applied to any plant, preferably higher plants, pertaining to the classes of Angiospermae and Gymnospermae. Plants of the subclasses of the Dicotylodenae and the Monocotyledonae are particularly suitable. Dicotyledonous plants belonging to the orders of the Magniolales, Illiciales, Laurales, Piperales Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales, for example, are also suitable. Monocotyledonous plants belonging to the orders of the Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales also may be useful in embodiments of the present invention. Further examples include, but are not limited to, plants belonging to the class of the Gymnospermae are Pinales, Ginkgoales, Cycadales and Gnetales.

The methods of the present invention are preferably used in plants that are important or interesting for agriculture, horticulture, biomass for bioconversion and/or forestry. Non-limiting examples include, for instance, tobacco, oilseed rape, sugar beet, potatoes, tomatoes, cucumbers, peppers, beans, peas, citrus fruits, avocados, peaches, apples, pears, berries, plumbs, melons, eggplants, cotton, soybean, sunflowers, roses, poinsettia, petunia, guayule, cabbages, spinach, alfalfa, artichokes, sugarcane, mimosa, Servicea lespedera, corn, wheat, rice, rye, barley, sorghum and grasses such as switch grass, giant reed, Bermuda grass, Johnson grass or turf grass, millet, hemp, bananas, poplars, eucalyptus trees and conifers.

Homologues Encompassed by the Invention

It is known in the art that one or more amino acids in a sequence can be substituted with other amino acid(s), the charge and polarity of which are similar to that of the substituted amino acid, i.e. a conservative amino acid substitution, resulting in a biologically/functionally silent change. Conservative substitutes for an amino acid within the polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as serine, threonine, tyrosine, asparagine, and glutamine; and (4) neutral nonpolar (hydrophobic) amino acids such as glycine, alanine, leucine, isoleucine, valine, proline, phenylalanine, tryptophan, cysteine, and methionine.

Nucleic acid molecules of the present invention can comprise sequences that differ from those encoding a protein or fragment thereof selected from the group consisting of the nucleotide sequences in the sequence listing due to the fact that the different nucleic acid sequence encodes a protein having one or more conservative amino acid changes.

Biologically functional equivalents of the polypeptides, or fragments thereof, of the present invention can have about 10 or fewer conservative amino acid changes, more preferably about 7 or fewer conservative amino acid changes, and most preferably about 5 or fewer conservative amino acid changes. In a preferred embodiment of the present invention, the polypeptide has between about 5 and about 500 conservative changes, more preferably between about 10 and about 300 conservative changes, even more preferably between about 25 and about 150 conservative changes, and most preferably between about 5 and about 25 conservative changes or between 1 and about 5 conservative changes.

Identification of Useful Nucleic Acid Molecules and Their Corresponding Nucleotide Sequences

The nucleic acid molecules, and nucleotide sequences thereof, of the present invention were identified by use of a variety of screens that are predictive of nucleotide sequences that provide plants with altered size, vegetative growth, organ number, plant architecture and/or biomass. One or more of the following screens were, therefore, utilized to identify the nucleotide (and amino acid) sequences of the present invention.

The present invention is further exemplified by the following examples. The examples are not intended to in any way limit the scope of the present application and its uses.

6. Experiments Confirming the Usefulness of the Polynucleotides and Polypeptides of the Invention

6.1 General Protocols

Agrobacterium-Mediated Transformation of Arabidopsis

Wild-type Arabidopsis thaliana Wassilewskija (WS) plants are transformed with Ti plasmids containing clones in the sense orientation relative to the 35S promoter. A Ti plasmid vector useful for these constructs, CRS 338, contains the Ceres-constructed, plant selectable marker gene phosphinothricin acetyltransferase (PAT), which confers herbicide resistance to transformed plants.

Ten independently transformed events are typically selected and evaluated for their qualitative phenotype in the T₁ generation.

Preparation of Soil Mixture: 24L SunshineMix #5 soil (Sun Gro Horticulture, Ltd., Bellevue, Wash.) is mixed with 16L Therm-O-Rock vermiculite (Therm-O-Rock West, Inc., Chandler, Ariz.) in a cement mixer to make a 60:40 soil mixture. To the soil mixture is added 2 Tbsp Marathon 1% granules (Hummert, Earth City, Mo.), 3 Tbsp OSMOCOTE® 14-14-14 (Hummert, Earth City, Mo.) and 1 Tbsp Peters fertilizer 20-20-20 (J. R. Peters, Inc., Allentown, Pa.), which are first added to 3 gallons of water and then added to the soil and mixed thoroughly. Generally, 4-inch diameter pots are filled with soil mixture. Pots are then covered with 8-inch squares of nylon netting.

Planting: Using a 60 mL syringe, 35 mL of the seed mixture is aspirated. 25 drops are added to each pot. Clear propagation domes are placed on top of the pots that are then placed under 55% shade cloth and subirrigated by adding 1 inch of water.

Plant Maintenance: 3 to 4 days after planting, lids and shade cloth are removed. Plants are watered as needed. After 7-10 days, pots are thinned to 20 plants per pot using forceps. After 2 weeks, all plants are subirrigated with Peters fertilizer at a rate of 1 Tsp per gallon of water. When bolts are about 5-10 cm long, they are clipped between the first node and the base of stem to induce secondary bolts. Dipping infiltration is performed 6 to 7 days after clipping.

Preparation of Agrobacterium: To 150 mL fresh YEB is added 0.1 mL each of carbenicillin, spectinomycin and rifampicin (each at 100 mg/ml stock concentration). Agrobacterium starter blocks are obtained (96-well block with Agrobacterium cultures grown to an OD₆₀₀ of approximately 1.0) and inoculated one culture vessel per construct by transferring 1 mL from appropriate well in the starter block. Cultures are then incubated with shaking at 27° C. Cultures are spun down after attaining an OD₆₀₀ of approximately 1.0 (about 24 hours). 200 mL infiltration media is added to resuspend Agrobacterium pellets. Infiltration media is prepared by adding 2.2 g MS salts, 50 g sucrose, and 5 μl 2 mg/ml benzylaminopurine to 900 ml water.

Dipping Infiltration: The pots are inverted and submerged for 5 minutes so that the aerial portion of the plants are in the Agrobacterium suspension. Plants are allowed to grow normally and seed is collected.

High-Throughput Phenotypic Screening of Misexpression Mutants:

Seed is evenly dispersed into water-saturated soil in pots and placed into a dark 4° C. cooler for two nights to promote uniform germination. Pots are then removed from the cooler and covered with 55% shade cloth for 4-5 days. Cotyledons are fully expanded at this stage. FINALE® (Sanofi Aventis, Paris, France) is sprayed on plants (3 ml FINALE® diluted into 48 oz. water)and repeated every 3-4 days until only transformants remain.

Screening is routinely performed at four stages: Seedling, Rosette, Flowering, and Senescence.
Seedling—the time after the cotyledons have emerged, but before the 3^{rd} true leaf begins to form.
Rosette—the time from the emergence of the 3^{rd} true leaf through just before the primary bolt begins to elongate.
Flowering—the time from the emergence of the primary bolt to the onset of senescence (with the exception of noting the flowering time itself, most observations should be made at the stage where approximately 50% of the flowers have opened).
Senescence—the time following the onset of senescence (with the exception of “delayed senescence”, most observations should be made after the plant has completely dried). Seeds are then collected.

Screens: Screening for increased size, vegetative growth, biomass, lethality, sterility and other modulated characteristics is performed by taking measurements, specifically T₂ measurements were taken as follows:

Days to Bolt=number of days between sowing of seed and emergence of first inflorescence.

Rosette Leaf Number at Bolt=number of rosette leaves present at time of emergence of first inflorescence.

Rosette Area=area of rosette at time of initial inflorescence emergence, using formula ((L×W)*3.14)/4.

Height=length of longest inflorescence from base to apex. This measurement was taken at the termination of flowering/onset of senescence.

Primary Inflorescence Thickness=diameter of primary inflorescence 2.5 cm up from base. This measurement was taken at the termination of flowering/onset of senescence.

Inflorescence Number=total number of unique inflorescences. This measurement was taken at the termination of flowering/onset of senescence.

PCR was used to amplify the cDNA insert in one randomly chosen T₂ plant. This PCR product was then sequenced to confirm the sequence in the plants.

Results

Plants transformed with the genes of interest were screened as described above for modulated growth and phenotype characteristics. The observations include those with respect to the entire plant, as well as parts of the plant, such as the roots and leaves. The observations for transformants with each polynucleotide sequence are noted in the Sequence listing for each of the tested nucleotide sequences and the corresponding encoded polypeptide. The modulated characteristics (i.e. observed phenotypes) are noted by an entry in the “miscellaneous features” field for each respective sequence. The “Phenotype” noted in the Sequence Listing for each relevant sequence further includes a statement of the useful utility of that sequence based on the observations.

The observations made for the various transformants can be categorized, depending upon the relevant plant tissue for the observation and the consequent utility/usefulness of the nucleotide sequence/polypeptide used to make that transformant. Table 1 correlates the shorthand notes in the sequence listing to the observations noted for each transformant (the “description” column), the tissue of the observation, the phenotype thereby associated with the transformant, and the consequent utility/usefulness of the inserted nucleotide sequence and encoded polypeptide (the “translation” column).

For some of the polynucleotides/polypeptides of the invention, the sequence listing further includes (in a “miscellaneous feature” section) an indication of important identified dominant(s) and the corresponding function of the domain or identified by comparison to the publicly available pfam database.

TABLE 1

| | PHENOTYPE | | | |
|---|---|---|---|---|
| TISSUE | QUALIFIER | PHENOTYPE | DESCRIPTION | TRANSLATION |
| WHOLE | Senescence Time | Early | the plant senesces | Useful for accelerating |
| PLANT | | Senescence | significantly early | crop development and |
| | | | (note the approximate | harvest |
| | | | number of days early | |
| | | | it started to senesce | |
| | | | in the comments) | |
| INFLORESCENCE | Flowering Time | Early Flowering | the plant flowers | Useful for accelerating |
| | | | significantly early | flowering time |
| | | | (note the approximate | |
| | | | number of days early | |
| | | | it flowered in the | |
| | | | comments) | |
| INFLORESCENCE | Flowering Time | Late Flowering | the plant flowers | Useful for delaying |
| | | | significantly late | flowering time |
| | | | (note the approximate | |
| | | | number of days late it | |
| | | | flowered in the | |
| | | | comments) | |
| INFLORESCENCE | Flowering Time | Dtb | days to bolt | Useful for delaying |
| | | | | flowering time |
| WHOLE | Senescence Time | Late Senescence | the plant senesces | Useful for delaying |
| PLANT | | | significantly late | senescence |
| | | | (note the approximate | |
| | | | number of days late it | |
| | | | started to senesce in | |
| | | | the comments) | |
| COTYLEDONS | Silver | Silver | cotyledons have a | Useful for drought or |
| | | | gray/silver colored | stress tolerance |
| | | | surface; This | |
| | | | phenotype is often | |
| | | | accompanied by a | |
| | | | small size mutation, | |
| | | | but not always | |
| WHOLE | Dark Green | Dark Green | plant is visibly darker | Useful for increasing |
| SEEDLING | | | green | chlorophyll and |
| | | | | photosynthetic capacity |
| WHOLE | Color | Dark Green | the plant is | Useful for increasing |
| PLANT | | | abnormally dark | chlorophyll and |
| | | | green | photosynthetic capacity |
| WHOLE | High | High | the plant is purple in | Useful for increasing |
| SEEDLING | Anthocyanin | Anthocyanin | color | increasing anthocyanin |
| | | | | content |
| WHOLE | Color | High | the plant is purple in | Useful for increasing |
| PLANT | | Anthocyanin | color | increasing anthocyanin |
| | | | | content |
| ROOT | No Growth in | No Growth in | roots grow along the | Useful for increasing root |
| | Soil | Soil | soil surface instead of | growth eg to enhance |
| | | | into the soil | nutrient uptake |
| ROOT | Other | Other | this correlates with | Useful for increasing root |
| | | | any root mutant | growth eg to enhance |
| | | | phenotypes which do | nutrient uptake |
| | | | not fit into the above | |
| | | | categories (a picture | |
| | | | should be taken for | |
| | | | documentation) | |
| LATERAL | Number | Less Lateral | there is an | Useful for increasing root |
| ROOTS | | Roots | abnormally low | growth eg to enhance |
| | | | number of lateral | nutrient uptake |
| | | | roots | |
| LATERAL | Other | Other | this correlates with | Useful for increasing root |
| ROOTS | | | any lateral root | growth eg to enhance |
| | | | mutant phenotypes | nutrient uptake |
| | | | which do not fit into | |
| | | | the above categories | |
| | | | (a picture should be | |
| | | | taken for | |
| | | | documentation) | |
| ROOT | Classic | Classic | there is a lack of | Useful for increasing root |
| | | | lateral roots (buds | growth eg to enhance |
| | | | may appear but do | nutrient uptake |
| | | | not elongate) | |
| ROOT | Dwarf | Dwarf | there is a stunted root | Useful for increasing root |
| | | | system | growth eg to enhance |
| | | | | nutrient uptake |
| ROOT | Mid-Section | Mid-Section | there are lateral roots | Useful for increasing root |
| | | | in the top and bottom | growth eg to enhance |
| | | | quarters of the whole | nutrient uptake |
| | | | root, but none in the | |
| | | | middle | |
| ROOT | Split | Split | appears as “classic” | Useful for increasing root |
| | | | but with two primary | growth eg to enhance |
| | | | roots, both | nutrient uptake |
| | | | originating from the | |
| | | | hypocotyl base | |
| ROOT | Other | Other | this correlates with | Useful for increasing root |
| | | | any overall root | growth eg to enhance |
| | | | structure mutant | nutrient uptake |
| | | | phenotypes which do | |
| | | | not fit into the above | |
| | | | categories (a picture | |
| | | | should be taken for | |
| | | | documentation) | |
| PRIMARY | Other | Other | this correlates with | Useful for increasing root |
| ROOT | | | any primary root | growth eg to enhance |
| | | | mutant phenotypes | nutrient uptake |
| | | | which do not fit into | |
| | | | the above categories | |
| | | | (a picture should be | |
| | | | taken for | |
| | | | documentation) | |
| ROOT | Length | Longer Root | the root hairs are | Useful for increasing root |
| HAIRS | | Hair | abnormally long | growth eg to enhance |
| | | | | nutrient uptake |
| ROOT | Length | Smaller Root | the root hairs are | Useful for increasing root |
| HAIRS | | Hair | abnormally short | growth eg to enhance |
| | | | | nutrient uptake |
| ROOT | Number | Less root hairs | there is an | Useful for increasing root |
| HAIRS | | | abnormally low | growth eg to enhance |
| | | | number of root hairs | nutrient uptake |
| ROOT | Other | Other | this correlates with | Useful for increasing root |
| HAIRS | | | any root hair mutant | growth eg to enhance |
| | | | phenotypes which do | nutrient uptake |
| | | | not fit into the above | |
| | | | categories (a picture | |
| | | | should be taken for | |
| | | | documentation) | |
| ROOT | Bulbous Root | Bulbous Root | Bulbous Root Hairs | Useful for increasing root |
| HAIRS | Hairs | Hairs | | growth eg to enhance |
| | | | | nutrient uptake |
| ROOT | Bearded | Bearded | the lateral roots are | Useful for increasing root |
| | (Nitrogen) | (Nitrogen) | long in high nitrogen, | growth eg to enhance |
| | | | and they are short in | nutrient uptake |
| | | | low nitrogen | |
| PRIMARY | Thickness | Thicker Primary | the primary root is | Useful for increasing root |
| ROOT | | Root | abnormally thick | growth eg to enhance |
| | | | | nutrient uptake |
| WHOLE | Stress | Root | Identify plants with | Useful for increasing root |
| PLANT | | Architecture | increased root mass | growth eg to enhance |
| | | | | nutrient uptake |
| PRIMARY | Thickness | Thinner Primary | the primary root is | Useful for increasing root |
| ROOT | | Root | abnormally thin | growth eg to enhance |
| | | | | nutrient uptake |
| PRIMARY | Wavy | Wavy | there is a consistent | Useful for increasing root |
| ROOT | | | and gentle wavy | growth eg to enhance |
| | | | appearance | nutrient uptake |
| LATERAL | Length | Longer Lateral | the lateral roots are | Useful for increasing root |
| ROOTS | | Root | abnormally long | growth eg to enhance |
| | | | | nutrient uptake |
| LATERAL | Number | More Lateral | there is an | Useful for increasing root |
| ROOTS | | Roots | abnormally high | growth eg to enhance |
| | | | number of lateral | nutrient uptake |
| | | | roots | |
| ROOT | Number | More root hairs | there is an | Useful for increasing root |
| HAIRS | | | abnormally high | growth eg to enhance |
| | | | number of root hairs | nutrient uptake |
| | | | | Useful for increasing seed |
| | | | | carbon or nitrogen |
| SEED | Seed Weight | Weight | weight of seed | Useful for increasing seed |
| | | | | weight |
| SILIQUES | Length | Long | siliques are | Useful for increasing |
| | | | abnormally long (the | seed/fruit yield or |
| | | | percent difference in | modifying fruit content |
| | | | length compared to | |
| | | | the control should be | |
| | | | noted in the | |
| | | | comments) | |
| SILIQIUES | Length | Short | siliques are | Useful for increasing |
| | | | abnormally short | seed/fruit yield or |
| | | | (the percent | modifying fruit content |
| | | | difference in length | |
| | | | compared to the | |
| | | | control should be | |
| | | | noted in the | |
| | | | comments) | |
| SILIQUES | Other | Other | this correlates with | Useful for increasing |
| | | | any silique mutant | seed/fruit yield or |
| | | | phenotypes which do | modifying fruit content |
| | | | not fit into the above | |
| | | | categories (a picture | |
| | | | should be taken for | |
| | | | documentation) | |
| ROSETTE | Size | Large | rosette leaves are | Useful for increasing |
| LEAVES | | | abnormally large | vegetative growth and |
| | | | (the percent | enhancing foliage |
| | | | difference in size | |
| | | | compared to the | |
| | | | control should be | |
| | | | noted in the | |
| | | | comments) | |
| | | | | Useful for making |
| | | | | nutraceuticals/pharmaceuticals |
| | | | | in plants |
| HYPOCOTYL | Other | Other | this correlates with | Useful for making larger |
| | | | any hypocotyl mutant | plants |
| | | | phenotypes which do | |
| | | | not fit into the above | |
| | | | categories (a picture | |
| | | | should be taken for | |
| | | | documentation) | |
| WHOLE | Other | Other | this correlates with | Useful for making larger |
| SEEDLING | | | any whole plant | plants |
| | | | mutant phenotypes | |
| | | | which do not fit into | |
| | | | the above categories | |
| | | | (a picture should be | |
| | | | taken for | |
| | | | documentation) | |
| WHOLE | Other | Other | this correlates with | Useful for making larger |
| PLANT | | | any whole plant | plants |
| | | | mutant phenotypes | |
| | | | which do not fit into | |
| | | | the above categories | |
| | | | (a picture should be | |
| | | | taken for | |
| | | | documentation) | |
| CAULINE | Petiole Length | Long Petioles | the cauline petioles | Useful for making larger |
| LEAVES | | | are abnormally long | plants |
| | | | (the percent | |
| | | | difference in size | |
| | | | compared to the | |
| | | | control should be | |
| | | | noted in the | |
| | | | comments) | |
| WHOLE | Size | Large | plant is abnormally | Useful for making larger |
| SEEDLING | | | large (the percent | plants |
| | | | difference in size | |
| | | | compared to the | |
| | | | control should be | |
| | | | noted in the | |
| | | | comments) | |
| WHOLE | Size | Large | plant is abnormally | Useful for making larger |
| PLANT | | | large (the percent | plants |
| | | | difference in size | |
| | | | compared to the | |
| | | | control should be | |
| | | | noted in the | |
| | | | comments) | |
| SEED | Lethal | Lethal | the seed is inviable | Useful for making lethal |
| | | | and appears as a | plants for genetic |
| | | | small, dark, raisin- | confinement systems |
| | | | like seed in the | |
| | | | mature silique | |
| WHOLE | Germination | No Germination | none of the seed | Useful for making lethal |
| SEEDLING | | | germinates | plants for genetic |
| | | | | confinement systems |
| WHOLE | Germination | Poor | a portion of the seed | Useful for making lethal |
| SEEDLING | | Germination | never germinates | plants for genetic |
| | | | | confinement systems |
| WHOLE | Germination | Slow | a portion of the seed | Useful for making lethal |
| SEEDLING | | Germination | germinates | plants for genetic |
| | | | significantly later | confinement systems |
| | | | than the rest of the | |
| | | | seed in the pot | |
| ROSETTE | Vitrified | Vitrified | leaves are somewhat | Useful for making lethal |
| LEAVES | | | translucent or ?water | plants for genetic |
| | | | soaked? | confinement systems |
| CAULINE | Vitrified | Vitrified | leaves are somewhat | Useful for making lethal |
| LEAVES | | | translucent or ?water | plants for genetic |
| | | | soaked? | confinement systems |
| COTYLEDONS | Albino | Opaque Albino | plant is opaque and | Useful for making lethal |
| | | | devoid of pigment | plants for genetic |
| | | | | confinement systems |
| COTYLEDONS | Albino | Translucent | plant is translucent | Useful for making lethal |
| | | Albino | and devoid of | plants for genetic |
| | | | pigment | confinement systems |
| WHOLE | Lethal | Seedling Lethal | cotyledons emerge | Useful for making lethal |
| SEEDLING | | | (although they are | plants for genetic |
| | | | often small), but then | confinement systems |
| | | | the plant ceases to | |
| | | | develop further; No | |
| | | | true leaves appear | |
| | | | and the plant dies | |
| | | | early (These differ | |
| | | | from yellow-green | |
| | | | lethals in that the | |
| | | | cotyledons are wild- | |
| | | | type in color and may | |
| | | | not look differ | |
| WHOLE | Lethal | Yellow-Green | cotyledons are small | Useful for making lethal |
| SEEDLING | | Lethal | and pale yellow- | plants for genetic |
| | | | green in color, but | confinement systems |
| | | | NOT totally devoid | |
| | | | of pigment; In | |
| | | | addition to yellow- | |
| | | | green cotyledons, | |
| | | | these plants produce | |
| | | | no or severely | |
| | | | reduced size true | |
| | | | leaves, which, if | |
| | | | present, are also | |
| | | | yellow-green; These | |
| | | | plants die prem | |
| WHOLE | Meristem Mutant | Meristem Mutant | this term | Useful for making lethal |
| SEEDLING | | | encompasses a | plants for genetic |
| | | | variety of | confinement systems |
| | | | phenotypes, all of | |
| | | | which have one thing | |
| | | | in common, i.e., they | |
| | | | all have something | |
| | | | significantly wrong | |
| | | | with how the | |
| | | | meristem is | |
| | | | producing its leaves; | |
| | | | Depending on the | |
| | | | severity of the | |
| | | | phenotype, the plants | |
| | | | in this category | |
| WHOLE | Seedling | Seedling | this term | Useful for making lethal |
| SEEDLING | Defective | Defective | encompasses a | plants for genetic |
| | | | variety of phenotypes | confinement systems |
| | | | which share similar | |
| | | | characteristics, i.e., | |
| | | | they are small, have | |
| | | | distorted structures, | |
| | | | and are prone to early | |
| | | | death; For example, | |
| | | | patterning mutants | |
| | | | would be a class of | |
| | | | mutants which fall | |
| | | | under this category | |
| WHOLE | Color | Yellow-Green | the leaves and | Useful for making lethal |
| PLANT | | Viable 1 | cotyledons are | plants for genetic |
| | | | yellow-green in | confinement systems |
| | | | color, but this is not a | |
| | | | lethal phenotype | |
| WHOLE | Color | Yellow-Green | the leaves are yellow- | Useful for making lethal |
| PLANT | | Viable 2 | green in color but the | plants for genetic |
| | | | cotyledons are a | confinement systems |
| | | | wild-type green in | |
| | | | color | |
| WHOLE | Color | Yellow-Green | the leaves start out | Useful for making lethal |
| PLANT | | Viable 3 | wild-type green and | plants for genetic |
| | | | gradually turn | confinement systems |
| | | | yellow-green in | |
| | | | color, while the | |
| | | | cotyledons stay wild- | |
| | | | type green | |
| WHOLE | Color | Yellow-Green | the leaves appear | Useful for making lethal |
| PLANT | | Viable 4 | wild-type green, but | plants for genetic |
| | | | slowly turn yellow- | confinement systems |
| | | | green over time, | |
| | | | while the cotyledons | |
| | | | appear and remain | |
| | | | yellow-green | |
| WHOLE | Stress | Seed Bleaching | Identify plants whose | Useful for making low |
| PLANT | | | seed coats do not | fiber seeds with increased |
| | | | bleach out under long | digestability |
| | | | bleach soaking | |
| ROSETTE | Fused | Leaf Fused to | the leaf is fused to an | Useful for making |
| LEAVES | | Inflorescence | inflorescence | ornamental plants with |
| | | | | flowers and leaves fused |
| ROSETTE | Interveinal | Interveinal | the leaf tissue is | Useful for making |
| LEAVES | Chlorosis | Chlorosis | chlorotic between its | ornamental plants with |
| | | | veins | modified color |
| CAULINE | Interveinal | Interveinal | the leaf tissue is | Useful for making |
| LEAVES | Chlorosis | Chlorosis | chlorotic between its | ornamental plants with |
| | | | veins | modified color |
| FLOWER | Organ | Fused Sepals | the sepals are fused | Useful for making |
| | Morphology | | together and won?t | ornamental plants with |
| | | | open naturally, but | modified flowers |
| | | | the flower is | |
| | | | otherwise wild-type | |
| FLOWER | Organ | Narrow Petals | the petals are | Useful for making |
| | Morphology | | abnormally narrow | ornamental plants with |
| | | | | modified flowers |
| FLOWER | Organ | Narrow Sepals | the sepals are | Useful for making |
| | Morphology | | abnormally narrow | ornamental plants with |
| | | | | modified flowers |
| FLOWER | Organ | Short Petals | the petals are | Useful for making |
| | Morphology | | abnormally short | ornamental plants with |
| | | | | modified flowers |
| FLOWER | Organ | Short Sepals | the sepals are | Useful for making |
| | Morphology | | abnormally short | ornamental plants with |
| | | | | modified flowers |
| FLOWER | Size | Large | flower is abnormally | Useful for making |
| | | | large (the percent | ornamental plants with |
| | | | difference in size | modified flowers |
| | | | compared to the | |
| | | | control should be | |
| | | | noted in the | |
| | | | comments) | |
| FLOWER | Size | Small | flower is abnormally | Useful for making |
| | | | small (the percent | ornamental plants with |
| | | | difference in size | modified flowers |
| | | | compared to the | |
| | | | control should be | |
| | | | noted in the | |
| | | | comments) | |
| FLOWER | Other | Other | this correlates with | Useful for making |
| | | | any flower mutant | ornamental plants with |
| | | | phenotypes which do | modified flowers |
| | | | not fit into the above | |
| | | | categories (a picture | |
| | | | should be taken for | |
| | | | documentation) | |
| INFLORESCENCE | Aerial Rosette | Aerial Fosette | rosette forms at or | Useful for making |
| | | | above the first | ornamental plants with |
| | | | internode | modified flowers |
| INFLORESCENCE | Appearance | Corkscrew | the inflorescence is | Useful for making |
| | | Appearance | really twisted, almost | ornamental plants with |
| | | | like a corkscrew, but | modified flowers |
| | | | somewhat more | |
| | | | irregular | |
| INFLORESCENCE | Appearance | Curved | the inflorescence has | Useful for making |
| | | Appearance | a slight, irregular | ornamental plants with |
| | | | curve upwards, | modified flowers |
| | | | greater than that of | |
| | | | the control plants | |
| INFLORESCENCE | Appearance | Multi- | the inflorescence is | Useful for making |
| | | Inflorescence | fused to another | ornamental plants with |
| | | Fusion | inflorescence, | modified flowers |
| | | | creating a celery-like | |
| | | | appearance | |
| INFLORESCENCE | Appearance | Undulate | the inflorescence is | Useful for making |
| | | Appearance | wavy in appearance | ornamental plants with |
| | | | | modified flowers |
| INFLORESCENCE | Branching | Acauline | first branching is not | Useful for making |
| | | Branching | subtended by a | ornamental plants with |
| | | | cauline leaf | modified flowers |
| INFLORESCENCE | Wax | Glaucous | inflorescence is | Useful for making |
| | | | abnormally dull in | ornamental plants with |
| | | | appearance | modified flowers |
| INFLORESCENCE | Wax | Glossy | inflorescence is | Useful for making |
| | | | shiny/glossy in | ornamental plants with |
| | | | appearance | modified flowers |
| INFLORESCENCE | Other | Other | this correlates with | Useful for making |
| | | | any inflorescence | ornamental plants with |
| | | | mutant phenotypes | modified flowers |
| | | | which do not fit into | |
| | | | the above categories | |
| | | | (a picture should be | |
| | | | taken for | |
| | | | documentation) | |
| COTYLEDONS | Asymmetric | Asymmetric | the shape of the | Useful for making |
| | | | cotyledon is | ornamental plants with |
| | | | asymmetric in | modified foliage |
| | | | reference to the | |
| | | | vertical axis | |
| ROSETTE | Other | Other | this correlates with | Useful for making |
| LEAVES | | | any leaf mutant | ornamental plants with |
| | | | phenotypes which do | modified leaves |
| | | | not fit into the above | |
| | | | categories (a picture | |
| | | | should be taken for | |
| | | | documentation) | |
| CAULINE | Other | Other | this correlates with | Useful for making |
| LEAVES | | | any cauline mutant | ornamental plants with |
| | | | phenotypes which do | modified leaves |
| | | | not fit into the above | |
| | | | categories (a picture | |
| | | | should be taken for | |
| | | | documentation) | |
| FLOWER | Homeotic | Homeotic | the flower has one or | Useful for making plants |
| | Mutant | Mutant | more of its organs | sterile and for genetic |
| | | | converted to another | confinement |
| | | | type of organ | |
| | | | (specific details | |
| | | | should be noted in | |
| | | | the comments) | |
| FLOWER | Organ | Aberrant Organ | there is an abnormal | Useful for making plants |
| | Morphology | Number | number of some or | sterile and for genetic |
| | | | all of the flowers | confinement |
| | | | organs | |
| FLOWER | Organ | Short Stamens | the stamens are | Useful for making plants |
| | Morphology | | abnormally short; | sterile and for genetic |
| | | | This often leads to | confinement |
| | | | mechanical problems | |
| | | | with fertility | |
| FLOWER | Fertility | Aborted fertility | the ovule is | Useful for making plants |
| | | | unfertilized and | sterile and for genetic |
| | | | appears as a brown or | confinement |
| | | | white speck in the | |
| | | | mature silique | |
| FLOWER | Fertility | Female-sterile | there is a problem | Useful for making plants |
| | | | with the ovules such | sterile and for genetic |
| | | | that no fertilization is | confinement |
| | | | occurring | |
| FLOWER | Fertility | Male-sterile | there is a problem | Useful for making plants |
| | | | with the pollen such | sterile and for genetic |
| | | | that no fertilization is | confinement |
| | | | occurring | |
| FLOWER | Fertility | Reduced fertility | a reduced number of | Useful for making plants |
| | | | successful | sterile and for genetic |
| | | | fertilization events, | confinement |
| | | | and therefore seeds, | |
| | | | are being produced | |
| | | | by the plant | |
| FLOWER | Fertility | Sterile | no successful | Useful for making plants |
| | | | fertilization events, | sterile and for genetic |
| | | | and therefore no seed | confinement |
| | | | is being produced by | |
| | | | the plant; The reason | |
| | | | for this sterility is not | |
| | | | known at the time of | |
| | | | the observation | |
| FLOWER | Fertility | Other | this correlates with | Useful for making plants |
| | | | any fertility mutant | sterile and for genetic |
| | | | phenotypes which do | confinement |
| | | | not fit into the above | |
| | | | categories (a picture | |
| | | | should be taken for | |
| | | | documentation) | |
| WHOLE | Stress | Early Flowering | Identify plants that | Useful for making plants |
| PLANT | | | flower early | that flower early |
| COTYLEDONS | Petiole Length | Long Petioles | the cotyledon petioles | Useful for making plants |
| | | | are abnormally long | that grow and better in |
| | | | (the percent | shade |
| | | | difference in size | |
| | | | compared to the | |
| | | | control should be | |
| | | | noted in the | |
| | | | comments) | |
| ROSETTE | Petiole Length | Varying Petiole | the leaf petioles vary | Useful for making plants |
| LEAVES | | Lengths | in length throughout | that grow better in shade |
| | | | the rosette | |
| ROSETTE | Petiole Length | Long Petioles | the leaf petioles are | Useful for making plants |
| LEAVES | | | abnormally long (the | that grow better in shade |
| | | | percent difference in | |
| | | | size compared to the | |
| | | | control should be | |
| | | | noted in the | |
| | | | comments) | |
| | | | | Useful for making plants |
| | | | | tolerant to biotic stress |
| WHOLE | Stress | | Identify plants able to | Useful for making plants |
| PLANT | | | tolerate high density | tolerant to density and |
| | | | and no phosphate and | low fertilizer |
| | | | nitrogen, possible | |
| | | | lead assay for vigor | |
| | | | under population | |
| | | | density and low | |
| | | | nutrient conditions | |
| WHOLE | Stress | pH (high) | Identify plants | Useful for making plants |
| PLANT | | | tolerant to high PH, | tolerant to high pH or low |
| | | | and possibly low | phosphate |
| | | | phosphate | |
| WHOLE | Stress | Low Nitrate | Identify plants | Useful for making plants |
| PLANT | | | tolerant to low | tolerant to low nitrogen |
| | | | nitrogen/nitrate | |
| | | | growth media | |
| WHOLE | Stress | LNABA | Identify plants | Useful for making plants |
| PLANT | | | tolerant to low | tolerant to low nitrogen |
| | | | nitrogen and high | |
| | | | ABA concentrations | |
| WHOLE | Stress | No Nitrogen | Identify plants with | Useful for making plants |
| PLANT | | | increased vigor under | tolerant to low nitrogen |
| | | | no nitrogen | |
| | | | conditions | |
| WHOLE | Stress | MSX | Identify plants | Useful for making plants |
| PLANT | | | tolerant to nitrogen | tolerant to low nitrogen |
| | | | assimilation inhibitor, | |
| | | | and possibly low | |
| | | | nitrogen tolerance | |
| | | | and/or seed nitrogen | |
| | | | accumulation | |
| WHOLE | Stress | No N, No PO4 | Identify plants | Useful for making plants |
| PLANT | | | tolerant to no | tolerant to low |
| | | | nitrogen and no | nitrogen/low phosphate |
| | | | phosphate growth | |
| | | | media | |
| WHOLE | Stress | Oxidative | Identify plants | Useful for making plants |
| PLANT | | | tolerant to oxidative | tolerant to oxidative |
| | | | stress | stresses |
| ROSETTE | Trichomes | Few Trichomes | trichomes are sparse | Useful for making plants |
| LEAVES | | | but present on the | with enhanced chemical |
| | | | leaves | composition |
| ROSETTE | Trichomes | Glabrous | trichomes are totally | Useful for making plants |
| LEAVES | | | absent | with enhanced chemical |
| | | | | composition |
| ROSETTE | Trichomes | Abnormal | the trichomes are | Useful for making plants |
| LEAVES | | Trichome Shape | abnormally shaped | with enhanced chemical |
| | | | | composition |
| CAULINE | Trichomes | Few Trichomes | trichomes are sparse | Useful for making plants |
| LEAVES | | | but present on the | with enhanced chemical |
| | | | leaves | composition |
| CAULINE | Trichomes | Glabrous | trichomes are totally | Useful for making plants |
| LEAVES | | | absent | with enhanced chemical |
| | | | | composition |
| CAULINE | Trichomes | Abnormal | the trichomes are | Useful for making plants |
| LEAVES | | Trichome Shape | abnormally shaped | with enhanced chemical |
| | | | | composition |
| INFLORESCENCE | Trichomes | Glabrous | trichomes are totally | Useful for making plants |
| | | | absent | with enhanced chemical |
| | | | | composition |
| INFLORESCENCE | Trichomes | Abnormal | the trichomes are | Useful for making plants |
| | | Trichome Shape | abnormally shaped | with enhanced chemical |
| | | | | composition |
| ROSETTE | Curled | Corkscrew | leaves appear as | Useful for making plants |
| LEAVES | | | “Curled 5”, with the | with altered leaf shape eg |
| | | | additional attribute of | curled leaves |
| | | | twisting like a | |
| | | | corkscrew, instead of | |
| | | | uniformly curling | |
| | | | from both sides of the | |
| | | | leaf | |
| ROSETTE | Curled | Cup-shaped | leaves are curled up | Useful for making plants |
| LEAVES | | | at the leaf margins | with altered leaf shape eg |
| | | | such that they form a | curled leaves |
| | | | cup or bowl-like | |
| | | | shape | |
| ROSETTE | Curled | Curled 1 | leaves are abnormally | Useful for making plants |
| LEAVES | | | curled slightly up or | with altered leaf shape eg |
| | | | down at the leaf | curled leaves |
| | | | margins, but do not | |
| | | | fall under the “cup- | |
| | | | shaped” description | |
| | | | (least severe type) | |
| ROSETTE | Curled | Curled 2 | leaves are abnormally | Useful for making plants |
| LEAVES | | | curled up or down at | with altered leaf shape eg |
| | | | the leaf margins, but | curled leaves |
| | | | do not fall under the | |
| | | | “cup-shaped” | |
| | | | description (more | |
| | | | severe than Curled 1, | |
| | | | but less severe than | |
| | | | Curled 3) | |
| ROSETTE | Curled | Curled 3 | leaves are abnormally | Useful for making plants |
| LEAVES | | | curled up or down at | with altered leaf shape eg |
| | | | the leaf margins, but | curled leaves |
| | | | do not fall under the | |
| | | | “cup-shaped” | |
| | | | description (more | |
| | | | severe than Curled 2, | |
| | | | but less severe than | |
| | | | Curled 4) | |
| ROSETTE | Curled | Curled 4 | leaves are abnormally | Useful for making plants |
| LEAVES | | | curled/rolled up or | with altered leaf shape eg |
| | | | down at the leaf | curled leaves |
| | | | margins (more | |
| | | | severe than Curled 3, | |
| | | | but less severe than | |
| | | | Curled 5) | |
| ROSETTE | Curled | Curled 5 | leaves are completely | Useful for making plants |
| LEAVES | | | curled/rolled up or | with altered leaf shape eg |
| | | | down at the leaf | curled leaves |
| | | | margins (most severe | |
| | | | type) | |
| CAULINE | Curled | Corkscrew | leaves appear as | Useful for making plants |
| LEAVES | | | “Curled 5”, with the | with altered leaf shape eg |
| | | | additional attribute of | curled leaves |
| | | | twisting like a | |
| | | | corkscrew, instead of | |
| | | | uniformly curling | |
| | | | from both sides of the | |
| | | | leaf | |
| CAULINE | Curled | Cup-shaped | the cauline leaves are | Useful for making plants |
| LEAVES | | | curled up at the leaf | with altered leaf shape eg |
| | | | margins such that | curled leaves |
| | | | they form a cup or | |
| | | | bowl-like shape | |
| CAULINE | Curled | Curled 1 | the cauline leaves are | Useful for making plants |
| LEAVES | | | abnormally curled | with altered leaf shape eg |
| | | | slightly up or down at | curled leaves |
| | | | the leaf margins, but | |
| | | | do not fall under the | |
| | | | “cup-shaped” | |
| | | | description (least | |
| | | | severe type) | |
| CAULINE | Curled | Curled 2 | the cauline leaves are | Useful for making plants |
| LEAVES | | | abnormally curled up | with altered leaf shape eg |
| | | | or down at the leaf | curled leaves |
| | | | margins, but do not | |
| | | | fall under the “cup- | |
| | | | shaped” description | |
| | | | (more severe than | |
| | | | Curled 1, but less | |
| | | | severe than Curled 3) | |
| CAULINE | Curled | Curled 3 | the cauline leaves are | Useful for making plants |
| LEAVES | | | abnormally curled up | with altered leaf shape eg |
| | | | or down at the leaf | curled leaves |
| | | | margins, but do not | |
| | | | fall under the “cup- | |
| | | | shaped” description | |
| | | | (more severe than | |
| | | | Curled 2, but less | |
| | | | severe than Curled 4) | |
| CAULINE | Curled | Curled 4 | the cauline leaves are | Useful for making plants |
| LEAVES | | | abnormally | with altered leaf shape eg |
| | | | curled/rolled up or | curled leaves |
| | | | down at the leaf | |
| | | | margins (more | |
| | | | severe than Curled 3, | |
| | | | but less severe than | |
| | | | Curled 5) | |
| CAULINE | Curled | Curled 5 | the cauline leaves are | Useful for making plants |
| LEAVES | | | completely | with altered leaf shape eg |
| | | | curled/rolled up or | curled leaves |
| | | | down at the leaf | |
| | | | margins (most severe | |
| | | | type) | |
| ROSETTE | Size | Small | rosette leaves are | Useful for making plants |
| LEAVES | | | abnormally small | with decreased vegetative |
| | | | (the percent | growth |
| | | | difference in size | |
| | | | compared to the | |
| | | | control should be | |
| | | | noted in the | |
| | | | comments) | |
| COTYLEDONS | Wilted | Wilted | cotyledons appear | Useful for making plants |
| | | | wilted, i.e., they look | with enhanced abiotic |
| | | | as though they have | stress tolerance |
| | | | suffered from | |
| | | | drought conditions | |
| ROSETTE | Wax | Glaucous | leaves are abnormally | Useful for making plants |
| LEAVES | | | dull in appearance | with enhanced abiotic |
| | | | | stress tolerance |
| ROSETTE | Wax | Glossy | leaves are | Useful for making plants |
| LEAVES | | | shiny/glossy in | with enhanced abiotic |
| | | | appearance | stress tolerance |
| CAULINE | Wax | Glaucous | leaves are abnormally | Useful for making plants |
| LEAVES | | | dull in appearance | with enhanced abiotic |
| | | | | stress tolerance |
| CAULINE | Wax | Glossy | leaves are | Useful for making plants |
| LEAVES | | | shiny/glossy in | with enhanced abiotic |
| | | | appearance | stress tolerance |
| WHOLE | Stress | Metabolic | Identify plants with | Useful for making plants |
| PLANT | | Profiling | altered metabolic | with enhanced metabolite |
| | | | profiles as defined in | accumulation |
| | | | 4a | |
| WHOLE | Stress | Plant | Identify plants with | Useful for making plants |
| PLANT | | Architecture | improved architecture | with enhanced plant |
| | | | | architecture |
| WHOLE | Stress | ABA | Identify plants | Useful for making plants |
| PLANT | | | tolerant to ABA, and | with enhanced tolerance |
| | | | possibly drought | to drought |
| | | | and/or other stresses | |
| WHOLE | Stress | Mannitol | Identify plants | Useful for making plants |
| PLANT | | | tolerant to mannitol, | with enhanced tolerance |
| | | | and possibly drought | to drought |
| | | | stress | |
| WHOLE | Stress | Dessication | Identify plants | Useful for making plants |
| PLANT | | | tolerant to water loss, | with enhanced tolerance |
| | | | possibly drought | to drought |
| | | | stress tolerant | |
| WHOLE | Stress | High Sucrose | Identify plants | Useful for making plants |
| PLANT | | | tolerant to high | with enhanced tolerance |
| | | | sucrose conditions | to drought |
| | | | (possible Lead assay | |
| | | | for C/N partitioning) | |
| WHOLE | Stress | Heat | Identify plants with | Useful for making plants |
| PLANT | | | thermotolerance | with enhanced tolerance |
| | | | | to heat |
| WHOLE | Stress | High Nitrogen | Identify plants | Useful for making plants |
| PLANT | | | tolerant to high | with enhanced tolerance |
| | | | nitrogen conditions | to high nitrogen |
| WHOLE | Stress | Etiolation | Identify plants with | Useful for making plants |
| PLANT | | | increased vigor in the | with enhanced tolerance |
| | | | dark | to light stress |
| ROSETTE | Disorganized | Disorganized | rosette leaves do not | Useful for making plants |
| LEAVES | Rosette | Rosette | appear in the normal | with increased biomass |
| | | | fashion, i.e., their | |
| | | | phyllotaxy may be | |
| | | | abnormal or too | |
| | | | many leaves may be | |
| | | | emerging in | |
| | | | comparison to the | |
| | | | control | |
| INFLORESCENCE | Phyllotaxy | Even Phyllotaxy | a phyllotaxy mutant | Useful for making plants |
| | | | whose new branches | with increased biomass |
| | | | emerge at exactly the | |
| | | | same height as each | |
| | | | other, i.e., there is no | |
| | | | internode between | |
| | | | them | |
| COTYLEDONS | Shape | Elliptic Shape | cotyledons are quite | Useful for making plants |
| | | | narrow and pointed, | with increased biomass |
| | | | more so than | and foliage |
| | | | lanceolate | |
| ROSETTE | Fused | Leaf Fused to | the leaf is fused to its | Useful for making plants |
| LEAVES | | Petiole | petiole | with increased biomass |
| | | | | and foliage |
| ROSETTE | Shape | Cordate Shaped | similar to ovate, | Useful for making plants |
| LEAVES | | | except the leaf is not | with increased biomass |
| | | | rounded at its base | and foliage |
| ROSETTE | Shape | Elliptic Shaped | leaves are quite | Useful for making plants |
| LEAVES | | | narrow and pointed, | with increased biomass |
| | | | more so that | and foliage |
| | | | lanceolate | |
| ROSETTE | Shape | Lanceolate | leaves are narrow and | Useful for making plants |
| LEAVES | | Shaped | come to a dull point | with increased biomass |
| | | | at the apex | and foliage |
| ROSETTE | Shape | Lobed Shaped | leaves have very deep | Useful for making plants |
| LEAVES | | | and rounded | with increased biomass |
| | | | serrations, giving an | and foliage |
| | | | appearance of many | |
| | | | lobes forming the | |
| | | | margins of the leaves | |
| ROSETTE | Shape | Oval Shaped | leaves are much | Useful for making plants |
| LEAVES | | | rounder than wild- | with increased biomass |
| | | | type | and foliage |
| ROSETTE | Shape | Ovate Shaped | leaves are wider at | Useful for making plants |
| LEAVES | | | base than at apex, | with increased biomass |
| | | | otherwise similar to | and foliage |
| | | | wild-type | |
| ROSETTE | Shape | Serrate Margins | leaf margins have | Useful for making plants |
| LEAVES | | | little ?teeth? on them, | with increased biomass |
| | | | i.e., they are serrated | and foliage |
| ROSETTE | Shape | Trident Shaped | leaves look | Useful for making plants |
| LEAVES | | | somewhat like a | with increased biomass |
| | | | trident, i.e., they have | and foliage |
| | | | a sharp point at the | |
| | | | apex, and a sharp | |
| | | | point on each side | |
| ROSETTE | Shape | Undulate Shaped | leaves are wavy | Useful for making plants |
| LEAVES | | | | with increased biomass |
| | | | | and foliage |
| WHOLE | Rosette Shape | Bushy Rosette | the different petioles | Useful for making plants |
| PLANT | | Shaped | have very varied | with increased biomass |
| | | | liminal angles, giving | and foliage |
| | | | the plant a very | |
| | | | bushy appearance; | |
| | | | This is often | |
| | | | accompanied by a | |
| | | | “Disorganized | |
| | | | Rosette” phenotype | |
| WHOLE | Rosette Shape | Flat Rosette | the petioles have a | Useful for making plants |
| PLANT | | Shaped | very small liminal | with increased biomass |
| | | | angle, i.e., the rosette | and foliage |
| | | | appears flat instead of | |
| | | | having its usual slight | |
| | | | vertical angle | |
| WHOLE | Rosette Shape | Standing Rosette | the petioles have a | Useful for making plants |
| PLANT | | Shaped | very large liminal | with increased biomass |
| | | | angle, i.e., it appears | and foliage |
| | | | as though the leaves | |
| | | | are standing up | |
| | | | instead of having | |
| | | | their usual small | |
| | | | vertical angle from | |
| | | | the soil | |
| CAULINE | Fused | Leaf Fused to | the cauline leaf is | Useful for making plants |
| LEAVES | | Inflorescence | fused to an | with increased biomass |
| | | | inflorescence or | and foliage |
| | | | branch | |
| CAULINE | Fused | Leaf Fused to | the cauline leaf is | Useful for making plants |
| LEAVES | | Leaf | fused to itself or | with increased biomass |
| | | | another cauline leaf | and foliage |
| CAULINE | Shape | Cordate Shaped | similar to ovate, | Useful for making plants |
| LEAVES | | | except the leaf is not | with increased biomass |
| | | | rounded at its base | and foliage |
| CAULINE | Shape | Elliptic Shaped | leaves are quite | Useful for making plants |
| LEAVES | | | narrow and pointed, | with increased biomass |
| | | | more so that | and foliage |
| | | | lanceolate | |
| CAULINE | Shape | Lanceolate | leaves are narrow and | Useful for making plants |
| LEAVES | | Shaped | come to a dull point | with increased biomass |
| | | | at the apex | and foliage |
| CAULINE | Shape | Lobed Shaped | leaves have very deep | Useful for making plants |
| LEAVES | | | and rounded | with increased biomass |
| | | | serrations, giving an | and foliage |
| | | | appearance of many | |
| | | | lobes forming the | |
| | | | margins of the leaves | |
| CAULINE | Shape | Oval Shaped | leaves are much | Useful for making plants |
| LEAVES | | | rounder than wild- | with increased biomass |
| | | | type | and foliage |
| CAULINE | Shape | Ovate Shaped | leaves are wider at | Useful for making plants |
| LEAVES | | | base than at apex, | with increased biomass |
| | | | otherwise similar to | and foliage |
| | | | wild-type | |
| CAULINE | Shape | Serrate Margins | leaf margins have | Useful for making plants |
| LEAVES | | | little ?teeth? on them, | with increased biomass |
| | | | i.e., they are serrated | and foliage |
| CAULINE | Shape | Trident Shaped | leaves look | Useful for making plants |
| LEAVES | | | somewhat like a | with increased biomass |
| | | | trident, i.e., they have | and foliage |
| | | | a sharp point at the | |
| | | | apex, and a sharp | |
| | | | point on each side | |
| CAULINE | Shape | Undulate Shaped | leaves are wavy | Useful for making plants |
| LEAVES | | | | with increased biomass |
| | | | | and foliage |
| CAULINE | Size | Large | cauline is abnormally | Useful for making plants |
| LEAVES | | | large (the percent | with increased biomass |
| | | | difference in size | and foliage |
| | | | compared to the | |
| | | | control should be | |
| | | | noted in the | |
| | | | comments) | |
| CAULINE | Size | Small | cauline is abnormally | Useful for making plants |
| LEAVES | | | small (the percent | with increased biomass |
| | | | difference in size | and foliage |
| | | | compared to the | |
| | | | control should be | |
| | | | noted in the | |
| | | | comments) | |
| LATERAL | Length | Smaller Lateral | the lateral roots are | Useful for making plants |
| ROOTS | | Root | abnormally short | with increased root |
| | | | | growth to prevent lodging |
| | | | | or enhance nutrient |
| | | | | uptake |
| PRIMARY | Length | Long Primary | the primary root is | Useful for making plants |
| ROOT | | Root | abnormally long | with increased root |
| | | | (the percent | growth to prevent lodging |
| | | | difference in size | or enhance nutrient |
| | | | compared to the | uptake |
| | | | control should be | |
| | | | noted in the | |
| | | | comments) | |
| PRIMARY | Length | Short Primary | the primary root is | Useful for making plants |
| ROOT | | Root | abnormally short | with increased root |
| | | | (the percent | growth to prevent lodging |
| | | | difference in size | or enhance nutrient |
| | | | compared to the | uptake |
| | | | control should be | |
| | | | noted in the | |
| | | | comments) | |
| WHOLE | Stress | Plant Size | Identify plants of | Useful for making plants |
| PLANT | | | increased size | with increased size and |
| | | | compared to wild | biomass |
| | | | type | |
| WHOLE | Stress | Starch | Identify plants with | Useful for making plants |
| PLANT | | | increased starch | with increased starch |
| | | | accumulation | content |
| WHOLE | Stress | Cold | Identify plants that | Useful for making plants |
| PLANT | | Germination | germinate better at | with increased tolerance |
| | | | cold temperatures | to cold stress |
| WHOLE | Stress | Cold Growth | Identify plants that | Useful for making plants |
| PLANT | | | grow faster at cold | with increased tolerance |
| | | | temperatures | to cold stress |
| WHOLE | Stress | Soil Drought | Identify plants with | Useful for making plants |
| PLANT | | | increased tolerance to | with increased tolerance |
| | | | soil drought | to drought |
| WHOLE | Stress | Soil Drought - | Identify plants that | Useful for making plants |
| PLANT | | Desiccation | are tolerant to low | with increased tolerance |
| | | tolerance | soil moisture and | to drought |
| | | | resist wilting | |
| WHOLE | Stress | PEG | Identify plants | Useful for making plants |
| PLANT | | | tolerant to PEG, and | with increased tolerance |
| | | | possibly drought | to drought |
| | | | stress | |
| SEED | Size | Large | the seed is | Useful for making plants |
| | | | abnormally large | with larger seeds |
| | | | (the percent | |
| | | | difference in size | |
| | | | compared to the | |
| | | | control should be | |
| | | | noted in the | |
| | | | comments) | |
| INFLORESCENCE | Branching | Asecondary | the plant does not | Useful for making plants |
| | | Branching | form any secondary | with modified flowers |
| | | | inflorescences | |
| SEED | Size | Small | the seed is | Useful for making plants |
| | | | abnormally small | with smaller seeds or no |
| | | | (the percent | seeds |
| | | | difference in size | |
| | | | compared to the | |
| | | | control should be | |
| | | | noted in the | |
| | | | comments) | |
| WHOLE | Stress | C/N Content | Identify plants/seeds | Useful for making seeds |
| PLANT | | | with altered | with altered |
| | | | carbon/nitrogen | carbon/nitrogen levels |
| | | | levels | |
| INFLORESCENCE | Internode Length | Short Internode | the internode is | Useful for making shorter |
| | | | abnormally short | plants and plants with |
| | | | (the percent | modified flowers |
| | | | difference in length | |
| | | | compared to the | |
| | | | control should be | |
| | | | noted in the | |
| | | | comments) | |
| WHOLE | Dwarf | Brassino-Steroid | these plants are small | Useful for making smaller |
| PLANT | | Dwarf | in stature, dark green, | plants |
| | | | have oval leaves, | |
| | | | strong bolts, and are | |
| | | | often sterile | |
| WHOLE | Dwarf | Misc. Dwarf | these are dwarf plants | Useful for making smaller |
| PLANT | | | the do not fall under | plants |
| | | | the brassino-steroid | |
| | | | dwarf category | |
| HYPOCOTYL | Length | Short | hypocotyl is visibly | Useful for making smaller |
| | | | shorter than in wild- | plants |
| | | | type (the percent | |
| | | | difference in size | |
| | | | compared to the | |
| | | | control should be | |
| | | | noted in the | |
| | | | comments) | |
| INFLORESCENCE | Height | Short | the inflorescences of | Useful for making smaller |
| | | | the plants are | plants |
| | | | abnormally short | |
| | | | (plant height is | |
| | | | encompassed under | |
| | | | the whole plant size | |
| | | | category, but this | |
| | | | entry would be used | |
| | | | if the height of the | |
| | | | plant is abnormal, but | |
| | | | is otherwise of | |
| | | | normal size) (the | |
| | | | percent difference in | |
| | | | size | |
| WHOLE | Size | Small | plant is abnormally | Useful for making smaller |
| SEEDLING | | | small (the percent | plants |
| | | | difference in size | |
| | | | compared to the | |
| | | | control should be | |
| | | | noted in the | |
| | | | comments) | |
| ROSETTE | Petiole Length | Short Petioles | the leaf petioles are | Useful for making smaller |
| LEAVES | | | abnormally short | plants |
| | | | (the percent | |
| | | | difference in size | |
| | | | compared to the | |
| | | | control should be | |
| | | | noted in the | |
| | | | comments) | |
| WHOLE | Size | Small | plant is abnormally | Useful for making smaller |
| PLANT | | | small (the percent | plants |
| | | | difference in size | |
| | | | compared to the | |
| | | | control should be | |
| | | | noted in the | |
| | | | comments) | |
| CAULINE | Petiole Length | Short Petioles | the cauline petioles | Useful for making smaller |
| LEAVES | | | are abnormally short | plants |
| | | | (the percent | |
| | | | difference in size | |
| | | | compared to the | |
| | | | control should be | |
| | | | noted in the | |
| | | | comments) | |
| INFLORESCENCE | Strength | Strong | the primary | Useful for making |
| | | | inflorescence appears | stronger plants |
| | | | significantly stronger, | |
| | | | whether by thickness | |
| | | | or rigidity | |
| INFLORESCENCE | Strength | Weak | the primary | Useful for making |
| | | | inflorescence appears | stronger plants |
| | | | significantly weaker, | |
| | | | whether by thickness | |
| | | | or rigidity | |
| INFLORESCENCE | Inflorescence | Thickness | thickness of the | Useful for making |
| | | | primary inflorescence | stronger plants |
| HYPOCOTYL | Length | Long | hypocotyl is visibly | Useful for making taller |
| | | | longer than in wild- | plants |
| | | | type (the percent | |
| | | | difference in size | |
| | | | compared to the | |
| | | | control should be | |
| | | | noted in the | |
| | | | comments) | |
| INFLORESCENCE | Internode Length | Long Internode | the internode is | Useful for making taller |
| | | | abnormally long (the | plants and plants with |
| | | | percent difference in | longer flowers |
| | | | length compared to | |
| | | | the control should be | |
| | | | noted in the | |
| | | | comments) | |
| INFLORESCENCE | Height | Tall | the inflorescences of | Useful for making taller |
| | | | the plants are | plants and plants with |
| | | | abnormally long | longer inflorescences |
| | | | (plant height is | |
| | | | encompassed under | |
| | | | the whole plant size | |
| | | | category, but this | |
| | | | entry would be used | |
| | | | if the height of the | |
| | | | plant is abnormal, but | |
| | | | is otherwise of | |
| | | | normal size) (the | |
| | | | percent difference in | |
| | | | size | |
| SEED | Color | Dark Color | the seed is | Useful for modifying |
| | | | abnormally dark | fiber content in seed |
| SEED | Color | Light Color | the seed is | Useful for modifying |
| | | | abnormally light; | fiber content in seed |
| | | | Transparent Testa is | |
| | | | an example of this | |
| | | | phenotype | |
| SILIQUES | Shape | Bent | the silique has sharp | Useful for modifying fruit |
| | | | bend to it part of the | shape, composition and |
| | | | way down the length | seed yield |
| | | | of the silique; this | |
| | | | bend can be as much | |
| | | | as approaching 90 | |
| | | | degrees | |
| SILIQUES | Shape | Bulging | the seeds in the | Useful for modifying fruit |
| | | | silique appears | shape, composition and |
| | | | “shrink-wrapped”, | seed yield |
| | | | giving the silique a | |
| | | | bulging appearance | |
| SILIQUES | Shape | Clubbed | the silique is | Useful for modifying fruit |
| | | | somewhat bulbous at | shape, composition and |
| | | | its terminal end | seed yield |
| SILIQUES | Shape | Sickle | the silique is curved, | Useful for modifying fruit |
| | | | much like the blade | shape, composition and |
| | | | of a sickle | seed yield |
| INFLORESCENCE | Branching | No Branching | there is no branching | Useful for modifying |
| | | | at all | plant architecture, ie |
| | | | | amount of branching |
| INFLORESCENCE | Branching | Horizontal | new branches arise at | Useful for modifying |
| | | Branching | a 90 degree angle | plant architecture, ie |
| | | | from the bolt they are | branch angle |
| | | | emerging from | |
| COTYLEDONS | Horizontally | Horizontally | cotyledon is visibly | Useful for modifying |
| | Oblong | Oblong | wider than it is long, | plant architecture, ie leaf |
| | | | and it is also | structure |
| | | | symmetrical (or very | |
| | | | close to it) when cut | |
| | | | along its horizontal | |
| | | | axis | |
| INFLORESCENCE | Branching | Two Leaf | two cauline leaves | Useful for modifying |
| | | Branching | subtend branches | plant architecture, ie |
| | | | instead of one | reducing foliage |
| INFLORESCENCE | Branching | Reduced Apical | the dominance of the | Useful for modifying |
| | | Dominance | primary inflorescence | plant structure, ie |
| | | | is diminished, with | increased branching |
| | | | the secondaries | |
| | | | appearing as | |
| | | | dominant or nearly as | |
| | | | dominant | |
| SEED | Seed | Stacked | the seeds/embryos | Useful for modifying seed |
| | Arrangement | Arrangement | are stacked one on | content |
| | | | top of the other | |
| | | | within the silique, | |
| | | | instead of having the | |
| | | | usual side-by-side | |
| | | | distribution | |
| SEED | Other | Other | this correlates with | Useful for modifying seed |
| | | | any seed mutant | content |
| | | | phenotypes which do | |
| | | | not fit into the above | |
| | | | categories (a picture | |
| | | | should be taken for | |
| | | | documentation) | |
| SEED | Shape | Oval Shape | the seeds are much | Useful for modifying seed |
| | | | more rounded on the | structure and composition |
| | | | ends, giving the seed | |
| | | | a true oval | |
| | | | appearance | |
| SEED | Shape | Ridged Shape | the seeds have small | Useful for modifying seed |
| | | | ridges or bumps on | structure and composition |
| | | | them | |
| SEED | Shape | Tapered Shape | the ends of the seeds | Useful for modifying seed |
| | | | narrow down to a | structure and composition |
| | | | much sharper point | |
| | | | than usual | |
| COTYLEDONS | Cotyledon | Single Cotyledon | Only one cotyledon | Useful for modifying seed |
| | Number | | appears after | structure and content |
| | | | germination; This is | |
| | | | simply one cotyledon | |
| | | | that had formed | |
| | | | instead of two, and is | |
| | | | not related to the | |
| | | | fused phenotype; | |
| | | | With this exception, | |
| | | | the plant is often | |
| | | | otherwise wild-type | |
| | | | in appearance | |
| COTYLEDONS | Cotyledon | Tricot | three cotyledons | Useful for modifying seed |
| | Number | | emerge instead of | structure and content |
| | | | two; With this | |
| | | | exception, the plant is | |
| | | | often otherwise wild- | |
| | | | type in appearance | |
| COTYLEDONS | Curled | Cup-shaped | cotyledons are curled | Useful for modifying seed |
| | | | up at the cotyledon | structure and content |
| | | | margins such that | |
| | | | they form a cup or | |
| | | | bowl-like shape | |
| COTYLEDONS | Curled | Curled 1 | cotyledons are | Useful for modifying seed |
| | | | abnormally curled | structure and content |
| | | | slightly up or down at | |
| | | | he cotyledon | |
| | | | margins, but do not | |
| | | | fall under the “cup- | |
| | | | shaped” description | |
| | | | (least severe type) | |
| COTYLEDONS | Curled | Curled 2 | cotyledons are | Useful for modifying seed |
| | | | abnormally curled up | structure and content |
| | | | or down at the | |
| | | | cotyledon margins, | |
| | | | but do not fall under | |
| | | | the “cup-shaped” | |
| | | | description (more | |
| | | | severe than Curled 1, | |
| | | | but less severe than | |
| | | | Curled 3) | |
| COTYLEDONS | Curled | Curled 3 | cotyledons are | Useful for modifying seed |
| | | | abnormally curled up | structure and content |
| | | | or down at the | |
| | | | cotyledon margins, | |
| | | | but do not fall under | |
| | | | the “cup-shaped” | |
| | | | description (more | |
| | | | severe than Curled 2, | |
| | | | but less severe than | |
| | | | Curled 4) | |
| COTYLEDONS | Curled | Curled 4 | cotyledons are | Useful for modifying seed |
| | | | abnormally | structure and content |
| | | | curled/rolled up or | |
| | | | down at the | |
| | | | cotyledon margins | |
| | | | (more severe than | |
| | | | Curled 3, but less | |
| | | | severe than Curled 5) | |
| COTYLEDONS | Curled | Curled 5 | cotyledons are | Useful for modifying seed |
| | | | completely | structure and content |
| | | | curled/rolled up or | |
| | | | down at the | |
| | | | cotyledon margins | |
| | | | (most severe type) | |
| COTYLEDONS | Dimorphic | Dimorphic | one cotyledon is | Useful for modifying seed |
| | Cotyledons | Cotyledons | significantly larger | structure and content |
| | | | than the other | |
| COTYLEDONS | Fused | Fused 1 | cotyledons are fused | Useful for modifying seed |
| | | | to each other, | structure and content |
| | | | creating one | |
| | | | cotyledon structure | |
| | | | (least severe type) | |
| COTYLEDONS | Fused | Fused 2 | cotyledons are fused | Useful for modifying seed |
| | | | to each other, | structure and content |
| | | | creating one | |
| | | | cotyledon structure | |
| | | | (more severe than | |
| | | | Fused 1, but less | |
| | | | severe than Fused 3) | |
| COTYLEDONS | Fused | Fused 3 | cotyledons are fused | Useful for modifying seed |
| | | | to each other, | structure and content |
| | | | creating one | |
| | | | cotyledon structure | |
| | | | (more severe than | |
| | | | Fused 2, but less | |
| | | | severe than Fused 4) | |
| COTYLEDONS | Fused | Fused 4 | cotyledons are fused | Useful for modifying seed |
| | | | to each other, | structure and content |
| | | | creating one | |
| | | | cotyledon structure | |
| | | | (more severe than | |
| | | | Fused 3, but less | |
| | | | severe than Fused 5) | |
| COTYLEDONS | Fused | Fused 5 | cotyledons are fused | Useful for modifying seed |
| | | | to each other, | structure and content |
| | | | creating one | |
| | | | cotyledon structure | |
| | | | (most severe type) | |
| COTYLEDONS | Other | Other | this correlates with | Useful for modifying seed |
| | | | any cotyledon mutant | structure and content |
| | | | phenotypes which do | |
| | | | not fit into the above | |
| | | | categories (a picture | |
| | | | should be taken for | |
| | | | documentation) | |
| ROSETTE | Fused | Leaf Fused to | the leaf is fused to | Useful for plants with |
| LEAVES | | Leaf | itself or another leaf | fused leaves eg |
| | | | | ornamentals |
| COTYLEDONS | Petiole Length | Short Petioles | the cotyledon petioles | Useful for shade |
| | | | are abnormally short | avoidance and for making |
| | | | (the percent | smaller plants |
| | | | difference in size | |
| | | | compared to the | |
| | | | control should be | |
| | | | noted in the | |
| | | | comments) | |
| PRIMARY | Agravitropic | Agravitropic | the primary root does | |
| ROOT | | | not appear to have a | |
| | | | gravitropic response | |
| PRIMARY | Kinked | Kinked | there is a sharp bend | |
| ROOT | | | in the root | |
| ROSETTE | Rosette Diameter | Diameter | diameter of rosette | |
| LEAVES | | | | |
| WHOLE | Plant Weight | Plant Weight | weight of whole plant | |
| PLANT | | | | |
| WHOLE | Plant Height | Height | height of whole plant | |
| PLANT | | | | |
| WHOLE | Plant DTH | Dth | days to harvest of | |
| PLANT | | | plant | |
| WHOLE | Plant Harvest | Harvest Index | harvest index of plant | — |
| PLANT | Index | | | |
| CAULINE | Fused | Leaf Fused to | the cauline leaf is | |
| LEAVES | | Petiole | fused to its petiole | |
| N/A | N/A | N/A | N/A | |
| WHOLE | HERBICIDE | HERBICIDE | herbicide segregation | |
| PLANT | SEGREGATION | SEGREGATION | ratio | |
| WHOLE | N/A | No Mutant | The plants were | |
| PLANT | | Phenotype | screened at all | |
| | | Observed | appropriate stages | |
| | | | and showed no | |
| | | | mutant phenotype, | |
| | | | i.e., they looked like | |
| | | | normal, wild type | |
| | | | Arabidopsis plants | |

From the results reported in Table 1 and the Sequence Listing, it can be seen that the nucleotides/polypeptides of the inventions are useful, depending upon the respective individual sequence, to make plants with modified growth and phenotype characteristics, including:
1. modulated plant size, including increased and decreased height or length;
2. modulated vegetative growth (increased or decreased);
3. modulated organ number;
4. increased biomass;
5. sterility;
6. seedling lethality;
7. accelerated crop development or harvest;
8. accelerated flowering time;
9. delayed flowering time;
10. delayed senescence;
11. enhanced drought or stress tolerance;
12. increased chlorophyll and photosynthetic capacity;
13. increased anthocyanin content;
14. increased root growth, and increased nutrient uptake;
15. increased or decreased seed weight or size, increased seed carbon or nitrogen content;
16. modified, including increased, seed/fruit yield or modified fruit content;
17. enhanced foliage;
18. usefulness for making nutratceuticals/pharmaceuticals in plants;
19. plant lethality;
20. decrease seed fiber content to provide increased digestability;
21. modified ornamental appearance with modified leaves, flowers, color or foliage;
22. modified sterility in plants;
23. enhanced ability to grow in shade;
24. enhanced biotic stress tolerance;
25. increased tolerance to density and low fertilizer;
26. enhanced tolerance to high or low pH, to low or high nitrogen or phosphate;
27. enhanced tolerance to oxidative stress;
28. enhanced chemical composition;
29. altered leaf shape;
30. enhanced abiotic stress tolerance;
31. increased tolerance to cold stress;
32. increased starch content;
33. reduced number or no seeds;
34. enhanced plant strength;
35. modified flower length;
36. longer inflorescences;
37. modified seed fiber content;
38. modified fruit shape;
39. modified fruit composition;
40. modified seed yield;
41. modified plant architecture, such as modified amount or angle of branching, modified leaf structure, or modified seed structure; and
42. enhanced shade avoidance.

According to another aspect, the nucleotide sequences of the invention encode polypeptides that can be utilized as herbicide targets, those useful in the screening of new herbicide compounds. Thus, the proteins encoded by the nucleotide sequences provide the bases for assays designed to easily and rapidly identify novel herbicides.

According to yet another aspect, the present invention provides a method of identifying a herbicidal compound, comprising: (a) combining a polypeptide comprising an amino acid sequence at least 85% identical to an amino acid sequence selected from the group consisting of the polypeptides described in FIGS. 1-73 with a compound to be tested for the ability to inhibit the activity of said polypeptide, under conditions conducive to inhibition; (b) selecting a compound identified in (a) that inhibits the activity of said polypeptide; (c) applying a compound selected in (b) to a plant to test for herbicidal activity; (d) selecting a compound identified in (c) that has herbicidal activity. The polypeptide can alternatively comprise an amino acid sequence at least 90%, or at least 95%, or at least 99% identical to an amino acid sequence selected from the group consisting of the polypeptides in FIGS. 1-73. The present invention also provides a method for killing or inhibiting the growth or viability of a plant, comprising applying to the plant a herbicidal compound identified according to this method.

Determination of Functional Homolog Sequences

The “Lead” sequences described in the Sequence Listing **-** and identified in FIGS. 1-73 with a Lead number, *** are utilized to identify functional homologs of the lead sequences and, together with those sequences, are utilized to determine a consensus sequence for a given group of lead and functional homolog sequences.

A subject sequence is considered a functional homolog of a query sequence if the subject and query sequences encode proteins having a similar function and/or activity. A process known as Reciprocal BLAST (Rivera et al, Proc. Natl Acad. Sci. USA, 1998, 95:6239-6244) is used to identify potential functional homolog sequences from databases consisting of all available public and proprietary peptide sequences, including NR from NCBI and peptide translations from Ceres clones.

Before starting a Reciprocal BLAST process, a specific query polypeptide is searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides are designated as a cluster.

The main Reciprocal BLAST process consists of two rounds of BLAST searches; forward search and reverse search. In the forward search step, a query polypeptide sequence, “polypeptide A,” from source species S^{A} is BLASTed against all protein sequences from a species of interest. Top hits are determined using an E-value cutoff of 10⁻⁵ and an identity cutoff of 35%. Among the top hits, the sequence having the lowest E-value is designated as the best hit, and considered a potential functional homolog. Any other top hit that had a sequence identity of 80% or greater to the best hit or to the original query polypeptide is considered a potential functional homolog as well. This process is repeated for all species of interest.

In the reverse search round, the top hits identified in the forward search from all species are used to perform a BLAST search against all protein or polypeptide sequences from the source species S^{A}. A top hit from the forward search that returned a polypeptide from the aforementioned cluster as its best hit is also considered as a potential functional homolog.

Functional homologs are identified by manual inspection of potential functional homolog sequences. Representative functional homologs are shown in FIGS. 1-5. Each Figure represents a grouping of a lead/query sequence aligned with the corresponding identified functional homolog subject sequences. Lead sequences and their corresponding functional homolog sequences are aligned to identify conserved amino acids and to determine a consensus sequence that contains a frequently occurring amino acid residue at particular positions in the aligned sequences, as shown in FIGS. 1-73.

Each consensus sequence then is comprised of the identified and numbered conserved regions or domains, with some of the conserved regions being separated by one or more amino acid residues, represented by a dash (-), between conserved regions.

Useful polypeptides of the inventions, therefore, include each of the lead and functional homolog sequences shown in FIGS. 1-73, as well as the consensus sequences shown in those Figures. The invention also encompasses other useful polypeptides constructed based upon the consensus sequence and the identified conserved regions. Thus, useful polypeptides include those which comprise one or more of the numbered conserved regions in each alignment table in an individual Figure depicted in FIGS. 1-73, wherein the conserved regions may be separated by dashes. Useful polypeptides also include those which comprise all of the numbered conserved regions in an individual alignment table selected from FIGS. 1-73, alternatively comprising all of the numbered conserved regions in an individual alignment table and in the order as depicted in an individual alignment table selected from FIGS. 1-73. Useful polypeptides also include those which comprise all of the numbered conserved regions in an individual alignment table and in the order as depicted in an individual alignment table selected from FIGS. 1-73, wherein the conserved regions are separated by dashes, wherein each dash between two adjacent conserved regions is comprised of the amino acids depicted in the alignment table for lead and/or functional homolog sequences at the positions which define the particular dash. Such dashes in the consensus sequence can be of a length ranging from length of the smallest number of dashes in one of the aligned sequences up to the length of the highest number of dashes in one of the aligned sequences.

Such useful polypeptides can also have a length (a total number of amino acid residues) equal to the length identified for a consensus sequence or of a length ranging from the shortest to the longest sequence in any given family of lead and functional homolog sequences identified in an individual alignment table selected from FIGS. 1-73.

The Sequence Listing sets forth the polypeptide and polynucleotide sequences of the invention, including the Lead, ortholog and consensus sequences presented in FIGS. 1-73.

Table 2 correlates the sequences in the Sequence Listing with those shown in the alignment tables of FIGS. 1-73. As noted above, each Figure represents the alignment table for a particular “Lead” sequence and shows the group of functional homologs for that “Lead” sequence. Some identified homologs are not presented in the Figures but are listed in the Sequence Listing. So Table 2 also groups together the functional homologs by correlating each homolog with the relevant “Lead” sequence (referred to in Table 2 as the “query identifier”) and the table also presents other information for each of the functional homologs, including the % identity of the homolog relative to the query/Lead sequence, the corresponding E-value, the plant species for the homolog, the Sequence ID No. in the Sequence Listing, and an indication of whether or not the sequence is presented in the corresponding alignment table in one of the Figures.

The present invention further encompasses nucleotides that encode the above described polypeptides, as well as the complements thereof, and including alternatives thereof based upon the degeneracy of the genetic code.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

TABLE 2

| | | | | | | IN |
|---|---|---|---|---|---|---|
| LEAD | FUNCTIONAL | PERCENT | | | SEQ ID | ALIGNMENT |
| SEQ ID | HOMOLOG ID | IDENTITY | E-VALUE | SPECIES | NO | TABLE |
| 12321246 | 1442604 | 54.57 | 7.5E−127 | Populus balsamifera subsp. trichocarpa | 83 | YES |
| 12321246 | 1442608 | 51.10 | 2.2E−113 | Populus balsamifera subsp. trichocarpa | 85 | NO |
| 12321246 | 1452827 | 50.63 | 2.6E−124 | Populus balsamifera subsp. trichocarpa | 87 | NO |
| 12321246 | 1442612 | 50.00 | 1.8E−116 | Populus balsamifera subsp. trichocarpa | 89 | NO |
| 12321246 | 522267 | 47.27 | 1.8E−119 | Glycine max | 90 | YES |
| 12321246 | 474116 | 47.57 | 1.3E−111 | Glycine max | 91 | NO |
| 12330770 | 151087 | 100.00 | 0 | Arabidopsis thaliana | 92 | NO |
| 12330770 | 1504145 | 67.40 | 3.3E−133 | Populus balsamifera subsp. trichocarpa | 96 | YES |
| 12330770 | 1005083 | 62.85 | 0 | Triticum aestivum | 97 | YES |
| 12330770 | 50910970 | 63.61 | 1.1E−130 | Oryza sativa subsp. japonica | 98 | YES |
| 12330770 | 337070 | 60.42 | 2E−122 | Zea mays | 99 | YES |
| 12330770 | 1504146 | 58.55 | 1.1E−88 | Populus balsamifera subsp. trichocarpa | 101 | NO |
| 23363031 | 1480518 | 96.82 | 1.2E−199 | Populus balsamifera subsp. trichocarpa | 105 | YES |
| 23363031 | 1039306 | 96.57 | 0 | Brassica napus | 106 | YES |
| 23363031 | 581299 | 96.56 | 1.5E−199 | Glycine max | 107 | YES |
| 7090414 | 21436457 | 90.88 | 1.1E−167 | Arabidopsis thaliana | 114 | NO |
| 7090414 | 1346028 | 81.18 | 4.4E−153 | Lupinus albus | 115 | YES |
| 7090414 | 20135548 | 81.18 | 4.4E−153 | Malus x domestica | 116 | YES |
| 7090414 | 34013692 | 80.29 | 1.8E−149 | Hevea brasiliensis | 117 | YES |
| 7090414 | 1346029 | 80.00 | 1.5E−150 | Lupinus albus | 118 | NO |
| 7090414 | 62199628 | 79.41 | 3.8E−147 | Vitis vinifera | 119 | YES |
| 12676463 | 58397752 | 51.33 | 8.5E−28 | Teucrium chamaedrys | 122 | NO |
| 12676463 | 3582021 | 46.31 | 2.7E−113 | Nepeta racemosa | 123 | YES |
| 12676463 | 46947673 | 46.04 | 8.5E−103 | Ammi majus | 124 | YES |
| 12676463 | 117188 | 45.95 | 2.3E−107 | Persea americana | 125 | NO |
| 12676463 | 34904242 | 45.58 | 2.1E−99 | Oryza sativa subsp. japonica | 126 | YES |
| 12676463 | 921721 | 45.25 | 4.7E−102 | Triticum aestivum | 127 | NO |
| 12676463 | 703961 | 45.25 | 4.7E−102 | Triticum aestivum | 128 | YES |
| 12676463 | 25282608 | 45.25 | 2.8E−111 | Persea americana | 129 | YES |
| 36531424 | 79501393 | 80.95 | 2.1E−218 | Arabidopsis thaliana | 153 | NO |
| 36531424 | 1509745 | 57.33 | 3.6E−143 | Populus balsamifera subsp. trichocarpa | 155 | YES |
| 36531424 | 1456553 | 56.31 | 2.7E−147 | Populus balsamifera subsp. trichocarpa | 157 | NO |
| 36531424 | 365873 | 49.13 | 1.7E−113 | Zea mays | 158 | YES |
| 36531424 | 511739 | 48.80 | 2.6E−117 | Glycine max | 159 | YES |
| 36531424 | 770598 | 47.90 | 1.5E−123 | Triticum aestivum | 160 | YES |
| 36531424 | 1450731 | 46.68 | 2.2E−120 | Populus balsamifera subsp. trichocarpa | 162 | NO |
| 36531424 | 34906258 | 45.06 | 3.2E−105 | Oryza sativa subsp. japonica | 163 | YES |
| 12718491 | 1443044 | 67.12 | 5.5E−163 | Populus balsamifera subsp. trichocarpa | 167 | YES |
| 12718491 | 64180315 | 41.47 | 3E−92 | Taxus cuspidata | 168 | YES |
| 12718491 | 53759170 | 41.47 | 8.9E−92 | Taxus chinensis | 169 | NO |
| 12718491 | 60459952 | 39.57 | 1.4E−86 | Taxus x media | 170 | YES |
| 12718491 | 38481843 | 35.64 | 6.8E−83 | Taxus chinensis | 171 | NO |
| 12718491 | 67633430 | 39.10 | 9.4E−80 | Taxus canadensis | 172 | YES |
| 12718491 | 34559857 | 34.78 | 3.7E−82 | Taxus cuspidata | 173 | NO |
| 12718491 | 59800276 | 38.46 | 8.2E−81 | Picea sitchensis | 174 | NO |
| 12718491 | 59800274 | 38.25 | 5E−81 | Picea sitchensis | 175 | YES |
| 12718491 | 50937811 | 33.62 | 2E−74 | Oryza sativa subsp. japonica | 176 | YES |
| 12718491 | 63108254 | 35.20 | 3.4E−15 | Eschscholzia californica | 177 | NO |
| 12718491 | 45260636 | 31.91 | 6.5E−62 | Nicotiana tabacum | 178 | NO |
| 12370997 | 1471370 | 76.61 | 8.7E−181 | Populus balsamifera subsp. trichocarpa | 182 | NO |
| 12370997 | 1444471 | 74.24 | 1E−193 | Populus balsamifera subsp. trichocarpa | 184 | YES |
| 12370997 | 1438451 | 73.32 | 6.3E−178 | Populus balsamifera subsp. trichocarpa | 185 | NO |
| 12370997 | 1438451 | 73.32 | 6.3E−178 | Populus balsamifera subsp. trichocarpa | 186 | NO |
| 12370997 | 1447690 | 72.17 | 5.8E−175 | Populus balsamifera subsp. trichocarpa | 188 | NO |
| 12370997 | 1491278 | 71.46 | 7.3E−184 | Populus balsamifera subsp. trichocarpa | 190 | NO |
| 12370997 | 624225 | 68.64 | 0 | Glycine max | 191 | NO |
| 12370997 | 2739008 | 67.24 | 0 | Glycine max | 192 | YES |
| 12370997 | 779234 | 65.34 | 0 | Triticum aestivum | 193 | YES |
| 12370997 | 50948231 | 63.20 | 0 | Oryza sativa subsp. japonica | 194 | YES |
| 12370997 | 50725143 | 62.81 | 0 | Oryza sativa subsp. japonica | 195 | NO |
| 12370997 | 1551657 | 62.60 | 5.7E−160 | Zea mays | 196 | NO |
| 12370997 | 1601442 | 55.71 | 9.7E−28 | Zea mays | 197 | NO |
| 12370997 | 1600726 | 56.51 | 4.3E−76 | Zea mays | 198 | YES |
| 12370997 | 5921925 | 56.50 | 0 | Pinus radiata | 199 | YES |
| 12370997 | 22758273 | 56.35 | 0 | Oryza sativa subsp. japonica | 200 | NO |
| 12558789 | 68164961 | 87.48 | 2.7E−241 | Malus x domestica | 203 | YES |
| 12558789 | 1470719 | 87.27 | 1.5E−206 | Populus balsamifera subsp. trichocarpa | 205 | YES |
| 12558789 | 1479959 | 87.24 | 6.6E−206 | Populus balsamifera subsp. trichocarpa | 207 | NO |
| 12558789 | 1543728 | 87.04 | 5.2E−206 | Populus balsamifera subsp. trichocarpa | 209 | NO |
| 12558789 | 16555877 | 86.73 | 0 | Lithospermum erythrorhizon | 210 | YES |
| 12575176 | 1444156 | 52.00 | 1.5E−112 | Populus balsamifera subsp. trichocarpa | 228 | YES |
| 12575176 | 1444154 | 51.76 | 1.2E−110 | Populus balsamifera subsp. trichocarpa | 230 | NO |
| 12575176 | 1497097 | 51.52 | 1.6E−110 | Populus balsamifera subsp. trichocarpa | 232 | NO |
| 12660455 | 1525729 | 74.13 | 5.7E−177 | Populus balsamifera subsp. trichocarpa | 255 | NO |
| 12660455 | 1470773 | 70.47 | 2.6E−158 | Populus balsamifera subsp. trichocarpa | 257 | NO |
| 12660455 | 1524187 | 70.40 | 1.9E−169 | Populus balsamifera subsp. trichocarpa | 259 | YES |
| 12660455 | 11934677 | 63.80 | 0 | Cucurbita maxima | 260 | YES |
| 12660455 | 27764531 | 63.99 | 0 | Pisum sativum | 261 | YES |
| 12660455 | 13022042 | 57.26 | 0 | Hordeum vulgare subsp. vulgare | 262 | YES |
| 12660455 | 703821 | 39.69 | 3.4E−33 | Triticum aestivum | 263 | YES |
| 12660455 | 47498770 | 54.89 | 0 | Ginkgo biloba | 264 | YES |
| 12660455 | 391105 | 53.78 | 0 | Zea mays | 265 | YES |
| 12660455 | 5915847 | 53.78 | 0 | Zea mays | 266 | NO |
| 12605081 | 1453454 | 84.96 | 5E−169 | Populus balsamifera subsp. trichocarpa | 270 | YES |
| 12605081 | 473273 | 79.72 | 0 | Glycine max | 271 | YES |
| 12605081 | 2738998 | 80.36 | 0 | Glycine max | 272 | YES |
| 12605081 | 22651519 | 78.74 | 0 | Ocimum basilicum | 273 | YES |
| 12605081 | 1528108 | 79.11 | 2.3E−157 | Populus balsamifera subsp. trichocarpa | 275 | NO |
| 12605081 | 1474685 | 79.11 | 1.2E−153 | Populus balsamifera subsp. trichocarpa | 276 | NO |
| 12605081 | 22651521 | 78.54 | 0 | Ocimum basilicum | 278 | YES |
| 12605081 | 46947675 | 75.59 | 0 | Ammi majus | 279 | YES |
| 12654761 | 1457794 | 48.84 | 1.3E−124 | Populus balsamifera subsp. trichocarpa | 283 | YES |
| 12654761 | 1548098 | 47.72 | 4.2E−117 | Zea mays | 284 | YES |
| 12654761 | 77552864 | 46.71 | 1.3E−120 | Oryza sativa subsp. japonica | 285 | YES |
| 12654761 | 50940049 | 45.36 | 3.2E−108 | Oryza sativa subsp. japonica | 286 | NO |
| 12654761 | 13661758 | 42.05 | 6.2E−105 | Lolium rigidum | 287 | NO |
| 12654761 | 13661756 | 42.58 | 3.7E−107 | Lolium rigidum | 288 | YES |
| 12654761 | 1463878 | 44.25 | 9.8E−86 | Populus balsamifera subsp. trichocarpa | 290 | NO |
| 12654761 | 818090 | 34.65 | 2.3E−11 | Triticum aestivum | 291 | YES |
| 12654761 | 57863822 | 42.67 | 2.7E−106 | Oryza sativa subsp. japonica | 292 | NO |
| 12724226 | 1510416 | 82.29 | 7E−195 | Populus balsamifera subsp. trichocarpa | 296 | YES |
| 12724226 | 1541253 | 79.48 | 6.1E−196 | Populus balsamifera subsp. trichocarpa | 298 | NO |
| 12724226 | 71834076 | 74.11 | 1.3E−185 | Zinnia elegans | 299 | YES |
| 12724226 | 60677681 | 73.89 | 0 | Oryza sativa subsp. japonica | 300 | YES |
| 12724226 | 34902330 | 69.31 | 0 | Oryza sativa subsp. japonica | 301 | NO |
| 12724226 | 1578373 | 73.72 | 0 | Zea mays | 302 | YES |
| 12724226 | 1583137 | 73.39 | 0 | Zea mays | 303 | NO |
| 12724226 | 50058152 | 45.96 | 1.9E−101 | Oryza sativa subsp. japonica | 304 | NO |
| 12724226 | 390429 | 44.21 | 2E−99 | Zea mays | 305 | NO |
| 12724226 | 234510 | 44.73 | 8.6E−99 | Zea mays | 306 | NO |
| 12724226 | 1472214 | 46.47 | 4.7E−102 | Populus balsamifera subsp. trichocarpa | 308 | NO |
| 12724226 | 690176 | 43.95 | 9.9E−98 | Glycine max | 309 | YES |
| 12724226 | 45260636 | 42.86 | 5.8E−93 | Nicotiana tabacum | 310 | YES |
| 13499809 | 21388658 | 54.24 | 3.3E−07 | Physcomitrella patens | 313 | NO |
| 13499809 | 4704605 | 52.63 | 2.6E−07 | Picea glauca | 314 | NO |
| 13499809 | 10799202 | 50.67 | 5.2E−09 | Sorghum bicolor | 315 | NO |
| 13499809 | 1605245 | 50.67 | 8.8E−07 | Parthenium argentatum | 316 | NO |
| 13499809 | 9957568 | 50.00 | 9.7E−08 | Capsella bursa-pastoris | 317 | NO |
| 12323989 | 1493656 | 61.83 | 8.4E−72 | Zea mays | 324 | NO |
| 12323989 | 50942745 | 55.70 | 5E−70 | Oryza sativa subsp. japonica | 325 | YES |
| 12323989 | 938587 | 37.50 | 1.5E−10 | Triticum aestivum | 326 | YES |
| 12323989 | 328171 | 49.80 | 1.2E−51 | Zea mays | 327 | YES |
| 11407753 | 746644 | 55.88 | 6.5E−36 | Triticum aestivum | 330 | YES |
| 11407753 | 56126414 | 52.80 | 3.3E−38 | Euphorbia esula | 331 | YES |
| 11407753 | 1644686 | 50.56 | 4.4E−36 | Glycine max | 332 | YES |
| 11407753 | 23899378 | 47.46 | 3.9E−35 | Lycopersicon esculentum | 333 | YES |
| 11407753 | 311199 | 46.58 | 4.6E−24 | Zea mays | 334 | YES |
| 11407753 | 359810 | 44.44 | 2.9E−23 | Zea mays | 335 | NO |
| 11407753 | 1476453 | 40.00 | 2.2E−10 | Populus balsamifera subsp. trichocarpa | 337 | NO |
| 11407753 | 70906129 | 38.46 | 2.1E−18 | Medicago truncatula | 338 | YES |
| 11407753 | 31432625 | 37.77 | 7.8E−21 | Oryza sativa subsp. japonica | 339 | NO |
| 4927725 | 37907 | 90.22 | 1.6E−157 | Arabidopsis thaliana | 344 | NO |
| 4927725 | 20465357 | 87.67 | 4.5E−176 | Arabidopsis thaliana | 345 | NO |
| 4927725 | 21593306 | 87.64 | 1.4E−174 | Arabidopsis thaliana | 346 | NO |
| 4927725 | 5139329 | 87.64 | 1.7E−174 | Arabidopsis thaliana | 347 | NO |
| 4927725 | 1213069 | 84.62 | 2E−157 | Nicotiana tabacum | 348 | YES |
| 4927725 | 14575543 | 84.42 | 1.4E−142 | Nicotiana sylvestris | 349 | YES |
| 4927725 | 1524384 | 82.78 | 1.4E−139 | Populus balsamifera subsp. trichocarpa | 351 | YES |
| 4927725 | 1470977 | 81.96 | 1.5E−144 | Populus balsamifera subsp. trichocarpa | 353 | NO |
| 4927725 | 1043166 | 81.07 | 6.6E−143 | Glycine max | 354 | YES |
| 11014624 | 8439547 | 83.67 | 7.7E−220 | Solanum tuberosum | 357 | YES |
| 11014624 | 1199827 | 82.86 | 1.4E−218 | Arabidopsis thaliana | 358 | NO |
| 11014624 | 1448917 | 82.86 | 1.4E−218 | Arabidopsis thaliana | 359 | NO |
| 11014624 | 4914408 | 82.86 | 1.4E−218 | Arabidopsis thaliana | 360 | NO |
| 11014624 | 42573081 | 82.86 | 1.4E−218 | Arabidopsis thaliana | 361 | NO |
| 11014624 | 578495 | 78.70 | 9.5E−206 | Glycine max | 362 | YES |
| 11014624 | 280346 | 74.65 | 5.3E−196 | Zea mays | 363 | YES |
| 11014624 | 34911416 | 74.35 | 2.7E−192 | Oryza sativa subsp. japonica | 364 | YES |
| 4987967 | 1460794 | 89.25 | 4.8E−189 | Populus balsamifera subsp. trichocarpa | 368 | NO |
| 4987967 | 1450365 | 88.97 | 3.2E−192 | Populus balsamifera subsp. trichocarpa | 370 | YES |
| 4987967 | 593648 | 82.38 | 5.8E−206 | Glycine max | 371 | YES |
| 4987967 | 237870 | 79.52 | 3.8E−186 | Zea mays | 372 | NO |
| 4987967 | 1378809 | 75.81 | 5.3E−189 | Zea mays | 373 | YES |
| 4987967 | 697349 | 74.11 | 6.5E−191 | Triticum aestivum | 374 | YES |
| 4987967 | 50907773 | 72.92 | 9.9E−188 | Oryza sativa subsp. japonica | 375 | YES |
| 3039543 | 17815 | 93.98 | 1.4E−252 | Brassica napus | 378 | YES |
| 3039543 | 46095337 | 93.57 | 1E−249 | Brassica rapa | 379 | YES |
| 3039543 | 18251236 | 93.24 | 5.1E−255 | Orychophragmus violaceus | 380 | YES |
| 3039543 | 48526086 | 83.55 | 7.9E−202 | Conyza canadensis | 381 | YES |
| 7090814 | 15825883 | 97.12 | 5.1E−255 | Arabidopsis thaliana | 384 | NO |
| 7090814 | 8439547 | 84.48 | 2.3E−227 | Solanum tuberosum | 385 | YES |
| 7090814 | 578495 | 82.95 | 2.3E−211 | Glycine max | 386 | YES |
| 7090814 | 1187996 | 82.86 | 1.4E−218 | Arabidopsis thaliana | 387 | NO |
| 7090814 | 20466326 | 82.86 | 1.4E−218 | Arabidopsis thaliana | 388 | NO |
| 7090814 | 280346 | 78.80 | 1.1E−197 | Zea mays | 389 | YES |
| 7090814 | 50932643 | 77.55 | 4.1E−198 | Oryza sativa subsp. japonica | 390 | YES |
| 7094546 | 1496106 | 74.25 | 1.1E−178 | Populus balsamifera subsp. trichocarpa | 394 | NO |
| 7094546 | 1505326 | 74.05 | 3E−194 | Populus balsamifera subsp. trichocarpa | 396 | YES |
| 7094546 | 49035694 | 70.99 | 3.5E−176 | Medicago truncatula | 397 | YES |
| 7094546 | 15485155 | 56.09 | 1.7E−128 | Brassica juncea | 398 | YES |
| 7094546 | 25956262 | 54.50 | 5.8E−135 | Cucumis sativus | 399 | YES |
| 7094546 | 15485153 | 54.48 | 2.2E−126 | Brassica juncea | 400 | NO |
| 7094546 | 50912665 | 54.36 | 4.6E−126 | Oryza sativa subsp. japonica | 401 | YES |
| 7094546 | 12331173 | 54.35 | 4.5E−128 | Brassica juncea | 402 | NO |
| 12336276 | 34365731 | 83.00 | 0 | Arabidopsis thaliana | 407 | NO |
| 12336276 | 34903888 | 61.52 | 0 | Oryza sativa subsp. japonica | 408 | YES |
| 12336276 | 34903880 | 59.55 | 0 | Oryza sativa subsp. japonica | 409 | NO |
| 12336276 | 820398 | 54.74 | 2E−22 | Triticum aestivum | 410 | YES |
| 12336276 | 34903874 | 58.33 | 0 | Oryza sativa subsp. japonica | 411 | NO |
| 12336276 | 34903876 | 58.52 | 0 | Oryza sativa subsp. japonica | 412 | NO |
| 12336276 | 779326 | 57.55 | 0 | Triticum aestivum | 413 | NO |
| 1807504 | 14719883 | 73.62 | 9.8E−117 | Medicago truncatula | 418 | YES |
| 1807504 | 45504723 | 73.22 | 3E−131 | Nicotiana tabacum | 419 | YES |
| 1807504 | 9972157 | 73.18 | 2.3E−124 | Pisum sativum | 420 | YES |
| 1807504 | 5230656 | 71.67 | 3.4E−130 | Lycopersicon esculentum | 421 | YES |
| 1807504 | 60476424 | 70.20 | 1.6E−123 | Glycine max | 422 | YES |
| 1807504 | 60476408 | 70.18 | 1.2E−118 | Lotus japonicus | 423 | YES |
| 1807504 | 30314006 | 70.03 | 6.1E−124 | Eschscholzia californica subsp. californica | 424 | YES |
| 1807504 | 3183617 | 69.68 | 5.5E−123 | Antirrhinum majus | 425 | YES |
| 1807504 | 60476426 | 67.93 | 4.5E−112 | Glycine max | 426 | NO |
| 1807504 | 60476410 | 67.27 | 4.6E−103 | Lotus japonicus | 427 | NO |
| 3096137 | 1535623 | 76.75 | 1.3E−163 | Populus balsamifera subsp. trichocarpa | 431 | YES |
| 3096137 | 1482129 | 76.75 | 1.9E−153 | Populus balsamifera subsp. trichocarpa | 433 | NO |
| 3096137 | 932657 | 68.18 | 2.8E−144 | Triticum aestivum | 434 | NO |
| 3096137 | 50912345 | 67.76 | 2.2E−151 | Oryza sativa subsp. japonica | 435 | NO |
| 3096137 | 34898706 | 67.39 | 9.5E−151 | Oryza sativa subsp. japonica | 436 | NO |
| 3096137 | 229480 | 67.00 | 1.6E−141 | Zea mays | 437 | NO |
| 3096137 | 259302 | 65.90 | 6.7E−150 | Zea mays | 438 | YES |
| 3096137 | 51535770 | 65.83 | 5.8E−151 | Oryza sativa subsp. japonica | 439 | NO |
| 3096137 | 257896 | 65.23 | 2.4E−136 | Zea mays | 440 | NO |
| 3096137 | 1496626 | 64.51 | 1.1E−97 | Populus balsamifera subsp. trichocarpa | 442 | NO |
| 3096137 | 557220 | 64.03 | 4.6E−135 | Triticum aestivum | 443 | YES |
| 3096137 | 50726342 | 63.97 | 3.5E−50 | Oryza sativa subsp. japonica | 444 | NO |
| 3096137 | 50905855 | 62.70 | 1.9E−145 | Oryza sativa subsp. japonica | 445 | YES |
| 3096137 | 1443691 | 61.64 | 3.2E−105 | Populus balsamifera subsp. trichocarpa | 447 | NO |
| 7082162 | 25991347 | 93.70 | 6.5E−278 | Brassica napus | 450 | YES |
| 7082162 | 3283433 | 92.79 | 8.6E−276 | Sinapis alba | 451 | YES |
| 7082162 | 20198148 | 85.40 | 9.5E−254 | Arabidopsis thaliana | 452 | NO |
| 7082162 | 42569237 | 85.40 | 9.5E−254 | Arabidopsis thaliana | 453 | NO |
| 7082162 | 5915822 | 59.67 | 3.1E−168 | Sorghum bicolor | 454 | YES |
| 7082162 | 1470714 | 59.23 | 3.2E−151 | Populus balsamifera subsp. trichocarpa | 456 | YES |
| 7082162 | 1470707 | 58.80 | 5.3E−149 | Populus balsamifera subsp. trichocarpa | 458 | NO |
| 7082162 | 1449045 | 58.37 | 1.3E−152 | Populus balsamifera subsp. trichocarpa | 460 | NO |
| 7082162 | 532331 | 56.67 | 4E−152 | Glycine max | 461 | YES |
| 7082162 | 47156051 | 54.60 | 1.7E−142 | Lotus japonicus | 462 | YES |
| 7082162 | 6739530 | 54.17 | 1.1E−156 | Manihot esculenta | 463 | YES |
| 7082162 | 56553508 | 53.79 | 4.8E−156 | Manihot esculenta | 464 | NO |
| 7082162 | 47156049 | 53.66 | 2.8E−142 | Lotus japonicus | 465 | NO |
| 7082162 | 6739527 | 53.07 | 1.2E−159 | Manihot esculenta | 466 | NO |
| 13647376 | 951785 | 61.11 | 4E−14 | Brassica napus | 471 | YES |
| 13647376 | 1440346 | 47.46 | 6.8E−07 | Populus balsamifera subsp. trichocarpa | 473 | YES |
| 13647710 | 556472 | 41.34 | 1.6E−26 | Glycine max | 476 | YES |
| 13647710 | 18650662 | 70.17 | 4.5E−54 | Lycopersicon esculentum | 477 | YES |
| 13647710 | 685191 | 62.42 | 3.5E−38 | Triticum aestivum | 478 | YES |
| 13647710 | 19507 | 63.52 | 3.1E−46 | Lupinus polyphyllus | 479 | YES |
| 13647710 | 314589 | 63.58 | 5E−39 | Zea mays | 480 | YES |
| 13621103 | 20269055 | 54.65 | 7.4E−38 | Populus tremula x Populus tremuloides | 489 | YES |
| 13621103 | 1524883 | 55.03 | 2.6E−37 | Populus balsamifera subsp. trichocarpa | 491 | YES |
| 13621103 | 1497918 | 54.76 | 7.2E−35 | Populus balsamifera subsp. trichocarpa | 493 | NO |
| 13621103 | 1471472 | 53.64 | 7.3E−26 | Populus balsamifera subsp. trichocarpa | 495 | NO |
| 13621103 | 20269053 | 52.35 | 1E−33 | Populus tremula x Populus tremuloides | 496 | NO |
| 13621103 | 675127 | 46.86 | 4.8E−34 | Glycine max | 497 | YES |
| 13621103 | 50912269 | 46.47 | 5.6E−24 | Oryza sativa subsp. japonica | 498 | NO |
| 13621103 | 742023 | 36.00 | 4.3E−19 | Triticum aestivum | 499 | NO |
| 13621103 | 32400272 | 36.00 | 4.3E−19 | Triticum aestivum | 500 | NO |
| 13621103 | 962494 | 32.45 | 1.7E−15 | Brassica napus | 501 | NO |
| 13621103 | 32396299 | 30.29 | 1.2E−17 | Pinus taeda | 502 | NO |
| 13621103 | 32396293 | 35.93 | 6E−18 | Pinus taeda | 503 | NO |
| 13621103 | 29465672 | 36.00 | 9.9E−19 | Vitis vinifera | 504 | NO |
| 12733452 | 482437 | 62.57 | 3.6E−52 | Glycine max | 507 | YES |
| 12733452 | 52077327 | 67.26 | 2.3E−53 | Oryza sativa subsp. japonica | 508 | YES |
| 12733452 | 1548279 | 64.50 | 7.8E−53 | Zea mays | 509 | YES |
| 12733452 | 727056 | 69.57 | 3.2E−21 | Triticum aestivum | 510 | YES |
| 12734583 | 50949065 | 34.30 | 1.2E−29 | Oryza sativa | 513 | NO |
| 12734583 | 1316822 | 33.88 | 1.2E−36 | Triticum aestivum | 514 | YES |
| 12734583 | 55168346 | 64.81 | 2.1E−32 | Oryza sativa subsp. japonica | 515 | NO |
| 12734583 | 81686872 | 63.25 | 3.6E−39 | Oryza sativa subsp. japonica | 516 | YES |
| 12734583 | 28070968 | 27.08 | 1.3E−21 | Lycopersicon esculentum | 517 | YES |
| 12734583 | 1472175 | 57.50 | 6.3E−20 | Glycine max | 518 | NO |
| 12734583 | 1508018 | 55.00 | 1.3E−18 | Glycine max | 519 | YES |
| 12734583 | 61217028 | 54.76 | 9.7E−22 | Petunia x hybrida | 520 | YES |
| 12734583 | 61216997 | 50.00 | 9E−20 | Antirrhinum majus | 521 | YES |
| 12734583 | 39841617 | 38.93 | 3.8E−34 | Zea mays | 522 | NO |
| 12734583 | 61217580 | 42.98 | 7.2E−34 | Zea mays | 523 | YES |
| 12734583 | 325979 | 51.19 | 7.8E−34 | Zea mays | 524 | NO |
| 12734583 | 3955019 | 13.89 | 4.8E−11 | Populus tremula x Populus tremuloides | 525 | NO |
| 12734583 | 40233103 | 15.21 | 4.8E−11 | Populus tomentosa | 526 | NO |
| 13607033 | 34904200 | 20.25 | 0.0000001 | Oryza sativa subsp. japonica | 529 | NO |
| 13607033 | 56784164 | 50.63 | 8.7E−08 | Oryza sativa subsp. japonica | 530 | NO |
| 13607033 | 1467355 | 49.46 | 1.1E−29 | Populus balsamifera subsp. trichocarpa | 531 | NO |
| 13607033 | 1467355 | 49.46 | 1.1E−29 | Populus balsamifera subsp. trichocarpa | 532 | NO |
| 13607033 | 914912 | 45.88 | 1.3E−07 | Triticum aestivum | 533 | NO |
| 13607033 | 1237838 | 31.58 | 7.5E−29 | Glycine max | 534 | NO |
| 13592772 | 1512677 | 68.38 | 1.9E−59 | Populus balsamifera subsp. trichocarpa | 540 | YES |
| 13592772 | 1459412 | 68.38 | 1.9E−59 | Populus balsamifera subsp. trichocarpa | 542 | NO |
| 13592772 | 523802 | 56.67 | 7.7E−59 | Glycine max | 543 | YES |
| 13592772 | 22773261 | 46.20 | 1.1E−45 | Oryza sativa subsp. japonica | 544 | YES |
| 13614632 | 1523115 | 56.62 | 2E−85 | Populus balsamifera subsp. trichocarpa | 548 | YES |
| 13593033 | 563805 | 57.18 | 2.3E−82 | Glycine max | 551 | YES |
| 13593033 | 50252324 | 43.43 | 1.3E−49 | Oryza sativa subsp. japonica | 552 | YES |
| 13593033 | 50946029 | 44.84 | 9.3E−53 | Oryza sativa subsp. japonica | 553 | YES |
| 13593033 | 359116 | 33.22 | 3.1E−30 | Zea mays | 554 | NO |
| 13593033 | 1466509 | 46.63 | 1.8E−29 | Populus balsamifera subsp. trichocarpa | 556 | NO |
| 13593033 | 29466635 | 19.03 | 1.6E−08 | Oryza sativa | 557 | YES |
| 13593033 | 1479796 | 42.63 | 1.2E−32 | Populus balsamifera subsp. trichocarpa | 559 | YES |
| 13610698 | 1510814 | 66.43 | 3E−146 | Populus balsamifera subsp. trichocarpa | 563 | YES |
| 13610698 | 1457602 | 65.92 | 2.5E−144 | Populus balsamifera subsp. trichocarpa | 565 | NO |
| 13610698 | 1465272 | 65.63 | 1.7E−154 | Populus balsamifera subsp. trichocarpa | 567 | NO |
| 13610698 | 50942577 | 52.86 | 2.1E−118 | Oryza sativa subsp. japonica | 568 | YES |
| 23505182 | 50906279 | 65.83 | 8.6E−103 | Oryza sativa subsp. japonica | 571 | YES |
| 23505182 | 498454 | 59.71 | 8.1E−91 | Zea mays | 572 | YES |
| 23505182 | 565294 | 58.12 | 9.6E−88 | Glycine max | 573 | YES |
| 13645995 | 1503065 | 86.96 | 8E−20 | Populus balsamifera subsp. trichocarpa | 577 | NO |
| 13645995 | 1450024 | 86.96 | 8E−20 | Populus balsamifera subsp. trichocarpa | 579 | NO |
| 13645995 | 1458507 | 86.96 | 8E−20 | Populus balsamifera subsp. trichocarpa | 581 | NO |
| 13645995 | 1476818 | 86.96 | 8E−20 | Populus balsamifera subsp. trichocarpa | 583 | NO |
| 13645995 | 56783710 | 85.00 | 1.2E−29 | Oryza sativa subsp. japonica | 584 | NO |
| 13645995 | 34903284 | 40.57 | 1.3E−27 | Oryza sativa subsp. japonica | 585 | NO |
| 13645995 | 1669341 | 85.00 | 3.1E−27 | Cucurbita maxima | 586 | NO |
| 13645995 | 1479325 | 81.67 | 1.3E−26 | Populus balsamifera subsp. trichocarpa | 588 | NO |
| 13592165 | 1455805 | 65.34 | 6E−134 | Populus balsamifera subsp. trichocarpa | 592 | YES |
| 13592165 | 1529744 | 64.60 | 6.7E−119 | Populus balsamifera subsp. trichocarpa | 594 | NO |
| 13592165 | 1476297 | 64.36 | 3.5E−97 | Populus balsamifera subsp. trichocarpa | 596 | NO |
| 13592165 | 62734646 | 50.74 | 5.2E−95 | Oryza sativa subsp. japonica | 597 | YES |
| 13592165 | 218213 | 45.15 | 1.7E−84 | Zea mays | 598 | YES |
| 13592165 | 50948139 | 50.47 | 9.7E−88 | Oryza sativa subsp. japonica | 599 | YES |
| 23495481 | 980164 | 84.48 | 8.6E−48 | Brassica napus | 602 | YES |
| 23495481 | 37536722 | 62.00 | 1.8E−22 | Oryza sativa subsp. japonica | 603 | YES |
| 23495481 | 37536720 | 61.62 | 2.2E−24 | Oryza sativa subsp. japonica | 604 | NO |
| 23495481 | 373282 | 60.38 | 1.5E−25 | Zea mays | 605 | YES |
| 23495481 | 37536718 | 60.19 | 5.2E−25 | Oryza sativa subsp. japonica | 606 | NO |
| 23495481 | 60542797 | 59.65 | 3.3E−30 | Capsicum chinense | 607 | YES |
| 23495481 | 620364 | 59.26 | 4.3E−30 | Glycine max | 608 | YES |
| 23495481 | 46095207 | 57.89 | 1.4E−29 | Lycopersicon esculentum | 609 | YES |
| 23495481 | 1447245 | 56.90 | 9.1E−28 | Populus balsamifera subsp. trichocarpa | 611 | YES |
| 23495481 | 4454097 | 56.52 | 1.3E−28 | Catharanthus roseus | 612 | NO |
| 23495481 | 1199774 | 56.52 | 5.6E−28 | Populus nigra | 613 | YES |
| 23495481 | 407410 | 55.65 | 2.7E−28 | Catharanthus roseus | 614 | NO |
| 23495481 | 10798758 | 54.46 | 1.8E−29 | Nicotiana tabacum | 615 | YES |
| 23495481 | 18316 | 54.39 | 8.2E−27 | Daucus carota | 616 | YES |
| 23495481 | 60459393 | 54.39 | 8.2E−27 | Capsicum annuum | 617 | YES |
| 23531413 | 1104601 | 71.83 | 4.1E−20 | Brassica napus | 622 | NO |
| 23531413 | 1100450 | 75.81 | 2.5E−16 | Brassica napus | 623 | YES |
| 23531413 | 1467420 | 72.09 | 1E−12 | Populus balsamifera subsp. trichocarpa | 625 | NO |
| 23531413 | 1483277 | 70.83 | 2.3E−22 | Populus balsamifera subsp. trichocarpa | 627 | YES |
| 23531413 | 2921332 | 65.00 | 2.8E−10 | Gossypium hirsutum | 628 | NO |
| 23531413 | 51872289 | 65.00 | 2.8E−10 | Gossypium arboreum | 629 | NO |
| 23531413 | 711042 | 64.06 | 1.1E−17 | Glycine max | 630 | NO |
| 23531413 | 54290864 | 54.55 | 4.5E−14 | Oryza sativa subsp. japonica | 631 | NO |
| 23531413 | 15042122 | 62.50 | 7.3E−10 | Zea luxurians | 632 | NO |
| 13606025 | 1083282 | 88.28 | 6.3E−64 | Brassica napus | 635 | YES |
| 13606025 | 1068274 | 84.83 | 8.5E−60 | Brassica napus | 636 | NO |
| 13606025 | 1064745 | 65.87 | 5.3E−35 | Zea mays | 637 | YES |
| 13606025 | 627586 | 56.62 | 1.1E−29 | Glycine max | 638 | YES |
| 13606025 | 1169018 | 58.76 | 9.4E−22 | Glycine max | 639 | YES |
| 13606025 | 232678 | 40.50 | 8.4E−14 | Zea mays | 640 | NO |
| 13606025 | 443590 | 35.92 | 4.8E−11 | Zea mays | 641 | NO |
| 13606025 | 678915 | 50.89 | 2.2E−16 | Triticum aestivum | 642 | YES |
| 13606025 | 1048159 | 51.00 | 3.1E−14 | Triticum aestivum | 643 | NO |
| 13606025 | 53793564 | 47.76 | 2.9E−07 | Oryza sativa subsp. japonica | 644 | YES |
| 13606025 | 34909878 | 32.43 | 1.9E−07 | Oryza sativa subsp. japonica | 645 | NO |
| 13606025 | 20149050 | 30.69 | 0.0000011 | Capsicum annuum | 646 | YES |
| 13606025 | 10185818 | 31.78 | 1.7E−08 | Tulipa gesneriana | 647 | NO |
| 23364445 | 1497025 | 58.49 | 8.1E−43 | Populus balsamifera subsp. trichocarpa | 651 | YES |
| 23364445 | 1659056 | 56.25 | 6.2E−36 | Glycine max | 652 | YES |
| 23509199 | 1471610 | 58.57 | 6.2E−13 | Populus balsamifera subsp. trichocarpa | 658 | YES |
| 23509199 | 34895596 | 41.62 | 8.8E−28 | Oryza sativa subsp. japonica | 659 | YES |
| 23509199 | 963612 | 45.88 | 8.9E−25 | Brassica napus | 660 | YES |
| 23509199 | 1449284 | 44.44 | 1.3E−26 | Populus balsamifera subsp. trichocarpa | 662 | NO |
| 23509199 | 1060169 | 41.96 | 1.8E−13 | Glycine max | 663 | YES |
| 23509199 | 1688030 | 41.57 | 4.9E−20 | Zea mays | 664 | YES |
| 23509199 | 18390109 | 30.46 | 6.1E−12 | Sorghum bicolor | 665 | NO |
| 12667412 | 1445379 | 52.17 | 1.6E−30 | Populus balsamifera subsp. trichocarpa | 669 | YES |
| 12667412 | 1044811 | 50.62 | 9E−34 | Glycine max | 670 | YES |
| 12667412 | 522952 | 48.02 | 4.7E−34 | Glycine max | 671 | NO |
| 12667412 | 479801 | 47.52 | 4.7E−34 | Glycine max | 672 | NO |
| 12667412 | 1449468 | 48.59 | 5.5E−28 | Populus balsamifera subsp. trichocarpa | 674 | NO |
| 12667412 | 1461090 | 47.73 | 2.4E−18 | Populus balsamifera subsp. trichocarpa | 676 | NO |
| 12667412 | 276476 | 30.39 | 2.3E−18 | Zea mays | 677 | YES |
| 12385780 | 1464833 | 82.76 | 9E−24 | Populus balsamifera subsp. trichocarpa | 681 | YES |
| 12385780 | 4567313 | 29.11 | 1.2E−17 | Arabidopsis thaliana | 682 | YES |
| 12385780 | 1452647 | 81.08 | 5.2E−15 | Populus balsamifera subsp. trichocarpa | 684 | YES |
| 12385780 | 1458150 | 79.66 | 3.6E−26 | Populus balsamifera subsp. trichocarpa | 686 | YES |
| 12385780 | 50933653 | 36.36 | 6.5E−22 | Oryza sativa subsp. japonica | 687 | YES |
| 12385780 | 375181 | 31.11 | 2.7E−21 | Zea mays | 688 | YES |
| 12385780 | 393033 | 36.65 | 4.8E−25 | Zea mays | 689 | YES |
| 12385780 | 666751 | 34.23 | 3.3E−24 | Glycine max | 690 | YES |
| 23521525 | 1491996 | 50.37 | 4E−25 | Populus balsamifera subsp. trichocarpa | 702 | NO |
| 23521525 | 1439136 | 50.00 | 2.2E−24 | Populus balsamifera subsp. trichocarpa | 704 | NO |
| 23521525 | 57117314 | 25.96 | 3.6E−16 | Populus x canescens | 705 | NO |
| 23521525 | 647103 | 34.43 | 5.7E−23 | Glycine max | 706 | NO |
| 23521525 | 819214 | 30.88 | 3.4E−25 | Triticum aestivum | 707 | YES |
| 23521525 | 708708 | 33.33 | 5E−15 | Glycine max | 708 | YES |
| 23521525 | 28558782 | 35.05 | 1.5E−24 | Cucumis melo | 709 | NO |
| 23521525 | 23451086 | 16.03 | 2.3E−17 | Medicago sativa | 710 | NO |
| 23521525 | 957229 | 29.19 | 5.8E−17 | Brassica napus | 711 | NO |
| 23521525 | 50900320 | 32.26 | 2.7E−23 | Oryza sativa subsp. japonica | 712 | NO |
| 23521525 | 1603708 | 31.67 | 8.8E−18 | Parthenium argentatum | 713 | NO |
| 23521525 | 398008 | 22.75 | 5E−21 | Zea mays | 714 | NO |
| 13576188 | 1404062 | 79.22 | 5.9E−100 | Zea mays | 717 | YES |
| 13576188 | 1541512 | 57.26 | 1.2E−64 | Populus balsamifera subsp. trichocarpa | 719 | YES |
| 13576188 | 715530 | 52.85 | 3.4E−65 | Glycine max | 720 | YES |
| 13576188 | 1455981 | 55.64 | 5.8E−65 | Populus balsamifera subsp. trichocarpa | 722 | NO |
| 13576188 | 62734221 | 54.80 | 1.5E−56 | Oryza sativa subsp. japonica | 723 | YES |
| 13576188 | 772319 | 47.73 | 6E−59 | Triticum aestivum | 724 | YES |
| 13576188 | 224054 | 47.73 | 5E−55 | Zea mays | 725 | NO |
| 23360146 | 1443950 | 50.52 | 1.9E−50 | Populus balsamifera subsp. trichocarpa | 732 | YES |
| 23360146 | 1486315 | 49.13 | 6.8E−46 | Populus balsamifera subsp. trichocarpa | 734 | NO |
| 23360146 | 712340 | 45.26 | 8.3E−41 | Glycine max | 735 | YES |
| 23360146 | 1235862 | 55.41 | 1.3E−15 | Glycine max | 736 | NO |
| 23360146 | 335314 | 32.27 | 8E−24 | Zea mays | 737 | YES |
| 23358032 | 23429649 | 35.60 | 5.6E−32 | Lycopersicon esculentum | 740 | YES |
| 13575362 | 1486224 | 59.67 | 5.3E−78 | Populus balsamifera subsp. trichocarpa | 748 | YES |
| 13575362 | 1444021 | 58.03 | 5.3E−71 | Populus balsamifera subsp. trichocarpa | 750 | NO |
| 13575362 | 23451086 | 53.93 | 1.6E−56 | Medicago sativa | 751 | YES |
| 13575362 | 474127 | 54.46 | 6E−75 | Glycine max | 752 | YES |
| 12670870 | 1362011 | 95.02 | 0 | Arabidopsis thaliana | 759 | NO |
| 12670870 | 1485236 | 70.63 | 7.9E−155 | Populus balsamifera subsp. trichocarpa | 761 | YES |
| 12670870 | 60593177 | 68.00 | 2.5E−143 | Medicago truncatula | 762 | YES |
| 12670870 | 1446740 | 67.95 | 4.5E−152 | Populus balsamifera subsp. trichocarpa | 764 | NO |
| 12670870 | 30526087 | 67.13 | 0 | Pisum sativum | 765 | YES |
| 12670870 | 28624856 | 64.97 | 0 | Lotus japonicus | 766 | YES |
| 12670870 | 30526089 | 66.67 | 0 | Pisum sativum | 767 | NO |
| 12670870 | 4101570 | 66.20 | 0 | Pisum sativum | 768 | NO |
| 12670870 | 42795315 | 61.82 | 0 | Mimulus lewisii | 769 | YES |
| 12670870 | 547307 | 63.55 | 1.2E−142 | Antirrhinum majus | 770 | YES |
| 12670870 | 42795317 | 60.92 | 0 | Mimulus guttatus | 771 | YES |
| 23495291 | 1439158 | 44.44 | 1.4E−56 | Populus balsamifera subsp. trichocarpa | 775 | YES |
| 23495291 | 1492026 | 44.14 | 2.6E−55 | Populus balsamifera subsp. trichocarpa | 777 | NO |
| 23495291 | 928574 | 39.87 | 3.4E−44 | Triticum aestivum | 778 | YES |
| 23495291 | 57900395 | 39.19 | 2.7E−44 | Oryza sativa subsp. japonica | 779 | YES |
| 13612399 | 473933 | 49.85 | 8.5E−60 | Glycine max | 782 | YES |
| 13612399 | 1653608 | 47.76 | 1.4E−24 | Glycine max | 783 | YES |
| 13612399 | 398141 | 35.63 | 6.5E−30 | Zea mays | 784 | YES |
| 23522373 | 1221348 | 80.65 | 4.7E−150 | Zea mays | 787 | YES |
| 23522373 | 1538994 | 71.26 | 3.6E−120 | Populus balsamifera subsp. trichocarpa | 789 | YES |
| 23522373 | 3336903 | 64.19 | 6.4E−118 | Petroselinum crispum | 790 | YES |
| 23522373 | 1500081 | 69.50 | 1E−113 | Populus balsamifera subsp. trichocarpa | 792 | NO |
| 23522373 | 545441 | 68.66 | 3E−123 | Glycine max | 793 | YES |
| 23522373 | 5381313 | 64.99 | 3.6E−124 | Catharanthus roseus | 794 | YES |
| 23522373 | 3336906 | 64.84 | 7.9E−120 | Petroselinum crispum | 795 | NO |
| 23522373 | 13775109 | 64.63 | 3.8E−120 | Phaseolus vulgaris | 796 | YES |
| 23522373 | 1447080 | 65.76 | 3.8E−116 | Populus balsamifera subsp. trichocarpa | 798 | NO |
| 12672729 | 1343575 | 82.20 | 0 | Arabidopsis thaliana | 803 | NO |
| 12672729 | 20259635 | 82.20 | 0 | Arabidopsis thaliana | 804 | NO |
| 12672729 | 66932877 | 81.94 | 1.5E−185 | Lotus japonicus | 805 | YES |
| 12672729 | 4558462 | 78.04 | 0 | Medicago sativa subsp. x varia | 806 | YES |
| 12672729 | 7158292 | 77.61 | 0 | Medicago truncatula | 807 | YES |
| 12672729 | 66932879 | 78.43 | 2.2E−184 | Pisum sativum | 808 | YES |
| 12672729 | 1500350 | 78.24 | 1.3E−188 | Populus balsamifera subsp. trichocarpa | 810 | YES |
| 4984839 | 71834749 | 74.19 | 1E−60 | Brassica rapa subsp. pekinensis | 813 | YES |
| 4984839 | 71834747 | 69.35 | 2.2E−58 | Brassica rapa subsp. pekinensis | 814 | NO |
| 4984839 | 31580813 | 60.71 | 1E−46 | Brassica napus | 815 | YES |
| 4984839 | 15667638 | 32.18 | 1.5E−21 | Cryptomeria japonica | 816 | YES |
| 4984839 | 17933458 | 60.20 | 4E−45 | Brassica napus | 817 | NO |
| 4984839 | 73915377 | 60.00 | 2.3E−45 | Arabidopsis arenosa | 818 | YES |
| 4984839 | 17933450 | 59.39 | 1.5E−45 | Brassica napus | 819 | NO |
| 4984839 | 1065387 | 59.39 | 1.2E−45 | Brassica napus | 820 | NO |
| 36817505 | 1459700 | 61.87 | 2.6E−229 | Populus balsamifera subsp. trichocarpa | 824 | YES |
| 36817505 | 1512967 | 61.62 | 6.7E−222 | Populus balsamifera subsp. trichocarpa | 826 | NO |
| 36817505 | 50928937 | 56.80 | 1.1E−175 | Oryza sativa subsp. japonica | 827 | YES |
| 13610436 | 21554247 | 98.44 | 1.1E−64 | Arabidopsis thaliana | 832 | NO |
| 13610436 | 112157 | 89.06 | 1.4E−56 | Arabidopsis thaliana | 833 | NO |
| 13610436 | 150107 | 87.50 | 1.7E−55 | Arabidopsis thaliana | 834 | NO |
| 13610436 | 1118497 | 77.34 | 8.1E−48 | Brassica napus | 835 | YES |
| 13610436 | 1265409 | 80.67 | 5.1E−46 | Brassica napus | 836 | NO |
| 13610436 | 963126 | 75.78 | 9.2E−47 | Brassica napus | 837 | NO |
| 13610436 | 968344 | 76.56 | 1.3E−49 | Brassica napus | 838 | NO |
| 13489667 | 951261 | 90.60 | 1.4E−50 | Brassica napus | 841 | NO |
| 13489667 | 1258526 | 89.51 | 3.1E−62 | Brassica napus | 842 | YES |
| 13489667 | 1380957 | 87.94 | 1.4E−59 | Zea mays | 843 | YES |
| 13489667 | 973721 | 84.68 | 3.4E−49 | Brassica napus | 844 | NO |
| 13489667 | 587233 | 78.46 | 2.1E−40 | Glycine max | 845 | NO |
| 13489667 | 1115876 | 70.68 | 2.3E−41 | Glycine max | 846 | NO |
| 13489667 | 615004 | 51.88 | 2.9E−27 | Glycine max | 847 | NO |
| 13489667 | 1610049 | 68.04 | 9.1E−27 | Parthenium argentatum | 848 | YES |
| 13489667 | 665805 | 64.35 | 3.1E−30 | Glycine max | 849 | NO |
| 13489667 | 685101 | 48.33 | 4.8E−18 | Triticum aestivum | 850 | NO |
| 13489667 | 50908919 | 41.98 | 2.9E−20 | Oryza sativa subsp. japonica | 851 | YES |
| 13489667 | 58737210 | 49.00 | 1.2E−17 | Oryza sativa | 852 | NO |
| 13489667 | 1330739 | 39.69 | 3E−18 | Triticum aestivum | 853 | YES |
| 13489667 | 50923897 | 44.04 | 2.1E−18 | Oryza sativa subsp. japonica | 854 | YES |
| 13489667 | 1707981 | 42.27 | 5.6E−12 | Ricinus communis | 855 | YES |
| 12332453 | 1065020 | 93.78 | 3.8E−105 | Zea mays | 858 | YES |
| 12332453 | 1381401 | 91.22 | 7.3E−102 | Zea mays | 859 | NO |
| 12332453 | 1473760 | 84.86 | 2E−87 | Populus balsamifera subsp. trichocarpa | 861 | YES |
| 12332453 | 51090974 | 77.72 | 1.1E−76 | Oryza sativa subsp. japonica | 862 | YES |
| 12332453 | 558051 | 68.90 | 1.2E−76 | Glycine max | 863 | NO |
| 12332453 | 1047194 | 75.74 | 2.5E−86 | Glycine max | 864 | NO |
| 12332453 | 1248638 | 65.57 | 6.9E−42 | Glycine max | 865 | YES |
| 12332453 | 615686 | 67.63 | 2.3E−75 | Triticum aestivum | 866 | YES |
| 12332453 | 524043 | 71.97 | 1.7E−61 | Glycine max | 867 | YES |
| 12700063 | 1497958 | 78.00 | 9.1E−170 | Populus balsamifera subsp. trichocarpa | 875 | YES |
| 12700063 | 1444972 | 78.00 | 3E−162 | Populus balsamifera subsp. trichocarpa | 877 | NO |
| 12700063 | 1471743 | 77.75 | 3.5E−168 | Populus balsamifera subsp. trichocarpa | 879 | NO |
| 12700063 | 1043309 | 73.11 | 8.6E−158 | Glycine max | 880 | YES |
| 12601981 | 4894170 | 55.79 | 0 | Cicer arietinum | 881 | NO |
| 12601981 | 521542 | 54.85 | 0 | Glycine max | 881 | YES |
| 12601981 | 33521521 | 54.49 | 0 | Medicago truncatula | 881 | YES |
| 12601981 | 81157970 | 0.00 | 0 | Sesamum radiatum | 881 | NO |
| 12601981 | 81157968 | 0.00 | 0 | Sesamum indicum | 881 | NO |
| 12601981 | 3059131 | 51.35 | 1.5E−121 | Helianthus tuberosus | 881 | NO |
| 12601981 | 7415996 | 51.02 | 0 | Lotus japonicus | 881 | YES |
| 12601981 | 2443348 | 50.61 | 0 | Glycyrrhiza echinata | 881 | YES |
| 12601981 | 3059129 | 50.41 | 1.3E−120 | Helianthus tuberosus | 881 | YES |
| 12601981 | 4200044 | 50.41 | 0 | Glycyrrhiza echinata | 881 | NO |
| 12601981 | 81157972 | 0.00 | 0 | Sesamum alatum | 881 | YES |
| 12601981 | 37726104 | 48.97 | 1.8E−125 | Pisum sativum | 881 | YES |
| 12695887 | 1480956 | 64.10 | 1.1E−32 | Glycine max | 881 | YES |
| 12700063 | 1058118 | 70.66 | 7.6E−150 | Glycine max | 881 | NO |
| 12721393 | 627596 | 66.73 | 0 | Glycine max | 881 | YES |
| 12721393 | 1173624 | 0.66 | 0 | Phalaenopsis sp. SM9108 | 881 | NO |
| 12721393 | 50939101 | 54.65 | 0 | Oryza sativa subsp. japonica | 881 | YES |
| 12721393 | 906986 | 57.47 | 9E−75 | Triticum aestivum | 881 | NO |
| 12721393 | 779234 | 50.29 | 1.1E−128 | Triticum aestivum | 881 | YES |
| 12721393 | 1551657 | 54.00 | 3.8E−131 | Zea mays | 881 | YES |
| 12721393 | 1600726 | 46.07 | 9.8E−54 | Zea mays | 881 | NO |
| 12721393 | 1601442 | 53.28 | 3E−131 | Zea mays | 881 | NO |
| 12721393 | 5921925 | 0.51 | 0 | Pinus radiata | 881 | YES |
| 12724333 | 963612 | 83.91 | 3.3E−74 | Brassica napus | 881 | YES |
| 12724333 | 34895596 | 47.86 | 1.1E−36 | Oryza sativa subsp. japonica | 881 | YES |
| 12724333 | 1688030 | 52.27 | 8.5E−21 | Zea mays | 881 | YES |
| 12724333 | 18390109 | 26.64 | 1.3E−15 | Sorghum bicolor | 881 | YES |
| 23498145 | 903520 | 57.47 | 8.8E−116 | Triticum aestivum | 881 | YES |
| 23498145 | 1601097 | 57.28 | 1.9E−129 | Zea mays | 881 | YES |
| 23498145 | 54290354 | 55.00 | 0 | Oryza sativa subsp. japonica | 881 | YES |
| 23498145 | 479101 | 54.83 | 0 | Glycine max | 881 | YES |
| 23498145 | 34912880 | 55.05 | 0 | Oryza sativa subsp. japonica | 881 | NO |
| 23498145 | 1589607 | 55.09 | 2.5E−143 | Zea mays | 881 | NO |
| 23498145 | 21842133 | 54.24 | 0 | Zea mays | 881 | NO |
| 23513037 | 251685 | 92.20 | 4.1E−68 | Arabidopsis thaliana | 881 | NO |
| 23513037 | 11994638 | 89.14 | 1.3E−80 | Arabidopsis thaliana | 881 | NO |
| 12700063 | 233103 | 64.36 | 7.3E−97 | Zea mays | 882 | YES |
| 12721393 | 1471370 | 0.00 | 0 | Populus balsamifera subsp. trichocarpa | 882 | NO |
| 12721393 | 1500987 | 0.00 | 0 | Populus balsamifera subsp. trichocarpa | 882 | NO |
| 12721393 | 1444471 | 0.00 | 0 | Populus balsamifera subsp. trichocarpa | 882 | NO |
| 12721393 | 1490915 | 0.00 | 0 | Populus balsamifera subsp. trichocarpa | 882 | NO |
| 12721393 | 1438105 | 0.00 | 0 | Populus balsamifera subsp. trichocarpa | 882 | YES |
| 23498145 | 1482371 | 0.00 | 0 | Populus balsamifera subsp. trichocarpa | 882 | YES |
| 23498145 | 1482362 | 0.00 | 0 | Populus balsamifera subsp. trichocarpa | 882 | NO |
| 23498145 | 1489077 | 0.00 | 0 | Populus balsamifera subsp. trichocarpa | 882 | NO |
| 23498145 | 1482356 | 0.00 | 0 | Populus balsamifera subsp. trichocarpa | 882 | NO |
| 23498145 | 1484293 | 0.00 | 0 | Populus balsamifera subsp. trichocarpa | 882 | NO |
| 12700063 | 34914854 | 63.21 | 1.2E−121 | Oryza sativa subsp. japonica | 883 | YES |
| 12700063 | 900752 | 62.87 | 2.4E−121 | Triticum aestivum | 884 | YES |
| 12730465 | 1459998 | 69.10 | 6.9E−53 | Populus balsamifera subsp. trichocarpa | 888 | YES |
| 12730465 | 1513263 | 68.60 | 4.1E−48 | Populus balsamifera subsp. trichocarpa | 890 | NO |
| 12730465 | 545208 | 68.59 | 8.8E−59 | Glycine max | 891 | YES |
| 12730465 | 50933031 | 57.50 | 7.5E−46 | Oryza sativa subsp. japonica | 892 | YES |
| 12730465 | 336092 | 59.09 | 1.7E−48 | Zea mays | 893 | YES |
| 12730465 | 771679 | 49.72 | 5.5E−21 | Triticum aestivum | 894 | YES |
| 12730465 | 28558779 | 40.54 | 1.1E−26 | Cucumis melo | 895 | YES |
| 12559673 | 50949165 | 74.89 | 2.5E−174 | Oryza sativa subsp. japonica | 900 | YES |
| 12559673 | 50935893 | 71.90 | 7.9E−171 | Oryza sativa subsp. japonica | 901 | NO |
| 12559673 | 364564 | 71.30 | 3.8E−171 | Zea mays | 902 | YES |
| 12559673 | 1514988 | 70.14 | 3.8E−155 | Populus balsamifera subsp. trichocarpa | 904 | YES |
| 12559673 | 1461702 | 69.16 | 6.4E−137 | Populus balsamifera subsp. trichocarpa | 906 | NO |
| 12663374 | 464433 | 68.80 | 1.6E−142 | Glycine max | 909 | YES |
| 23419575 | 1081216 | 81.62 | 8.5E−52 | Brassica napus | 912 | YES |
| 23419575 | 1448041 | 54.29 | 9.2E−19 | Populus balsamifera subsp. trichocarpa | 914 | NO |
| 23419575 | 1438056 | 47.01 | 1.2E−14 | Populus balsamifera subsp. trichocarpa | 916 | NO |
| 23419575 | 1438055 | 42.52 | 6E−15 | Populus balsamifera subsp. trichocarpa | 918 | NO |
| 23419575 | 50918565 | 37.60 | 2.4E−15 | Oryza sativa subsp. japonica | 919 | YES |
| 23778739 | 53792455 | 70.14 | 1.7E−110 | Oryza sativa subsp. japonica | 922 | YES |
| 23778739 | 34910130 | 69.94 | 6.8E−98 | Oryza sativa subsp. japonica | 923 | NO |
| 23778739 | 1465903 | 64.37 | 9.7E−47 | Populus balsamifera subsp. trichocarpa | 925 | YES |
| 23778739 | 527538 | 38.06 | 1.2E−45 | Glycine max | 926 | YES |
| 23778739 | 53749368 | 52.68 | 1.5E−54 | Oryza sativa subsp. japonica | 927 | NO |
| 23778739 | 954923 | 26.26 | 2.2E−20 | Brassica napus | 928 | NO |
| 23778739 | 11045087 | 26.26 | 2.1E−20 | Brassica napus | 929 | NO |
| 23778739 | 21741062 | 44.05 | 3E−45 | Oryza sativa subsp. japonica | 930 | NO |
| 23778739 | 861529 | 27.44 | 6.4E−23 | Triticum aestivum | 931 | NO |
| 23778739 | 1448710 | 42.38 | 4.2E−46 | Populus balsamifera subsp. trichocarpa | 933 | NO |
| 23778739 | 77999289 | 41.77 | 0.0000048 | Solanum tuberosum | 934 | NO |
| 23800158 | 1464833 | 79.03 | 5.2E−27 | Populus balsamifera subsp. trichocarpa | 938 | YES |
| 23800158 | 77378044 | 71.43 | 8.3E−28 | Gossypium hirsutum | 939 | YES |
| 23800158 | 62733300 | 67.01 | 2.4E−32 | Oryza sativa subsp. japonica | 940 | YES |
| 23800158 | 393033 | 39.08 | 7.2E−39 | Zea mays | 941 | YES |
| 23802651 | 1452212 | 81.65 | 6E−51 | Populus balsamifera subsp. trichocarpa | 945 | YES |
| 23802651 | 1456223 | 81.55 | 1.8E−49 | Populus balsamifera subsp. trichocarpa | 947 | NO |
| 23802651 | 31980093 | 51.10 | 3.2E−45 | Populus tremula x Populus tremuloides | 948 | YES |
| 23802651 | 1443195 | 77.98 | 1.8E−49 | Populus balsamifera subsp. trichocarpa | 950 | NO |
| 23802651 | 50948869 | 51.56 | 2.4E−47 | Oryza sativa subsp. japonica | 951 | YES |
| 23802651 | 520052 | 50.22 | 1.9E−45 | Glycine max | 952 | YES |
| 23802651 | 56783716 | 77.88 | 1.6E−46 | Oryza sativa subsp. japonica | 953 | NO |
| 23802651 | 782178 | 74.44 | 1.6E−34 | Triticum aestivum | 954 | YES |
| 23802651 | 6979341 | 55.11 | 2.9E−51 | Oryza sativa | 955 | YES |
| 23802651 | 1083737 | 60.75 | 3.8E−33 | Brassica napus | 956 | YES |
| 23802651 | 1603814 | 48.89 | 2.2E−30 | Parthenium argentatum | 957 | YES |
| 23803323 | 389639 | 100.00 | 0 | Zea mays | 958 | YES |
| 23513037 | 251685 | 92.20 | 4.1E−68 | Arabidopsis thaliana | 965 | NO |
| 23513037 | 11994638 | 89.10 | 1.3E−80 | Arabidopsis thaliana | 966 | NO |

### REFERENCES

(1) Zhang et al. (2004) Plant Physiol. 135:615.
(2) Salomon et al. (1984) EMBO J. 3:141.
(3) Herrera-Estrella et al. (1983) EMBO J. 2:987.
(4) Escudero et al. (1996) Plant J. 10:355.
(5) Ishida et al. (1996) Nature Biotechnology 14:745.
(6) May et al. (1995) Bio/Technology 13:486)
(7) Armaleo et al. (1990) Current Genetics 17:97.
(8) Smith. T. F. and Waterman, M. S. (1981) Adv. App. Math. 2:482.
(9) Needleman and Wunsch (1970) J. Mol. Biol. 48:443.
(10) Pearson and Lipman (1988) Proc. Natl. Acad. Sci. (USA) 85: 2444.
(11) Yamauchi et al. (1996) Plant Mol. Biol. 30:321-9.
(12) Xu et al. (1995) Plant Mol. Biol. 27:237.
(13) Yamamoto et al. (1991) Plant Cell 3:371.
(14) P. Tijessen, “Hybridization with Nucleic Acid Probes” In Laboratory Techniques in Biochemistry and Molecular Biology, P. C. vand der Vliet, ed., c. 1993 by Elsevier, Amsterdam.
(15) Bonner et al., (1973) J. Mol. Biol. 81:123.
(16) Sambrook et al., Molecular Cloning: A Laboratory Manual, Second Edition, Cold Spring Harbor Laboratory Press, 1989, New York.
(17) Shizuya et al. (1992) Proc. Natl. Acad. Sci. USA, 89: 8794-8797.
(18) Hamilton et al. (1996) Proc. Natl. Acad. Sci. USA, 93: 9975-9979.
(19) Burke et al. (1987) Science, 236:806-812.
(20) Sternberg N. et al. (1990) Proc Natl Acad Sci USA., 87:103-7.
(21) Bradshaw et al. (1995) Nucl Acids Res, 23: 4850-4856.
(22) Frischauf et al. (1983) J, Mol Biol, 170: 827-842.
(23) Huynh et al., Glover N M (ed) DNA Cloning: A practical Approach, Vol. 1 Oxford: IRL Press (1985).
(24) Walden et al. (1990) Mol Cell Biol 1: 175-194.
(25) Vissenberg et al. (2005) Plant Cell Physiol 46:192.
(26) Husebye et al. (2002) Plant Physiol 128:1180.
(27) Plesch et al. (2001) Plant J 28:455.
(28) Weising et al. (1988) Ann. Rev. Genet., 22:421.
(29) Christou (1995) Euphytica, v. 85, n.1-3:13-27.
(30) Newell (2000)
(31) Griesbach (1987) Plant Sci. 50:69-77.
(32) Fromm et al. (1985) Proc. Natl. Acad. Sci. USA 82:5824.
(33) Paszkowski et al. (1984) EMBO J. 3:2717.
(34) Klein et al. (1987) Nature 327:773.
(35) Willmitzer, L. (1993) Transgenic Plants. In: iotechnology, A Multi-Volume Comprehensive treatise (H. J. Rehm, G. Reed, A. Püler, P. Stadler, eds., Vol. 2, 627-659, VCH Weinheim-New York-Basel-Cambridge).
(36) Crit. Rev. Plant. Sci. 4:1-46.
(37) Fromm et al. (1990) Biotechnology 8:833-844.
(38) Cho et al. (2000) Planta 210:195-204.
(39) Brootghaerts et al. (2005) Nature 433:629-633.
(40) Lincoln et al. (1998) Plant Mol. Biol. Rep. 16:1-4.
(41) Lacomme et al. (2001), “Genetically Engineered Viruses” (C. J. A. Ring and E. D. Blair, Eds). Pp. 59-99, BIOS Scientific Publishers, Ltd. Oxford, UK.

What is claimed is:

1. An isolated nucleic acid molecule comprising:
(a) a nucleotide sequence encoding an amino acid sequence that is at least 85% identical to any one of the polypeptides in FIGS. 1-73;
(b) a nucleotide sequence that is complementary to any one of the nucleotide sequences according to paragraph (a);
(c) a nucleotide sequence according to any one of the nucleotide sequences in the Sequence Listing;
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(g) a nucleotide sequence encoding any one of the amino acid sequences corresponding to FIGS. 1-73.
(h) a nucleotide sequence encoding any one of the lead, functional homolog or consensus sequences in FIGS. 1-73.

2. A vector, comprising:
a) a first nucleic acid having a regulatory region encoding a plant transcription and/or translation signal; and
a second nucleic acid having a nucleotide sequence according to any one the nucleotide sequences of claim 1, wherein said first and second nucleic acids are operably linked.

3. A method of modulating plant growth and phenotype characteristics, said method comprising introducing into a plant cell an isolated nucleic acid comprising a nucleotide sequence selected from the group consisting of:
(a) a nucleotide sequence encoding an amino acid sequence that is at least 85% identical to any one of the polypeptides in FIGS. 1-73.
(b) a nucleotide sequence that is complementary to any one of the nucleotide sequences according to paragraph (a);
(c) a nucleotide sequence according to any one of the nucleotide sequences in the Sequence Listing
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(g) a nucleotide sequence encoding any one of the amino acid sequences in FIGS. 1-73; or
(h) a nucleotide sequence encoding any one of the lead, functional homolog or consensus sequences in FIGS. 1-73, wherein said plant produced from said plant cell has modulated plant growth and phenotype characteristics as compared to the corresponding level in tissue of a control plant that does not comprise said nucleic acid.

4. The method according to claim 3, wherein said consensus sequence comprises one or more of the conserved regions identified in any one of the alignment tables in FIGS. 1-73.

5. The method according to claim 4, wherein said consensus sequence comprises all of the conserved regions identified in any one of the alignment tables in FIGS. 1-73.

6. The method according to claim 5, wherein said consensus sequence comprises all of the conserved regions and in the order identified in any one of the alignment tables in FIGS. 1-73.

7. The method according to claim 6, wherein said conserved regions are separated by one or more amino acid residues.

8. The method according to claim 7, wherein said conserved regions are separated by one or more amino acids consisting in number and kind of the amino acids depicted in the alignment table for the lead and/or functional homolog sequences at the corresponding positions.

9. The method according to claim 8, wherein said consensus sequence has a length in terms of total number of amino acids that is equal to the length identified for a consensus sequence in one of FIGS. 1-73, or equal to a length ranging from the shortest to the longest sequence in any individual alignment table in any one of FIGS. 1-73.

10. The method of claim 3, wherein the modulated plant growth and phenotype characteristics comprise a modulation in plant size, vegetative growth (increased or decreased), organ number, biomass, sterility, seedling lethality, accelerated crop development or harvest, accelerated flowering time, delayed flowering time, delayed senescence, enhanced drought or stress tolerance, increased chlorophyll and photosynthetic capacity, increased anthocyanin content, increased root growth, increased nutrient uptake, increased seed weight, increased seed carbon or nitrogen content, increased seed/fruit yield, modified fruit content, enhanced foliage, making nutratceuticals/pharmaceuticals in plants, increase plant size, lethality, low fiber seeds with increased digestability, ornamental appearance with modified leaves, flowers, color or foliage, sterile plants, enhanced ability to grow in shade, enhanced biotic stress tolerance, increased tolerance to density and low fertilizer, enhanced tolerance to high or low pH, enhanced tolerance to low nitrogen or phosphate, enhanced tolerance to oxidative stress, enhanced chemical composition, altered leaf shape, enhanced abiotic stress tolerance, increased tolerance to cold stress, increased starch content, larger seeds, smaller seeds, fewer or no seeds, shorter plants, enhances plant strength, increased plant height, modified flower length, longer inflorescences, modified seed fiber content, modified fruit shape, modified fruit composition, modified seed yield, modified plant architecture, modified amount or angle of branching, modified leaf structure, modified seed structure or content, and enhanced shade avoidance as compared to the corresponding characteristic of a control plant that does not comprise said nucleic acid.

11. The method of claim 3, wherein said isolated nucleic acid is operably linked to a regulatory region.

12. The method of claim 11, wherein said regulatory region is a promoter selected from the group consisting of YP0092 (SEQ ID NO: **), PT0676 (SEQ ID NO: **), PT0708 (SEQ ID NO: **), PT0613 (SEQ ID NO: **), PT0672 (SEQ ID NO: **), PT0678 (SEQ ID NO: **), PT0688 (SEQ ID NO: **), PT0837 (SEQ ID NO: **), the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, the barley hordein gene promoter, p326 (SEQ ID NO: **), YP0144 (SEQ ID NO: **), YP0190 (SEQ ID NO: **), p13879 (SEQ ID NO: **), YP0050 (SEQ ID NO: **), p32449 (SEQ ID NO: **), 21876 (SEQ ID NO: **), YP0158 (SEQ ID NO: **), YP0214 (SEQ ID NO: **), YP0380 (SEQ ID NO: **), PT0848 (SEQ ID NO: **), and PTO633 (SEQ ID NO: **), the cauliflower mosaic virus (CaMV) ³⁵S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, ubiquitin promoters such as the maize ubiquitin-1 promoter, ribulose-1,5-bisphosphate carboxylase (RbcS) promoters such as the RbcS promoter from eastern larch (Larix laricina), the pine cab6 promoter, the Cab-1 gene promoter from wheat , the CAB-1 promoter from spinach, the cab1R promoter from rice, the pyruvate orthophosphate dikinase (PPDK) promoter from corn, the tobacco Lhcb1*2 promoter, the Arabidopsis thaliana SUC2 sucrose-H+ symporter promoter, and thylakoid membrane protein promoters from spinach (psaD, psaF, psaE, PC, FNR, atpC, atpD, cab, rbcS, PT0535 (SEQ ID NO:), PT0668 (SEQ ID NO:), PT0886 (SEQ ID NO:), PR0924 (SEQ ID NO:), YP0144 (SEQ ID NO:), YP0380 (SEQ ID NO:) and PT0585 (SEQ ID NO:),

13. A plant cell comprising an isolated nucleic acid comprising a nucleotide sequence selected from the group consisting of:
(a) a nucleotide sequence encoding an amino acid sequence that is at least 85% identical to any one of the polypeptides in FIGS. 1-73.
(b) a nucleotide sequence that is complementary to any one of the nucleotide sequences according to paragraph (a);
(c) a nucleotide sequence according to any one of the nucleotide sequences in the Sequence Listing;
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(g) a nucleotide sequence encoding any one of the amino acid sequences in FIGS. 1-73, or
(g) a nucleotide sequence encoding any one of the lead, functional homolog or consensus sequences in FIGS. 1-73.

14. A transgenic plant comprising the plant cell of claim 13.

15. Progeny of the plant of claim 14, wherein said progeny has modulated plant size, modulated vegetative growth, modulated plant architecture, modulated biomass, modulated sterility or modulated seedling lethality as compared to the corresponding level in tissue of a control plant that does not comprise said nucleic acid.

16. Seed from a transgenic plant according to claim 14.

17. Vegetative tissue from a transgenic plant according to claim 14.

18. A food product comprising vegetative tissue from a transgenic plant according to claim 14.

19. A feed product comprising vegetative tissue from a transgenic plant according to claim 14.

20. A method for detecting a nucleic acid in a sample, comprising:
providing an isolated nucleic acid according to claim 1;
contacting said isolated nucleic acid with a sample under conditions that permit a comparison of the nucleotide sequence of the isolated nucleic acid with a nucleotide sequence of nucleic acid in the sample; and
analyzing the comparison.

21. A method for promoting increased biomass in a plant, comprising:
(a) transforming a plant with a nucleic acid molecule comprising a nucleotide sequence encoding any one of the lead, functional homolog or consensus sequences in any one of FIGS. 1-73; and
(b) expressing said nucleotide sequence in said transformed plant, whereby said transformed plant has an increased biomass as compared to a plant that has not been transformed with said nucleotide sequence.

22. A method for modulating the biomass of a plant, said method comprising altering the level of expression in said plant of a nucleic acid molecule according to claim 1.
